(19) United States
(12) Patent Application Publication
(10) Pub. No.: US 2024/0092761 A1
(43) Pub. Date: Mar. 21, 2024

(54) QUINAZOLINE COMPOUNDS AND METHODS OF USE

(71) Applicant: IAMBIC THERAPEUTICS, INC., Los Angeles, CA (US)

(72) Inventors: William VERNIER, Vista, CA (US); Quynh Nhu NGUYEN, Redwood City, CA (US); Nomaan REZAYEE, San Diego, CA (US); Laurent GOMEZ, San Diego, CA (US); Chao Zhang, San Diego, CA (US); Thomas Francis MILLER, III, San Diego, CA (US); Frederick Roy MANBY, Bristol (GB)

(21) Appl. No.: 18/354,296

(22) Filed: Jul. 18, 2023

Related U.S. Application Data
(60) Provisional application No. 63/402,857, filed on Aug. 31, 2022.
(60) Provisional application No. 63/390,250, filed on Jul. 18, 2022.

(51) Int. Cl. C07D 401/14; C07D 471/10; C07D 487/10; C07D 495/10; C07D 491/107; C07D 413/14; A61P 35/04

(52) U.S. Cl. CPC C07D 401/14; C07D 471/10; C07D 487/10; C07D 495/10; C07D 491/107; C07D 413/14; A61P 35/04

(57) ABSTRACT

Substituted quinazoline compounds, conjugates, and pharmaceutical compositions for use in the treatment of cancer are disclosed herein. The disclosed compounds are useful, among other things, in the inhibition of CDK. In certain aspects, the disclosure generally relates to substituted quinolinone amide compounds or salts of Formula (I), (IA), (IAA), (IAAA), (IB), (IBB), (IC), (ICC), (II), or (IIA) and pharmaceutical compositions thereof.

### CROSS REFERENCE

This application claims the benefit of U.S. Provisional Application No. 63/402,857 filed on Aug. 31, 2022 and US Provisional Application No. 63/390,250 filed on Jul. 18, 2022, which are incorporated herein by reference in their entirety.

### BACKGROUND OF THE INVENTION

Division and proliferation of mammalian cells mediated by the cell cycle is an important and fundamental biological process, which controls production and generation of cells with critical biological functions. Cell cycle is a highly regulated process and responds to a complex set of cell signals within the cell and externally. The complex network of cell signaling, including components promoting and suppressing cancer, plays a key role controlling the cell cycle. Gain-of-function of tumor-promoting components or loss-of-function of tumor-suppressing products can lead to unregulated cell cycle and subsequently tumorigenesis.

Cyclins and cyclin-dependent kinases (CDKs) are crucial for driving and controlling cell cycle transitions and cell division (34176404). Cyclin is a family of proteins whose expression levels vary at different stages in the cell cycle. Cyclins bind and activate CDKs during different stages of cell cycle, of which the progression is tightly synchronized involving sequential activation of several cyclin-CDK complexes. Of more than 20 CDKs discovered so far, CDK1, 2, 4, 6 have been reported to play a direct role in cell cycle progression. CDK4-cyclin D and CDK6-cyclin D complexes are essential for entry in G1 phase of cell cycle. CDK2-cyclin E complex regulates progression from G1 into S phase, while CDK2-cyclin A is required during S phase. CDK1-cyclin A complex promotes entry into M phase, and mitosis is further regulated by CDK1-cyclin B complex. Progressive phosphorylation of retinoblastoma (Rb) by CDK4-cyclin D, CDK6-cyclin D and CDK2-cyclin E releases the GI transcription factor, E2F, and promotes S-phase entry. Activation of CDK2-cyclin A during early S-phase promotes phosphorylation of endogenous substrates that permit DNA replication and inactivation of E2F, for S-phase completion.

Dysregulation of cell-cycle machinery is a hallmark of cancer, leading to overactivation of CDKs and uncontrolled cell division and proliferation. Genetic alterations of the genes encoding cyclin D, CDK4/6, and CDK4/6-inhibiting proteins (such as p21, p27) all contribute to tumorigenesis. Cyclin E, the regulatory cyclin for CDK2, is frequently overexpressed in cancer. Since tumor development is closely related to gene mutation and deregulation of CDK and its regulators, CDK inhibitors are useful for anticancer therapy. CDK inhibitors have been developed as cancer therapy since the early 90s, with multiple FDA-approved drugs (Palbociclib, ribociclib and abemaciclib). However, these early generation CDK inhibitors on the market have poor selectivity and high toxicity (such as myelosuppression), leading to adverse effects limiting clinical dosing level for further patient benefit. There remains an unmet medical need to develop novel CDK inhibitors with better selectivity and less side effects for normal cells.

### SUMMARY OF THE INVENTION

The present disclosure generally relates to substituted quinazoline compounds or salts of Formula (I), (IA), (IAA), (IB), (IBB), (IC), (ICC), (II), or (IIA) and pharmaceutical compositions thereof. The substituted quinazoline compounds or salts of Formula (I), (IA), (IAA), (IB), (IBB), (IC), (ICC), (II), or (IIA) disclosed herein may be used for the treatment of abnormal cell growth, such as cancer, in a subject in need thereof.

In some aspects, methods of treating cancer may comprise administering a compound or pharmaceutically acceptable salt of any one of Formula (I), (IA), (IAA), (IB), (IBB), (IC), (ICC), (II), or (IIA) in an individual in need thereof.

In certain aspects, the disclosure provides a compound represented by Formula (I):

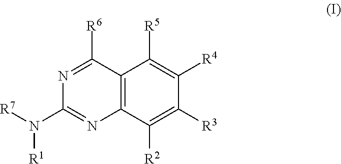

wherein,
R¹ is selected from optionally substituted pyrazole, optionally substituted indazole, optionally substituted tetrahydroisoquinoline, optionally substituted pyrrolpyrimidine, optionally substituted 2-pyridine, optionally substituted azabicyclo[3.1.0]hexane, optionally substituted indole, optionally substituted isoindole, and optionally substituted azetidine;
R² is selected from optionally substituted cycloalkyl and optionally substituted heterocycle;
each of R³, R⁴, R⁵, R⁶, is independently selected from hydrogen, halogen, —CN, optionally substituted C₁₋₄ alkyl, optionally substituted C₃₋₄ carbocycle, and optionally substituted 3- to 4-membered heterocycloalkyl;
R⁷ is selected from hydrogen and optionally substituted C₁₋₄ alkyl;
wherein if R¹ is an optionally substituted pyrazole, R² is not piperidine.

In certain aspects, the disclosure provides a pharmaceutical composition comprising a compound described herein and a pharmaceutically acceptable excipient.

In certain aspects, the disclosure provides a method of treating cancer comprising administering to a subject in need thereof a compound or pharmaceutical composition described herein. In certain aspects, the disclosure provides a method of inhibiting a cyclin dependent kinase (CDK) in a cell with a compound or pharmaceutically acceptable salt or the pharmaceutical composition described herein.

### DETAILED DESCRIPTION OF THE INVENTION

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

Basic functions for cell regulation, cell division, and cell proliferation are controlled by cyclin-dependent kinases (CDKs) activated by regulatory subunits such as cyclins. CDK inhibitors are useful in the treatment of cancer due to CDKs role in cell regulation. It has been shown that increased activity or transient abnormal activation of CDKs leads to the development of tumors; development of tumors are often associated with changes in the CDKs or regulators of CDKs.

CDKs bind to cyclin, which a regulatory protein, and without cyclin, it has little kinase activity. The cyclin-CDK complex is an active kinase typically modulated by phosphorylation and other binding proteins. There are currently 21 CDKs and 5 CDK-like genes that are known in the human genome. While many of the CDKs have been linked to transcription, CDK2, CDK4, and CDK6 are associated with the cell cycle. CDK2 is associated with DNA replication in higher eukaryotes whereas CDK4 and CDK6 are associated with various growth-regulatory signals.

CDK2 overexpression is associated with abnormal regulation of the cell cycle. Cyclin E, the cyclin partner of CDK2, binds to CDK2 to form an active kinase complex. The CDK2-cyclin E complex is important in the regulation of the G1/S transition, centrosome replication, and histone biosynthesis. Progressive phosphorylation can release the G1 transcription factor E2F and promote entry into the S phase. Another cyclin partner of CDK2, cyclin A, can bind and activate CDK2 during the initial phase of the S phase, and promote endogenous substrate phsophorlation, which allows DNA replication and E2F inactivation to complete the S phase.

CDK4 and CDK6 are also associated with the cell cycle. CDK4 and CDK6 inhibitors can arrest the cell cycle form the G1 to S phase by blocking phosphorylation of Rb protein and inhibiting proliferation of Rb-positive tumor cells. Besides cell cycle activity, CDK4 and CDK6 inhibitors can also suppress tumor growth through other mechanisms including, but not limited to inducing senescence, promoting anti-tumor immune responses, regulation of cell metabolism, and enhancing cytostasis caused by signaling pathway inhibitors.

### Definitions

Unless defined otherwise, all technical and scientific terms used herein have the same meaning as is commonly understood by one of skill in the art to which this invention belongs.

As used in the specification and claims, the singular form “a”, “an” and “the” includes plural references unless the context clearly dictates otherwise.

As used in the specification and appended claims, unless specified to the contrary, the following terms have the meaning indicated below.

“Amino” refers to the —NH₂ radical.

“Cyano” refers to the —CN radical.

“Nitro” refers to the —NO₂ radical.

“Oxa” refers to the —O— radical.

“Oxo” refers to the ═O radical.

“Thioxo” refers to the ═S radical.

“Imino” refers to the ═N—H radical.

“Oximo” refers to the ═N—OH radical.

“Hydrazino” refers to the ═N—NH₂ radical.

“Alkyl” refers to a straight or branched hydrocarbon chain radical consisting solely of carbon and hydrogen atoms, containing no unsaturation, having from one to fifteen carbon atoms (e.g., C₁-C₁₅ alkyl). In certain embodiments, an alkyl comprises one to thirteen carbon atoms (e.g., C₁-C₁₃ alkyl). In certain embodiments, an alkyl comprises one to eight carbon atoms (e.g., C₁-C₈ alkyl). In other embodiments, an alkyl comprises one to five carbon atoms (e.g., C₁-C₈ alkyl). In other embodiments, an alkyl comprises one to four carbon atoms (e.g., C₁-C₄ alkyl). In other embodiments, an alkyl comprises one to three carbon atoms (e.g., C₁-C₃ alkyl). In other embodiments, an alkyl comprises one to two carbon atoms (e.g., C₁-C₂ alkyl). In other embodiments, an alkyl comprises one carbon atom (e.g., C₁ alkyl). In other embodiments, an alkyl comprises five to fifteen carbon atoms (e.g., C₅-C₁₅ alkyl). In other embodiments, an alkyl comprises five to eight carbon atoms (e.g., C₅-C₈alkyl). In other embodiments, an alkyl comprises two to five carbon atoms (e.g., C₂-C₅ alkyl). In other embodiments, an alkyl comprises three to five carbon atoms (e.g., C₃-C₅ alkyl). In other embodiments, the alkyl group is selected from methyl, ethyl, 1-propyl (n-propyl), 1-methylethyl (iso-propyl), 1-butyl (n-butyl), 1-methylpropyl (sec-butyl), 2-methylpropyl (iso-butyl), 1,1-dimethylethyl (tert-butyl), 1-pentyl (n-pentyl). The alkyl is attached to the rest of the molecule by a single bond.

“Heteroalkyl” refers to an alkyl group, as defined above, having from one or more carbon atoms replaced with a heteroatom, such as wherein the heteroatom is individually selected from N, O and S at each replacement location. Additional heteroatoms can also be useful, including, but not limited to, B. Al, Si and P. The heteroatoms can also be oxidized, such as, but not limited to, —S(O)— and —S(O)₂—. For example, heteroalkyl can include ethers, thioethers and alkyl-amines. Hetoroalkyl consisting of the stated number of carbon atoms and may include one or more heteroatoms selected from the group consisting of O, N, Si and S, wherein the nitrogen heteroatom may optionally be quaternized. The heteroatom(s) O, N and S may be placed at any interior position of the heteroalkyl group. The heteroatom Si may be placed at any position of the heteroalkyl group, including the position at which the alkyl group is attached to the remainder of the molecule. Two heteroatoms may be consecutive, such as, for example, —CH₂NHOCH₃ and —CH₂OSi(CH₃)₃. Heteroalkyl can include any stated number of carbon atoms as defined herein and in the definition of alkyl.

“Alkoxy” refers to a radical bonded through an oxygen atom of the formula —O-alkyl, where alkyl is an alkyl chain as defined above.

“Alkenyl” refers to a straight or branched hydrocarbon chain radical group consisting solely of carbon and hydrogen atoms, containing at least one carbon-carbon double bond, and having from two to twelve carbon atoms. In certain embodiments, an alkenyl comprises two to eight carbon atoms. In other embodiments, an alkenyl comprises two to four carbon atoms. The alkenyl is attached to the rest of the molecule by a single bond, for example, ethenyl (i.e., vinyl), prop-1-enyl (i.e., allyl), but-1-enyl, pent-1-enyl, penta-1,4-dienyl, and the like.

“Alkynyl” refers to a straight or branched hydrocarbon chain radical group consisting solely of carbon and hydrogen atoms, containing at least one carbon-carbon triple bond, having from two to twelve carbon atoms. In certain embodiments, an alkynyl comprises two to eight carbon atoms. In other embodiments, an alkynyl comprises two to six carbon atoms. In other embodiments, an alkynyl comprises two to four carbon atoms. The alkynyl is attached to the rest of the molecule by a single bond, for example, ethynyl, propynyl, butynyl, pentynyl, hexynyl, and the like.

“Alkylene” or “alkylene chain” refers to a straight or branched divalent hydrocarbon chain linking the rest of the molecule to a radical group, consisting solely of carbon and hydrogen, containing no unsaturation and having from one to twelve carbon atoms, for example, methylene, ethylene, propylene, n-butylene, and the like. The alkylene chain is attached to the rest of the molecule through a single bond and to the radical group through a single bond. The points of attachment of the alkylene chain to the rest of the molecule and to the radical group are through one carbon in the alkylene chain or through any two carbons within the chain. In certain embodiments, an alkylene comprises one to eight carbon atoms (e.g., C₁-C₅ alkylene). In other embodiments, an alkylene comprises one to five carbon atoms (e.g., C₁-C₅ alkylene). In other embodiments, an alkylene comprises one to four carbon atoms (e.g., C₁-C₄ alkylene). In other embodiments, an alkylene comprises one to three carbon atoms (e.g., C₁-C₃ alkylene). In other embodiments, an alkylene comprises one to two carbon atoms (e.g., C₁-C₂ alkylene). In other embodiments, an alkylene comprises one carbon atom (e.g., C₁ alkylene). In other embodiments, an alkylene comprises five to eight carbon atoms (e.g., C₅-C₈alkylene). In other embodiments, an alkylene comprises two to five carbon atoms (e.g., C₂-C₅ alkylene). In other embodiments, an alkylene comprises three to five carbon atoms (e.g., C₃-C₅ alkylene).

“Alkenylene” or “alkenylene chain” refers to a straight or branched divalent hydrocarbon chain linking the rest of the molecule to a radical group, consisting solely of carbon and hydrogen, containing at least one carbon-carbon double bond, and having from two to twelve carbon atoms. The alkenylene chain is attached to the rest of the molecule through a single bond and to the radical group through a single bond. In certain embodiments, an alkenylene comprises two to eight carbon atoms (e.g., C₂-C₈ alkenylene). In other embodiments, an alkenylene comprises two to five carbon atoms (e.g., C₂-C₅ alkenylene). In other embodiments, an alkenylene comprises two to four carbon atoms (e.g., C₂-C₄ alkenylene). In other embodiments, an alkenylene comprises two to three carbon atoms (e.g., C₂-C₃ alkenylene). In other embodiments, an alkenylene comprises five to eight carbon atoms (e.g., C₅-C₈alkenylene). In other embodiments, an alkenylene comprises two to five carbon atoms (e.g., C₂-C₅ alkenylene). In other embodiments, an alkenylene comprises three to five carbon atoms (e.g., C₃-C₅ alkenylene).

“Alkynylene” or “alkynylene chain” refers to a straight or branched divalent hydrocarbon chain linking the rest of the molecule to a radical group, consisting solely of carbon and hydrogen, containing at least one carbon-carbon triple bond, and having from two to twelve carbon atoms. The alkynylene chain is attached to the rest of the molecule through a single bond and to the radical group through a single bond. In certain embodiments, an alkynylene comprises two to eight carbon atoms (e.g., C₂-C₈ alkynylene). In other embodiments, an alkynylene comprises two to five carbon atoms (e.g., C₂-C₅ alkynylene). In other embodiments, an alkynylene comprises two to four carbon atoms (e.g., C₂-C₄ alkynylene). In other embodiments, an alkynylene comprises two to three carbon atoms (e.g., C₂-C₃ alkynylene). In other embodiments, an alkynylene comprises two carbon atoms (e.g., C₂ alkylene). In other embodiments, an alkynylene comprises five to eight carbon atoms (e.g., C₅-C₈alkynylene). In other embodiments, an alkynylene comprises three to five carbon atoms (e.g., C₃-C₅ alkynylene).

“Heteroalkylene” refers to a straight or branched divalent heteroalkyl chain linking the rest of the molecule to a radical group, consisting of heteroatoms such as N, O and S. Additional heteroatoms can also be useful, including, but not limited to, B, Al, Si and P. The heteroalkylene chain is attached to the rest of the molecule through a single bond and to the radical group through a single bond. In certain embodiments, a heteroalkylene comprises one heteroatom. In certain embodiments, a heteroalkylene comprises two heteroatoms. In certain embodiments, a heteralkylene comprises three heteroatoms. In certain embodiments, a heteralkylene comprises four heteroatoms. In certain embodiments, a heteralkylene comprises five heteroatoms in certain embodiments, the heteroatoms can be N, O, S, Si, or P. or a combination thereof. In certain embodiments, the heteroatoms can be N, O, or S, or a combination thereof. In certain embodiments, the heteroatoms can be N, O, or a combination thereof.

The term “C_{x-y}” or “Cₓ-C_{y}” when used in conjunction with a chemical moiety, such as alkyl, alkenyl, or alkynyl is meant to include groups that contain from x to y carbons in the chain. For example, the term “C₁₋₆alkyl” refers to substituted or unsubstituted saturated hydrocarbon groups, including straight-chain alkyl and branched-chain alkyl groups that contain from 1 to 6 carbons.

The terms “C_{x-y}alkenyl” and “C_{x-y}alkynyl” refer to substituted or unsubstituted unsaturated aliphatic groups analogous in length and possible substitution to the alkyls described above, but that contain at least one double or triple bond, respectively.

The term “carbocycle” as used herein refers to a saturated, unsaturated or aromatic ring in which each atom of the ring is carbon. Carbocycle includes 3- to 10-membered monocyclic rings, 5- to 12-membered bicyclic rings, 5- to 12-membered spiro bicycles, and 5- to 12-membered bridged rings. Each ring of a bicyclic carbocycle may be selected from saturated, unsaturated, and aromatic rings. In an exemplary embodiment, an aromatic ring, e.g., phenyl, may be fused to a saturated or unsaturated ring, e.g., cyclohexane, cyclopentane, or cyclohexene. A bicyclic carbocycle includes any combination of saturated, unsaturated and aromatic bicyclic rings, as valence permits. A bicyclic carbocycle further includes spiro bicyclic rings such as spiropentane. A bicyclic carbocycle includes any combination of ring sizes such as 3-3 spiro ring systems, 4-4 spiro ring systems, 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Exemplary carbocycles include cyclopentyl, cyclohexyl, cyclohexenyl, adamantyl, phenyl, indanyl, naphthyl, and bicyclo[1.1.1]pentanyl.

The term “aryl” refers to an aromatic monocyclic or aromatic multicyclic hydrocarbon ring system. The aromatic monocyclic or aromatic multicyclic hydrocarbon ring system contains only hydrogen and carbon and from five to eighteen carbon atoms, where at least one of the rings in the ring system is aromatic, i.e., it contains a cyclic, delocalized (4n+2) π-electron system in accordance with the Hückel theory. The ring system from which aryl groups are derived include, but are not limited to, groups such as benzene, fluorene, indane, indene, tetralin and naphthalene.

The term “cycloalkyl” refers to a saturated ring in which each atom of the ring is carbon. Cycloalkyl may include monocyclic and polycyclic rings such as 3- to 10-membered monocyclic rings, 5- to 12-membered bicyclic rings, 5- to 12-membered spiro bicycles, and 5- to 12-membered bridged rings. In certain embodiments, a cycloalkyl comprises three to ten carbon atoms. In other embodiments, a cycloalkyl comprises five to seven carbon atoms. The cycloalkyl may be attached to the rest of the molecule by a single bond. Examples of monocyclic cycloalkyls include, e.g., cyclopropyl, cyclobutyl, cyclopentyl, cyclohexyl, cycloheptyl, and cyclooctyl. Polycyclic cycloalkyl radicals include, for example, adamantyl, spiropentane, norbornyl (i.e., bicyclo[2.2.1]heptanyl), decalinyl, 7,7 dimethyl bicyclo[2.2.1]heptanyl, bicyclo[1.1.1]pentanyl, and the like.

The term “cycloalkenyl” refers to a saturated ring in which each atom of the ring is carbon and there is at least one double bond between two ring carbons. Cycloalkenyl may include monocyclic and polycyclic rings such as 3- to 10-membered monocyclic rings, 6- to 12-membered bicyclic rings, and 5- to 12-membered bridged rings. In other embodiments, a cycloalkenyl comprises five to seven carbon atoms. The cycloalkenyl may be attached to the rest of the molecule by a single bond. Examples of monocyclic cycloalkenyls include, e.g., cyclopentenyl, cyclohexenyl, cycloheptenyl, and cyclooctenyl.

The term “halo” or, alternatively, “halogen” or “halide,” means fluoro, chloro, bromo or iodo. In some embodiments, halo is fluoro, chloro, or bromo.

The term “haloalkyl” refers to an alkyl radical, as defined above, that is substituted by one or more halo radicals, for example, trifluoromethyl, dichloromethyl, bromomethyl, 2,2,2-trifluoroethyl, 1-chloromethyl-2-fluoroethyl, and the like. In some embodiments, the alkyl part of the haloalkyl radical is optionally further substituted as described herein.

The term “heterocycle” as used herein refers to a saturated, unsaturated or aromatic ring comprising one or more heteroatoms. Exemplary heteroatoms include N, O, Si, P, B, and S atoms. Heterocycles include 3- to 10-membered monocyclic rings, 6- to 12-membered bicyclic rings, 5- to 12-membered spiro bicycles, and 5- to 12-membered bridged rings. A monocylic heterocycle includes any saturated, unsatured, and aromatic rings as valence permits. A monocyclice heterocycle includes but is not limited to, oxetane, azetidine, furan, tetrahydrofuran, pyrrole, pyrrolidine, pyran, piperidine, piperazine, imidazole, thiazole, morpholine, pyridine, and pyrimidine. A bicyclic heterocycle includes any combination of saturated, unsaturated and aromatic bicyclic rings, as valence permits. In an exemplary embodiment, an aromatic ring, e.g., pyridyl, may be fused to a saturated or unsaturated ring, e.g., cyclohexane, cyclopentane, morpholine, piperidine or cyclohexene. A bicyclic heterocycle includes any combination of ring sizes such as 4-5 fused ring systems, 5-5 fused ring systems, 5-6 fused ring systems, 6-6 fused ring systems, 5-7 fused ring systems, 6-7 fused ring systems, 5-8 fused ring systems, and 6-8 fused ring systems. Examples of fused ring systems include, but are not limited to, isoindoline, isoquinoline, tetrahydroisoquinoline, 3-azabicyclo[3.1.0]hexane and 6-oxa-3-azabicyclo[3.1.1]heptane. A bicyclic heterocycle further includes spiro bicyclic rings, e.g., 5 to 12-membered spiro bicycles, such as but not limited to 2-azaspiro[3.3]heptane, 5-azaspiro[2.4]heptane, 2-oxa-6-azaspiro[3.3]heptane, 2,6-diazaspiro[3.3]heptane, 1-thia-6-azaspiro[3.3]heptane, 6-azaspiro[3.4]octane, 2,6-diazaspiro[3.4]octane, 2-thia-6-azaspiro[3.4]octane, 2-thia-6-azaspiro[3.4]octane 2,2-dioxide, 4-oxa-7-azaspiro[2.5]octane, 2-azaspiro[4.4]nonane, 2,7-diazaspiro[4.4]nonane, 2-oxa-6-azaspiro[3.5]nonane, 7-oxa-2-azaspiro[3.5]nonane, 2-azaspiro[4.5]decane, 2,8-diazaspiro[4.5]decane, 8-oxa-2-azaspiro[4.5]decane, and 2-oxa-7-azaspiro[4.5]decane.

The term “heteroaryl” refers to a radical derived from a 5 to 18 membered aromatic ring radical that comprises two to seventeen carbon atoms and from one to six heteroatoms selected from nitrogen, oxygen and sulfur. As used herein, the heteroaryl radical is a monocyclic, bicyclic, tricyclic or tetracyclic ring system, wherein at least one of the rings in the ring system is aromatic, i.e., it contains a cyclic, delocalized (4n+2) π-electron system in accordance with the Hückel theory. Heteroaryl includes fused or bridged ring systems. The heteroatom(s) in the heteroaryl radical is optionally oxidized. One or more nitrogen atoms, if present, are optionally quaternized. The heteroaryl is attached to the rest of the molecule through any atom of the ring(s). Examples of heteroaryls include, but are not limited to, azepinyl, benzimidazolyl, 1,3-benzodioxolyl, benzofuranyl, benzoxazolyl, benzo[d]thiazolyl, benzodioxinyl, benzopyranyl, benzopyranonyl, benzofuranyl, benzofuranonyl, benzothienyl (benzothiophenyl), benzotriazolyl, furanyl, isothiazolyl, imidazolyl, indazolyl, indolyl, indazolyl, isoindolyl, indolinyl, isoindolinyl, pyrrolyl, pyrazolyl, pyridinyl, pyridopyrimidinyl, pyrazinyl, pyrimidinyl, pyridazinyl, pyrrolyl, quinazolinyl, quinolinyl, isoquinolinyl, tetrahydroquinolinyl, and thiophenyl (i.e. thienyl).

The term “heterocycloalkyl” refers to a saturated ring with carbon atoms and at least one heteroatom. Exemplary heteroatoms include N, O, Si, P, B, and S atoms. Heterocycloalkyl may include monocyclic and polycyclic rings such as 3- to 10-membered monocyclic rings, 6- to 12-membered bicyclic rings, 5- to 12-membered spiro bicycles, and 5- to 12-membered bridged rings. The heteroatoms in the heterocycloalkyl radical are optionally oxidized. One or more nitrogen atoms, if present, are optionally quaternized. The heterocycloalkyl is attached to the rest of the molecule through any atom of the heterocycloalkyl, valence permitting, such as any carbon or nitrogen atoms of the heterocycloalkyl. Examples of heterocycloalkyl radicals include, but are not limited to, azetidinyl, dioxolanyl, decahydroisoquinolyl, imidazolinyl, imidazolidinyl, isothiazolidinyl, isoxazolidinyl, morpholinyl, octahydroindolyl, octahydroisoindolyl, oxazolidinyl, oxetanyl, piperidinyl, piperazinyl, pyrrolidinyl, pyrazolidinyl, quinuclidinyl, thiazolidinyl, tetrahydrofuryl, trithianyl, tetrahydropyranyl, thiomorpholinyl, 3-azabicyclo[3.1.0]hexane, 2-azaspiro[3.3]heptane, 5-azaspiro[2.4]heptane, 2-oxa-6-azaspiro[3.3]heptane, 2,6-diazaspiro[3.3]heptane, 6-oxa-3-azabicyclo[3.1.1]heptane, 1-thia-6-azaspiro[3.3]heptane, 6-azaspiro[3.4]octane, 2,6-diazaspiro[3.4]octane, 2-thia-6-azaspiro[3.4]octane, 2-thia-6-azaspiro[3.4]octane 2,2-dioxide, 4-oxa-7-azaspiro[2.5]octane, 2-azaspiro[4.4]nonane, 2,7-diazaspiro[4.4]nonane, 2-oxa-6-azaspiro[3.5]nonane, 7-oxa-2-azaspiro[3.5]nonane, 2-azaspiro[4.5]decane, 2,8-diazaspiro[4.5]decane, 8-oxa-2-azaspiro[4.5]decane, 2-oxa-7-azaspiro[4.5]decane, and 1,1-dioxo-thiomorpholinyl.

The term “heterocycloalkenyl” refers to an unsaturated ring with carbon atoms and at least one heteroatom and there is at least one double bond between two ring carbons. Heterocycloalkenyl does not include heteroaryl rings. Exemplary heteroatoms include N, O, Si, P, B, and S atoms. Heterocycloalkenyl may include monocyclic and polycyclic rings such as 3- to 10-membered monocyclic rings, 6- to 12-membered bicyclic rings, and 5- to 12-membered bridged rings. In other embodiments, a heterocycloalkenyl comprises five to seven ring atoms. The heterocycloalkenyl may be attached to the rest of the molecule by a single bond. Examples of monocyclic cycloalkenyls include, e.g., pyrroline (dihydropyrrole), pyrazoline (dihydropyrazole), imidazoline (dihydroimidazole), triazoline (dihydrotriazole), dihydrofuran, dihydrothiophene, oxazoline (dihydrooxazole), 1lydrazine11ne (dihydroisoxazole), thiazoline (dihydrothiazole), isothiazoline (dihydroisothiazole), oxadiazoline (dihydrooxadiazole), thiadiazoline (dihydrothiadiazole), dihydropyridine, tetrahydropyridine, dihydropyridazine, tetrahydropyridazine, dihydropyrimidine, tetrahydropyrimidine, dihydropyrazine, tetrahydropyrazine, pyran, dihydropyran, thiopyran, dihydrothiopyran, dioxine, dihydrodioxine, oxazine, dihydrooxazine, thiazine, and dihydrothiazine.

The term “substituted” refers to moieties having substituents replacing a hydrogen on one or more carbons or substitutable heteroatoms, e.g., an NH or NH₂ of a compound. It will be understood that “substitution” or “substituted with” includes the implicit proviso that such substitution is in accordance with permitted valence of the substituted atom and the substituent, and that the substitution results in a stable compound, i.e., a compound which does not spontaneously undergo transformation such as by rearrangement, cyclization, elimination, etc. In certain embodiments, substituted refers to moieties having substituents replacing two hydrogen atoms on the same carbon atom, such as substituting the two hydrogen atoms on a single carbon with an oxo, imino or thioxo group. As used herein, the term “substituted” is contemplated to include all permissible substituents of organic compounds. In a broad aspect, the permissible substituents include acyclic and cyclic, branched and unbranched, carbocyclic and heterocyclic, spirocyclic and non-spirocyclic, aromatic and non-aromatic substituents of organic compounds. The permissible substituents can be one or more and the same or different for appropriate organic compounds.

In some embodiments, each substituent may individually include any substituents described herein, for example: halogen, hydroxy, oxo (═O), thioxo (═S), cyano (—CN), nitro (—NO₂), imino (═N—H), oximo (═N—OH), 11ydrazine (═N—NH₂), —R^{b}—OR^{a}, —R^{b}—OC(O)—R^{a}, —R—OC(O)—OR^{a}, —R^{b}—OC(O)—N(R^{a})₂, —R—N(R^{a})₂, —R—C(O)R^{a}, —R—C(O)OR^{a}, —R—C(O)N(R^{a})₂, —R^{b}—O—R^{c}—C(O)N(R^{a})₂, —R—N(R^{a})C(O)OR^{a}, —R—N(R^{a})C(O)R^{a}, —R—N(R^{a})S(O)ₜR^{a} (where t is 1 or 2), —R—S(O)ₜR^{a} (where t is 1 or 2), —R—S(O)ₜOR^{a} (where t is 1 or 2), and —R—S(O)ₜN(R^{a})₂ (where t is 1 or 2); and alkyl, alkenyl, alkynyl, aryl, aralkyl, aralkenyl, aralkynyl, cycloalkyl, cycloalkylalkyl, heterocycloalkyl, heterocycloalkylalkyl, heteroaryl, and heteroarylalkyl, any of which may be optionally substituted by alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), R^{a}, —R^{b}—OR^{a}, —R^{b}—OC(O)—R^{a}, —R^{b}—OC(O)—OR^{a}, —R^{b}—OC(O)—N(R^{a})₂, —R^{b}—N(R^{a})₂, —R^{b}—C(O)R^{a}, —R^{b}—C(O)OR^{a}, —R^{b}—C(O)N(R^{a})₂, —R^{b}—O—R^{c}—C(O)N(R^{a})₂, —R—N(R^{a})C(O)OR^{a}, —R—N(R^{a})C(O)R^{a}, —R—N(R^{a})S(O)ₜR^{a} (where t is 1 or 2), —R—S(O)ₜR^{a} (where t is 1 or 2), —R—S(O)ₜOR^{a} (where t is 1 or 2) and —R—S(O)ₜN(R^{a})₂ (where t is 1 or 2); wherein each R^{a} is independently selected from hydrogen, alkyl, cycloalkyl, cycloalkylalkyl, aryl, aralkyl, heterocycloalkyl, heterocycloalkylalkyl, heteroaryl, or heteroarylalkyl, wherein each R^{a}, valence permitting, may be optionally substituted with alkyl, alkenyl, alkynyl, halogen, haloalkyl, haloalkenyl, haloalkynyl, oxo (═O), thioxo (═S), cyano (—CN), nitro (—NO₂), imino (═N—H), oximo (═N—OH), hydrazine (═N—NH₂), —R—OR^{a}, —R—OC(O)—R^{a}, —R—OC(O)—OR^{a}, —R—OC(O)—N(R^{a})₂, —R—N(R^{a})₂, —R—C(O)R^{a}, —R—C(O)OR^{a}, —R—C(O)N(R^{a})₂, —R^{b}—O—R^{c}—C(O)N(R^{a})₂, —R—N(R^{a})C(O)OR^{a}, —R—N(R^{a})C(O)R^{a}, —R—N(R^{a})S(O)ₜR^{a} (where t is 1 or 2), —R^{b}—S(O)ₜR^{a} (where t is 1 or 2), —R^{b}—S(O)ₜOR^{a} (where t is 1 or 2) and —R^{b}—S(O)ₜN(R^{a})₂ (where t is 1 or 2); and wherein each R^{b} is independently selected from a direct bond or a straight or branched alkylene, alkenylene, or alkynylene chain, and each R^{c} is a straight or branched alkylene, alkenylene or alkynylene chain.

Double bonds to oxygen atoms, such as oxo groups, are represented herein as both “═O” and “(O)”. Double bonds to nitrogen atoms are represented as both “═NR” and “(NR)”. Double bonds to sulfur atoms are represented as both “═S” and “(S)”.

The phrases “parenteral administration” and “administered parenterally” as used herein means modes of administration other than enteral and topical administration, usually by injection, and includes, without limitation, intravenous, intramuscular, intra-arterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The phrase “pharmaceutically acceptable excipient” or “pharmaceutically acceptable carrier” as used herein means a pharmaceutically acceptable material, composition or vehicle, such as a liquid or solid filler, diluent, excipient, solvent or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose, and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

The term “salt” or “pharmaceutically acceptable salt” refers to salts derived from a variety of organic and inorganic counter ions well known in the art. Pharmaceutically acceptable acid addition salts can be formed with inorganic acids and organic acids. Inorganic acids from which salts can be derived include, for example, hydrochloric acid, hydrobromic acid, sulfuric acid, nitric acid, phosphoric acid, and the like. Organic acids from which salts can be derived include, for example, acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like. Pharmaceutically acceptable base addition salts can be formed with inorganic and organic bases. Inorganic bases from which salts can be derived include, for example, sodium, potassium, lithium, ammonium, calcium, magnesium, iron, zinc, copper, manganese, aluminum, and the like. Organic bases from which salts can be derived include, for example, primary, secondary, and tertiary amines, substituted amines including naturally occurring substituted amines, cyclic amines, basic ion exchange resins, and the like, specifically such as isopropylamine, trimethylamine, diethylamine, triethylamine, tripropylamine, and ethanolamine. In some embodiments, the pharmaceutically acceptable base addition salt is chosen from ammonium, potassium, sodium, calcium, and magnesium salts.

As used herein, “treatment” or “treating” refers to an approach for obtaining beneficial or desired results with respect to a disease, disorder, or medical condition including but not limited to a therapeutic benefit and/or a prophylactic benefit. A therapeutic benefit can include, for example, the eradication or amelioration of the underlying disorder being treated. Also, a therapeutic benefit can include, for example, the eradication or amelioration of one or more of the physiological symptoms associated with the underlying disorder such that an improvement is observed in the subject, notwithstanding that the subject may still be afflicted with the underlying disorder. In certain embodiments, for prophylactic benefit, the compositions are administered to a subject at risk of developing a particular disease, or to a subject reporting one or more of the physiological symptoms of a disease, even though a diagnosis of this disease may not have been made. Treatment via administration of a compound described herein does not require the involvement of a medical professional.

Table of Abbreviations

| | |
|---|---|
| ACN, MeCN | Acetonitrile |
| ° C. | Degree Celsius |
| BOC | tert-butoxycarbonyl |
| Cmpd | Compound |
| DCM | Dichloromethane |
| DIEA or DIPEA | Diisopropylethylamine |
| DMF | Dimethylformamide |
| DMSO | Dimethylsulfoxide |
| EA, ETOAc, ETAC | Ethyl acetate |
| EDC, EDAC or EDCI | 1-Ethyl-3-(3-dimethylaminopropyl)carbodiimide |
| ESI | Electrospray ionization |
| HATU | Hexafluorophosphate Azabenzotriazole Tetramethyl |
| | Uronium |
| HOBt | Hydroxybenzotriazole |
| HPLC | High Performance Liquid Chromatography |
| IC₅₀ | Half minimal (50%) inhibitory concentration |
| LCMS | Liquid Chromatography Mass Spectrometry |
| [M+H+]+ or (MH)+ | Mass peak plus hydrogen |
| [M−H−]− or (MH)− | Mass peak minus hydrogen |
| Me | Methyl |
| MeOH | Methanol |
| MS | Mass spectrometry |
| N | Normal |
| PE | Petroleum ether |
| PMB | (4-methoxyphenyl)methanamine or para-methoxy |
| | benzyl |
| Prep-TLC | Preparative thin layer chromatography |
| PyBroP | bromotri(pyrrolidino)phosphonium |
| | hexafluorophosphate |
| RP | Reverse phase |
| RP-HPLC | Reverse phase high pressure liquid chromatography |
| RT or rt | Room temperature |
| TLC | Thin-layer chromatography |
| THF | Tetrahydrofuran |
| TFA | Trifluoroacetic acid |

### Compounds

The following is a discussion of compounds and salts thereof that may be used in the methods of the disclosure. In certain embodiments, the compounds and salts are described in Formula (I0), (I), (IA), (IAA), (IB), (IBB), (IC), (ICC), (II), or (IIA).

In one aspect, disclosed herein is a compound represented by Formula (I):

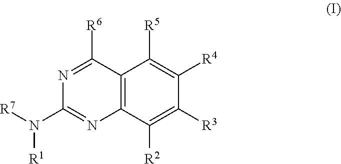

wherein,
R¹ is selected from

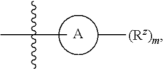

optionally substituted piperidine, optionally substituted phenyl, optionally substituted pyrazole, optionally substituted indazole, optionally substituted tetrahydroisoquinoline, optionally substituted pyrrolpyrimidine, optionally substituted 2-pyridine, optionally substituted azabicyclo[3.1.0]hexane, optionally substituted indole, optionally substituted isoindole, and optionally substituted azetidine;
A is a ring selected from optionally substituted C₃₋₆ carbocycle and optionally substituted 3- to 12-membered heterocycle;
R² is selected from optionally substituted cycloalkyl and optionally substituted heterocycle;
each of R³, R⁴, R⁵, R⁶, is independently selected from hydrogen, halogen, —CN, optionally substituted C₁₋₄ alkyl, optionally substituted C₃₋₄ carbocycle, and optionally substituted 3- to 4-membered heterocycloalkyl;
R⁷ is selected from hydrogen and optionally substituted C₁₋₄ alkyl
R^{z} is selected from halogen, —CN, —NO₂, optionally substituted alkyl, optionally substituted alkenyl, optionally substituted alkynyl, optionally substituted carbocycle, and optionally substituted heterocycle; and
m is selected from 0 to 9,
wherein if R¹ is an optionally substituted pyrazole, R² is not piperidine.

In one aspect, disclosed herein is a compound represented by Formula (I):

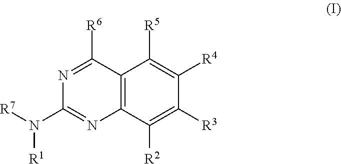

wherein,
R¹ is selected from optionally substituted indazole, optionally substituted tetrahydroisoquinoline, optionally substituted pyrrolpyrimidine, optionally substituted 2-pyridine, optionally substituted azabicyclo[3.1.0]hexane, optionally substituted indole, optionally substituted isoindole, and optionally substituted azetidine;
R² is selected from optionally substituted cycloalkyl and optionally substituted heterocycle;
each of R³, R⁴, R⁵, R⁶, is independently selected from hydrogen, halogen, —CN, optionally substituted C₁₋₄ alkyl, optionally substituted C₃₋₄ carbocycle, and optionally substituted 3- to 4-membered heterocycloalkyl;
R⁷ is selected from hydrogen and optionally substituted C₁₋₄ alkyl.

In one aspect, disclosed herein is a compound represented by Formula (I):

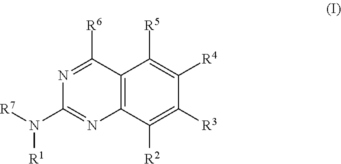

wherein,
R¹ is selected from optionally substituted piperidine, optionally substituted 2-pyridine, optionally substituted azabicyclo[3.1.0]hexane, optionally substituted indole, optionally substituted isoindole, and optionally substituted azetidine;
R² is selected from optionally substituted cycloalkyl and optionally substituted heterocycle;
each of R³, R⁴, R⁵, R⁶, is independently selected from hydrogen, halogen, —CN, optionally substituted C₁₋₄ alkyl, optionally substituted C₃₋₄ carbocycle, and optionally substituted 3- to 4-membered heterocycloalkyl; and
R⁷ is selected from hydrogen and optionally substituted C₁₋₄ alkyl.

R¹ can be any suitable functional group known by one of skill in the art. In some embodiments, R¹ is selected from optionally substituted piperidine, optionally substituted pyridine, optionally substituted azabicyclo[3.1.0]hexane, optionally substituted indole, optionally substituted isoindole, and optionally substituted azetidine. In some embodiments, R¹ is selected from optionally substituted piperidine, optionally substituted 2-pyridine, optionally substituted azabicyclo[3.1.0]hexane, optionally substituted indole, optionally substituted isoindole, and optionally substituted azetidine. In some embodiments, R¹ is selected from optionally substituted piperidine, optionally substituted pyridine, optionally substituted azetidine, optionally substituted azabicyclo[3.1.0]hexane, optionally substituted isoindole, and optionally substituted indole. In some embodiments, R¹ is selected from optionally substituted azabicyclo[3.1.0]hexane, optionally substituted isoindole, and optionally substituted indole. In some embodiments, R¹ is selected from optionally substituted azabicyclo[3.1.0]hexane, and optionally substituted isoindole. In some embodiments, R¹ is substituted with —SO₂R^{1a} or C₁₋₃ alkyl, wherein R^{1a} is selected from C₁₋₆ alkyl.

In some embodiments, R¹ is selected from optionally substituted piperidine, optionally substituted phenyl, optionally substituted pyrazole, optionally substituted indazole, optionally substituted tetrahydroisoquinoline, optionally substituted pyrrolpyrimidine, optionally substituted tetrahydroisoquinoline, optionally substituted 2-pyridine, optionally substituted azabicyclo[3.1.0]hexane, optionally substituted indole, optionally substituted isoindole, and optionally substituted azetidine. In some embodiments, R¹ is selected from optionally substituted indazole, optionally substituted tetrahydroisoquinoline, optionally substituted pyrrolpyrimidine, optionally substituted tetrahydroisoquinoline, optionally substituted 2-pyridine, optionally substituted azabicyclo[3.1.0]hexane, optionally substituted indole, optionally substituted isoindole, and optionally substituted azetidine.

In some embodiments, R is

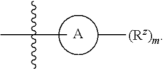

In some embodiments, A is a ring selected from optionally substituted C₃₋₆ carbocycle and optionally substituted 3- to 12-membered heterocycle. In some embodiments, A is selected from optionally substituted C₅₋₆ carbocycle and optionally substituted 5- to 10-membered heterocycle. In some embodiments, A is selected from phenyl, pyrazole, and tetrahydroisoquinoline. In some embodiments, m is selected from 0 to 4. In some embodiments, m is selected from 0 to 2. In some embodiments, R^{z} is selected from halogen, —CN, optionally substituted alkyl, optionally substituted carbocycle, and optionally substituted heterocycle. In some embodiments, R^{z} is selected from optionally substituted C₁₋₃ alkyl, and optionally substituted 5- to 8-membered heterocycle. In some embodiments, R^{z} is selected from C₁₋₃ alkyl, and substituted 5- to 8-membered heterocycle.

In some embodiments, R¹ is selected from optionally substituted pyrazole, optionally substituted indazole, optionally substituted tetrahydroisoquinoline, optionally substituted pyrrolpyrimidine, optionally substituted 2-pyridine, optionally substituted azabicyclo[3.1.0]hexane, optionally substituted indole, optionally substituted isoindole, and optionally substituted azetidine. In some embodiments, R¹ is selected from optionally substituted piperidine, optionally substituted indazole, optionally substituted tetrahydroisoquinoline, optionally substituted pyrrolpyrimidine, optionally substituted tetrahydroisoquinoline, optionally substituted 2-pyridine, optionally substituted azabicyclo[3.1.0]hexane, optionally substituted indole, optionally substituted isoindole, and optionally substituted azetidine. In some embodiments, R¹ is selected from optionally substituted piperidine, optionally substituted 2-pyridine, optionally substituted azabicyclo[3.1.0]hexane, optionally substituted indole, optionally substituted isoindole, and optionally substituted azetidine. In some embodiments, R¹ is optionally substituted piperidine. In some embodiments, R¹ is optionally substituted azabicyclo[3.1.0]hexane. In some embodiments, R¹ is optionally substituted indole. In some embodiments, R¹ is optionally substituted isoindole. In some embodiments, R¹ is optionally substituted azetidine. In some embodiments, R¹ is optionally substituted indazole. In some embodiments, R¹ is optionally substituted tetrahydroisoquinoline.

R² can be any suitable functional group known by one of skill in the art. In some embodiments, R² is selected from optionally substituted cycloalkyl and optionally substituted heterocycle. In some embodiments, R² is selected from optionally substituted C₃₋₆ cycloalkyl, optionally substituted pyrrolidine, optionally substituted piperidine, optionally substituted pyrazole, optionally substituted azetidine, optionally substituted oxetane, and optionally substituted morpholine.

In some embodiments, R² is selected from optionally substituted cycloalkyl and optionally substituted heterocycle. In some embodiments, R² is selected from optionally substituted cycloalkyl. In some embodiments, R² is selected from cycloalkyl. In some embodiments, R² is selected from optionally substituted C₃₋₆ cycloalkyl. In some embodiments, R² is selected from optionally substituted C₅₋₆ cycloalkyl. In some embodiments, R² is selected from optionally substituted heterocycloalkyl. In some embodiments, R² is selected from heterocycloalkyl. In some embodiments, R² is selected from optionally substituted 3- to 10-membered heterocycloalkyl. In some embodiments, R² is selected from optionally substituted 3- to 8-membered heterocycloalkyl. In some embodiments, R² is selected from optionally substituted 3- to 7-membered heterocycloalkyl. In some embodiments, R² is selected from optionally substituted 3- to 6-membered heterocycloalkyl. In some embodiments, R² is selected from optionally substituted azetidine, optionally substituted pyrrolidine, optionally substituted piperidine, optionally substituted piperazine, optionally substituted morpholine, optionally substituted 2-azaspiro[3.3]heptane, optionally substituted 5-azaspiro[2.4]heptane, optionally substituted 2-oxa-6-azaspiro[3.3]heptane, optionally substituted 2,6-diazaspiro[3.3]heptane, optionally substituted 1-thia-6-azaspiro[3.3]heptane, optionally substituted 6-azaspiro[3.4]octane, optionally substituted 2,6-diazaspiro[3.4]octane, optionally substituted 2-thia-6-azaspiro[3.4]octane, optionally substituted 2-thia-6-azaspiro[3.4]octane 2,2-dioxide, optionally substituted 4-oxa-7-azaspiro[2.5]octane, optionally substituted 2-azaspiro[4.4]nonane, optionally substituted 2,7-diazaspiro[4.4]nonane, optionally substituted 2-oxa-6-azaspiro[3.5]nonane, optionally substituted 7-oxa-2-azaspiro[3.5]nonane, optionally substituted 2-azaspiro[4.5]decane, optionally substituted 2,8-diazaspiro[4.5]decane, optionally substituted 8-oxa-2-azaspiro[4.5]decane, and optionally substituted 2-oxa-7-azaspiro[4.5]decane. In some embodiments, R² is 6-oxa-3-azabicyclo[3.1.1]heptane.

In some embodiments, R² is substituted with halogen, —SO₂R^{2a}, —NR^{2a}, oxo, —COR^{2a}, C₁₋₄ alkyl, C₁₋₃alkylene-C₁₋₃alkoxy, —OR^{2a}, —CN, —CH₂—CN, and an optionally substituted 3- to 6-membered heterocycloalkyl, wherein R is selected from hydrogen and C₁₋₆ alkyl. In some embodiments, R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, piperidine, piperazine, or azetidine, wherein R^{2a} is selected from C₁₋₆ alkyl. In some embodiments, R² is substituted with halogen, —SO₂R^{2a}, oxo, and C₁₋₄ alkyl, wherein R^{2a} is selected from C₁₋₆ alkyl. In some embodiments, R² is substituted with halogen, —SO₂R^{2a}, and —NR^{2a}, oxo, and C₁₋₃ alkyl, wherein R² is selected from C₁₋₃ alkyl. In some embodiments, R² is substituted with fluoro, —SO₂Me, oxo, acetyl, methyl, ethyl, propyl, cyclopropyl, —CH₂-cyclopropyl, —CH₂OH, —(CH₂)₂OMe, —(CH₂)₂OEt, —OH, —OMe, —OEt, —CN, —C—CN, oxetane, and azetidine. In some embodiments, R² is substituted with fluoro, —SO₂Me, oxo, acetyl, methyl, ethyl, cyclopropyl, —CH₂-cyclopropyl, —CH₂OH, —(CH₂)₂OMe, —OH, —OMe, —CN, —C—CN, and oxetane. In some embodiments, R² is substituted with fluoro, —SO₂Me, oxo, and methyl. In some embodiments, R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, methyl piperidine, or azetidine, wherein R^{2a} is selected from C₁₋₆ alkyl.

In some embodiments, R² is

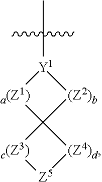

Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle; and
each R¹¹ is independently selected from hydrogen and optionally substituted C₁₋₄ alkyl.

In some embodiments, R² is selected from:

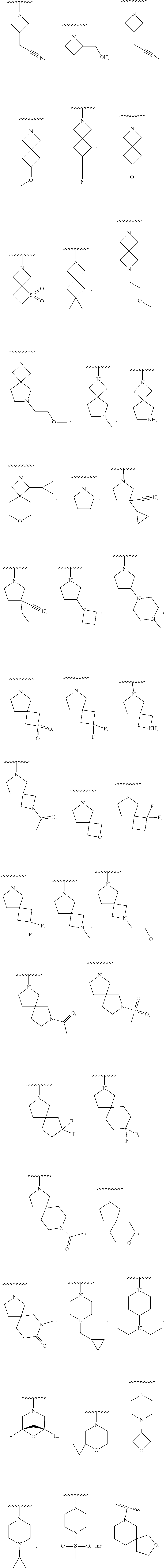

In some embodiments, R² is selected from optionally substituted cycloalkyl. In some embodiments, R² is selected from optionally substituted cyclopentane. In some embodiments, R² is selected fro and

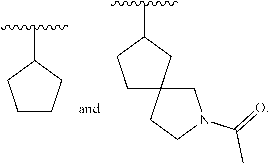 [SMILES: *C1CCCC1 ; *C1CCC2(CCN(C(C)=O)C2)C1]

R³ can be any suitable functional group known by one of skill in the art. In some embodiments, selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R³ is selected from hydrogen, halogen, —CN, optionally substituted C₃₋₄ carbocycle, and optionally substituted 3- to 4 membered heterocycloalkyl. In some embodiments, R³ is selected from hydrogen, fluoro, chloro, bromo, —CN, methyl, ethyl, propyl, butyl, cyclopropyl, cyclobutyl, optionally substituted oxetane, and optionally substituted azetidine. In some embodiments, R³ is selected from hydrogen, fluoro, —CN, cyclopropyl, cyclobutyl, optionally substituted oxetane, and optionally substituted azetidine. In some embodiments, R³ is selected from hydrogen, fluoro, and —CN. In some embodiments, R³ is selected from hydrogen and —CN.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁴ is selected from hydrogen, —CN, optionally substituted methyl, optionally substituted ethyl, optionally substituted propyl, optionally substituted cyclopropyl, optionally substituted cyclobutyl, optionally substituted oxetane, and optionally substituted azetidine. In some embodiments, R⁴ is selected from hydrogen, —CN, —CHF₂, cyclopropyl, cyclobutyl, optionally substituted oxetane, and optionally substituted azetidine. R⁴ is selected from hydrogen, —CN, and —CHF₂.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁵ is selected from hydrogen, halogen, —CN, optionally substituted C₃₋₄ carbocycle, and optionally substituted 3- to 4 membered heterocycloalkyl. In some embodiments, R⁵ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁵ is selected from hydrogen, fluoro, —CN, cyclopropyl, cyclobutyl, optionally substituted oxetane, and optionally substituted azetidine. In some embodiments, R⁵ is selected from hydrogen, methyl, ethyl, and propyl. In some embodiments, R⁵ is hydrogen.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁶ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁶ is selected from hydrogen, halogen, —CN, optionally substituted C₃₋₄ carbocycle, and optionally substituted 3- to 4 membered heterocycloalkyl. In some embodiments, R⁶ is selected from hydrogen, fluoro, —CN, cyclopropyl, cyclobutyl, optionally substituted oxetane, and optionally substituted azetidine. In some embodiments, R⁶ is selected from hydrogen, methyl, ethyl, and propyl. In some embodiments, R⁶ is hydrogen.

R⁷ can be any suitable functional group known by one of skill in the art. In some embodiments, R⁷ is selected from hydrogen, and optionally substituted C₁₋₄ alkyl. In some embodiments, R⁷ is selected from hydrogen, methyl, ethyl, and propyl. In some embodiments, R⁷ is hydrogen.

In some embodiments, the compound or pharmaceutically acceptable salt or solvate thereof, has the structure of Formula (IA):

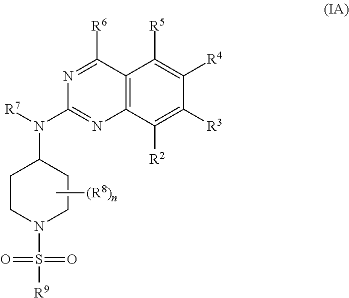

wherein,
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl; and
n is selected from 0 to 9.

In some embodiments, R² is selected from optionally substituted cycloalkyl and optionally substituted heterocycle. In some embodiments, R² is selected from optionally substituted cycloalkyl. In some embodiments, R² is selected from cycloalkyl. In some embodiments, R² is selected from optionally substituted C₃₋₆ cycloalkyl. In some embodiments, R² is selected from optionally substituted C₅₋₆ cycloalkyl. In some embodiments, R² is selected from optionally substituted heterocycloalkyl. In some embodiments, R² is selected from heterocycloalkyl. In some embodiments, R² is selected from optionally substituted 3- to 10-membered heterocycloalkyl. In some embodiments, R² is selected from optionally substituted 3- to 8-membered heterocycloalkyl. In some embodiments, R² is selected from optionally substituted 3- to 7-membered heterocycloalkyl. In some embodiments, R² is selected from optionally substituted 3- to 6-membered heterocycloalkyl. In some embodiments, R² is selected from optionally substituted azetidine, optionally substituted pyrrolidine, optionally substituted piperidine, optionally substituted piperazine, optionally substituted morpholine, optionally substituted 2-azaspiro[3.3]heptane, optionally substituted 5-azaspiro[2.4]heptane, optionally substituted 2-oxa-6-azaspiro[3.3]heptane, optionally substituted 2,6-diazaspiro[3.3]heptane, optionally substituted 1-thia-6-azaspiro[3.3]heptane, optionally substituted 6-azaspiro[3.4]octane, optionally substituted 2,6-diazaspiro[3.4]octane, optionally substituted 2-thia-6-azaspiro[3.4]octane, optionally substituted 2-thia-6-azaspiro[3.4]octane 2,2-dioxide, optionally substituted 4-oxa-7-azaspiro[2.5]octane, optionally substituted 2-azaspiro[4.4]nonane, optionally substituted 2,7-diazaspiro[4.4]nonane, optionally substituted 2-oxa-6-azaspiro[3.5]nonane, optionally substituted 7-oxa-2-azaspiro[3.5]nonane, optionally substituted 2-azaspiro[4.5]decane, optionally substituted 2,8-diazaspiro[4.5]decane, optionally substituted 8-oxa-2-azaspiro[4.5]decane, and optionally substituted 2-oxa-7-azaspiro[4.5]decane. In some embodiments, R² is 6-oxa-3-azabicyclo[3.1.1]heptane.

In some embodiments, R² is substituted with halogen, —SO₂R^{2a}, —NR^{2a}, oxo, —COR^{2a}, C₁₋₄ alkyl, C₁₋₃alkylene-C₁₋₃alkoxy, —OR^{2a}, —CN, —CH₂—CN, and an optionally substituted 3- to 6-membered heterocycloalkyl, wherein R^{2a} is selected from hydrogen and C₁₋₆ alkyl. In some embodiments, R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, piperidine, piperazine, or azetidine, wherein R² is selected from C₁₋₆ alkyl. In some embodiments, R² is substituted with halogen, —SO₂R^{2a}, oxo, and C₁₋₄ alkyl, wherein R² is selected from C₁₋₆ alkyl. In some embodiments, R² is substituted with halogen, —SO₂R^{2a}, and —NR^{2a}, oxo, and C₁₋₃ alkyl, wherein R is selected from C₁₋₆ alkyl. In some embodiments, R² is substituted with fluoro, —SO₂Me, oxo, acetyl, methyl, ethyl, propyl, cyclopropyl, —CH₂-cyclopropyl, —CH₂OH, —(CH₂)₂OMe, —(CH₂)₂OEt, —OH, —OMe, —OEt, —CN, —C—CN, oxetane, and azetidine. In some embodiments, R² is substituted with fluoro, —SO₂Me, oxo, acetyl, methyl, ethyl, cyclopropyl, —CH₂-cyclopropyl, —CH₂OH, —(CH₂)₂OMe, —OH, —OMe, —CN, —C—CN, and oxetane. In some embodiments, R² is substituted with fluoro, —SO₂Me, oxo, and methyl. In some embodiments, R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, methyl piperidine, or azetidine, wherein R^{2a} is selected from C₁₋₆ alkyl.

In some embodiments, R² is selected from optionally substituted C₃₋₆ cycloalkyl, optionally substituted pyrrolidine, optionally substituted piperidine, optionally substituted pyrazole, optionally substituted azetidine, optionally substituted oxetane, and optionally substituted morpholine. In some embodiments, R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, or azetidine, wherein R^{2a} is selected from C₁₋₆ alkyl. In some embodiments, R² is 6-oxa-3-azabicyclo[3.1.1]heptane.

In some embodiments, R² is selected from:

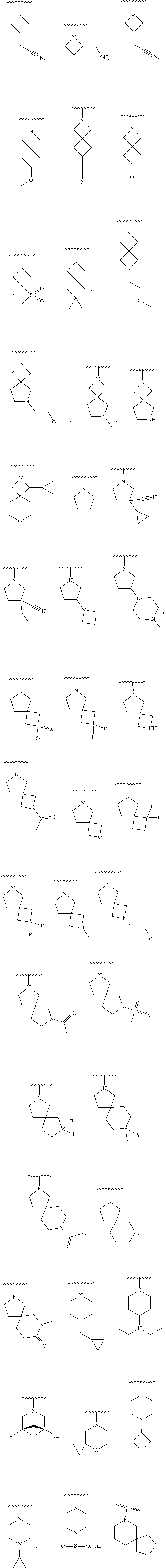

In some embodiments, R² is selected from optionally substituted cycloalkyl. In some embodiments, R² is selected from optionally substituted cyclopentane. In some embodiments, R² is selected from

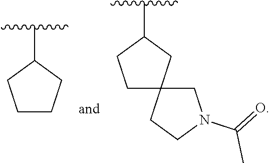 [SMILES: *C1CCCC1 ; *C1CCC2(CCN(C(C)=O)C2)C1]

R³ can be any suitable functional group known by one of skill in the art. In some embodiments, selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R³ is selected from hydrogen, halogen, —CN, optionally substituted C₃₋₄ carbocycle, and optionally substituted 3- to 4 membered heterocycloalkyl. In some embodiments, R³ is selected from hydrogen, fluoro, chloro, bromo, —CN, methyl, ethyl, propyl, butyl, cyclopropyl, cyclobutyl, optionally substituted oxetane, and optionally substituted azetidine. In some embodiments, R³ is selected from hydrogen, fluoro, —CN, cyclopropyl, cyclobutyl, optionally substituted oxetane, and optionally substituted azetidine. In some embodiments, R³ is selected from hydrogen, fluoro, and —CN. In some embodiments, R³ is selected from hydrogen and —CN.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁴ is selected from hydrogen, —CN, optionally substituted methyl, optionally substituted ethyl, optionally substituted propyl, optionally substituted cyclopropyl, optionally substituted cyclobutyl, optionally substituted oxetane, and optionally substituted azetidine. In some embodiments, R⁴ is selected from hydrogen, —CN, —CHF₂, cyclopropyl, cyclobutyl, optionally substituted oxetane, and optionally substituted azetidine. R⁴ is selected from hydrogen, —CN, and —CHF₂.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁵ is selected from hydrogen, halogen, —CN, optionally substituted C₃₋₄ carbocycle, and optionally substituted 3- to 4 membered heterocycloalkyl. In some embodiments, R⁵ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁵ is selected from hydrogen, fluoro, —CN, cyclopropyl, cyclobutyl, optionally substituted oxetane, and optionally substituted azetidine. In some embodiments, R⁵ is selected from hydrogen, methyl, ethyl, and propyl. In some embodiments, R⁵ is hydrogen.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁶ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁶ is selected from hydrogen, halogen, —CN, optionally substituted C₃₋₄ carbocycle, and optionally substituted 3- to 4 membered heterocycloalkyl. In some embodiments, R⁶ is selected from hydrogen, fluoro, —CN, cyclopropyl, cyclobutyl, optionally substituted oxetane, and optionally substituted azetidine. In some embodiments, R⁶ is selected from hydrogen, methyl, ethyl, and propyl. In some embodiments, R⁶ is hydrogen.

R⁷ can be any suitable functional group known by one of skill in the art. In some embodiments, R⁷ is selected from hydrogen, and optionally substituted C₁₋₄ alkyl. In some embodiments, R⁷ is selected from hydrogen, methyl, ethyl, and propyl. In some embodiments, R⁷ is hydrogen.

Variable n can be any suitable number known by one of skill in the art. In some embodiments, n is 0 to 9. In some embodiments, n is 0 to 5. In some embodiments, n is 0 to 3. In some embodiments, n is 0 or 1. In some embodiments n is 0. In some embodiments, n is 1.

R⁸ can be any suitable functional group known by one of skill in the art. In some embodiments, R⁸ is selected from halogen, —CN, and optionally substituted C₁₋₄ alkyl. In some embodiments, R⁸ is selected from halogen and optionally substituted C₁₋₄ alkyl. In some embodiments, R⁸ is fluoro, chloro, bromo, methyl, ethyl, or propyl.

R⁹ can be any suitable functional group known by one of skill in the art. In some embodiments, R⁹ is selected from optionally substituted C₁₋₄ alkyl, optionally substituted C₃₋₆ carbocycle, and 3- to 6-membered heterocycloalkyl. In some embodiments, R⁹ is selected from optionally substituted C₁₋₄ alkyl. In some embodiments, R⁹ is selected from methyl, ethyl, and propyl.

In some embodiments, the compound is selected from:

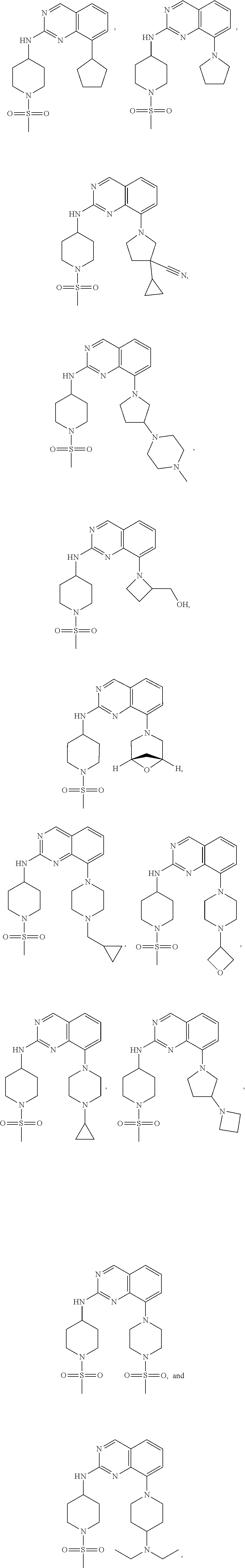

In some embodiments, the compound is selected from:

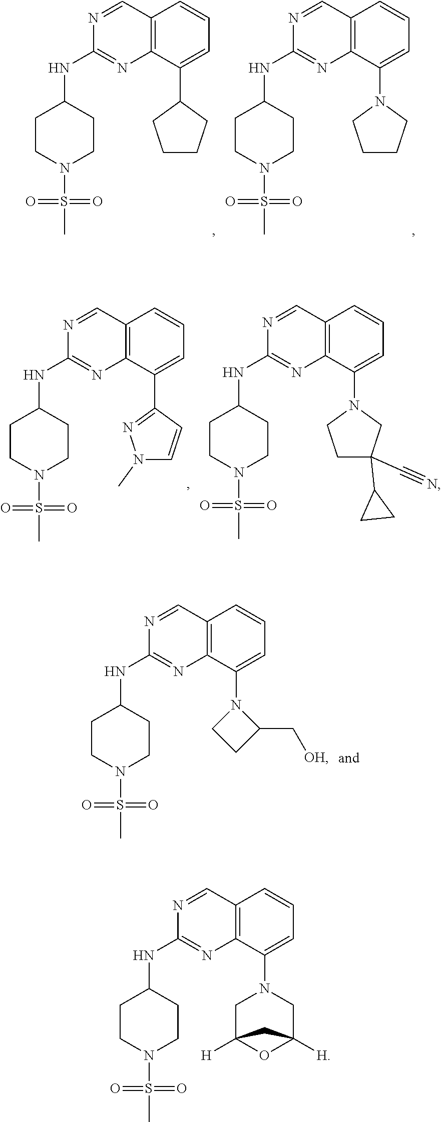

In some embodiments, the compound or pharmaceutical acceptable salt or solvate thereof, has the structure Formula (IAA):

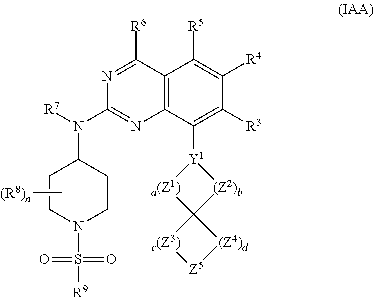

wherein,
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl;
n is selected from 0 to 9;
Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle; and
each R¹¹ is independently selected from hydrogen and optionally substituted C₁₋₄ alkyl.

R³ can be any suitable functional group known by one of skill in the art. In some embodiments, selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R³ is selected from hydrogen, halogen, —CN, optionally substituted C₃₋₄ carbocycle, and optionally substituted 3- to 4 membered heterocycloalkyl. In some embodiments, R³ is selected from hydrogen, fluoro, chloro, bromo, —CN, methyl, ethyl, propyl, butyl, cyclopropyl, cyclobutyl, optionally substituted oxetane, and optionally substituted azetidine. In some embodiments, R³ is selected from hydrogen, fluoro, —CN, cyclopropyl, cyclobutyl, optionally substituted oxetane, and optionally substituted azetidine. In some embodiments, R³ is selected from hydrogen, fluoro, and —CN. In some embodiments, R³ is selected from hydrogen and —CN.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁴ is selected from hydrogen, —CN, optionally substituted methyl, optionally substituted ethyl, optionally substituted propyl, optionally substituted cyclopropyl, optionally substituted cyclobutyl, optionally substituted oxetane, and optionally substituted azetidine. In some embodiments, R⁴ is selected from hydrogen, —CN, —CHF₂, cyclopropyl, cyclobutyl, optionally substituted oxetane, and optionally substituted azetidine. R⁴ is selected from hydrogen, —CN, and —CHF₂.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁵ is selected from hydrogen, halogen, —CN, optionally substituted C₃₋₄ carbocycle, and optionally substituted 3- to 4 membered heterocycloalkyl. In some embodiments, R⁵ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁵ is selected from hydrogen, fluoro, —CN, cyclopropyl, cyclobutyl, optionally substituted oxetane, and optionally substituted azetidine. In some embodiments, R⁵ is selected from hydrogen, methyl, ethyl, and propyl. In some embodiments, R⁵ is hydrogen.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁶ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁶ is selected from hydrogen, halogen, —CN, optionally substituted C₃₋₄ carbocycle, and optionally substituted 3- to 4 membered heterocycloalkyl. In some embodiments, R⁶ is selected from hydrogen, fluoro, —CN, cyclopropyl, cyclobutyl, optionally substituted oxetane, and optionally substituted azetidine. In some embodiments, R⁶ is selected from hydrogen, methyl, ethyl, and propyl. In some embodiments, R⁶ is hydrogen.

R⁷ can be any suitable functional group known by one of skill in the art. In some embodiments, R⁷ is selected from hydrogen, and optionally substituted C₁₋₄ alkyl. In some embodiments, R⁷ is selected from hydrogen, methyl, ethyl, and propyl. In some embodiments, R⁷ is hydrogen.

Variable n can be any suitable number known by one of skill in the art. In some embodiments, n is 0 to 9. In some embodiments, n is 0 to 5. In some embodiments, n is 0 to 3. In some embodiments, n is 0 or 1. In some embodiments n is 0. In some embodiments, n is 1.

R⁸ can be any suitable functional group known by one of skill in the art. In some embodiments, R⁸ is selected from halogen, —CN, and optionally substituted C₁₋₄ alkyl. In some embodiments, R⁸ is selected from halogen and optionally substituted C₁₋₄ alkyl. In some embodiments, R⁸ is fluoro, chloro, bromo, methyl, ethyl, or propyl.

R⁹ can be any suitable functional group known by one of skill in the art. In some embodiments, R⁹ is selected from optionally substituted C₁₋₄ alkyl, optionally substituted C₃₋₆ carbocycle, and 3- to 6-membered heterocycloalkyl. In some embodiments, R⁹ is selected from optionally substituted C₁₋₄ alkyl. In some embodiments, R⁹ is selected from methyl, ethyl, and propyl.

Y¹ can be any suitable atom known by one of skill in the art. In some embodiments, Y¹ is selected rom —N— and —CR¹⁰—. In some embodiments, Y¹ is —N—. In some embodiments, Y¹ is —CR¹⁰—.

Z¹, Z², Z³, Z⁴ and Z⁵ are each independently any suitable atom known by one of skill in the art. In some embodiments, each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond. In some embodiments, each of Z¹, Z², Z³, Z⁴ and Z⁵ are independently selected from —C(R¹⁰)₂—, —NR¹¹—, —N(C(O)R¹⁰)—, —NS(O₂)R¹¹, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3 and 4. In some embodiments, each of a, b, c, and d are independently selected from 1, 2, and 3. In some embodiments, each of a, c, and d are independently selected from 1 and 2.

R¹⁰ can be any suitable functional group known by one of skill in the art. In some embodiments, each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle. In some embodiments, each R¹⁰ is independently selected from hydrogen, halogen, —OH, optionally substituted C₁₋₃ alkyl, and optionally substituted C₃₋₆ cycloalkyl. In some embodiments, each R¹⁰ is independently selected from hydrogen, fluoro, chloro, bromo, —OH, methyl, ethyl, propyl, cyclopropyl and cyclobutyl. In some embodiments, each R¹⁰ is independently selected from hydrogen, fluoro, —OH, methyl, and cyclopropyl.

R¹¹ can be any suitable functional group known by one of skill in the art. In some embodiments, each R¹¹ is independently selected from hydrogen and optionally substituted C₁₋₄ alkyl. In some embodiments, each R¹¹ is independently selected from hydrogen and optionally substituted C₁₋₂ alkyl. In some embodiments, each R¹¹ is independently selected from hydrogen, methyl, and ethyl, wherein the methyl and ethyl are optionally substituted with —OMe, —OEt, and —OPr. In some embodiments, each R¹¹ is independently selected from hydrogen, methyl, and ethyl, wherein the ethyl is optionally substituted with —OMe.

In some embodiments, the compound or pharmaceutically acceptable salt or solvate thereof, has the structure of Formula (IAAA):

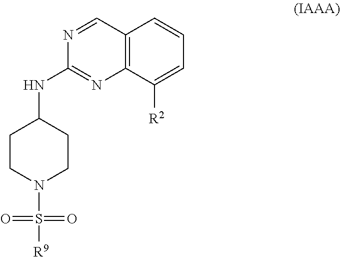

wherein,
R⁹ is selected from optionally substituted C₁₋₄ alkyl, optionally substituted C₃₋₆ carbocycle, and 3- to 6-membered heterocycloalkyl; and
R² is optionally substituted cycloalkyl and optionally substituted heterocycle.
   In some embodiments for the compound of Formula (IAAA), R² is selected from:

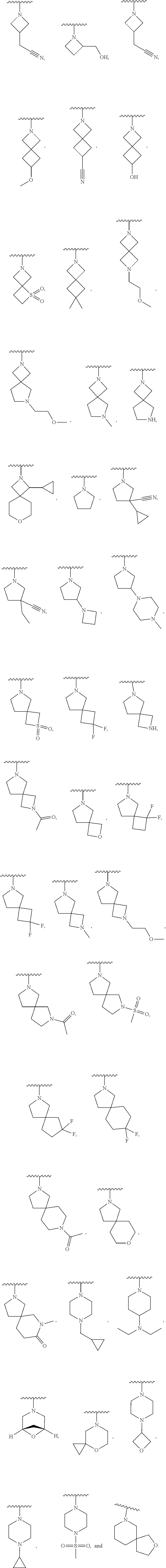

In some embodiments, the compound is selected from:

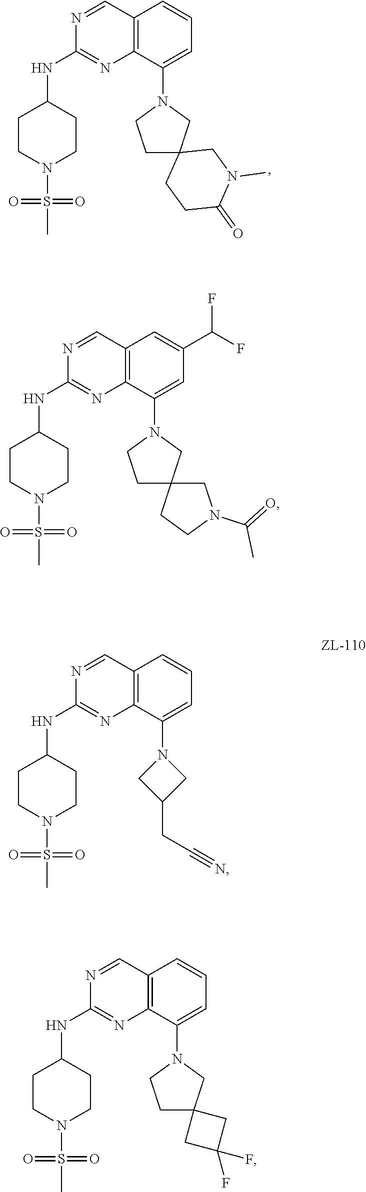 [SMILES: CN1CC2(CCC1=O)CCN(c1cccc3cnc(NC4CCN(S(C)(=O)=O)CC4)nc13)C2 ; CC(=O)N1CCC2(CCN(c3cc(C(F)F)cc4cnc(NC5CCN(S(C)(=O)=O)CC5)nc34)C2)C1 ; CS(=O)(=O)N1CCC(Nc2ncc3cccc(N4CC(CC#N)C4)c3n2)CC1 ; CS(=O)(=O)N1CCC(Nc2ncc3cccc(N4CCC5(C4)CC(F)(F)C5)c3n2)CC1]

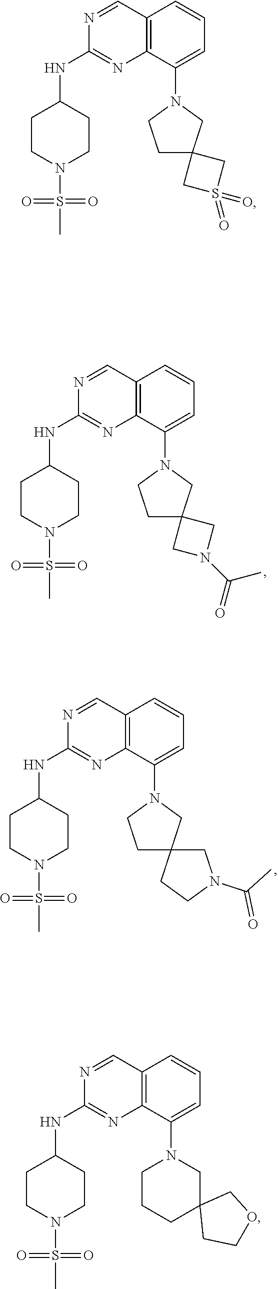 [SMILES: CS(=O)(=O)N1CCC(Nc2ncc3cccc(N4CCC5(C4)CS(=O)(=O)C5)c3n2)CC1 ; CC(=O)N1CC2(CCN(c3cccc4cnc(NC5CCN(S(C)(=O)=O)CC5)nc34)C2)C1 ; CC(=O)N1CCC2(CCN(c3cccc4cnc(NC5CCN(S(C)(=O)=O)CC5)nc34)C2)C1 ; CS(=O)(=O)N1CCC(Nc2ncc3cccc(N4CCCC5(CCOC5)C4)c3n2)CC1]

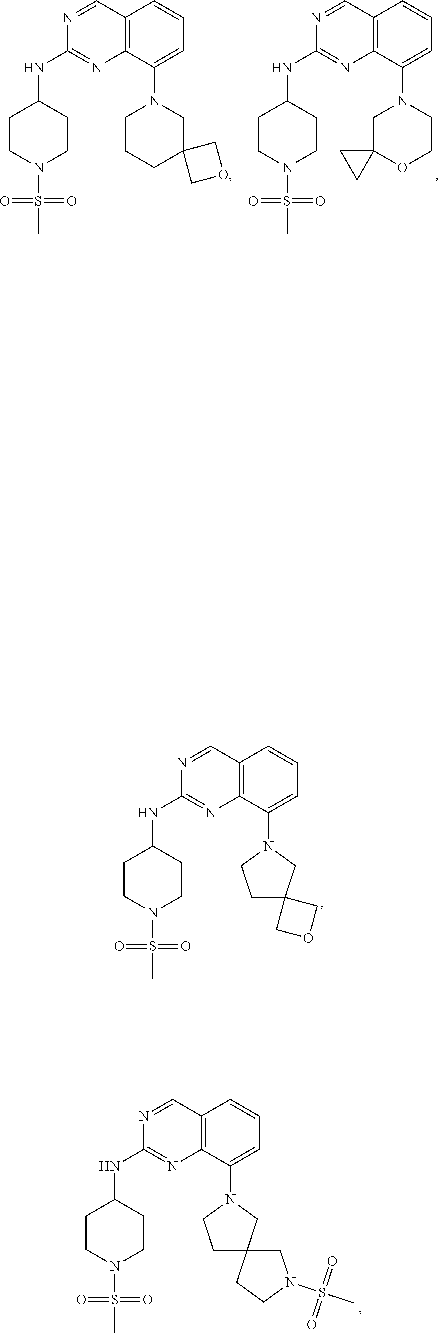

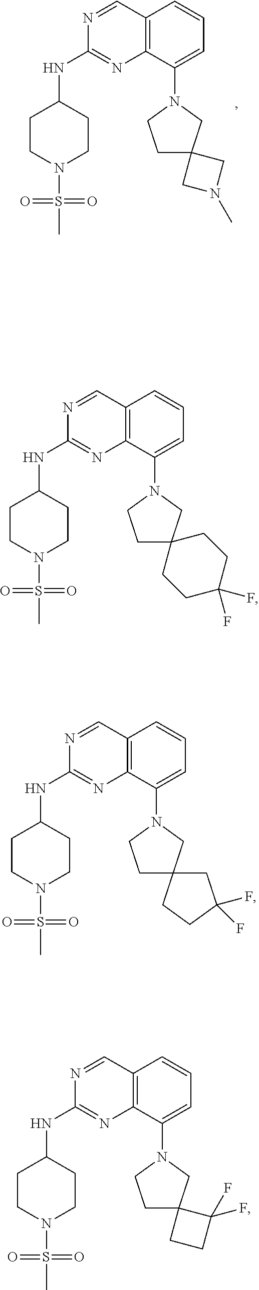 [SMILES: CN1CC2(CCN(c3cccc4cnc(NC5CCN(S(C)(=O)=O)CC5)nc34)C2)C1 ; CS(=O)(=O)N1CCC(Nc2ncc3cccc(N4CCC5(CCC(F)(F)CC5)C4)c3n2)CC1 ; CS(=O)(=O)N1CCC(Nc2ncc3cccc(N4CCC5(CCC(F)(F)C5)C4)c3n2)CC1 ; CS(=O)(=O)N1CCC(Nc2ncc3cccc(N4CCC5(CCC5(F)F)C4)c3n2)CC1]

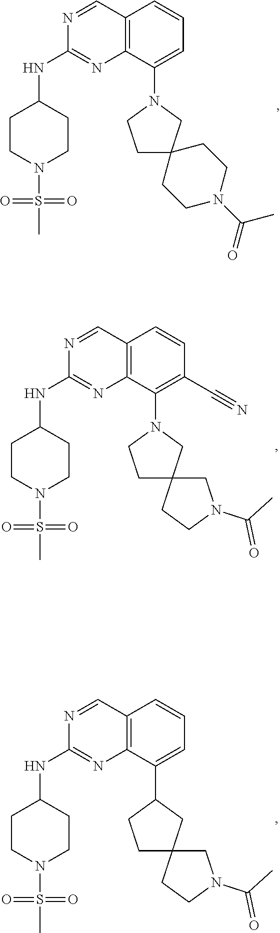 [SMILES: CC(=O)N1CCC2(CC1)CCN(c1cccc3cnc(NC4CCN(S(C)(=O)=O)CC4)nc13)C2 ; CC(=O)N1CCC2(CCN(c3c(C#N)ccc4cnc(NC5CCN(S(C)(=O)=O)CC5)nc34)C2)C1 ; CC(=O)N1CCC2(CCC(c3cccc4cnc(NC5CCN(S(C)(=O)=O)CC5)nc34)C2)C1]

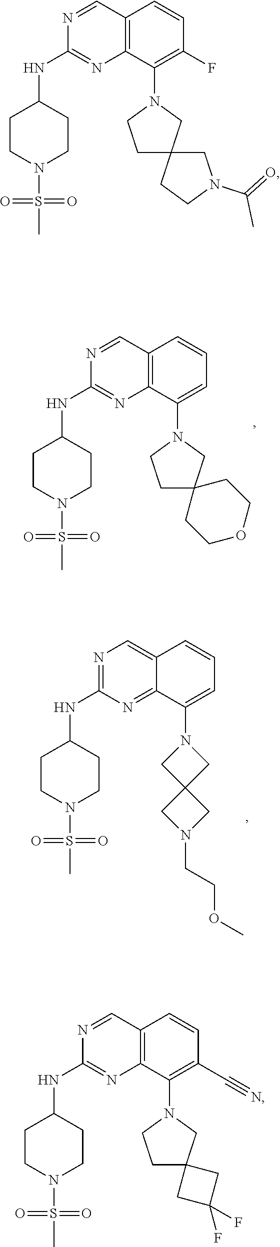 [SMILES: CC(=O)N1CCC2(CCN(c3c(F)ccc4cnc(NC5CCN(S(C)(=O)=O)CC5)nc34)C2)C1 ; CS(=O)(=O)N1CCC(Nc2ncc3cccc(N4CCC5(CCOCC5)C4)c3n2)CC1 ; COCCN1CC2(C1)CN(c1cccc3cnc(NC4CCN(S(C)(=O)=O)CC4)nc13)C2 ; CS(=O)(=O)N1CCC(Nc2ncc3ccc(C#N)c(N4CCC5(C4)CC(F)(F)C5)c3n2)CC1]

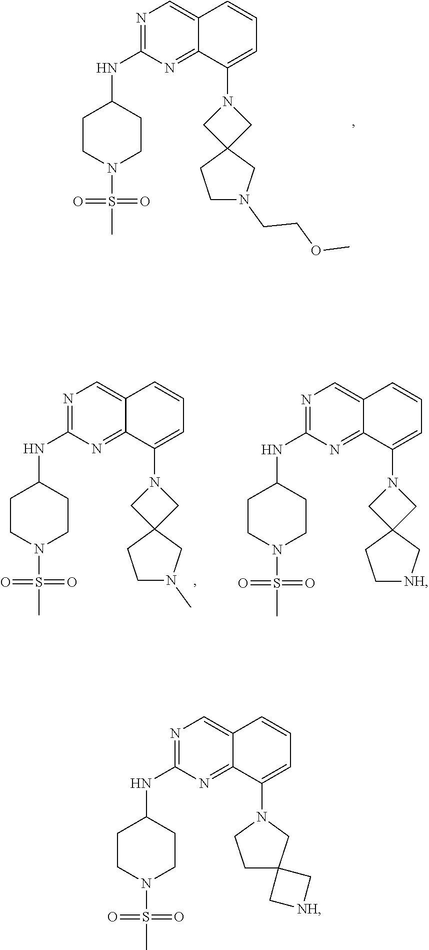 [SMILES: COCCN1CCC2(C1)CN(c1cccc3cnc(NC4CCN(S(C)(=O)=O)CC4)nc13)C2 ; CN1CCC2(C1)CN(c1cccc3cnc(NC4CCN(S(C)(=O)=O)CC4)nc13)C2 ; CS(=O)(=O)N1CCC(Nc2ncc3cccc(N4CC5(CCNC5)C4)c3n2)CC1 ; CS(=O)(=O)N1CCC(Nc2ncc3cccc(N4CCC5(CNC5)C4)c3n2)CC1]

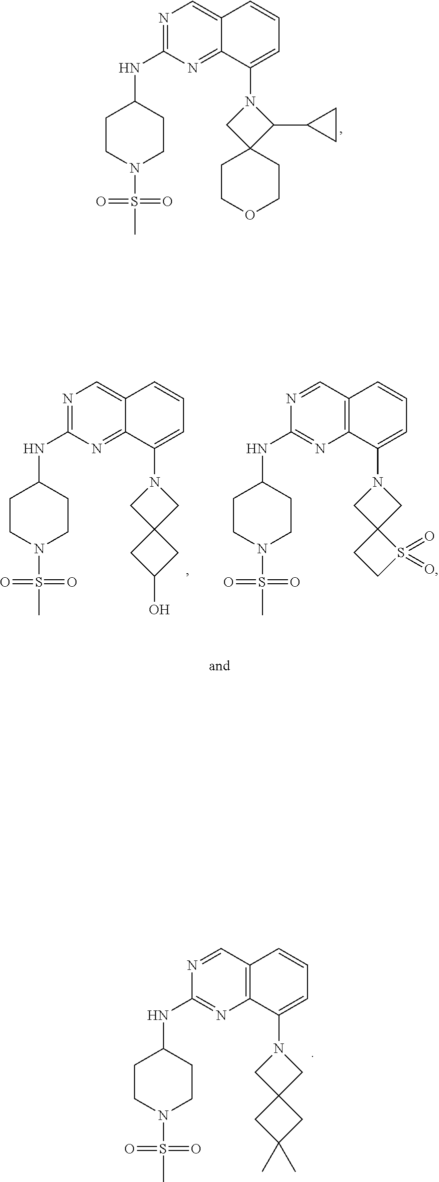 [SMILES: CS(=O)(=O)N1CCC(Nc2ncc3cccc(N4CC5(CCOCC5)C4C4CC4)c3n2)CC1 ; CS(=O)(=O)N1CCC(Nc2ncc3cccc(N4CC5(CC(O)C5)C4)c3n2)CC1 ; CS(=O)(=O)N1CCC(Nc2ncc3cccc(N4CC5(CCS5(=O)=O)C4)c3n2)CC1 ; CC1(C)CC2(CN(c3cccc4cnc(NC5CCN(S(C)(=O)=O)CC5)nc34)C2)C1]

In some embodiments, the compound is selected from:

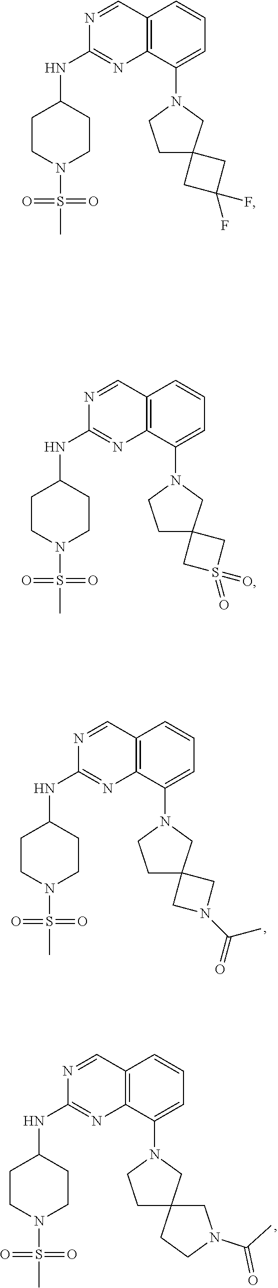 [SMILES: CS(=O)(=O)N1CCC(Nc2ncc3cccc(N4CCC5(C4)CC(F)(F)C5)c3n2)CC1 ; CS(=O)(=O)N1CCC(Nc2ncc3cccc(N4CCC5(C4)CS(=O)(=O)C5)c3n2)CC1 ; CC(=O)N1CC2(CCN(c3cccc4cnc(NC5CCN(S(C)(=O)=O)CC5)nc34)C2)C1 ; CC(=O)N1CCC2(CCN(c3cccc4cnc(NC5CCN(S(C)(=O)=O)CC5)nc34)C2)C1]

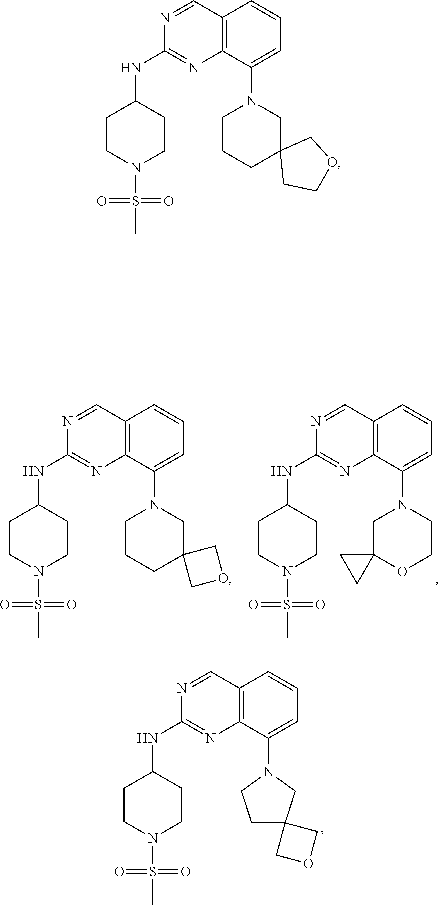

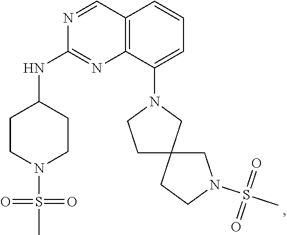 [SMILES: CS(=O)(=O)N1CCC(Nc2ncc3cccc(N4CCC5(CCN(S(C)(=O)=O)C5)C4)c3n2)CC1]

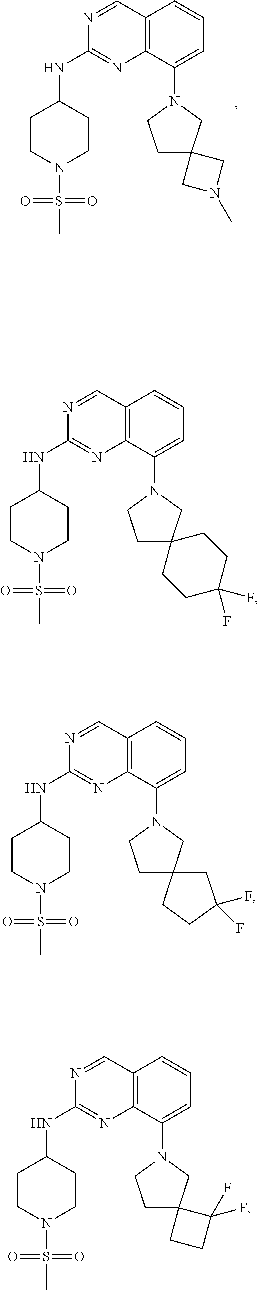 [SMILES: CN1CC2(CCN(c3cccc4cnc(NC5CCN(S(C)(=O)=O)CC5)nc34)C2)C1 ; CS(=O)(=O)N1CCC(Nc2ncc3cccc(N4CCC5(CCC(F)(F)CC5)C4)c3n2)CC1 ; CS(=O)(=O)N1CCC(Nc2ncc3cccc(N4CCC5(CCC(F)(F)C5)C4)c3n2)CC1 ; CS(=O)(=O)N1CCC(Nc2ncc3cccc(N4CCC5(CCC5(F)F)C4)c3n2)CC1]

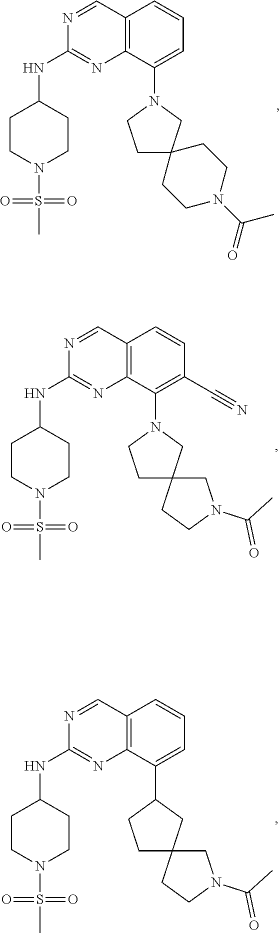 [SMILES: CC(=O)N1CCC2(CC1)CCN(c1cccc3cnc(NC4CCN(S(C)(=O)=O)CC4)nc13)C2 ; CC(=O)N1CCC2(CCN(c3c(C#N)ccc4cnc(NC5CCN(S(C)(=O)=O)CC5)nc34)C2)C1 ; CC(=O)N1CCC2(CCC(c3cccc4cnc(NC5CCN(S(C)(=O)=O)CC5)nc34)C2)C1]

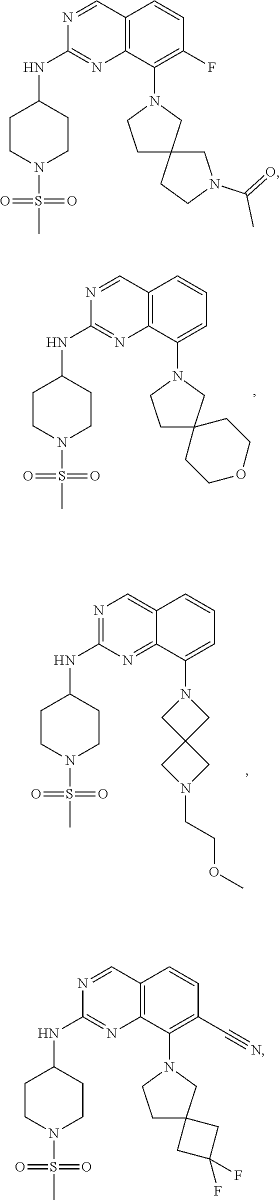 [SMILES: CC(=O)N1CCC2(CCN(c3c(F)ccc4cnc(NC5CCN(S(C)(=O)=O)CC5)nc34)C2)C1 ; CS(=O)(=O)N1CCC(Nc2ncc3cccc(N4CCC5(CCOCC5)C4)c3n2)CC1 ; COCCN1CC2(C1)CN(c1cccc3cnc(NC4CCN(S(C)(=O)=O)CC4)nc13)C2 ; CS(=O)(=O)N1CCC(Nc2ncc3ccc(C#N)c(N4CCC5(C4)CC(F)(F)C5)c3n2)CC1]

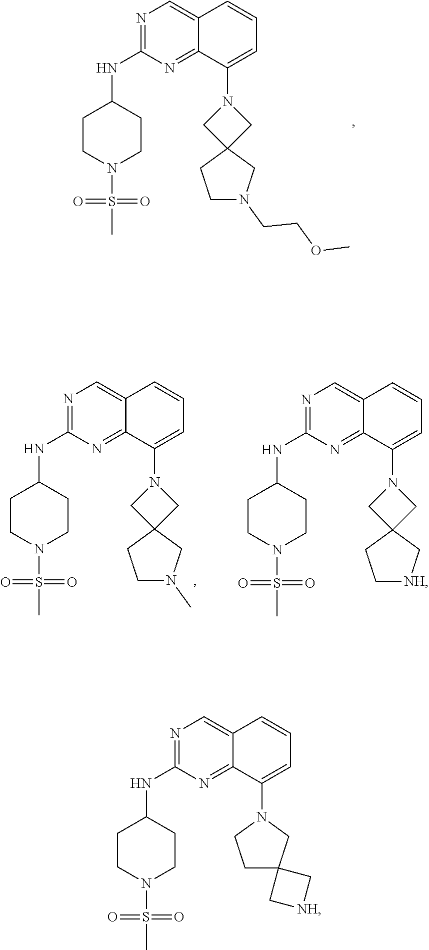 [SMILES: COCCN1CCC2(C1)CN(c1cccc3cnc(NC4CCN(S(C)(=O)=O)CC4)nc13)C2 ; CN1CCC2(C1)CN(c1cccc3cnc(NC4CCN(S(C)(=O)=O)CC4)nc13)C2 ; CS(=O)(=O)N1CCC(Nc2ncc3cccc(N4CC5(CCNC5)C4)c3n2)CC1 ; CS(=O)(=O)N1CCC(Nc2ncc3cccc(N4CCC5(CNC5)C4)c3n2)CC1]

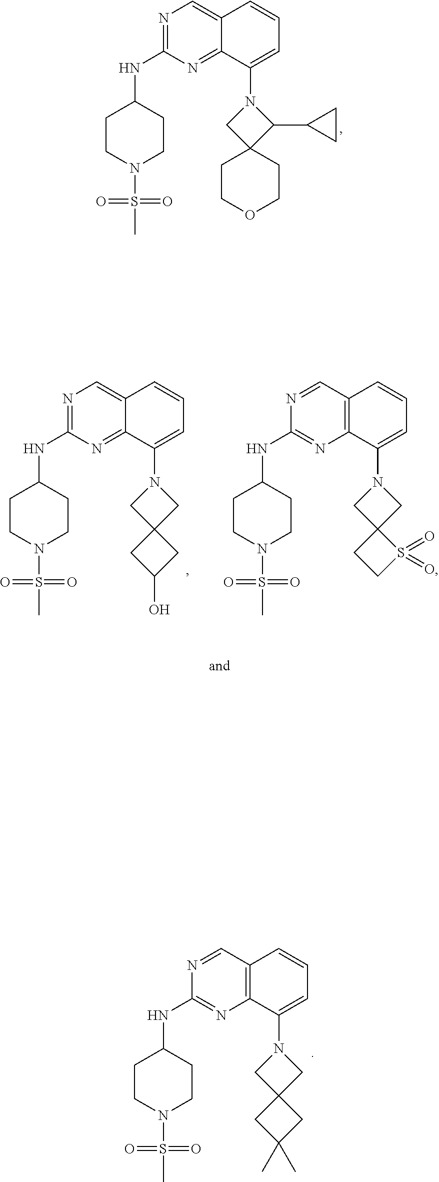 [SMILES: CS(=O)(=O)N1CCC(Nc2ncc3cccc(N4CC5(CCOCC5)C4C4CC4)c3n2)CC1 ; CS(=O)(=O)N1CCC(Nc2ncc3cccc(N4CC5(CC(O)C5)C4)c3n2)CC1 ; CS(=O)(=O)N1CCC(Nc2ncc3cccc(N4CC5(CCS5(=O)=O)C4)c3n2)CC1 ; CC1(C)CC2(CN(c3cccc4cnc(NC5CCN(S(C)(=O)=O)CC5)nc34)C2)C1]

In some embodiments, the compound is selected from:

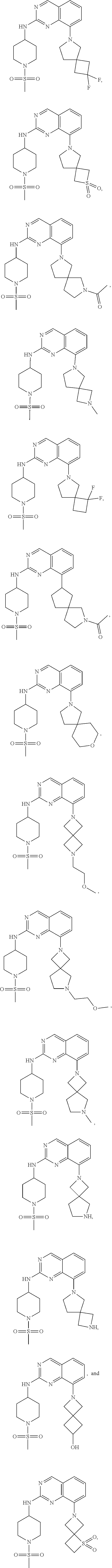

In some embodiments, the compound or pharmaceutically acceptable salt or solvate thereof, has the structure of Formula (IB):

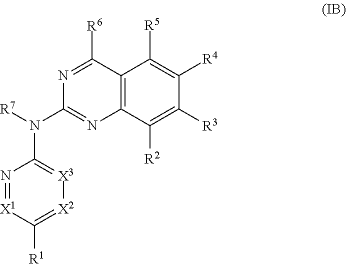

wherein,
each of X¹, X², and X³ is independently selected from N and CR³;
R¹² is selected from hydrogen, halogen, —CN, —NO₂, optionally substituted alkyl, optionally substituted alkenyl, optionally substituted alkynyl, optionally substituted carbocycle, and optionally substituted heterocycle, or R¹² comes together with R¹³ to form an optionally substituted ring; and
each R¹³ is independently selected from hydrogen, halogen, —CN, and optionally substituted C₁₋₄ alkyl.

In some embodiments R² is optionally substituted heterocycle.

In some embodiments, R² is selected from optionally substituted cycloalkyl and optionally substituted heterocycle. In some embodiments, R² is selected from optionally substituted cycloalkyl. In some embodiments, R² is selected from cycloalkyl. In some embodiments, R² is selected from optionally substituted C₃₋₆ cycloalkyl. In some embodiments, R² is selected from optionally substituted C₅₋₆ cycloalkyl. In some embodiments, R² is selected from optionally substituted heterocycloalkyl. In some embodiments, R² is selected from heterocycloalkyl. In some embodiments, R² is selected from optionally substituted 3- to 10-membered heterocycloalkyl. In some embodiments, R² is selected from optionally substituted 3- to 8-membered heterocycloalkyl. In some embodiments, R² is selected from optionally substituted 3- to 7-membered heterocycloalkyl. In some embodiments, R² is selected from optionally substituted 3- to 6-membered heterocycloalkyl. In some embodiments, R² is selected from optionally substituted azetidine, optionally substituted pyrrolidine, optionally substituted piperidine, optionally substituted piperazine, optionally substituted morpholine, optionally substituted 2-azaspiro[3.3]heptane, optionally substituted 5-azaspiro[2.4]heptane, optionally substituted 2-oxa-6-azaspiro[3.3]heptane, optionally substituted 2,6-diazaspiro[3.3]heptane, optionally substituted 1-thia-6-azaspiro[3.3]heptane, optionally substituted 6-azaspiro[3.4]octane, optionally substituted 2,6-diazaspiro[3.4]octane, optionally substituted 2-thia-6-azaspiro[3.4]octane, optionally substituted 2-thia-6-azaspiro[3.4]octane 2,2-dioxide, optionally substituted 4-oxa-7-azaspiro[2.5]octane, optionally substituted 2-azaspiro[4.4]nonane, optionally substituted 2,7-diazaspiro[4.4]nonane, optionally substituted 2-oxa-6-azaspiro[3.5]nonane, optionally substituted 7-oxa-2-azaspiro[3.5]nonane, optionally substituted 2-azaspiro[4.5]decane, optionally substituted 2,8-diazaspiro[4.5]decane, optionally substituted 8-oxa-2-azaspiro[4.5]decane, and optionally substituted 2-oxa-7-azaspiro[4.5]decane. In some embodiments, R² is 6-oxa-3-azabicyclo[3.1.1]heptane.

In some embodiments, R² is substituted with halogen, —SO₂R^{2a}, —NR^{2a}, oxo, —COR^{2a}, C₁₋₄ alkyl, C₁₋₃alkylene-C₁₋₃alkoxy, —OR^{2a}, —CN, —CH₂—CN, and an optionally substituted 3- to 6-membered heterocycloalkyl, wherein R^{2a} is selected from hydrogen and C₁₋₆ alkyl. In some embodiments, R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, piperidine, piperazine, or azetidine, wherein R^{2a} is selected from C₁₋₆ alkyl. In some embodiments, R² is substituted with halogen, —SO₂R^{2a}, oxo, and C₁₋₄ alkyl, wherein R^{2a} is selected from C₁₋₆ alkyl. In some embodiments, R² is substituted with halogen, —SO₂R^{2a}, and —NR^{2a}, oxo, and C₁₋₃ alkyl, wherein R^{2a} is selected from C₁₋₆ alkyl. In some embodiments, R² is substituted with fluoro, —SO₂Me, oxo, acetyl, methyl, ethyl, propyl, cyclopropyl, —CH₂-cyclopropyl, —CH₂OH, —(CH₂)₂OMe, —(CH₂)₂OEt, —OH, —OMe, —OEt, —CN, —C—CN, oxetane, and azetidine. In some embodiments, R² is substituted with fluoro, —SO₂Me, oxo, acetyl, methyl, ethyl, cyclopropyl, —CH₂-cyclopropyl, —CH₂OH, —(CH₂)₂OMe, —OH, —OMe, —CN, —C—CN, and oxetane. In some embodiments, R² is substituted with fluoro, —SO₂Me, oxo, and methyl. In some embodiments, R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, methyl piperidine, or azetidine, wherein R^{2a} is selected from C₁₋₆ alkyl.

In some embodiments, R² is selected from optionally substituted C₃₋₆ cycloalkyl, optionally substituted pyrrolidine, optionally substituted piperidine, optionally substituted pyrazole, optionally substituted azetidine, optionally substituted oxetane, and optionally substituted morpholine. In some embodiments, R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, or azetidine, wherein R is selected from C₁₋₆ alkyl. In some embodiments, R² is 6-oxa-3-azabicyclo[3.1.1]heptane.

In some embodiments, R² is selected from:

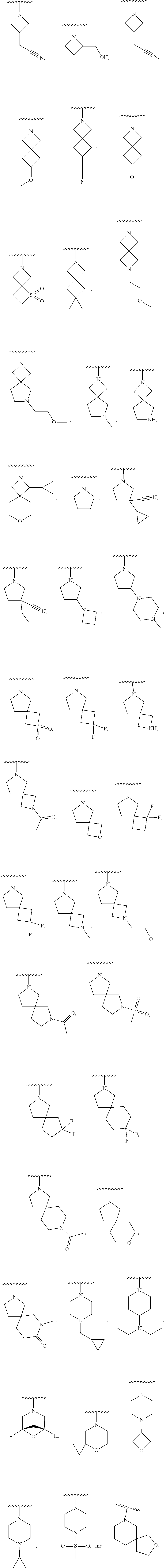

In some embodiments, R² is selected from:

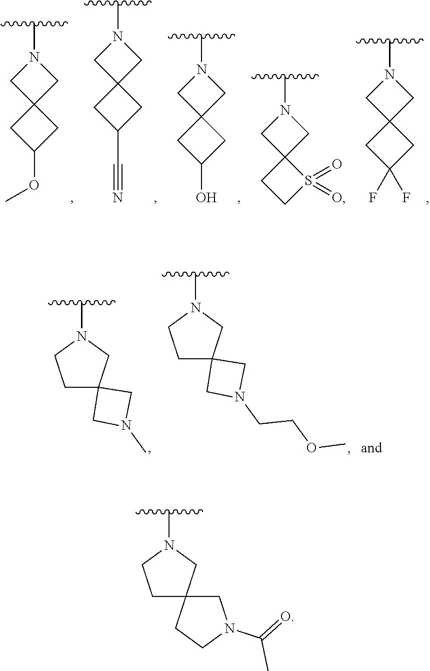

R³ can be any suitable functional group known by one of skill in the art. In some embodiments, selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R³ is selected from hydrogen, halogen, —CN, optionally substituted C₃₋₄ carbocycle, and optionally substituted 3- to 4 membered heterocycloalkyl. In some embodiments, R³ is selected from hydrogen, fluoro, chloro, bromo, —CN, methyl, ethyl, propyl, butyl, cyclopropyl, cyclobutyl, optionally substituted oxetane, and optionally substituted azetidine. In some embodiments, R³ is selected from hydrogen, fluoro, —CN, cyclopropyl, cyclobutyl, optionally substituted oxetane, and optionally substituted azetidine. In some embodiments, R³ is selected from hydrogen, fluoro, and —CN. In some embodiments, R³ is selected from hydrogen and —CN.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁴ is selected from hydrogen, —CN, optionally substituted methyl, optionally substituted ethyl, optionally substituted propyl, optionally substituted cyclopropyl, optionally substituted cyclobutyl, optionally substituted oxetane, and optionally substituted azetidine. In some embodiments, R⁴ is selected from hydrogen, —CN, —CHF₂, cyclopropyl, cyclobutyl, optionally substituted oxetane, and optionally substituted azetidine. R⁴ is selected from hydrogen, —CN, and —CHF₂.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁵ is selected from hydrogen, halogen, —CN, optionally substituted C₃₋₄ carbocycle, and optionally substituted 3- to 4 membered heterocycloalkyl. In some embodiments, R⁵ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁵ is selected from hydrogen, fluoro, —CN, cyclopropyl, cyclobutyl, optionally substituted oxetane, and optionally substituted azetidine. In some embodiments, R⁵ is selected from hydrogen, methyl, ethyl, and propyl. In some embodiments, R⁵ is hydrogen.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁶ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁶ is selected from hydrogen, halogen, —CN, optionally substituted C₃₋₄ carbocycle, and optionally substituted 3- to 4 membered heterocycloalkyl. In some embodiments, R⁶ is selected from hydrogen, fluoro, —CN, cyclopropyl, cyclobutyl, optionally substituted oxetane, and optionally substituted azetidine. In some embodiments, R⁶ is selected from hydrogen, methyl, ethyl, and propyl. In some embodiments, R⁶ is hydrogen.

R⁷ can be any suitable functional group known by one of skill in the art. In some embodiments, R⁷ is selected from hydrogen, and optionally substituted C₁₋₄ alkyl. In some embodiments, R⁷ is selected from hydrogen, methyl, ethyl, and propyl. In some embodiments, R⁷ is hydrogen.

Each of X¹, X², and X³ can be any suitable atom known by one of skill in the art. In some embodiments, each of X¹, X², and X³ is independently selected from N and CR³. In some embodiments, each of X¹, X², and X³ is independently N. In some embodiments, each of X¹, X², and X³ is independently selected form CR¹³. In some embodiments, X¹, X², and X³ are each CH.

R¹² can be any suitable functional group known by one of skill in the art. In some embodiments, R¹² is selected from hydrogen, halogen, —CN, —NO₂, optionally substituted alkyl, optionally substituted alkenyl, optionally substituted alkynyl, optionally substituted carbocycle, and optionally substituted heterocycle, or R¹² comes together with R¹³ to form an optionally substituted ring. In some embodiments, R¹² is an optionally substituted heterocycle. In some embodiments, R¹² is an optionally substituted 3- to 8-membered heterocycle. In some embodiments, R¹² is an optionally substituted 5- to 8-membered heterocycle. In some embodiments, R¹² is an optionally substituted 6- to 7-membered heterocycle.

In some embodiments, the compound or pharmaceutically acceptable salt or solvate thereof, has the structure of Formula (IBB):

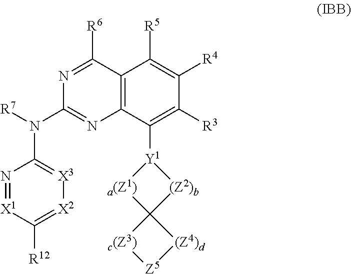

wherein,
Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle; and
each R¹¹ is independently selected from hydrogen and optionally substituted C₁₋₄ alkyl.

R³ can be any suitable functional group known by one of skill in the art. In some embodiments, selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R³ is selected from hydrogen, halogen, —CN, optionally substituted C₃₋₄ carbocycle, and optionally substituted 3- to 4 membered heterocycloalkyl. In some embodiments, R³ is selected from hydrogen, fluoro, chloro, bromo, —CN, methyl, ethyl, propyl, butyl, cyclopropyl, cyclobutyl, optionally substituted oxetane, and optionally substituted azetidine. In some embodiments, R³ is selected from hydrogen, fluoro, —CN, cyclopropyl, cyclobutyl, optionally substituted oxetane, and optionally substituted azetidine. In some embodiments, R³ is selected from hydrogen, fluoro, and —CN. In some embodiments, R³ is selected from hydrogen and —CN.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁴ is selected from hydrogen, —CN, optionally substituted methyl, optionally substituted ethyl, optionally substituted propyl, optionally substituted cyclopropyl, optionally substituted cyclobutyl, optionally substituted oxetane, and optionally substituted azetidine. In some embodiments, R⁴ is selected from hydrogen, —CN, —CHF₂, cyclopropyl, cyclobutyl, optionally substituted oxetane, and optionally substituted azetidine. R⁴ is selected from hydrogen, —CN, and —CHF₂.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁵ is selected from hydrogen, halogen, —CN, optionally substituted C₃₋₄ carbocycle, and optionally substituted 3- to 4 membered heterocycloalkyl. In some embodiments, R⁵ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁵ is selected from hydrogen, fluoro, —CN, cyclopropyl, cyclobutyl, optionally substituted oxetane, and optionally substituted azetidine. In some embodiments, R⁵ is selected from hydrogen, methyl, ethyl, and propyl. In some embodiments, R⁵ is hydrogen.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁶ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁶ is selected from hydrogen, halogen, —CN, optionally substituted C₃₋₄ carbocycle, and optionally substituted 3- to 4 membered heterocycloalkyl. In some embodiments, R⁶ is selected from hydrogen, fluoro, —CN, cyclopropyl, cyclobutyl, optionally substituted oxetane, and optionally substituted azetidine. In some embodiments, R⁶ is selected from hydrogen, methyl, ethyl, and propyl. In some embodiments, R⁶ is hydrogen.

R⁷ can be any suitable functional group known by one of skill in the art. In some embodiments, R⁷ is selected from hydrogen, and optionally substituted C₁₋₄ alkyl. In some embodiments, R⁷ is selected from hydrogen, methyl, ethyl, and propyl. In some embodiments, R⁷ is hydrogen.

Each of X¹, X², and X³ can be any suitable atom known by one of skill in the art. In some embodiments, each of X¹, X², and X³ is independently selected from N and CR³. In some embodiments, each of X¹, X², and X³ is independently N. In some embodiments, each of X¹, X², and X³ is independently selected form CR¹³. In some embodiments, X¹, X², and X³ are each CH.

R¹² can be any suitable functional group known by one of skill in the art. In some embodiments, R¹² is selected from hydrogen, halogen, —CN, —NO₂, optionally substituted alkyl, optionally substituted alkenyl, optionally substituted alkynyl, optionally substituted carbocycle, and optionally substituted heterocycle, or R¹² comes together with R¹³ to form an optionally substituted ring. In some embodiments, R¹² is an optionally substituted heterocycle. In some embodiments, R¹² is an optionally substituted 3- to 8-membered heterocycle. In some embodiments, R¹² is an optionally substituted 5- to 8-membered heterocycle. In some embodiments, R¹² is an optionally substituted 6- to 7-membered heterocycle.

Each R¹³ can be any suitable functional group known by one of skill in the art. In some embodiments, each R¹³ is independently selected from hydrogen, halogen, —CN, and optionally substituted C₁₋₄ alkyl, or R² comes together with R¹³ to form an optionally substituted ring. In some embodiments, each R¹³ is independently selected from hydrogen, fluoro, chloro, bromo, —CN, methyl, ethyl, and propyl. In some embodiments, each R¹³ is independently selected from hydrogen, fluoro, —CN, methyl, and ethyl. In some embodiments, each R¹³ is independently hydrogen.

Z¹, Z², Z³, Z⁴ and Z⁵ are each independently any suitable atom known by one of skill in the art. In some embodiments, each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond. In some embodiments, each of Z¹, Z², Z³, Z⁴ and Z⁵ are independently selected from —C(R¹⁰)₂—, —NR¹¹—, —N(C(O)R¹⁰)—, —NS(O₂)R¹¹, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3 and 4. In some embodiments, each of a, b, c, and d are independently selected from 1, 2, and 3. In some embodiments, each of a, b, c, and d are independently selected from 1 and 2.

R¹⁰ can be any suitable functional group known by one of skill in the art. In some embodiments, each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle. In some embodiments, each R¹⁰ is independently selected from hydrogen, halogen, —CN, —OH, —O—C₁₋₄ alkyl, and optionally substituted C₁₋₃ alkyl. In some embodiments, each R¹⁰ is independently selected from hydrogen, halogen, —CN, —OH, —OMe, —OEt, methyl, ethyl, propyl, and —CH₂CH₂OCH₃. In some embodiments, each R¹⁰ is independently selected from hydrogen, halogen, —CN, —OH, —OMe, methyl, and —CH₂CH₂OCH₃. In some embodiments, each R¹⁰ is independently selected from hydrogen, fluoro, —CN, —OH, —OMe, methyl, and —CH₂CH₂OCH₃.

R¹¹ can be any suitable functional group known by one of skill in the art. In some embodiments, each R¹¹ is independently selected from hydrogen and optionally substituted C₁₋₄ alkyl. In some embodiments, each R¹¹ is independently selected from hydrogen and optionally substituted C₁₋₂ alkyl. In some embodiments, each R¹¹ is independently selected from hydrogen, methyl, and ethyl, wherein the methyl and ethyl are optionally substituted with —OMe, —OEt, and —OPr. In some embodiments, each R¹¹ is independently selected from hydrogen, methyl, and ethyl, wherein the ethyl is optionally substituted with —OMe.

In some embodiments, the compound is selected from:

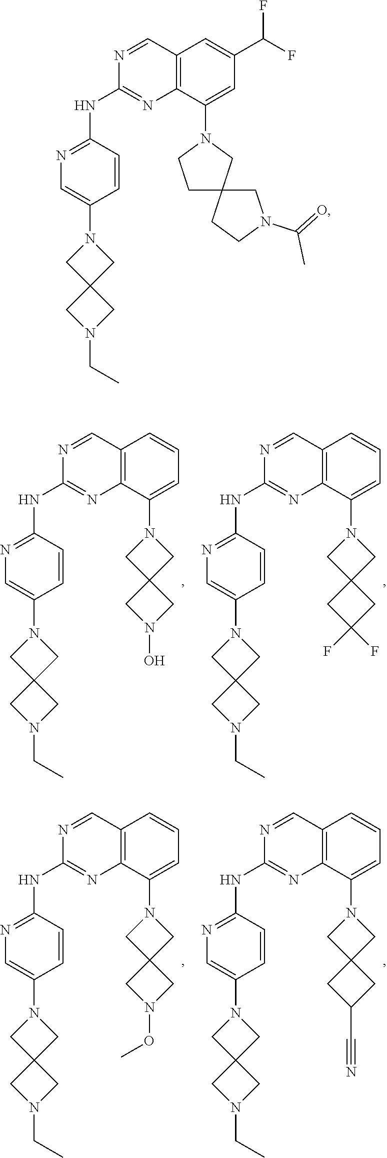

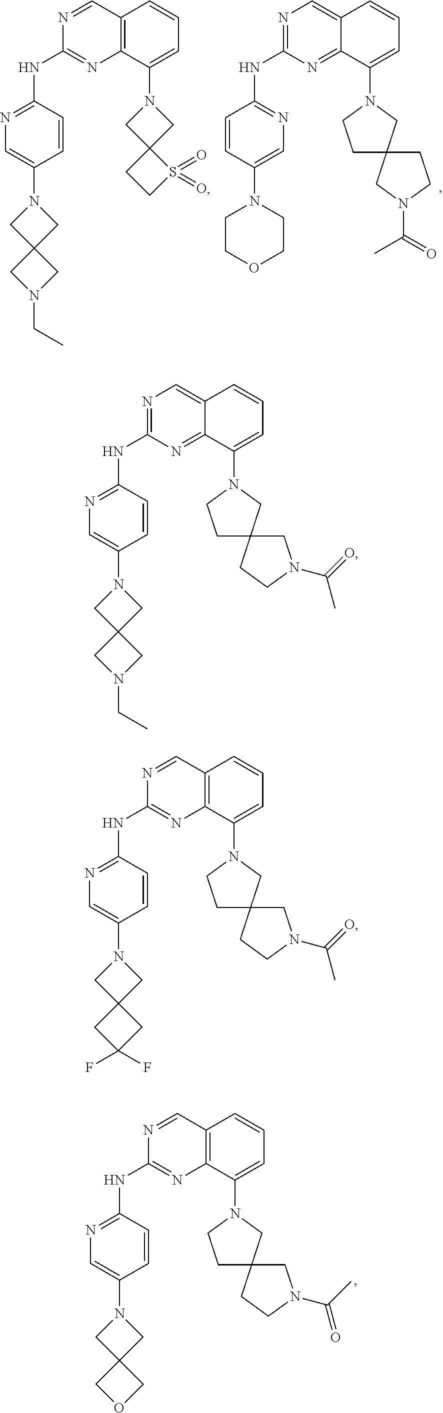

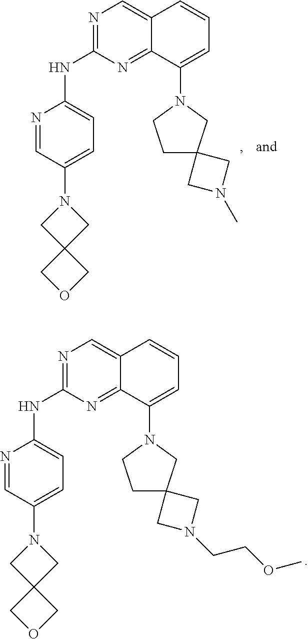 [SMILES: CN1CC2(CCN(c3cccc4cnc(Nc5ccc(N6CC7(COC7)C6)cn5)nc34)C2)C1 ; COCCN1CC2(CCN(c3cccc4cnc(Nc5ccc(N6CC7(COC7)C6)cn5)nc34)C2)C1]

In some embodiments, the compound is selected from:

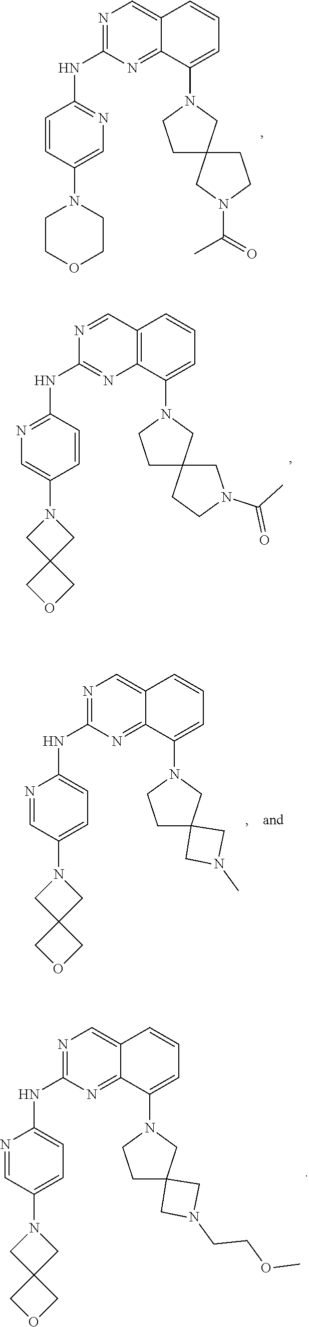 [SMILES: CC(=O)N1CCC2(CCN(c3cccc4cnc(Nc5ccc(N6CCOCC6)cn5)nc34)C2)C1 ; CC(=O)N1CCC2(CCN(c3cccc4cnc(Nc5ccc(N6CC7(COC7)C6)cn5)nc34)C2)C1 ; CN1CC2(CCN(c3cccc4cnc(Nc5ccc(N6CC7(COC7)C6)cn5)nc34)C2)C1 ; COCCN1CC2(CCN(c3cccc4cnc(Nc5ccc(N6CC7(COC7)C6)cn5)nc34)C2)C1]

In some embodiments, the compound or pharmaceutically acceptable salt or solvate thereof, has the structure of Formula (IC):

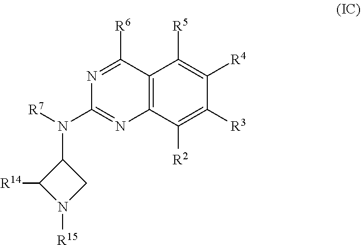

wherein,
   R¹⁴ is selected from halogen, —CN, optionally substituted C₁₋₄ alkyl, optionally substituted C₃₋₆ carbocycle, and optionally substituted 3- to 6-membered heterocycloalkyl, or R¹⁴ and R¹⁵ come together to form an optionally substituted heterocycle; and
R⁵ is selected from —S(O)₂R¹⁶—, optionally substituted C₁₋₄ alkyl, optionally substituted C₃₋₆ carbocycle, and optionally substituted 3- to 6-membered heterocycloalkyl.

In some embodiments R² is optionally substituted heterocycle.

In some embodiments, R² is selected from optionally substituted cycloalkyl and optionally substituted heterocycle. In some embodiments, R² is selected from optionally substituted cycloalkyl. In some embodiments, R² is selected from cycloalkyl. In some embodiments, R² is selected from optionally substituted C₃₋₆ cycloalkyl. In some embodiments, R² is selected from optionally substituted C₅₋₆ cycloalkyl. In some embodiments, R² is selected from optionally substituted heterocycloalkyl. In some embodiments, R² is selected from heterocycloalkyl. In some embodiments, R² is selected from optionally substituted 3- to 10-membered heterocycloalkyl. In some embodiments, R² is selected from optionally substituted 3- to 8-membered heterocycloalkyl. In some embodiments, R² is selected from optionally substituted 3- to 7-membered heterocycloalkyl. In some embodiments, R² is selected from optionally substituted 3- to 6-membered heterocycloalkyl. In some embodiments, R² is selected from optionally substituted azetidine, optionally substituted pyrrolidine, optionally substituted piperidine, optionally substituted piperazine, optionally substituted morpholine, optionally substituted 2-azaspiro[3.3]heptane, optionally substituted 5-azaspiro[2.4]heptane, optionally substituted 2-oxa-6-azaspiro[3.3]heptane, optionally substituted 2,6-diazaspiro[3.3]heptane, optionally substituted 1-thia-6-azaspiro[3.3]heptane, optionally substituted 6-azaspiro[3.4]octane, optionally substituted 2,6-diazaspiro[3.4]octane, optionally substituted 2-thia-6-azaspiro[3.4]octane, optionally substituted 2-thia-6-azaspiro[3.4]octane 2,2-dioxide, optionally substituted 4-oxa-7-azaspiro[2.5]octane, optionally substituted 2-azaspiro[4.4]nonane, optionally substituted 2,7-diazaspiro[4.4]nonane, optionally substituted 2-oxa-6-azaspiro[3.5]nonane, optionally substituted 7-oxa-2-azaspiro[3.5]nonane, optionally substituted 2-azaspiro[4.5]decane, optionally substituted 2,8-diazaspiro[4.5]decane, optionally substituted 8-oxa-2-azaspiro[4.5]decane, and optionally substituted 2-oxa-7-azaspiro[4.5]decane. In some embodiments, R² is 6-oxa-3-azabicyclo[3.1.1]heptane.

In some embodiments, R² is substituted with halogen, —SO₂R^{2a}, —NR^{2a}, oxo, —COR^{2a}, C₁₋₄ alkyl, C₁₋₃alkylene-C₁₋₃alkoxy, —OR^{2a}, —CN, —CH₂—CN, and an optionally substituted 3- to 6-membered heterocycloalkyl, wherein R^{2a} is selected from hydrogen and C₁₋₆ alkyl. In some embodiments, R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, piperidine, piperazine, or azetidine, wherein R^{2a} is selected from C₁₋₆ alkyl. In some embodiments, R² is substituted with halogen, —SO₂R^{2a}, oxo, and C₁₋₄ alkyl, wherein R^{2a} is selected from C₁₋₆ alkyl. In some embodiments, R² is substituted with halogen, —SO₂R^{2a}, and —NR^{2a}, oxo, and C₁₋₃ alkyl, wherein R^{2a} is selected from C₁₋₆ alkyl. In some embodiments, R² is substituted with fluoro, —SO₂Me, oxo, acetyl, methyl, ethyl, propyl, cyclopropyl, —CH₂-cyclopropyl, —CH₂OH, —(CH₂)₂OMe, —(CH₂)₂OEt, —OH, —OMe, —OEt, —CN, —C—CN, oxetane, and azetidine. In some embodiments, R² is substituted with fluoro, —SO₂Me, oxo, acetyl, methyl, ethyl, cyclopropyl, —CH₂-cyclopropyl, —CH₂OH, —(CH₂)₂OMe, —OH, —OMe, —CN, —C—CN, and oxetane. In some embodiments, R² is substituted with fluoro, —SO₂Me, oxo, and methyl. In some embodiments, R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, methyl piperidine, or azetidine, wherein R^{2a} is selected from C₁₋₆ alkyl.

In some embodiments, R² is selected from optionally substituted C₃₋₆ cycloalkyl, optionally substituted pyrrolidine, optionally substituted piperidine, optionally substituted pyrazole, optionally substituted azetidine, optionally substituted oxetane, and optionally substituted morpholine. In some embodiments, R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, or azetidine, wherein R^{2a} is selected from C₁₋₆ alkyl. In some embodiments, R² is 6-oxa-3-azabicyclo[3.1.1]heptane.

In some embodiments, R² is selected from:

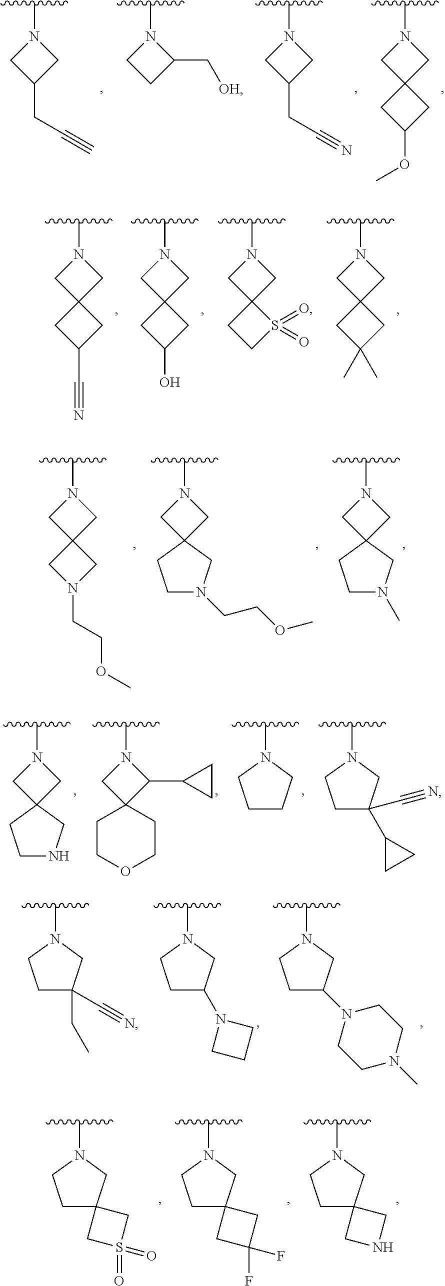

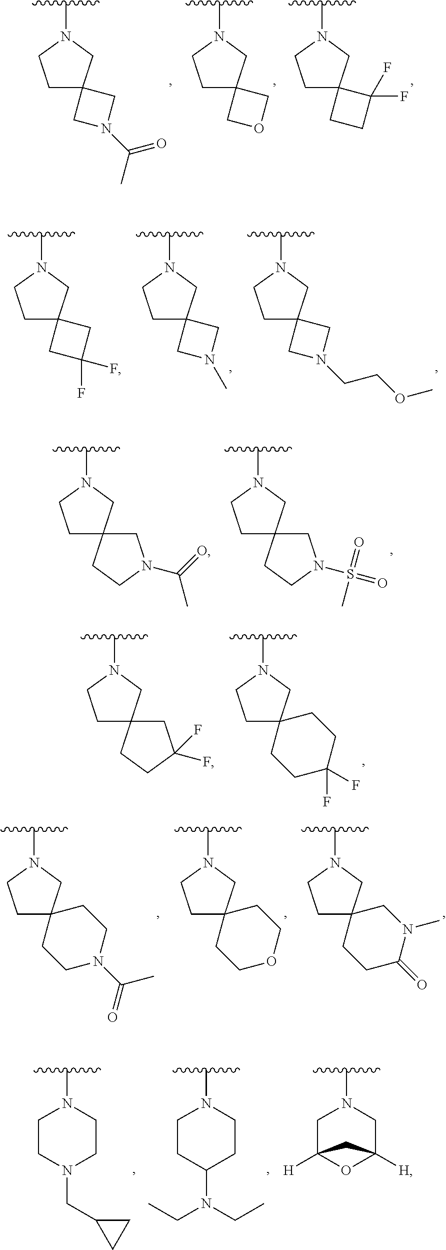

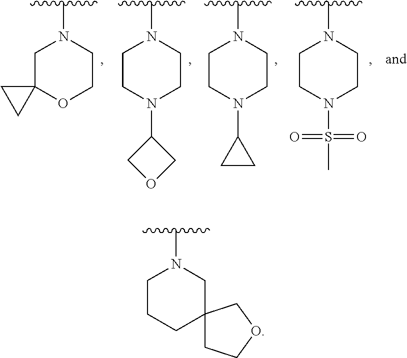

In some embodiments, R² is selected from:

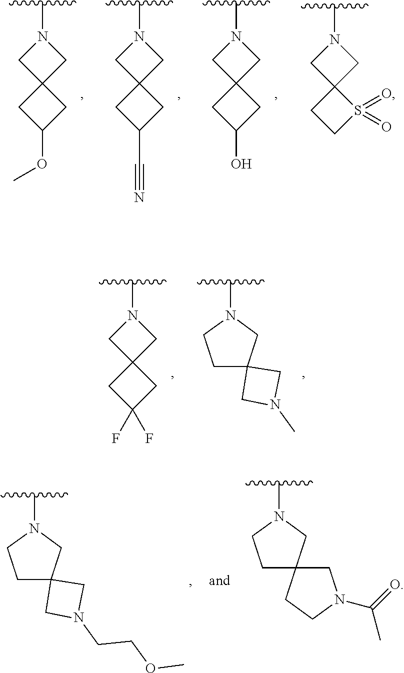

R³ can be any suitable functional group known by one of skill in the art. In some embodiments, selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R³ is selected from hydrogen, halogen, —CN, optionally substituted C₃₋₄ carbocycle, and optionally substituted 3- to 4 membered heterocycloalkyl. In some embodiments, R³ is selected from hydrogen, fluoro, chloro, bromo, —CN, methyl, ethyl, propyl, butyl, cyclopropyl, cyclobutyl, optionally substituted oxetane, and optionally substituted azetidine. In some embodiments, R³ is selected from hydrogen, fluoro, —CN, cyclopropyl, cyclobutyl, optionally substituted oxetane, and optionally substituted azetidine. In some embodiments, R³ is selected from hydrogen, fluoro, and —CN. In some embodiments, R³ is selected from hydrogen and —CN.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁴ is selected from hydrogen, —CN, optionally substituted methyl, optionally substituted ethyl, optionally substituted propyl, optionally substituted cyclopropyl, optionally substituted cyclobutyl, optionally substituted oxetane, and optionally substituted azetidine. In some embodiments, R⁴ is selected from hydrogen, —CN, —CHF₂, cyclopropyl, cyclobutyl, optionally substituted oxetane, and optionally substituted azetidine. R⁴ is selected from hydrogen, —CN, and —CHF₂.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁵ is selected from hydrogen, halogen, —CN, optionally substituted C₃₋₄ carbocycle, and optionally substituted 3- to 4 membered heterocycloalkyl. In some embodiments, R⁵ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁵ is selected from hydrogen, fluoro, —CN, cyclopropyl, cyclobutyl, optionally substituted oxetane, and optionally substituted azetidine. In some embodiments, R⁵ is selected from hydrogen, methyl, ethyl, and propyl. In some embodiments, R⁵ is hydrogen.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁶ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁶ is selected from hydrogen, halogen, —CN, optionally substituted C₃₋₄ carbocycle, and optionally substituted 3- to 4 membered heterocycloalkyl. In some embodiments, R⁶ is selected from hydrogen, fluoro, —CN, cyclopropyl, cyclobutyl, optionally substituted oxetane, and optionally substituted azetidine. In some embodiments, R⁶ is selected from hydrogen, methyl, ethyl, and propyl. In some embodiments, R⁶ is hydrogen.

R⁷ can be any suitable functional group known by one of skill in the art. In some embodiments, R⁷ is selected from hydrogen, and optionally substituted C₁₋₄ alkyl. In some embodiments, R⁷ is selected from hydrogen, methyl, ethyl, and propyl. In some embodiments, R⁷ is hydrogen.

R¹⁴ can be any suitable functional group known by one of skill in the art. In some embodiments, R¹⁴ is selected from hydrogen, halogen, —CN, optionally substituted C₁₋₄ alkyl, optionally substituted C₃₋₆ carbocycle, and optionally substituted 3- to 6-membered heterocycloalkyl, or R⁴ and R⁵ come together to form an optionally substituted heterocycle. In some embodiments, R¹⁴ is selected from halogen, —CN, and optionally substituted C₁₋₄ alkyl. In some embodiments, R¹⁴ is selected from fluoro, chlor, bromo, methyl, ethyl, and propyl.

R⁵ can be any suitable functional group known by one of skill in the art. In some embodiments, R⁵ is selected from —S(O)₂R¹⁶—, optionally substituted C₁₋₄ alkyl, optionally substituted C₃₋₆ carbocycle, and optionally substituted 3- to 6-membered heterocycloalkyl, or R⁵ and R¹⁴ come together to form an optionally substituted heterocycle. In some embodiments, R⁵ is selected from —S(O)₂R¹⁶— and optionally substituted C₁₋₄ alkyl. In some embodiments, R⁵ is optionally substituted C₃₋₆ carbocycle. In some embodiments, R¹⁵ is optionally substituted 3- to 6-membered heterocycloalkyl.

In some embodiments, the compound or pharmaceutically acceptable salt or solvate thereof, has the structure of Formula (ICC):

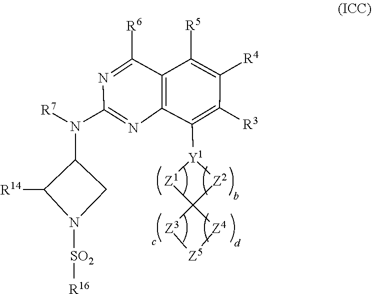

wherein,
Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle;
each R¹¹ is independently selected from hydrogen and optionally substituted C₁₋₄ alkyl; and
R¹⁶ is selected from —S(O)₂R¹⁶—, optionally substituted C₁₋₄ alkyl, optionally substituted C₃₋₆ carbocycle, and optionally substituted 3- to 6-membered heterocycloalkyl.

R³ can be any suitable functional group known by one of skill in the art. In some embodiments, selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R³ is selected from hydrogen, halogen, —CN, optionally substituted C₃₋₄ carbocycle, and optionally substituted 3- to 4 membered heterocycloalkyl. In some embodiments, R³ is selected from hydrogen, fluoro, chloro, bromo, —CN, methyl, ethyl, propyl, butyl, cyclopropyl, cyclobutyl, optionally substituted oxetane, and optionally substituted azetidine. In some embodiments, R³ is selected from hydrogen, fluoro, —CN, cyclopropyl, cyclobutyl, optionally substituted oxetane, and optionally substituted azetidine. In some embodiments, R³ is selected from hydrogen, fluoro, and —CN. In some embodiments, R³ is selected from hydrogen and —CN.

R⁴ can be any suitable functional group known by one of skill in the art. In some embodiments, R⁴ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁴ is selected from hydrogen, —CN, optionally substituted methyl, optionally substituted ethyl, optionally substituted propyl, optionally substituted cyclopropyl, optionally substituted cyclobutyl, optionally substituted oxetane, and optionally substituted azetidine. In some embodiments, R⁴ is selected from hydrogen, —CN, —CHF₂, cyclopropyl, cyclobutyl, optionally substituted oxetane, and optionally substituted azetidine. R⁴ is selected from hydrogen, —CN, and —CHF₂.

R⁵ can be any suitable functional group known by one of skill in the art. In some embodiments, R⁵ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁵ is selected from hydrogen, halogen, —CN, optionally substituted C₃₋₄ carbocycle, and optionally substituted 3- to 4 membered heterocycloalkyl. In some embodiments, R⁵ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁵ is selected from hydrogen, fluoro, —CN, cyclopropyl, cyclobutyl, optionally substituted oxetane, and optionally substituted azetidine. In some embodiments, R⁵ is selected from hydrogen, methyl, ethyl, and propyl. In some embodiments, R⁵ is hydrogen.

R⁶ can be any suitable functional group known by one of skill in the art. In some embodiments, R⁶ is selected from hydrogen, halogen, —CN, optionally substituted C₁₋₄ alkyl, optionally substituted C₃₋₄ carbocycle, and optionally substituted 3- to 4-membered heterocycloalkyl. In some embodiments, R⁶ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁶ is selected from hydrogen, halogen, —CN, optionally substituted C₃₋₄ carbocycle, and optionally substituted 3- to 4 membered heterocycloalkyl. In some embodiments, R⁶ is selected from hydrogen, fluoro, —CN, cyclopropyl, cyclobutyl, optionally substituted oxetane, and optionally substituted azetidine. In some embodiments, R⁶ is selected from hydrogen, methyl, ethyl, and propyl. In some embodiments, R⁶ is hydrogen.

R⁷ can be any suitable functional group known by one of skill in the art. In some embodiments, R⁷ is selected from hydrogen, and optionally substituted C₁₋₄ alkyl. In some embodiments, R⁷ is selected from hydrogen, methyl, ethyl, and propyl. In some embodiments, R⁷ is hydrogen.

Each of X¹, X², and X³ can be any suitable atom known by one of skill in the art. In some embodiments, each of X¹, X², and X³ is independently selected from N and CR³. In some embodiments, each of X¹, X², and X³ is independently N. In some embodiments, each of X¹, X², and X³ is independently selected form CR¹³. In some embodiments, X¹, X², and X³ are each CH.

Z¹, Z², Z³, Z⁴ and Z⁵ are each independently any suitable atom known by one of skill in the art. In some embodiments, each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond. In some embodiments, each of Z¹, Z², Z³, Z⁴ and Z⁵ are independently selected from —C(R¹⁰)₂—, —NR¹¹—, —N(C(O)R¹⁰)—, —NS(O₂)R¹¹, —O—, and —S(O)₂—, wherein Z⁵ is additionally selected from a bond.

Variables a, b, c, and d can be any suitable number known by one of skill in the art. In some embodiments, each of a, b, c, and d are independently selected from 1, 2, 3 and 4. In some embodiments, each of a, b, c, and d are independently selected from 1, 2, and 3. In some embodiments, each of a, b, c, and d are independently selected from 1 and 2.

R¹⁴ can be any suitable functional group known by one of skill in the art. In some embodiments, R¹⁴ is selected from hydrogen, halogen, —CN, optionally substituted C₁₋₄ alkyl, optionally substituted C₃₋₆ carbocycle, and optionally substituted 3- to 6-membered heterocycloalkyl. In some embodiments, R¹⁴ is selected from fluoro, chlor, bromo, methyl, ethyl, and propyl.

R¹⁶ can be any suitable functional group known by one of skill in the art. In some embodiments, R¹⁶ is selected from optionally substituted C₁₋₄ alkyl, optionally substituted C₃₋₆ carbocycle, and optionally substituted 3- to 6-membered heterocycloalkyl. In some embodiments, R¹⁶ is selected form optionally substituted C₁₋₄alkyl. In some embodiments, R¹⁶ is methyl, ethyl, or propyl. In some embodiments, R¹⁶ is methyl.

In some embodiments, the compound is selected from:

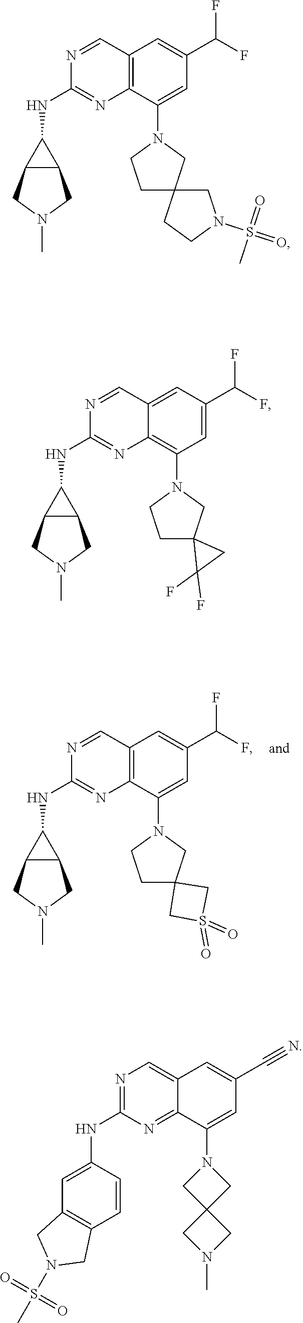 [SMILES: CN1C[C@@H]2[C@H](C1)[C@H]2Nc1ncc2cc(C(F)F)cc(N3CCC4(CCN(S(C)(=O)=O)C4)C3)c2n1 ; CN1C[C@@H]2[C@H](C1)[C@H]2Nc1ncc2cc(C(F)F)cc(N3CCC4(C3)CC4(F)F)c2n1 ; CN1C[C@@H]2[C@H](C1)[C@H]2Nc1ncc2cc(C(F)F)cc(N3CCC4(C3)CS(=O)(=O)C4)c2n1 ; "and" ; CN1CC2(C1)CN(c1cc(C#N)cc3cnc(Nc4ccc5c(c4)CN(S(C)(=O)=O)C5)nc13)C2]

In some embodiments the compound is selected from:

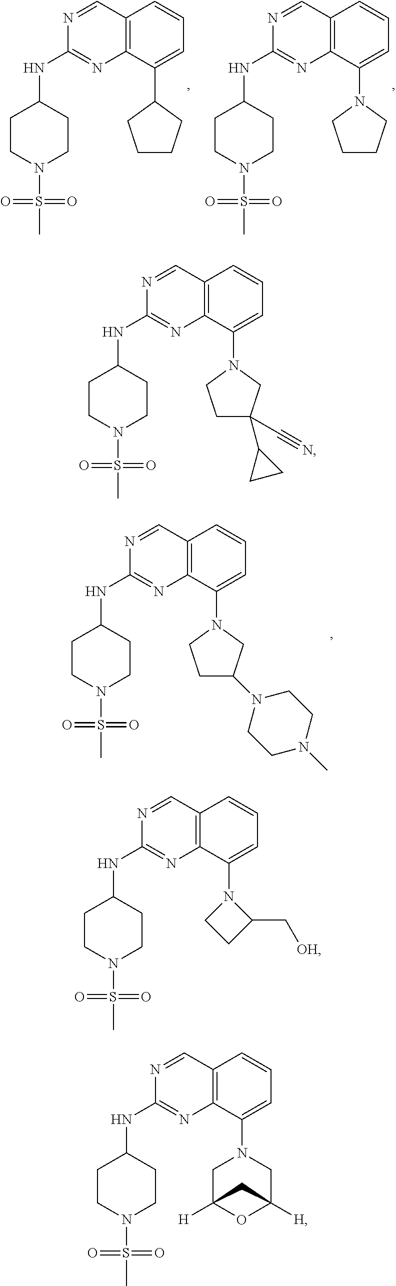

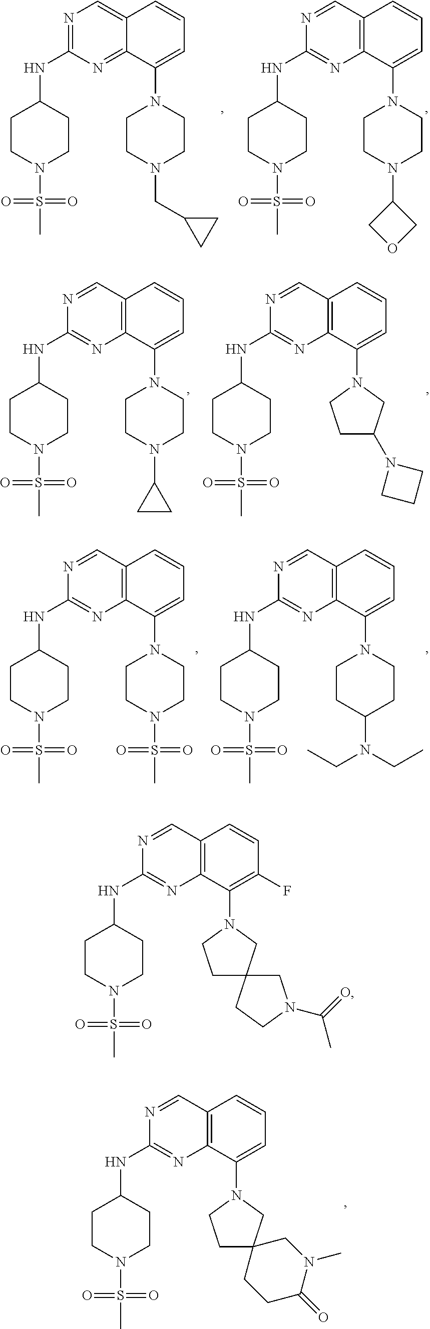

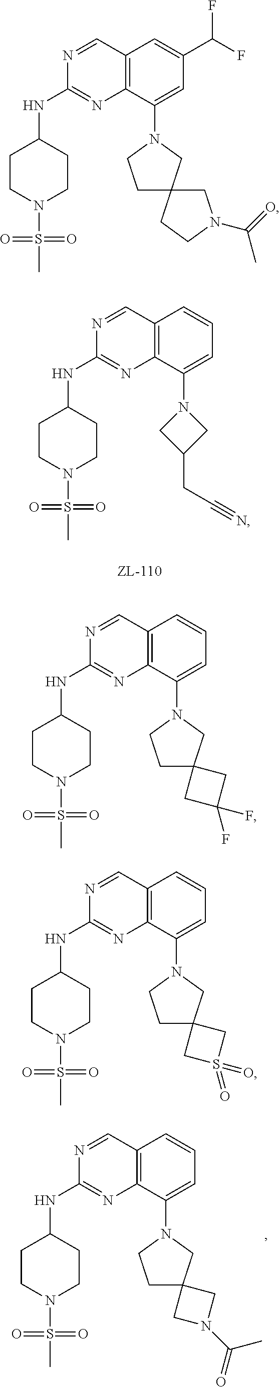 [SMILES: CC(=O)N1CCC2(CCN(c3cc(C(F)F)cc4cnc(NC5CCN(S(C)(=O)=O)CC5)nc34)C2)C1 ; CS(=O)(=O)N1CCC(Nc2ncc3cccc(N4CC(CC#N)C4)c3n2)CC1 ; CS(=O)(=O)N1CCC(Nc2ncc3cccc(N4CCC5(C4)CC(F)(F)C5)c3n2)CC1 ; CS(=O)(=O)N1CCC(Nc2ncc3cccc(N4CCC5(C4)CS(=O)(=O)C5)c3n2)CC1 ; CC(=O)N1CC2(CCN(c3cccc4cnc(NC5CCN(S(C)(=O)=O)CC5)nc34)C2)C1]

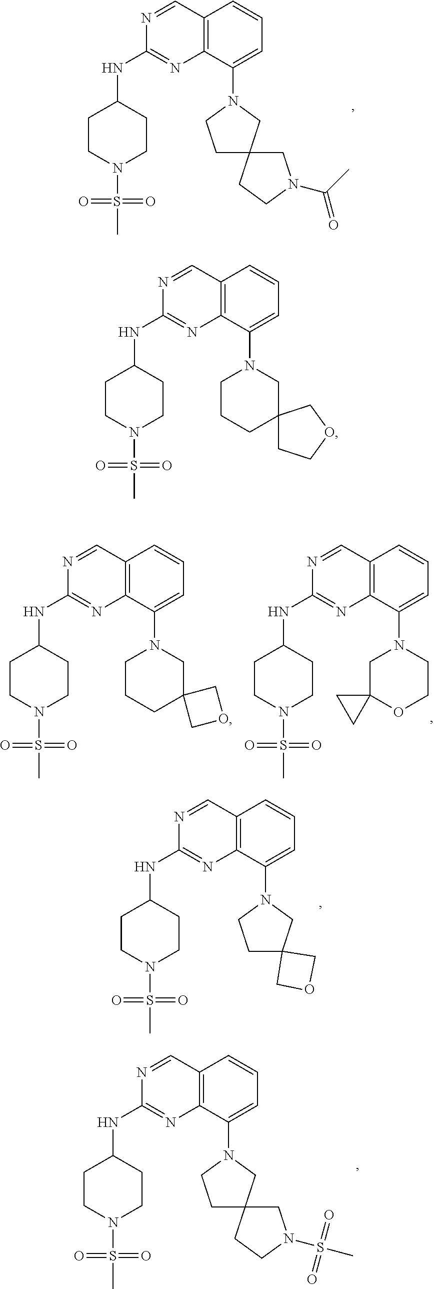

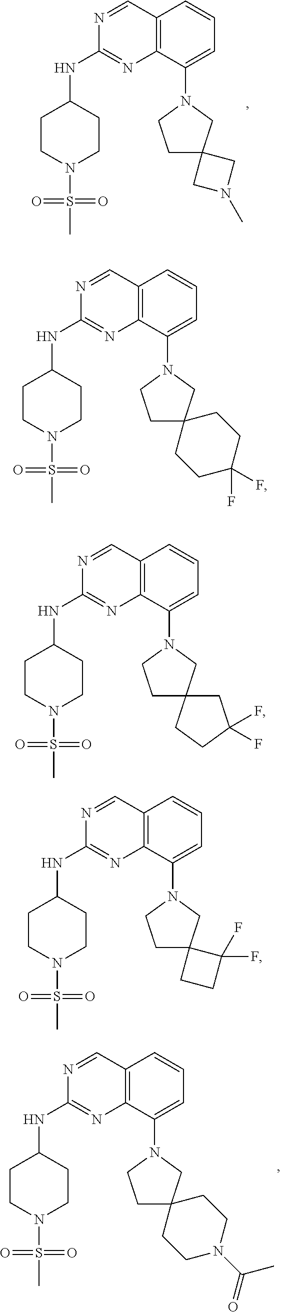

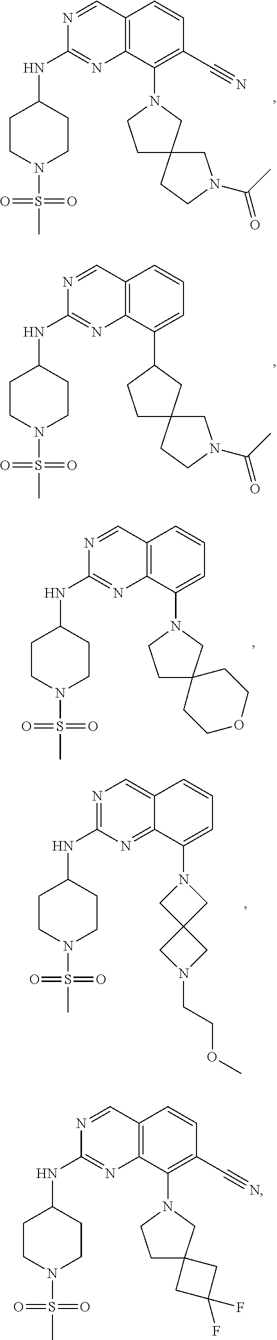 [SMILES: CC(=O)N1CCC2(CCN(c3c(C#N)ccc4cnc(NC5CCN(S(C)(=O)=O)CC5)nc34)C2)C1 ; CC(=O)N1CCC2(CCC(c3cccc4cnc(NC5CCN(S(C)(=O)=O)CC5)nc34)C2)C1 ; CS(=O)(=O)N1CCC(Nc2ncc3cccc(N4CCC5(CCOCC5)C4)c3n2)CC1 ; COCCN1CC2(C1)CN(c1cccc3cnc(NC4CCN(S(C)(=O)=O)CC4)nc13)C2 ; CS(=O)(=O)N1CCC(Nc2ncc3ccc(C#N)c(N4CCC5(C4)CC(F)(F)C5)c3n2)CC1]

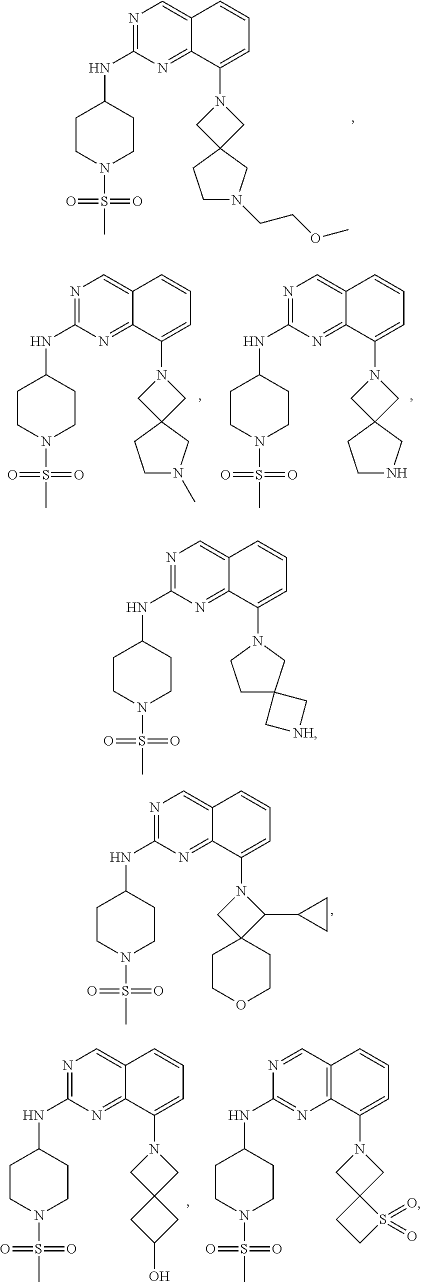

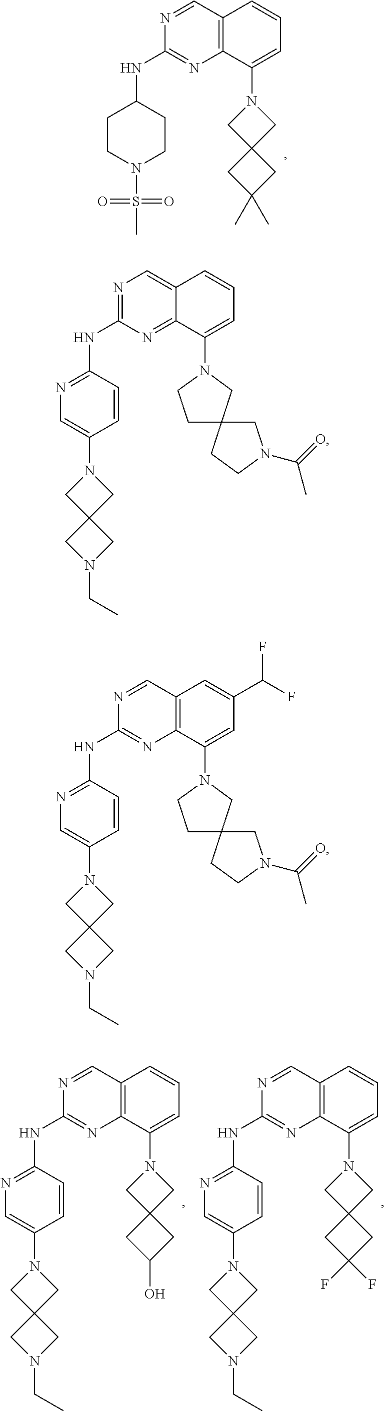

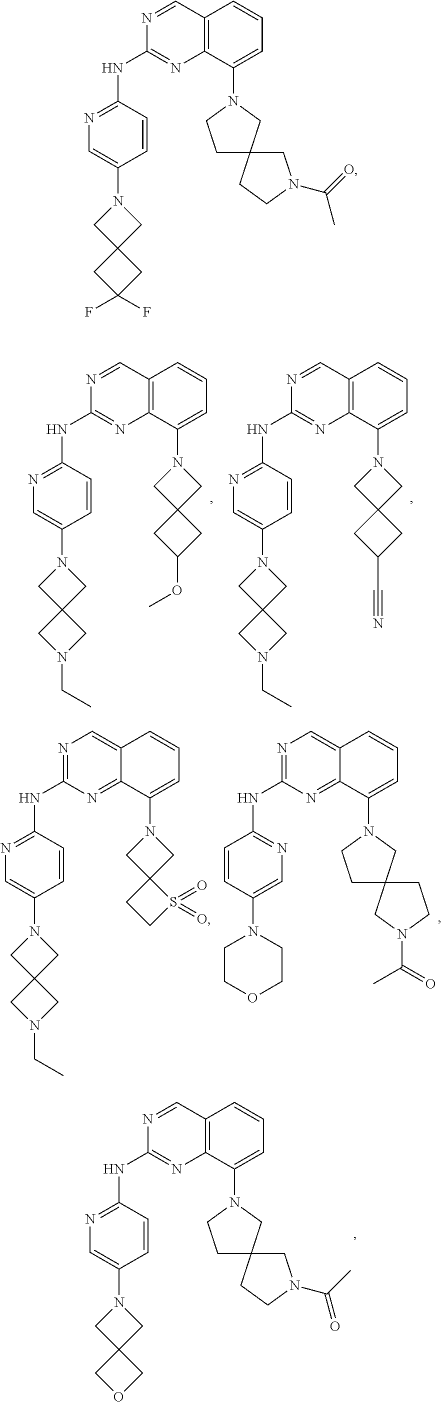

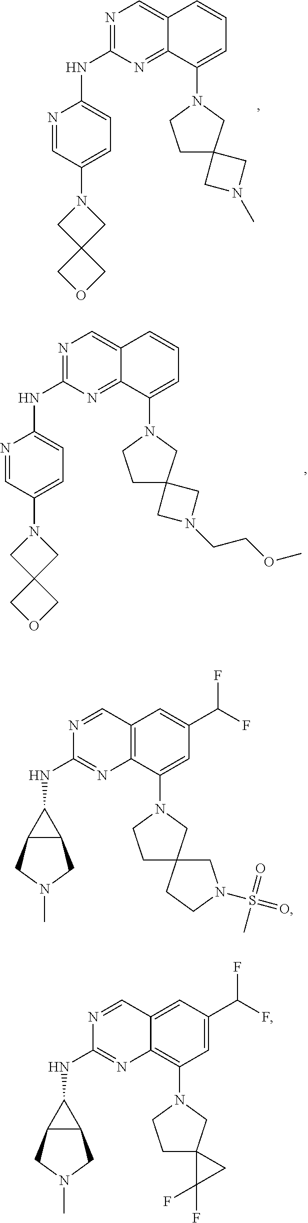

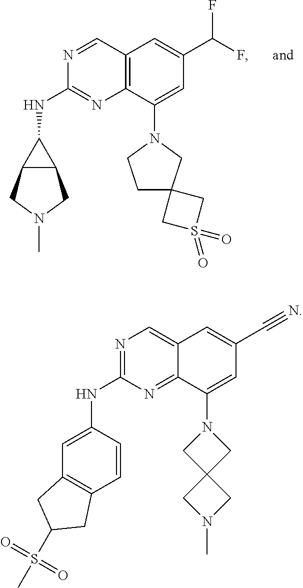 [SMILES: CN1C[C@@H]2[C@H](C1)[C@H]2Nc1ncc2cc(C(F)F)cc(N3CCC4(C3)CS(=O)(=O)C4)c2n1 ; CN1CC2(C1)CN(c1cc(C#N)cc3cnc(Nc4ccc5c(c4)CC(S(C)(=O)=O)C5)nc13)C2]

In some embodiments, the compound is selected from:

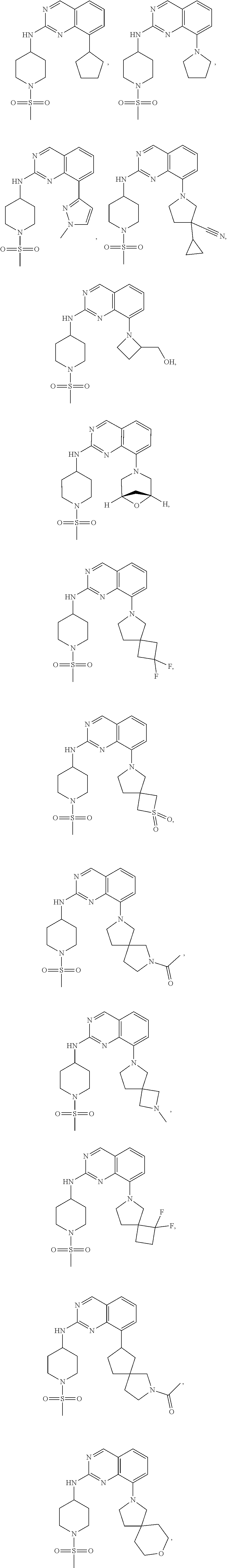

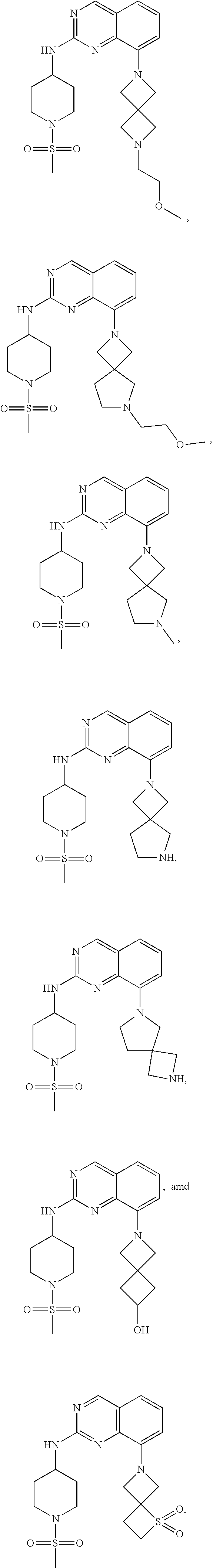

In one aspect, disclosed herein is a compound represented by Formula (II):

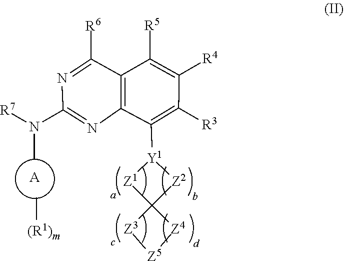

wherein,
A is a ring selected from optionally substituted C₃₋₆ carbocycle and optionally substituted 3- to 12-membered heterocycle;
   R¹ is selected from halogen, —CN, —NO₂, optionally substituted alkyl, optionally substituted alkenyl, optionally substituted alkynyl, optionally substituted carbocycle, and optionally substituted heterocycle;
m is selected from 0 to 9;
   each of R³, R⁴, R⁵, R⁶, is independently selected from hydrogen, halogen, —CN, and optionally substituted C₁₋₄ alkyl, optionally substituted C₃₋₄ carbocycle, and optionally substituted 3- to 4-membered heterocyclcoalkyl; and
R⁷ is selected from hydrogen and optionally substituted C₁₋₄ alkyl;
   Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle; and
each R¹¹ is independently selected from hydrogen and optionally substituted C₁₋₄ alkyl.

The A ring can by any suitable carbocycle and heterocycle known by one of skill in the art. In some embodiments, A is a ring selected from optionally substituted C₃₋₆ carbocycle and optionally substituted 3- to 12-membered heterocycle. In some embodiments, A is selected from optionally substituted C₅₋₆ carbocycle and optionally substituted 5- to 10-membered heterocycle. In some embodiments, A is selected from optionally substituted C₆ carbocycle and optionally substituted 5- to 10-membered heterocycle. In some embodiments, A is selected from phenyl, pyridine, pyrimidine, imidazole, pyrazole, tetrazole, thiazole, furan, pyran, tetrahydrofuran, dioxane, morpholine, piperidine, and tetrahydroisoquinoline. In some embodiments, A is selected from phenyl, pyridine, pyrimidine, imidazole, pyrazole, tetrazole, thiazole, furan, pyran, tetrahydrofuran, dioxane, morpholine, piperidine, and tetrahydroisoquinoline. In some embodiments, A is selected from phenyl, pyridine, pyrimidine, imidazole, pyrazole, furan, pyran, dioxane, morpholine, piperidine, and tetrahydroisoquinoline. In some embodiments, A is selected from phenyl, pyrazole, and tetrahydroisoquinoline.

Variable m can be any suitable number known by one of skill in the art. In some embodiments, m is selected from 0 to 9. In some embodiments, m is selected from 0 to 4. In some embodiments, m is selected from 0 to 2. In some embodiments, m is 1.

R¹ can be any suitable functional group known by one of skill in the art. R¹ can be any functional group as described previously herein. In some embodiments, R¹ is selected from halogen, —CN, optionally substituted alkyl, optionally substituted carbocycle, and optionally substituted heterocycle. In some embodiments, R¹ is selected from optionally substituted C₁₋₃ alkyl, and optionally substituted 5- to 8-membered heterocycle. In some embodiments, R¹ is selected from C₁₋₃ alkyl, and substituted 5- to 8-membered heterocycle. In some embodiments, R¹ is methyl, ethyl, propyl, isopropyl,

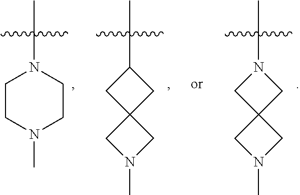

In some embodiments, R¹ is methyl, isopropyl,

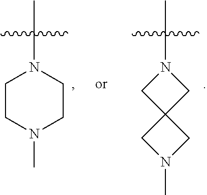

R³ can be any suitable functional group known by one of skill in the art. R³ can be any functional group as described previously herein. In some embodiments, R³ is selected from hydrogen, halogen, —CN, and optionally substituted C₁₋₄ alkyl, optionally substituted C₃₋₄ carbocycle, and optionally substituted 3- to 4-membered heterocyclcoalkyl. In some embodiments, R³ is selected from hydrogen, halogen, —CN, optionally substituted C₃₋₄ carbocycle, and optionally substituted 3- to 4 membered heterocycloalkyl. In some embodiments, R³ is selected from hydrogen, fluoro, chloro, bromo, —CN, methyl, ethyl, propyl, cyclopropyl, cyclobutyl, optionally substituted oxetane, and optionally substituted azetidine. In some embodiments, R³ is selected from hydrogen, fluoro, —CN, cyclopropyl, cyclobutyl, optionally substituted oxetane, and optionally substituted azetidine. In some embodiments, R³ is selected from hydrogen, fluoro, and —CN.

R⁴ can be any suitable functional group known by one of skill in the art. R⁴ can be any functional group as described previously herein. In some embodiments, R⁴ is selected from hydrogen, halogen, —CN, and optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, —CN, optionally substituted C₁ alkyl, optionally substituted C₃₋₄ carbocycle, and optionally substituted 3- to 4 membered heterocycloalkyl. In some embodiments, R⁴ is selected from hydrogen, fluoro, chloro, bromo, —CN, optionally substituted methyl, ethyl, propyl, cyclopropyl, cyclobutyl, optionally substituted oxetane, and optionally substituted azetidine. In some embodiments, R⁴ is selected from hydrogen, —CN, —CHF₂, cyclopropyl, cyclobutyl, optionally substituted oxetane, and optionally substituted azetidine. In some embodiments, R⁴ is selected from hydrogen, —CN, and —CHF₂.

R⁵ can be any suitable functional group known by one of skill in the art. R⁵ can be any functional group as described previously herein. In some embodiments, R⁵ selected from hydrogen, halogen, —CN, and optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, —CN, optionally substituted C₃₋₄ carbocycle, and optionally substituted 3- to 4 membered heterocycloalkyl. In some embodiments, R⁵ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁵ is selected from hydrogen, fluoro, —CN, cyclopropyl, cyclobutyl, optionally substituted oxetane, and optionally substituted azetidine. In some embodiments, R⁵ is hydrogen, fluoro, or —CN. In some embodiments, R⁵ is hydrogen.

R⁶ can be any suitable functional group known by one of skill in the art. R⁶ can be any functional group as described previously herein. In some embodiments, R⁶ is selected from hydrogen, halogen, —CN, and optionally substituted C₁₋₄ alkyl, optionally substituted C₃₋₄ carbocycle, and optionally substituted 3- to 4-membered heterocyclcoalkyl. In some embodiments, R⁵⁶ is selected from hydrogen, halogen, —CN, optionally substituted C₃₋₄ carbocycle, and optionally substituted 3- to 4 membered heterocycloalkyl. In some embodiments, R⁶ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁶ is selected from hydrogen, fluoro, —CN, cyclopropyl, cyclobutyl, optionally substituted oxetane, and optionally substituted azetidine. In some embodiments, R⁶ is hydrogen, fluoro, or —CN. In some embodiments, R⁶ is hydrogen.

R⁷ can be any suitable functional group known by one of skill in the art. R⁷ can be any functional group as described previously herein. In some embodiments, R⁷ is selected from hydrogen, and optionally substituted C₁₋₄ alkyl. In some embodiments, R⁷ is selected from hydrogen, methyl, ethyl, and propyl. In some embodiments, R⁷ is hydrogen.

Y¹ can be any suitable atom known by one of skill in the art. In some embodiments, Y¹ is selected rom —N— and —CR¹⁰—. In some embodiments, Y¹ is —N—. In some embodiments, Y¹ is —CR¹⁰—. Z¹, Z², Z³, Z⁴ and Z⁵ are each independently any suitable atom known by one of skill in the art. Z¹, Z², Z³, Z⁴ and Z⁵ can be any atom as described previously herein. In some embodiments, each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond. In some embodiments, each of Z¹, Z², Z³, Z⁴ and Z⁵ are independently selected from —C(R¹⁰)₂—, —NR¹¹—, —N(C(O)R¹⁰)—, —NS(O₂)R¹¹, —O—, and —S(O)₂—, wherein Z⁵ is additionally selected from a bond.

Variables a, b, c, and d can be any suitable number known by one of skill in the art. Variables a, b, c, and d can be any number as described previously herein. Variables a, b, c, and d can be any suitable number known by one of skill in the art. In some embodiments, each of a, b, c, and d are independently selected from 1, 2, 3 and 4. In some embodiments, each of a, b, c, and d are independently selected from 1, 2, and 3. In some embodiments, each of a, b, c, and d are independently selected from 1 and 2.

R¹⁰ can be any suitable functional group known by one of skill in the art. R¹⁰ can be any functional group as described previously herein. In some embodiments, each R¹⁰ is independently selected from hydrogen, halogen, —CN, —OH, —O—C₁₋₄ alkyl, and optionally substituted C₁₋₃ alkyl. In some embodiments, each R¹⁰ is independently selected from hydrogen, halogen, —CN, —OH, —OMe, —OEt, methyl, ethyl, propyl, and —CH₂CH₂OCH₃. In some embodiments, each R¹⁰ is independently selected from hydrogen, halogen, —CN, —OH, —OMe, methyl, and —CH₂CH₂OCH₃. In some embodiments, each R¹⁰ is independently selected from hydrogen, fluoro, —CN, —OH, —OMe, methyl, and —CH₂CH₂OCH₃.

R¹¹ can be any suitable functional group known by one of skill in the art. R¹¹ can be any functional group as described previously herein. In some embodiments, each R¹¹ is independently selected from hydrogen and optionally substituted C₁₋₂ alkyl. In some embodiments, each R¹¹ is independently selected from hydrogen, methyl, and ethyl, wherein the methyl and ethyl are optionally substituted with —OMe, —OEt, and —OPr. In some embodiments, each R¹¹ is independently selected from hydrogen, methyl, and ethyl, wherein the ethyl is optionally substituted with —OMe.

In some embodiments, the compound or pharmaceutically acceptable salt or solvate thereof, has the structure of Formula (IIA):

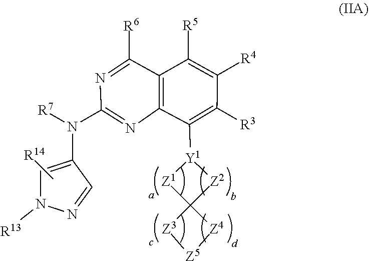

wherein,
   R³ is selected from optionally substituted C₁₋₄ alkyl, optionally substituted C₃₋₆ carbocycle, and optionally substituted 3- to 6-membered heterocycloalkyl, or R³ and R¹⁴ come together to form an optionally substituted heterocycle; and
   R¹⁴ is selected from halogen, —CN, optionally substituted C₁₋₄ alkyl, optionally substituted C₃₋₆ carbocycle, and optionally substituted 3- to 6-membered heterocycloalkyl.

R³ can be any suitable functional group known by one of skill in the art. R³ can be any functional group as described previously herein. In some embodiments, R³ is selected from hydrogen, halogen, —CN, and optionally substituted C₁₋₄ alkyl, optionally substituted C₃₋₄ carbocycle, and optionally substituted 3- to 4-membered heterocyclcoalkyl. In some embodiments, R³ is selected from hydrogen, halogen, —CN, optionally substituted C₃₋₄ carbocycle, and optionally substituted 3- to 4 membered heterocycloalkyl. In some embodiments, R³ is selected from hydrogen, fluoro, chloro, bromo, —CN, methyl, ethyl, propyl, cyclopropyl, cyclobutyl, optionally substituted oxetane, and optionally substituted azetidine. In some embodiments, R³ is selected from hydrogen, fluoro, —CN, cyclopropyl, cyclobutyl, optionally substituted oxetane, and optionally substituted azetidine. In some embodiments, R³ is selected from hydrogen, fluoro, and —CN.

R⁴ can be any suitable functional group known by one of skill in the art. R⁴ can be any functional group as described previously herein. In some embodiments, R⁴ is selected from hydrogen, halogen, —CN, and optionally substituted C₁₋₄ alkyl, optionally substituted C₃₋₄ carbocycle, and optionally substituted 3- to 4-membered heterocyclcoalkyl. In some embodiments, R⁴ is selected from hydrogen, halogen, —CN, optionally substituted C₁ alkyl, optionally substituted C₃₋₄ carbocycle, and optionally substituted 3- to 4 membered heterocycloalkyl. In some embodiments, R⁴ is selected from hydrogen, fluoro, chloro, bromo, —CN, optionally substituted methyl, ethyl, propyl, cyclopropyl, cyclobutyl, optionally substituted oxetane, and optionally substituted azetidine. In some embodiments, R⁴ is selected from hydrogen, —CN, —CHF₂, cyclopropyl, cyclobutyl, optionally substituted oxetane, and optionally substituted azetidine. In some embodiments, R⁴ is selected from hydrogen, —CN, and —CHF₂.

R⁵ can be any suitable functional group known by one of skill in the art. R⁵ can be any functional group as described previously herein. In some embodiments, R⁵ selected from hydrogen, halogen, —CN, and optionally substituted C₁₋₄ alkyl, optionally substituted C₃₋₄ carbocycle, and optionally substituted 3- to 4-membered heterocyclcoalkyl. In some embodiments, R⁵ is selected from hydrogen, halogen, —CN, optionally substituted C₃₋₄ carbocycle, and optionally substituted 3- to 4 membered heterocycloalkyl. In some embodiments, R⁵ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁵ is selected from hydrogen, fluoro, —CN, cyclopropyl, cyclobutyl, optionally substituted oxetane, and optionally substituted azetidine. In some embodiments, R⁵ is hydrogen, fluoro, or —CN. In some embodiments, R⁵ is hydrogen.

R⁶ can be any suitable functional group known by one of skill in the art. R⁶ can be any functional group as described previously herein. In some embodiments, R⁶ is selected from hydrogen, halogen, —CN, and optionally substituted C₁₋₄ alkyl, optionally substituted C₃₋₄ carbocycle, and optionally substituted 3- to 4-membered heterocyclcoalkyl. In some embodiments, R⁵⁶ is selected from hydrogen, halogen, —CN, optionally substituted C₃₋₄ carbocycle, and optionally substituted 3- to 4 membered heterocycloalkyl. In some embodiments, R⁶ is selected from hydrogen, fluoro, chloro, bromo, —CN, methyl, ethyl, propyl, cyclopropyl, cyclobutyl, optionally substituted oxetane, and optionally substituted azetidine. In some embodiments, R⁶ is selected from hydrogen, fluoro, —CN, cyclopropyl, cyclobutyl, optionally substituted oxetane, and optionally substituted azetidine. In some embodiments, R⁶ is hydrogen, fluoro, or —CN. In some embodiments, R⁶ is hydrogen.

R⁷ can be any suitable functional group known by one of skill in the art. R⁷ can be any functional group as described previously herein. In some embodiments, R⁷ is selected from hydrogen, and optionally substituted C₁₋₄ alkyl. In some embodiments, R⁷ is selected from hydrogen, methyl, ethyl, and propyl. In some embodiments, R⁷ is hydrogen.

Y¹ can be any suitable atom known by one of skill in the art. In some embodiments, Y¹ is selected rom —N— and —CR¹⁰—. In some embodiments, Y¹ is —N—. In some embodiments, Y¹ is —CR¹⁰—. Z¹, Z², Z³, Z⁴ and Z⁵ are each independently any suitable atom known by one of skill in the art. Z¹, Z², Z³, Z⁴ and Z⁵ can be any atom as described previously herein. In some embodiments, each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond. In some embodiments, each of Z¹, Z², Z³, Z⁴ and Z⁵ are independently selected from —C(R¹⁰)₂—, —NR¹¹—, —N(C(O)R¹⁰)—, —NS(O₂)R¹¹, —O—, and —S(O)₂—, wherein Z⁵ is additionally selected from a bond.

Variables a, b, c, and d can be any suitable number known by one of skill in the art. Variables a, b, c, and d can be any number as described previously herein. Variables a, b, c, and d can be any suitable number known by one of skill in the art. In some embodiments, each of a, b, c, and d are independently selected from 1, 2, 3 and 4. In some embodiments, each of a, b, c, and d are independently selected from 1, 2, and 3. In some embodiments, each of a, b, c, and d are independently selected from 1 and 2.

R¹⁰ can be any suitable functional group known by one of skill in the art. R¹⁰ can be any functional group as described previously herein. In some embodiments, each R¹⁰ is independently selected from hydrogen, halogen, —CN, —OH, —O—C₁₋₄ alkyl, and optionally substituted C₁₋₃ alkyl. In some embodiments, each R¹⁰ is independently selected from hydrogen, halogen, —CN, —OH, —OMe, —OEt, methyl, ethyl, propyl, and —CH₂CH₂OCH₃. In some embodiments, each R¹⁰ is independently selected from hydrogen, halogen, —CN, —OH, —OMe, methyl, and —CH₂CH₂OCH₃. In some embodiments, each R¹⁰ is independently selected from hydrogen, fluoro, —CN, —OH, —OMe, methyl, and —CH₂CH₂OCH₃.

R¹¹ can be any suitable functional group known by one of skill in the art. R¹¹ can be any functional group as described previously herein. In some embodiments, each R¹¹ is independently selected from hydrogen and optionally substituted C₁₋₂ alkyl. In some embodiments, each R¹¹ is independently selected from hydrogen, methyl, and ethyl, wherein the methyl and ethyl are optionally substituted with —OMe, —OEt, and —OPr. In some embodiments, each R¹¹ is independently selected from hydrogen, methyl, and ethyl, wherein the ethyl is optionally substituted with —OMe.

R¹³ can be any suitable functional group known by one of skill in the art. In some embodiments, R¹³ is selected from optionally substituted C₁₋₄ alkyl, optionally substituted C₃₋₆ carbocycle, and optionally substituted 3- to 6-membered heterocycloalkyl, or R³ and R¹⁴ come together to form an optionally substituted heterocycle. In some embodiments, R³ is selected from optionally substituted C₁₋₄ alkyl. In some embodiments, R¹³ is selected from optionally substituted C₃₋₆ carbocycle and optionally substituted 3- to 6-membered heterocycloalkyl. In some embodiments, R¹³ is selected from optionally substituted C₃₋₆ carbocycle. In some embodiments, R³ is selected from optionally substituted 3- to 6-membered heterocycloalkyl. In some embodiments, R¹³ and R¹⁴ come together to form an optionally substituted heterocycle.

R¹⁴ can be any suitable functional group known by one of skill in the art. In some embodiments, R¹⁴ is selected from halogen, —CN, optionally substituted C₁₋₄ alkyl, optionally substituted C₃₋₆ carbocycle, and optionally substituted 3- to 6-membered heterocycloalky, or R¹⁴ and R¹³ come together to form an optionally substituted heterocycle. In some embodiments, R¹⁴ is selected from halogen, —CN, and optionally substituted C₁₋₄ alkyl. In some embodiments, R¹⁴ is selected from optionally substituted C₃₋₆ carbocycle and optionally substituted 3- to 6-membered heterocycloalky. In some embodiments, R¹⁴ and R¹³ come together to form an optionally substituted heterocycle.

In some embodiments, the compound is selected from:

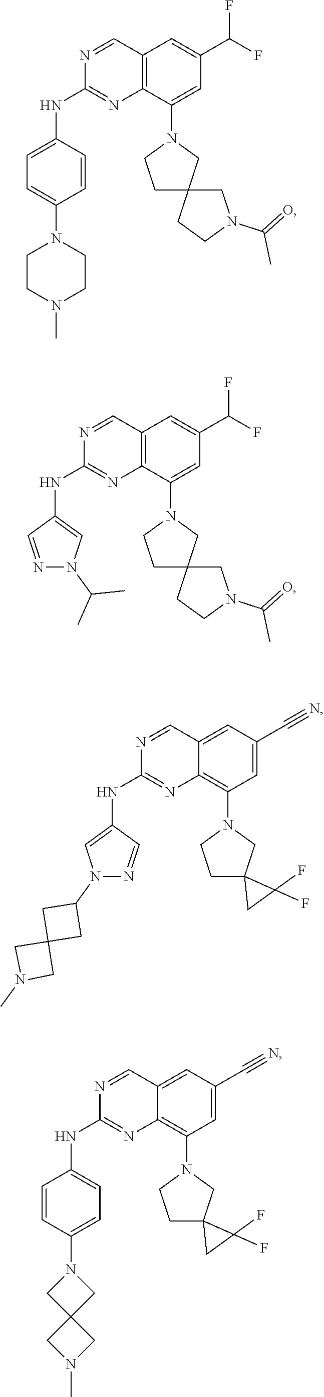 [SMILES: CC(=O)N1CCC2(CCN(c3cc(C(F)F)cc4cnc(Nc5ccc(N6CCN(C)CC6)cc5)nc34)C2)C1 ; CC(=O)N1CCC2(CCN(c3cc(C(F)F)cc4cnc(Nc5cnn(C(C)C)c5)nc34)C2)C1 ; CN1CC2(CC(n3cc(Nc4ncc5cc(C#N)cc(N6CCC7(C6)CC7(F)F)c5n4)cn3)C2)C1 ; CN1CC2(C1)CN(c1ccc(Nc3ncc4cc(C#N)cc(N5CCC6(C5)CC6(F)F)c4n3)cc1)C2]

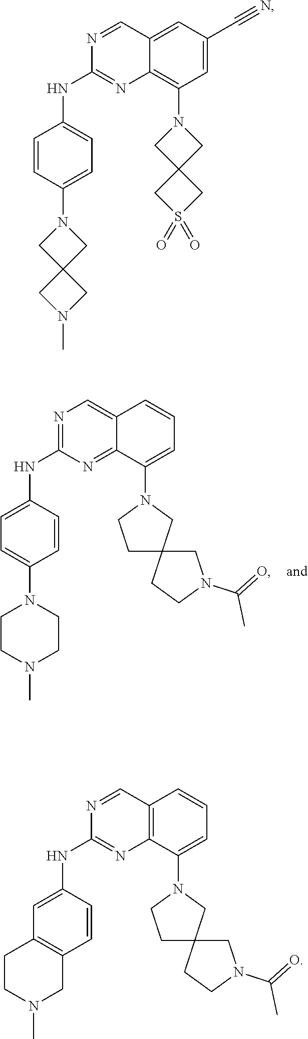 [SMILES: CN1CC2(C1)CN(c1ccc(Nc3ncc4cc(C#N)cc(N5CC6(C5)CS(=O)(=O)C6)c4n3)cc1)C2 ; CC(=O)N1CCC2(CCN(c3cccc4cnc(Nc5ccc(N6CCN(C)CC6)cc5)nc34)C2)C1 ; "and" ; CC(=O)N1CCC2(CCN(c3cccc4cnc(Nc5ccc6c(c5)CCN(C)C6)nc34)C2)C1]

In some embodiments, the compound is selected from:

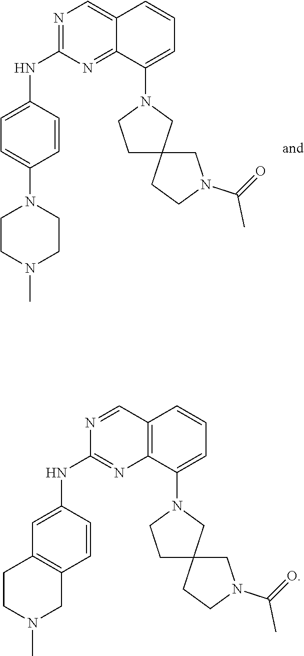 [SMILES: CC(=O)N1CCC2(CCN(c3cccc4cnc(Nc5ccc(N6CCN(C)CC6)cc5)nc34)C2)C1 ; "and" ; CC(=O)N1CCC2(CCN(c3cccc4cnc(Nc5ccc6c(c5)CCN(C)C6)nc34)C2)C1]

Additional embodiments of the compounds of this disclosure include the following:

Embodiment 1 of this disclosure relates to a compound, or a pharmaceutically acceptable salt or solvate thereof, having the structure of Formula (I):

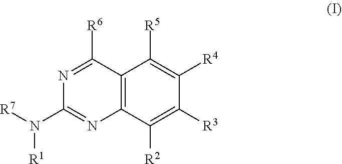

wherein,
R¹ is selected from

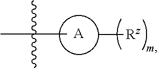

optionally substituted piperidine, optionally substituted phenyl, optionally substituted pyrazole, optionally substituted indazole, optionally substituted tetrahydroisoquinoline, optionally substituted of pyrrolpyrimidine, optionally substituted tetrahydroisoquinoline, optionally substituted 2-pyridine, optionally substituted azabicyclo[3.1.0]hexane, optionally substituted indole, optionally substituted isoindole, and optionally substituted azetidine;
A is a ring selected from optionally substituted C₃₋₆ carbocycle and optionally substituted 3- to 12-membered heterocycle;
R² is selected from optionally substituted cycloalkyl and optionally substituted heterocycle;
each of R³, R⁴, R⁵, R⁶, is independently selected from hydrogen, halogen, —CN, optionally substituted C₁₋₄ alkyl, optionally substituted C₃₋₄ carbocycle, and optionally substituted 3- to 4-membered heterocycloalkyl;
R⁷ is selected from hydrogen and optionally substituted C₁₋₄ alkyl
R^{z} is selected from halogen, —CN, —NO₂, optionally substituted alkyl, optionally substituted alkenyl, optionally substituted alkynyl, optionally substituted carbocycle, and optionally substituted heterocycle; and
m is selected from 0 to 9.

Embodiment 2 of this disclosure relates to the compound, or a pharmaceutically acceptable salt or solvate thereof, of Embodiment 1, wherein R¹ is selected from, optionally substituted piperidine, optionally substituted phenyl, optionally substituted pyrazole, optionally substituted indazole, optionally substituted tetrahydroisoquinoline, optionally substituted of pyrrolpyrimidine, optionally substituted tetrahydroisoquinoline, optionally substituted 2-pyridine, optionally substituted azabicyclo[3.1.0]hexane, optionally substituted indole, optionally substituted isoindole, and optionally substituted azetidine.

Embodiment 3 of this disclosure relates to the compound, or a pharmaceutically acceptable salt or solvate thereof, of Embodiment 1, wherein R¹ is

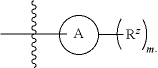

Embodiment 4 of this disclosure relates to the compound, or a pharmaceutically acceptable salt or solvate thereof, of Embodiment 3, wherein A is selected from optionally substituted C₅₋₆ carbocycle and optionally substituted 5- to 10-membered heterocycle.

Embodiment 5 of this disclosure relates to the compound, or a pharmaceutically acceptable salt or solvate thereof, of Embodiment 4, wherein A is selected from phenyl, pyrazole, and tetrahydroisoquinoline.

Embodiment 6 of this disclosure relates to the compound, or a pharmaceutically acceptable salt or solvate thereof, of any one of Embodiments 3 to 5, wherein m is selected from 0 to 4.

Embodiment 7 of this disclosure relates to the compound, or a pharmaceutically acceptable salt or solvate thereof, of Embodiment 6, wherein m is selected from 0 to 2.

Embodiment 8 of this disclosure relates to the compound, or a pharmaceutically acceptable salt or solvate thereof, of any one of Embodiments 3 to 7, wherein R^{z} is selected from halogen, —CN, optionally substituted alkyl, optionally substituted carbocycle, and optionally substituted heterocycle.

Embodiment 9 of this disclosure relates to the compound, or a pharmaceutically acceptable salt or solvate thereof, of Embodiment 8, wherein R^{z} is selected from optionally substituted C₁₋₃ alkyl, and optionally substituted 5- to 8-membered heterocycle.

Embodiment 10 of this disclosure relates to the compound, or a pharmaceutically acceptable salt or solvate thereof, of Embodiment 9, wherein R^{z} is selected from C₁₋₃ alkyl, and substituted 5- to 8-membered heterocycle.

Embodiment 11 of this disclosure relates to the compound, or a pharmaceutically acceptable salt or solvate thereof, of any one of the preceding Embodiments, wherein R² is

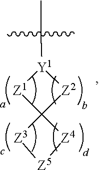

Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle; and
each R¹¹ is independently selected from hydrogen and optionally substituted C₁₋₄ alkyl.

Embodiment 12 of this disclosure relates to the compound, or a pharmaceutically acceptable salt or solvate thereof, of Embodiment 1 or 2 having the structure of one or more of the following Formulae:

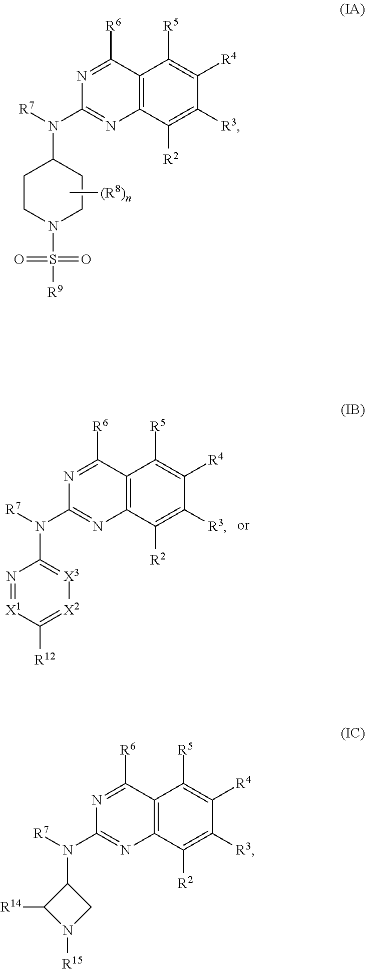

wherein,
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl;
n is selected from 0 to 9;
each of X¹, X², and X³ is independently selected from N and CR³;
R¹² is selected from hydrogen, halogen, —CN, —NO₂, optionally substituted alkyl, optionally substituted alkenyl, optionally substituted alkynyl, optionally substituted carbocycle, and optionally substituted heterocycle, or R² comes together with R¹³ to form an optionally substituted ring; and
each R¹³ is independently selected from hydrogen, halogen, —CN, and optionally substituted C₁₋₄ alkyl.
   R¹⁴ is selected from hydrogen, halogen, —CN, optionally substituted C₁₋₄ alkyl, optionally substituted C₃₋₆ carbocycle, and optionally substituted 3- to 6-membered heterocycloalkyl, or R⁴ and R⁵ come together to form an optionally substituted heterocycle; and
R¹⁵ is selected from —S(O)₂R¹⁶—, optionally substituted C₁₋₄ alkyl, optionally substituted C₃₋₆ carbocycle, and optionally substituted 3- to 6-membered heterocycloalkyl.

Embodiment 12(a) of this disclosure relates to the compound, or a pharmaceutically acceptable salt or solvate thereof, of Embodiments 12 having the structure of Formula (IA).

Embodiment 12(b) of this disclosure relates to the compound, or a pharmaceutically acceptable salt or solvate thereof, of Embodiments 12 having the structure of Formula (IB).

Embodiment 12(c) of this disclosure relates to the compound, or a pharmaceutically acceptable salt or solvate thereof, of Embodiments 12 having the structure of Formula (IC).

Embodiment 13 of this disclosure relates to the compound, or a pharmaceutically acceptable salt or solvate thereof, of any one of Embodiments 12, 12(a), 12(b), or 12(c), wherein R² is optionally substituted heterocycle.

Embodiment 14 of this disclosure relates to the compound, or a pharmaceutically acceptable salt or solvate thereof, of any one of Embodiments 12, 12(a), 12(b), or 12(c), wherein R² is selected from optionally substituted C₃₋₆ cycloalkyl, optionally substituted pyrrolidine, optionally substituted piperidine, optionally substituted pyrazole, optionally substituted azetidine, optionally substituted oxetane, and optionally substituted morpholine.

Embodiment 15 of this disclosure relates to the compound, or a pharmaceutically acceptable salt or solvate thereof, of Embodiment 13 or 14, wherein R² is substituted with —CN, —SO₂R^{2a}, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, or azetidine, wherein R^{2a} is selected from C₁₋₆ alkyl.

Embodiment 16 of this disclosure relates to the compound, or a pharmaceutically acceptable salt or solvate thereof, of Embodiment 1 having the structure of one or more of the following Formulae:

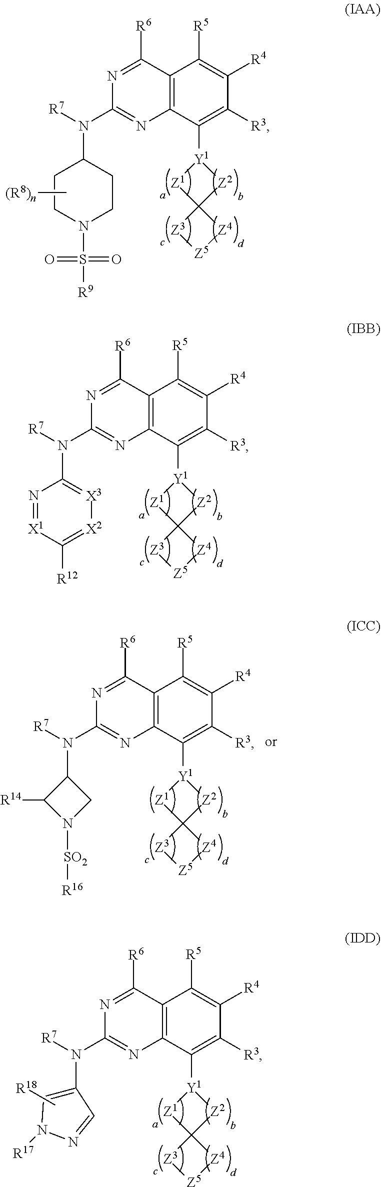

wherein,
Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl;
n is selected from 0 to 9;
   X¹, X², and X³ are each CH;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle;
each R¹¹ is independently selected from hydrogen and optionally substituted C₁₋₄ alkyl;
R¹⁶ is selected from optionally substituted C₁₋₄ alkyl, optionally substituted C₃₋₆ carbocycle, and optionally substituted 3- to 6-membered heterocycloalkyl;
R¹⁷ is selected from optionally substituted C₁₋₄ alkyl, optionally substituted C₃₋₆ carbocycle, and optionally substituted 3- to 6-membered heterocycloalkyl, or R³ and R¹⁴ come together to form an optionally substituted heterocycle; and
R¹⁸ is selected from halogen, —CN, optionally substituted C₁₋₄ alkyl, optionally substituted C₃₋₆ carbocycle, and optionally substituted 3- to 6-membered heterocycloalkyl.

Embodiment 16(a) of this disclosure relates to the compound, or a pharmaceutically acceptable salt or solvate thereof, of Embodiments 16 having the structure of Formula (IAA).

Embodiment 16(b) of this disclosure relates to the compound, or a pharmaceutically acceptable salt or solvate thereof, of Embodiments 16 having the structure of Formula (IBB).

Embodiment 16(c) of this disclosure relates to the compound, or a pharmaceutically acceptable salt or solvate thereof, of Embodiments 16 having the structure of Formula (ICC).

Embodiment 16(d) of this disclosure relates to the compound, or a pharmaceutically acceptable salt or solvate thereof, of Embodiments 16 having the structure of Formula (IDD).

Embodiment 17 of this disclosure relates to the compound, or a pharmaceutically acceptable salt or solvate thereof, of any one of Embodiments 11, 16, 16(a), 16(b), 16(c), or 16(d), wherein Y¹ is —N—.

Embodiment 18 of this disclosure relates to the compound, or a pharmaceutically acceptable salt or solvate thereof, of any one of Embodiments 11, 16, 16(a), 16(b), 16(c), or 16(d), wherein Y¹ is —CR¹⁰—.

Embodiment 19 of this disclosure relates to the compound, or a pharmaceutically acceptable salt or solvate thereof, of any one of Embodiments 11, 16, 16(a), 16(b), 16(c), or 16(d), wherein each of Z¹, Z², Z³, Z⁴ and Z⁵ are independently selected from —C(R¹⁰)₂—, —NR¹¹—, —N(C(O)R¹⁰)—, —NS(O₂)R¹¹, —O—, and —S(O)₂—, wherein Z⁵ is additionally selected from a bond.

Embodiment 20 of this disclosure relates to the compound, or a pharmaceutically acceptable salt or solvate thereof, of any one of Embodiments 11, 16, 16(a), 16(b), 16(c), 16(d), 17, 18 or 19, wherein each of a, b, c, and d are independently selected from 1, 2, and 3.

Embodiment 21 of this disclosure relates to the compound, or a pharmaceutically acceptable salt or solvate thereof, of Embodiment 20, wherein each of a, c, and d are independently selected from 1 and 2.

Embodiment 22 of this disclosure relates to the compound, or a pharmaceutically acceptable salt or solvate thereof, of any one of Embodiments 11, 16, 16(a), 16(b), 16(c), 16(d), 17, 18, 19, 20 or 21, wherein each R¹⁰ is independently selected from hydrogen, halogen, —CN, —OH, —O—C₁₋₄ alkyl, optionally substituted C₁₋₃ alkyl, and optionally substituted C₃₋₆ cycloalkyl.

Embodiment 23 of this disclosure relates to the compound, or a pharmaceutically acceptable salt or solvate thereof, of Embodiment 22, wherein each R¹⁰ is independently selected from hydrogen, halogen, —CN, —OH, methyl, —OMe, —CH₂CH₂OCH₃., and cyclopropyl.

Embodiment 24 of this disclosure relates to the compound, or a pharmaceutically acceptable salt or solvate thereof, of any one of Embodiments 11, 16, 16(a), 16(b), 16(c), 16(d), 17, 18, 19, 20, 21, 22 or 23, wherein each R¹¹ is independently selected from hydrogen and optionally substituted C₁₋₂ alkyl.

Embodiment 25 of this disclosure relates to the compound, or a pharmaceutically acceptable salt or solvate thereof, of Embodiment 24, wherein each R¹¹ is independently selected from hydrogen, methyl, and ethyl, wherein the ethyl is optionally substituted with —OMe.

Embodiment 26 of this disclosure relates to the compound, or a pharmaceutically acceptable salt or solvate thereof, of Embodiment 1, wherein R¹ is selected from optionally substituted azabicyclo[3.1.0]hexane, optionally substituted isoindole, and optionally substituted indole.

Embodiment 27 of this disclosure relates to the compound, or a pharmaceutically acceptable salt or solvate thereof, of Embodiment 26, wherein R¹ is selected from optionally substituted azabicyclo[3.1.0]hexane, and optionally substituted isoindole.

Embodiment 28 of this disclosure relates to the compound, or a pharmaceutically acceptable salt or solvate thereof, of Embodiment 26 or 27, wherein R¹ is substituted with —SO₂R^{1a} or C₁₋₃ alkyl, wherein R^{1a} is selected from C₁₋₆ alkyl.

Embodiment 29 of this disclosure relates to the compound, or a pharmaceutically acceptable salt or solvate thereof, of any one of Embodiments 1, 2, 26, 27 or 28, wherein R² is selected from optionally substituted heterocycle and optionally substituted cycloalkyl.

Embodiment 30 of this disclosure relates to the compound, or a pharmaceutically acceptable salt or solvate thereof, of Embodiment 29, wherein R² is optionally substituted heterocycloalkyl.

Embodiment 31 of this disclosure relates to the compound, or a pharmaceutically acceptable salt or solvate thereof, of Embodiment 30, wherein R² is selected from optionally substituted 3- to 6-membered heterocycloalkyl.

Embodiment 32 of this disclosure relates to the compound, or a pharmaceutically acceptable salt or solvate thereof, of Embodiment 31, wherein R² is selected from optionally substituted azetidine, optionally substituted pyrrolidine, optionally substituted piperidine, optionally substituted piperazine, and optionally substituted morpholine.

Embodiment 33 of this disclosure relates to the compound, or a pharmaceutically acceptable salt or solvate thereof, of any one of Embodiments 29 to 32, wherein R² is substituted with halogen, —SO₂R^{2a}, —NR^{2a}, —C(O)CH₃, —CN, optionally substituted 3- to 6-membered heterocycloalkyl, optionally substituted C₃₋₅ carbocycle, oxo, and optionally substituted C₁₋₃ alkyl, wherein R² is selected from C₁₋₆ alkyl.

Embodiment 34 of this disclosure relates to the compound, or a pharmaceutically acceptable salt or solvate thereof, of Embodiment 19, wherein R² is substituted with fluoro, —SO₂Me, oxo, and methyl.

Embodiment 35 of this disclosure relates to the compound, or a pharmaceutically acceptable salt or solvate thereof, of any one of Embodiments 1-10, 12, 12(a), 12(b), 12(c), or 29, wherein R² is selected from

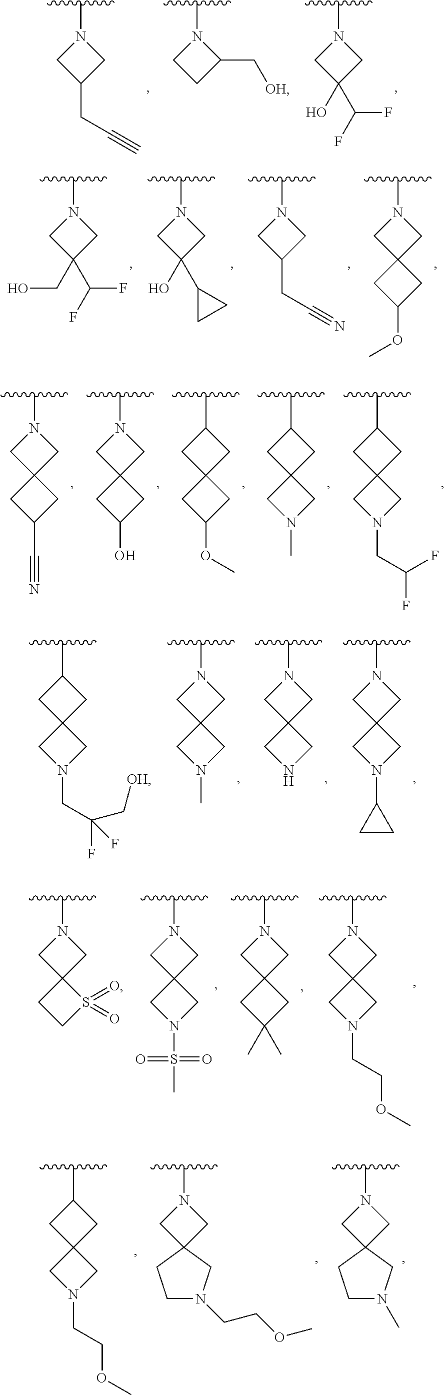

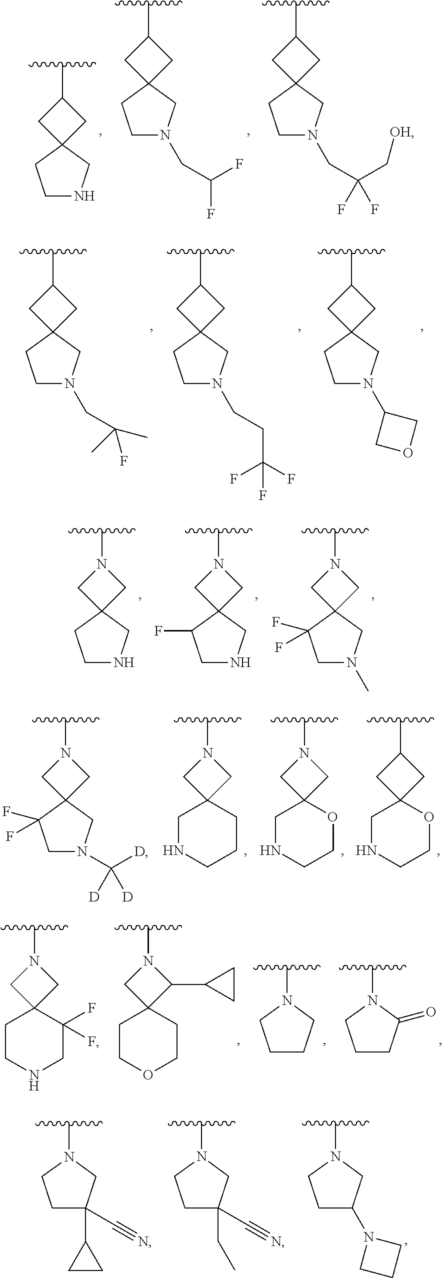

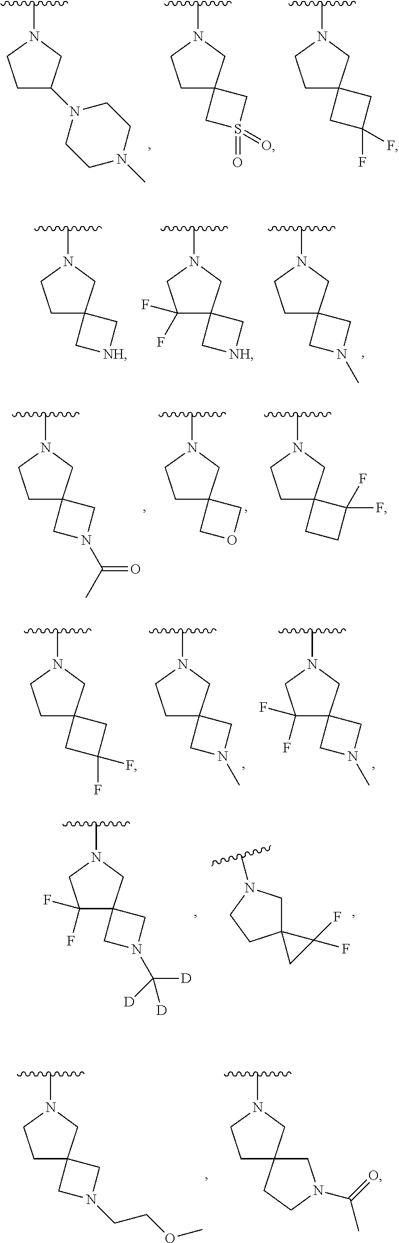

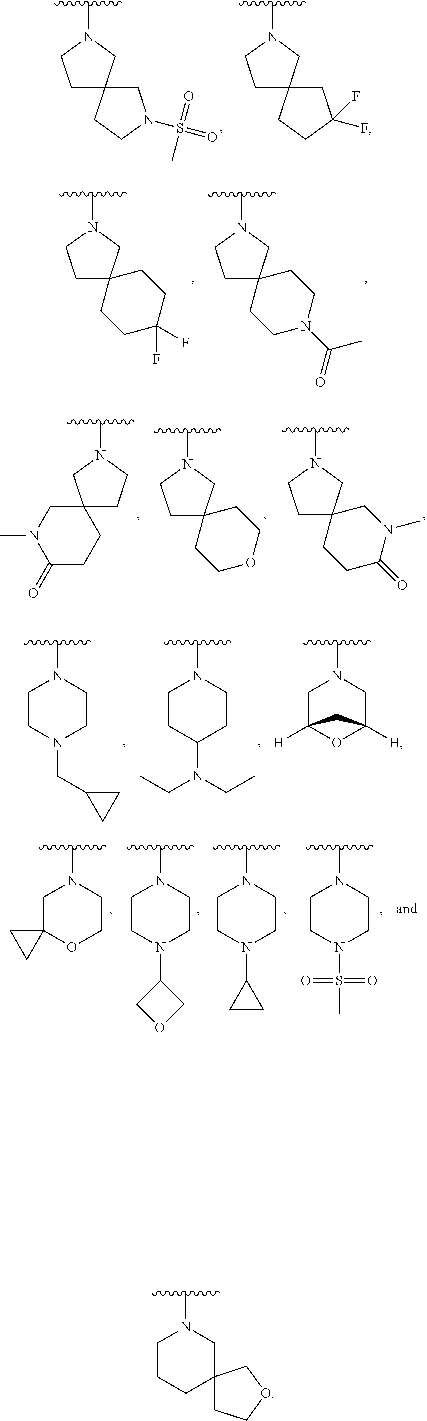

Embodiment 36 of this disclosure relates to the compound, or a pharmaceutically acceptable salt or solvate thereof, of Embodiment 35, wherein R² is selected from

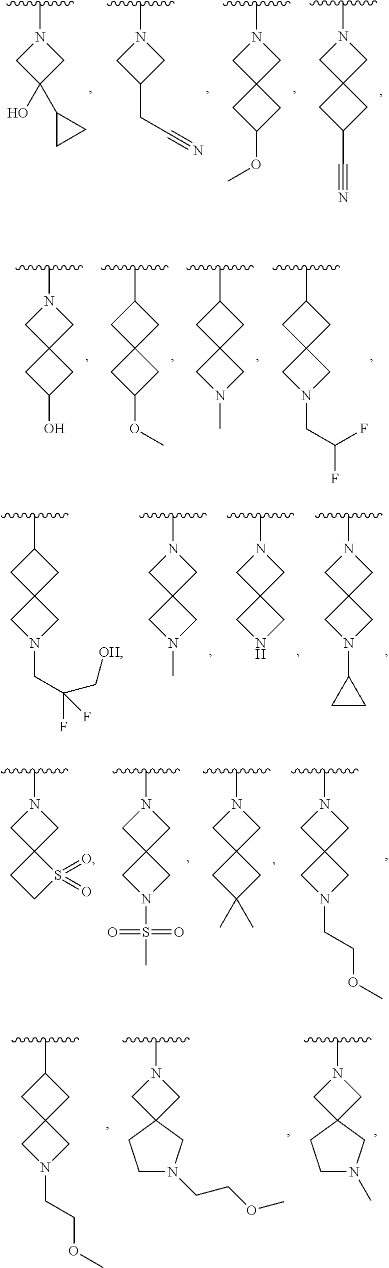

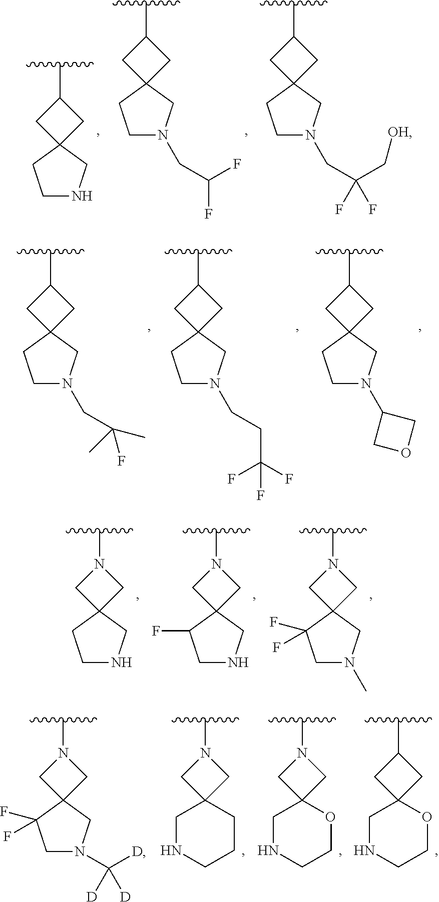

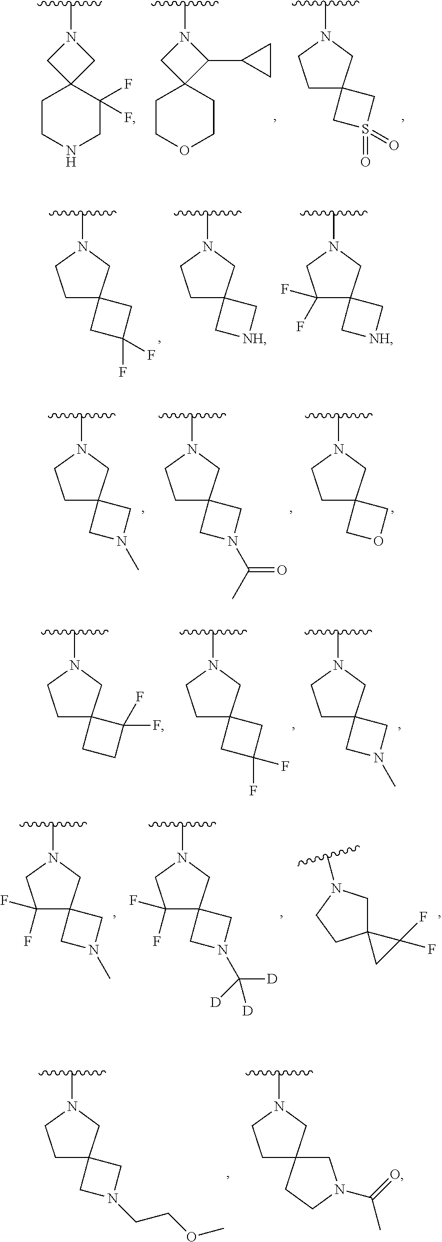

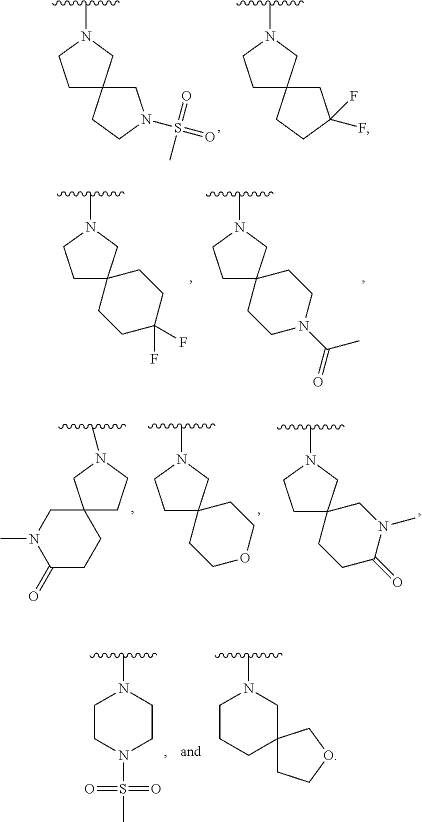

Embodiment 37 of the this disclosure relates to the compound, or a pharmaceutically acceptable salt or solvate thereof, of any one of Embodiments 1-10, 12, 12(a), 12(b), or 12(c), wherein R² is optionally substituted heterocycloalkyl.

Embodiment 38 of this disclosure relates to the compound, or a pharmaceutically acceptable salt or solvate thereof, of any of the preceding Embodiments, wherein R³ is selected from hydrogen, halogen, —CN, optionally substituted C₃₋₄ carbocycle, and optionally substituted 3- to 4 membered heterocycloalkyl.

Embodiment 39 of this disclosure relates to the compound, or a pharmaceutically acceptable salt or solvate thereof, of Embodiment 38, wherein R³ is selected from hydrogen, fluoro, —CN, cyclopropyl, cyclobutyl, optionally substituted oxetane, and optionally substituted azetidine.

Embodiment 40 of this disclosure relates to the compound, or a pharmaceutically acceptable salt or solvate thereof, of Embodiment 38, wherein R³ is selected from hydrogen, fluoro, and —CN.

Embodiment 41 of this disclosure relates to the compound, or a pharmaceutically acceptable salt or solvate thereof, of Embodiment 40, wherein R³ is selected from hydrogen and —CN.

Embodiment 42 of this disclosure relates to the compound, or a pharmaceutically acceptable salt or solvate thereof, of any one of the preceding Embodiments, wherein R⁴ is selected from hydrogen, halogen, —CN, optionally substituted C₁ alkyl, optionally substituted C₃₋₄ carbocycle, and optionally substituted 3- to 4 membered heterocycloalkyl.

Embodiment 43 of this disclosure relates to the compound, or a pharmaceutically acceptable salt or solvate thereof, of Embodiment 42, wherein R⁴ is selected from hydrogen, —CN, —CHF₂, —CF₃, cyclopropyl, cyclobutyl, optionally substituted oxetane, and optionally substituted azetidine.

Embodiment 44 of this disclosure relates to the compound, or a pharmaceutically acceptable salt or solvate thereof, of Embodiment 43, wherein R⁴ is selected from hydrogen, —CN, and —CHF₂.

Embodiment 45 of this disclosure relates to the compound, or a pharmaceutically acceptable salt or solvate thereof, of the preceding Embodiments, wherein R⁵ is selected from hydrogen, halogen, —CN, optionally substituted C₃₋₄ carbocycle, and optionally substituted 3- to 4 membered heterocycloalkyl.

Embodiment 46 of this disclosure relates to the compound, or a pharmaceutically acceptable salt or solvate thereof, of Embodiment 45, wherein R⁵ is selected from hydrogen, fluoro, —CN, cyclopropyl, cyclobutyl, optionally substituted oxetane, and optionally substituted azetidine.

Embodiment 47 of this disclosure relates to the compound, or a pharmaceutically acceptable salt or solvate thereof, of Embodiment 46, wherein R⁵ is hydrogen.

Embodiment 48 of this disclosure relates to the compound, or a pharmaceutically acceptable salt or solvate thereof, of any one of the preceding Embodiments wherein R⁶ is selected from hydrogen, halogen, —CN, optionally substituted C₃₋₄ carbocycle, and optionally substituted 3- to 4 membered heterocycloalkyl.

Embodiment 49 of this disclosure relates to the compound, or a pharmaceutically acceptable salt or solvate thereof, of Embodiment 48, wherein R⁶ is selected from hydrogen, fluoro, —CN, cyclopropyl, cyclobutyl, optionally substituted oxetane, and optionally substituted azetidine.

Embodiment 50 of this disclosure relates to the compound, or a pharmaceutically acceptable salt or solvate thereof, of Embodiment 49, wherein R⁶ is hydrogen.

Embodiment 51 of this disclosure relates to the compound, or a pharmaceutically acceptable salt or solvate thereof, of any one of the preceding Embodiments, wherein R⁷ is hydrogen.

Embodiment 52 of this disclosure relates to the compound, or a pharmaceutically acceptable salt thereof, of Embodiment 1 selected from one or more of the compounds in Table 1.

Embodiment 52(a) of this disclosure relates to the compound, or a pharmaceutically acceptable salt thereof, of Embodiment 1 selected from the compounds in Table 1.

Embodiment 53 of this disclosure relates to the compound, or a pharmaceutically acceptable salt thereof, of Embodiment 12 selected from the compounds in Table 1.

Embodiment 53(a) of this disclosure relates to Formula (IA), or a pharmaceutically acceptable salt thereof, of Embodiment 53 selected from the compounds in Table 1.

Embodiment 53(b) of this disclosure relates to Formula (IB), or a pharmaceutically acceptable salt thereof, of Embodiment 53 selected from the compounds in Table 1.

Embodiment 53(c) of this disclosure relates to Formula (IC), or a pharmaceutically acceptable salt thereof, of Embodiment 53 selected from the compounds in Table 1.

Embodiment 54 of this disclosure relates to the compound, or a pharmaceutically acceptable salt thereof, of Embodiment 16 selected from the compounds in Table 1.

Embodiment 54(a) of this disclosure relates to Formula (IAA), or a pharmaceutically acceptable salt thereof, of Embodiment 54 selected from the compounds in Table 1.

Embodiment 54(b) of this disclosure relates to Formula (IBB), or a pharmaceutically acceptable salt thereof, of Embodiment 54 selected from the compounds in Table 1.

Embodiment 54(c) of this disclosure relates to Formula (ICC), or a pharmaceutically acceptable salt thereof, of Embodiment 54 selected from the compounds in Table 1.

Embodiment 54(d) of this disclosure relates to Formula (IDD), or a pharmaceutically acceptable salt thereof, of Embodiment 54 selected from the compounds in Table 1. The compounds disclosed herein, in some embodiments, are used in different enriched isotopic forms, e.g., enriched in the content of ²H, ³H, ¹¹C, ¹³C and/or ¹⁴C. In one particular embodiment, the compound is deuterated in at least one position. Such deuterated forms can be made by the procedure described in U.S. Pat. Nos. 5,846,514 and 6,334,997. As described in U.S. Pat. Nos. 5,846,514 and 6,334,997, deuteration can improve the metabolic stability and or efficacy, thus increasing the duration of action of drugs.

Unless otherwise stated, compounds described herein are intended to include compounds which differ only in the presence of one or more isotopically enriched atoms. For example, compounds having the present structures except for the replacement of a hydrogen by a deuterium or tritium, or the replacement of a carbon by ³C- or ⁴C-enriched carbon are within the scope of the present disclosure.

The compounds of the present disclosure optionally contain unnatural proportions of atomic isotopes at one or more atoms that constitute such compounds. For example, the compounds may be labeled with isotopes, such as for example, deuterium (²H), tritium (³H), iodine-125 (¹²⁵I) or carbon-14 (¹⁴C). Isotopic substitution with ²H, ¹¹C, ¹³C, ¹⁴C, ¹⁵C, ¹²N, ¹³N, ¹⁵N, ¹⁶N, ¹⁶O, ¹⁷O, ¹⁴F, ¹⁵F, ¹⁶F, ¹⁷F, ¹⁸F, ³³S, ³⁴S, ³⁵S, ³⁶S, ³⁵Cl, ³⁷Cl, ⁷⁹Br, ⁸¹Br, and ¹²⁵I are all contemplated. All isotopic variations of the compounds of the present invention, whether radioactive or not, are encompassed within the scope of the present invention.

In certain embodiments, the compounds disclosed herein have some or all of the ¹H atoms replaced with ²H atoms. The methods of synthesis for deuterium-containing compounds are known in the art and include, by way of non-limiting example only, the following synthetic methods.

Deuterium substituted compounds are synthesized using various methods such as described in: Dean, Dennis C.; Editor. Recent Advances in the Synthesis and Applications of Radiolabeled Compounds for Drug Discovery and Development. [In: Curr., Pharm. Des., 2000; 6(10)] 2000, 110 pp; George W.; Varma, Rajender S. The Synthesis of Radiolabeled Compounds via Organometallic Intermediates, Tetrahedron, 1989, 45(21), 6601-21; and Evans, E. Anthony. Synthesis of radiolabeled compounds, J. Radioanal. Chem., 1981, 64(1-2), 9-32.

Deuterated starting materials are readily available and are subjected to the synthetic methods described herein to provide for the synthesis of deuterium-containing compounds. Large numbers of deuterium-containing reagents and building blocks are available commercially from chemical vendors, such as Aldrich Chemical Co.

Compounds of the present invention also include crystalline and amorphous forms of those compounds, pharmaceutically acceptable salts, and active metabolites of these compounds having the same type of activity, including, for example, polymorphs, pseudopolymorphs, solvates, hydrates, unsolvated polymorphs (including anhydrates), conformational polymorphs, and amorphous forms of the compounds, as well as mixtures thereof.

Included in the present disclosure are salts, particularly pharmaceutically acceptable salts, of the compounds described herein. The compounds of the present disclosure that possess a sufficiently acidic, a sufficiently basic, or both functional groups, can react with any of a number of inorganic bases, and inorganic and organic acids, to form a salt. Alternatively, compounds that are inherently charged, such as those with a quaternary nitrogen, can form a salt with an appropriate counterion, e.g., a halide such as bromide, chloride, or fluoride, particularly bromide.

The compounds described herein may in some cases exist as diastereomers, enantiomers, or other stereoisomeric forms. The compounds presented herein include all diastereomeric, enantiomeric, and epimeric forms as well as the appropriate mixtures thereof. Separation of stereoisomers may be performed by chromatography or by forming diastereomers and separating by recrystallization, or chromatography, or any combination thereof. (Jean Jacques, Andre Collet, Samuel H. Wilen, “Enantiomers, Racemates and Resolutions”, John Wiley And Sons, Inc., 1981, herein incorporated by reference for this disclosure). Stereoisomers may also be obtained by stereoselective synthesis.

The methods and compositions described herein include the use of amorphous forms as well as crystalline forms (also known as polymorphs). The compounds described herein may be in the form of pharmaceutically acceptable salts. As well, in some embodiments, active metabolites of these compounds having the same type of activity are included in the scope of the present disclosure. In addition, the compounds described herein can exist in unsolvated as well as solvated forms with pharmaceutically acceptable solvents such as water, ethanol, and the like. The solvated forms of the compounds presented herein are also considered to be disclosed herein.

In certain embodiments, compounds or salts of the compounds may be prodrugs, e.g., wherein a hydroxyl in the parent compound is presented as an ester or a carbonate, or carboxylic acid present in the parent compound is presented as an ester. The term “prodrug” is intended to encompass compounds which, under physiologic conditions, are converted into pharmaceutical agents of the present disclosure. One method for making a prodrug is to include one or more selected moieties which are hydrolyzed under physiologic conditions to reveal the desired molecule. In other embodiments, the prodrug is converted by an enzymatic activity of the host animal such as specific target cells in the host animal. For example, esters or carbonates (e.g., esters or carbonates of alcohols or carboxylic acids and esters of phosphonic acids) are preferred prodrugs of the present disclosure.

Prodrug forms of the herein described compounds, wherein the prodrug is metabolized in vivo to produce a compound as set forth herein are included within the scope of the claims. In some cases, some of the herein-described compounds may be a prodrug for another derivative or active compound.

Prodrugs are often useful because, in some situations, they may be easier to administer than the parent drug. They may, for instance, be bioavailable by oral administration whereas the parent is not. Prodrugs may help enhance the cell permeability of a compound relative to the parent drug. The prodrug may also have improved solubility in pharmaceutical compositions over the parent drug. Prodrugs may be designed as reversible drug derivatives, for use as modifiers to enhance drug transport to site-specific tissues or to increase drug residence inside of a cell.

In some embodiments, the design of a prodrug increases the lipophilicity of the pharmaceutical agent. In some embodiments, the design of a prodrug increases the effective water solubility. See, e.g., Fedorak et al., Am. J. Physiol., 269:G210-218 (1995); McLoed et al., Gastroenterol, 106:405-413 (1994); Hochhaus et al., Biomed. Chrom., 6:283-286 (1992); J. Larsen and H. Bundgaard, Int. J. Pharmaceutics, 37, 87 (1987); J. Larsen et al., Int. J. Pharmaceutics, 47, 103 (1988); Sinkula et al., J. Pharm. Sci., 64:181-210 (1975); T. Higuchi and V. Stella, Pro-drugs as Novel Delivery Systems, Vol. 14 of the A.C.S. Symposium Series; and Edward B. Roche, Bioreversible Carriers in Drug Design, American Pharmaceutical Association and Pergamon Press, 1987, all incorporated herein for such disclosure). According to another embodiment, the present disclosure provides methods of producing the above-defined compounds. The compounds may be synthesized using conventional techniques. Advantageously, these compounds are conveniently synthesized from readily available starting materials.

Synthetic chemistry transformations and methodologies useful in synthesizing the compounds described herein are known in the art and include, for example, those described in R. Larock, Comprehensive Organic Transformations (1989); T. W. Greene and P. G. M. Wuts, Protective Groups in Organic Synthesis, 2d. Ed. (1991); L. Fieser and M. Fieser, Fieser and Fieser's Reagents for Organic Synthesis (1994); and L. Paquette, ed., Encyclopedia of Reagents for Organic Synthesis (1995).

### Therapeutic Applications

Methods of administration of a compound or pharmaceutically acceptable salt of Formula (I), (IA), (IAA), (IB), (IBB), (IC), (ICC), (II), or (IIA) discussed herein may be used for the treatment of cancer. In some embodiments, disclosed herein are methods to treat solid tumors. Examples of cancer include but are not limited to ovarian cancer, breast cancer, colon cancer, and brain cancer.

In some embodiments, disclosed herein are methods to treat cancer by the administration of a compound or pharmaceutically acceptable salt of Formula (I), (IA), (IAA), (IB), (IBB), (IC), (ICC), (II), or (IIA). In some embodiments, disclosed herein is a method of treating cancer, comprising administering to a subject in need thereof the pharmaceutical composition described herein.

In some embodiments, disclosed herein is a method of inhibiting a cyclin dependent kinase (CDK) in a cell by the administration of a compound or pharmaceutically acceptable salt of Formula (I), (IA), (IAA), (IB), (IBB), (IC), (ICC), (II), or (IIA). In some embodiments, disclosed herein is a method of inhibiting a cyclin dependent kinase (CDK) in a cell with a compound or pharmaceutically acceptable salt of any one of the compounds described herein or the pharmaceutical composition described herein.

The CDK can be any suitable CDK known by one of skill in the art. In some embodiments, CDK is selected from CDK 2, CDK 4, CD6, or any combination thereof. In some embodiments, the CDK is selected from CDK2, CDK4, CDK6, CDK 2/4, CDK 2/6, CDK 4/6, and CDK 2/4/6. In some embodiments, the CDK is selected from CDK 2/4, CDK 2/6, CDK 4/6, and CDK 2/4/6.

Additional embodiments of the therapeutic applications of this disclosure include the following:

Embodiment 56 of this disclosure relates to a method of treating cancer, comprising administering to a subject in need thereof the pharmaceutical composition of Embodiment 33.

Embodiment 57 of this disclosure relates to the method of Embodiment 56, wherein the cancer is a solid tumor.

Embodiment 58 of this disclosure relates to the method of Embodiment 56 or 57, wherein the cancer is selected from ovarian cancer, breast cancer, colon cancer, and brain cancer.

Embodiment 59 of this disclosure relates to the method of Embodiment 58, wherein the cancer is ovarian cancer or breast cancer.

Embodiment 60 of this disclosure relates to a method of inhibiting a cyclin dependent kinase (CDK) in a cell with a compound or pharmaceutically acceptable salt of any one of Embodiments 1 to [0396] or the pharmaceutical composition of Embodiment 33.

Embodiment 61 of this disclosure relates to the method of Embodiment 37, wherein the CDK is selected from CDK 2, CDK 4, CD6, or any combination thereof.

Embodiment 62 of this disclosure relates to the method of Embodiment 37, wherein the CDK is selected from CDK 2/4, CDK 2/6, CDK 4/6, and CDK 2/4/6.

Embodiment 63 of this disclosure relates to the method of Embodiment 62, wherein the CDK is CDK 2/4/6.

### Pharmaceutical Formulations

The compositions and methods described herein may be considered useful as pharmaceutical compositions for administration to a subject in need thereof. Pharmaceutical compositions may comprise at least a compound or pharmaceutically acceptable salt of Formula (I), (IA), (IAA), (IB), (IBB), (IC), (ICC), (II), or (IIA) described herein and one or more pharmaceutically acceptable carriers, diluents, excipients, stabilizers, dispersing agents, suspending agents, and/or thickening agents. In some embodiments, disclosed herein is a pharmaceutical composition comprising a compound or pharmaceutically acceptable salt described herein and a pharmaceutically acceptable excipient. In some embodiments, disclosed herein is a pharmaceutical composition comprising a compound or pharmaceutically acceptable salt of Formula (I), (IA), (IAA), (IB), (IBB), (IC), (ICC), (II), or (IIA) described herein and a pharmaceutically acceptable excipient.

Pharmaceutical compositions comprising a compound or pharmaceutically acceptable salt of Formula (I), (IA), (IAA), (IB), (IBB), (IC), (ICC), (II), or (IIA) may be formulated using one or more physiologically-acceptable carriers comprising excipients and auxiliaries. Formulation may be modified depending upon the route of administration chosen. Pharmaceutical compositions comprising a compound, salt or conjugate may be manufactured, for example, by lyophilizing the compound, salt or conjugate, mixing, dissolving, emulsifying, encapsulating or entrapping the conjugate. The pharmaceutical compositions may also include the compounds, salts or conjugates in a free-base form or pharmaceutically-acceptable salt form.

Methods for formulation of a compound or pharmaceutically acceptable salt of Formula (I), (IA), (IAA), (IB), (IBB), (IC), (ICC), (II), or (IIA) may include formulating any of the compounds, salts or conjugates with one or more inert, pharmaceutically-acceptable excipients or carriers to form a solid, semi-solid, or liquid composition. Solid compositions may include, for example, powders, tablets, dispersible granules and capsules, and in some aspects, the solid compositions further contain nontoxic, auxiliary substances, for example wetting or emulsifying agents, pH buffering agents, and other pharmaceutically-acceptable additives. Alternatively, the compounds, salts or conjugates may be lyophilized or in powder form for re-constitution with a suitable vehicle, e.g., sterile pyrogen-free water, before use.

Pharmaceutical compositions comprising a compound or pharmaceutically acceptable salt of Formula (I), (IA), (IAA), (IB), (IBB), (IC), (ICC), (II), or (IIA) may comprise at least one active ingredient (e.g., a compound, salt or conjugate and other agents). The active ingredients may be entrapped in microcapsules prepared, for example, by coacervation techniques or by interfacial polymerization (e.g., hydroxymethylcellulose or gelatin microcapsules and poly-(methylmethacylate) microcapsules, respectively), in colloidal drug-delivery systems (e.g., liposomes, albumin microspheres, microemulsions, nano-particles and nanocapsules) or in macroemulsions.

The compositions and formulations may be sterilized. Sterilization may be accomplished by filtration through sterile filtration.

The compositions comprising a compound or pharmaceutically acceptable salt of Formula (I), (IA), (IAA), (IB), (IBB), (IC), (ICC), (II), or (IIA) may be formulated for administration as an injection. Non-limiting examples of formulations for injection may include a sterile suspension, solution or emulsion in oily or aqueous vehicles. Suitable oily vehicles may include, but are not limited to, lipophilic solvents or vehicles such as fatty oils or synthetic fatty acid esters, or liposomes. Aqueous injection suspensions may contain substances which increase the viscosity of the suspension. The suspension may also contain suitable stabilizers. Injections may be formulated for bolus injection or continuous infusion. Alternatively, the compositions may be lyophilized or in powder form for reconstitution with a suitable vehicle, e.g., sterile pyrogen-free water, before use.

For parenteral administration, a compound or pharmaceutically acceptable salt of Formula (I), (IA), (IAA), (IB), (IBB), (IC), (ICC), (II), or (IIA) may be formulated in a unit dosage injectable form (e.g., solution, suspension, emulsion) in association with a pharmaceutically acceptable parenteral vehicle. Such vehicles may be inherently non-toxic, and non-therapeutic. Vehicles may be water, saline, Ringer's solution, dextrose solution, and 5% human serum albumin. Non-aqueous vehicles such as fixed oils and ethyl oleate may also be used. Liposomes may be used as carriers. The vehicle may contain minor amounts of additives such as substances that enhance isotonicity and chemical stability (e.g., buffers and preservatives).

In one embodiment the invention relates to methods and compositions of Formula (I), (IA), (IAA), (IB), (IBB), (IC), (ICC), (II), or (IIA) formulated for oral delivery to a subject in need. In one embodiment a composition is formulated so as to deliver one or more pharmaceutically active agents to a subject through a mucosa layer in the mouth or esophagus. In another embodiment the composition is formulated to deliver one or more pharmaceutically active agents to a subject through a mucosa layer in the stomach and/or intestines.

In one embodiment compositions of Formula Formula (I), (IA), (IAA), (IB), (IBB), (IC), (ICC), (II), or (IIA) are provided in modified release dosage forms. Suitable modified release dosage vehicles include, but are not limited to, hydrophilic or hydrophobic matrix devices, water-soluble separating layer coatings, enteric coatings, osmotic devices, multi-particulate devices, and combinations thereof. The compositions may also comprise non-release controlling excipients.

In another embodiment compositions of Formula (I), (IA), (IAA), (IB), (IBB), (IC), (ICC), (II), or (IIA) are provided in enteric coated dosage forms. These enteric coated dosage forms can also comprise non-release controlling excipients. In one embodiment the compositions are in the form of enteric-coated granules, as controlled-release capsules for oral administration. The compositions can further comprise cellulose, disodium hydrogen phosphate, hydroxypropyl cellulose, pyridazine, lactose, mannitol, or sodium lauryl sulfate. In another embodiment the compositions are in the form of enteric-coated pellets, as controlled-release capsules for oral administration. The compositions can further comprise glycerol monostearate 40-50, hydroxypropyl cellulose, pyridazine, magnesium stearate, methacrylic acid copolymer type C, polysorbate 80, sugar spheres, talc, or triethyl citrate.

In another embodiment the compositions of Formula (I), (IA), (IAA), (IB), (IBB), (IC), (ICC), (II), or (IIA) are enteric-coated controlled-release tablets for oral administration. The compositions can further comprise carnauba wax, crospovidone, diacetylated monoglycerides, ethylcellulose, hydroxypropyl cellulose, pyridazine phthalate, magnesium stearate, mannitol, sodium hydroxide, sodium stearyl fumarate, talc, titanium dioxide, or yellow ferric oxide.

Sustained-release preparations comprising a compound or pharmaceutically acceptable salt of Formula (I), (IA), (IAA), (IB), (IBB), (IC), (ICC), (II), or (IIA) may also be prepared. Examples of sustained-release preparations may include semipermeable matrices of solid hydrophobic polymers that may contain the compound, salt or conjugate, and these matrices may be in the form of shaped articles (e.g., films or microcapsules). Examples of sustained-release matrices may include polyesters, hydrogels (e.g., poly(2-hydroxyethyl-methacrylate), or poly(vinyl alcohol)), polylactides, copolymers of L-glutamic acid and 7 ethyl-L-glutamate, non-degradable ethylene-vinyl acetate, degradable lactic acid-glycolic acid copolymers such as the LUPRON DEPO™ (i.e., injectable microspheres composed of lactic acid-glycolic acid copolymer and leuprolide acetate), and poly-D-(−)-3-hydroxybutyric acid.

Pharmaceutical formulations comprising a compound or pharmaceutically acceptable salt of Formula (I), (IA), (IAA), (IB), (IBB), (IC), (ICC), (II), or (IIA) may be prepared for storage by mixing a compound, salt or conjugate with a pharmaceutically acceptable carrier, excipient, and/or a stabilizer. This formulation may be a lyophilized formulation or an aqueous solution. Acceptable carriers, excipients, and/or stabilizers may be nontoxic to recipients at the dosages and concentrations used. Acceptable carriers, excipients, and/or stabilizers may include buffers such as phosphate, citrate, and other organic acids; antioxidants including ascorbic acid and methionine; preservatives, polypeptides; proteins, such as serum albumin or gelatin; hydrophilic polymers; amino acids; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes; and/or non-ionic surfactants or polyethylene glycol.

In another embodiment the compositions of Formula (I), (IA), (IAA), (IB), (IBB), (IC), (ICC), (II), or (IIA) can further comprise calcium stearate, crospovidone, hydroxypropyl methylcellulose, iron oxide, mannitol, methacrylic acid copolymer, polysorbate 80, povidone, propylene glycol, sodium carbonate, sodium lauryl sulfate, titanium dioxide, and triethyl citrate.

In another embodiment compositions of Formula (I), (IA), (IAA), (IB), (IBB), (IC), (ICC), (II), or (IIA) are provided in effervescent dosage forms. These effervescent dosage forms can also comprise non-release controlling excipients.

In another embodiment compositions of Formula (I), (IA), (IAA), (IB), (IBB), (IC), (ICC), (II), or (IIA) can be provided in a dosage form that has at least one component that can facilitate the immediate release of an active agent, and at least one component that can facilitate the controlled release of an active agent. In a further embodiment the dosage form can be capable of giving a discontinuous release of the compound in the form of at least two consecutive pulses separated in time from 0.1 up to 24 hours. The compositions can comprise one or more release controlling and non-release controlling excipients, such as those excipients suitable for a disruptable semi-permeable membrane and as swellable substances.

In another embodiment compositions Formula (I), (IA), (IAA), (IB), (IBB), (IC), (ICC), (II), or (IIA) are provided in a dosage form for oral administration to a subject, which comprise one or more pharmaceutically acceptable excipients or carriers, enclosed in an intermediate reactive layer comprising a gastric juice-resistant polymeric layered material partially neutralized with alkali and having cation exchange capacity and a gastric juice-resistant outer layer.

In some embodiments, the compositions of Formula (I), (IA), (IAA), (IB), (IBB), (IC), (ICC), (II), or (IIA) provided herein can be in unit-dosage forms or multiple-dosage forms. Unit-dosage forms, as used herein, refer to physically discrete units suitable for administration to human or non-human animal subjects and packaged individually. Each unit-dose can contain a predetermined quantity of an active ingredient(s) sufficient to produce the desired therapeutic effect, in association with the required pharmaceutical carriers or excipients. Examples of unit-dosage forms include, but are not limited to, ampoules, syringes, and individually packaged tablets and capsules. In some embodiments, unit-dosage forms may be administered in fractions or multiples thereof. A multiple-dosage form is a plurality of identical unit-dosage forms packaged in a single container, which can be administered in segregated unit-dosage form. Examples of multiple-dosage forms include, but are not limited to, vials, bottles of tablets or capsules, or bottles of pints or gallons. In another embodiment the multiple dosage forms comprise different pharmaceutically active agents.

In some embodiments, the compositions of Formula (I), (IA), (IAA), (IB), (IBB), (IC), (ICC), (II), or (IIA) may also be formulated as a modified release dosage form, including immediate-, delayed-, extended-, prolonged-, sustained-, pulsatile-, controlled-, extended, accelerated- and fast-, targeted-, programmed-release, and gastric retention dosage forms. These dosage forms can be prepared according to known methods and techniques (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126, which are herein incorporated by reference in their entirety).

Additional embodiments of the pharmaceutical formulations of this disclosure include the following:

Embodiment 55 of this disclosure relates to a pharmaceutical composition comprising a compound or pharmaceutically acceptable salt of any one of Embodiments 1 to 54, or any sub-embodiments thereof, and a pharmaceutically acceptable excipient.

### Combination Therapies

Also contemplated herein are combination therapies, for example, co-administering a disclosed compound and an additional active agent, as part of a specific treatment regimen intended to provide the beneficial effect from the co-action of these therapeutic agents. The beneficial effect of the combination includes, but is not limited to, pharmacokinetic or pharmacodynamic co-action resulting from the combination of therapeutic agents. Administration of these therapeutic agents in combination typically is carried out over a defined time period (usually hours, days, weeks, months or years depending upon the combination selected). Combination therapy is intended to embrace administration of multiple therapeutic agents in a sequential manner, that is, wherein each therapeutic agent is administered at a different time, as well as administration of these therapeutic agents, or at least two of the therapeutic agents, in a substantially simultaneous manner.

Substantially simultaneous administration is accomplished, for example, by administering to the subject a single formulation or composition, (e.g., a tablet or capsule having a fixed ratio of each therapeutic agent or in multiple, single formulations (e.g., capsules) for each of the therapeutic agents. Sequential or substantially simultaneous administration of each therapeutic agent is affected by any appropriate route including, but not limited to, oral routes, intravenous routes, intramuscular routes, and direct absorption through mucous membrane tissues. The therapeutic agents are administered by the same route or by different routes. For example, a first therapeutic agent of the combination selected is administered by intravenous injection while the other therapeutic agents of the combination are administered orally. Alternatively, for example, all therapeutic agents are administered orally or all therapeutic agents are administered by intravenous injection.

The components of the combination are administered to a patient simultaneously or sequentially. It will be appreciated that the components are present in the same pharmaceutically acceptable carrier and, therefore, are administered simultaneously. Alternatively, the active ingredients are present in separate pharmaceutical carriers, such as, conventional oral dosage forms, that are administered either simultaneously or sequentially.

### ADDITIONAL EMBODIMENTS

Embodiment 101. A compound, or a pharmaceutically acceptable salt or solvate thereof, having the structure of Formula (I):

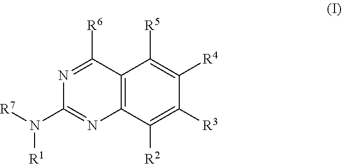

wherein,
R¹ is selected from optionally substituted piperidine, optionally substituted 2-pyridine, optionally substituted azabicyclo[3.1.0]hexane, optionally substituted indole, optionally substituted isoindole, and optionally substituted azetidine;
R² is selected from optionally substituted cycloalkyl and optionally substituted heterocycle;
each of R³, R⁴, R⁵, R⁶, is independently selected from hydrogen, halogen, —CN, optionally substituted C₁₋₄ alkyl, optionally substituted C₃₋₄ carbocycle, and optionally substituted 3- to 4-membered heterocycloalkyl; and
R⁷ is selected from hydrogen and optionally substituted C₁₋₄ alkyl.

Embodiment 102. The compound, or a pharmaceutically acceptable salt or solvate thereof, of embodiment 1 having the structure of Formula (IA):

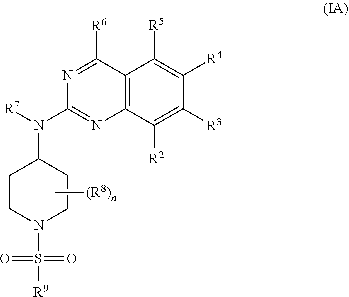

wherein,
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl; and
n is selected from 0 to 9.

Embodiment 103. The compound or salt of embodiment 2 wherein R² is selected from optionally substituted C₃₋₆ cycloalkyl, optionally substituted pyrrolidine, optionally substituted piperidine, optionally substituted pyrazole, optionally substituted azetidine, optionally substituted oxetane, and optionally substituted morpholine.

Embodiment 104. The compound or salt of embodiment 6, wherein R² is substituted with —CN, —SO₂R₂, —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, or azetidine, wherein R^{2a} is selected from C₁₋₆ alkyl.

Embodiment 105. The compound or salt of embodiment 6, wherein R² is 6-oxa-3-azabicyclo[3.1.1]heptane.

Embodiment 106. The compound or salt of any one of embodiments 1 to [0307], wherein the compound is selected from:

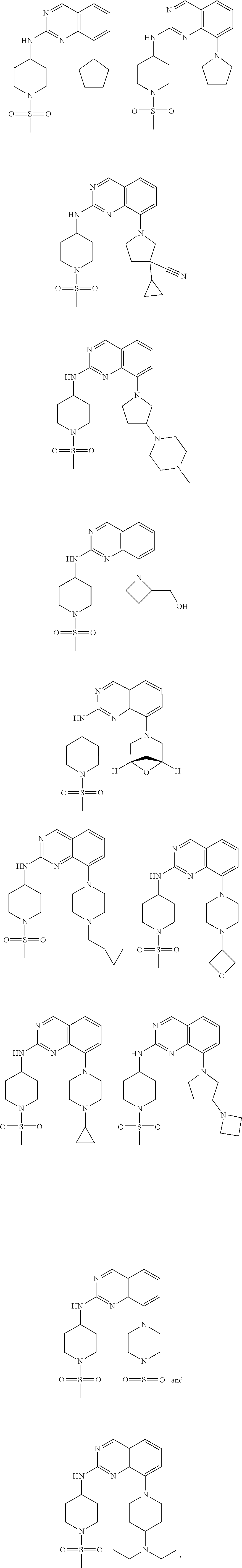

Embodiment 107. The compound or salt of any one of embodiments 1 to [0307], wherein the compound is selected from:

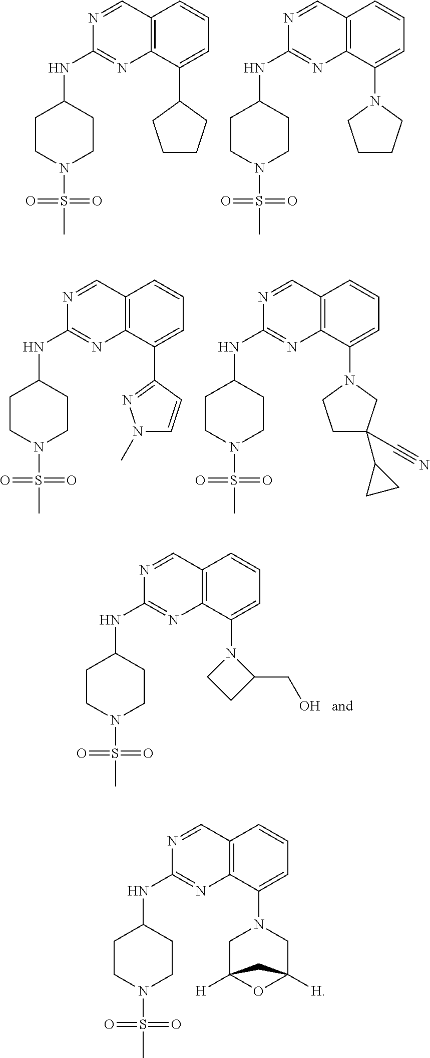

Embodiment 108. The compound, or a pharmaceutically acceptable salt or solvate thereof, of embodiment 1 or 2 having the structure of Formula (IAA):

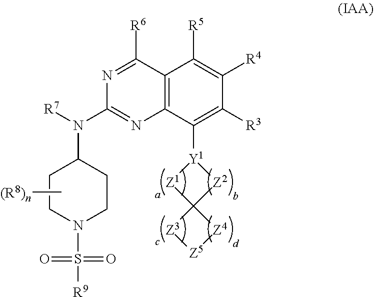

wherein,
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl;
n is selected from 0 to 9;
Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle; and
each R¹¹ is independently selected from hydrogen and optionally substituted C₁₋₄ alkyl.

Embodiment 109. The compound or salt of embodiment [0309], wherein Y¹ is —N—.

Embodiment 110. The compound or salt of embodiment 9, wherein Y¹ is —CR¹⁰—.

Embodiment 111. The compound or salt of embodiment 9, wherein each of Z¹, Z², Z³, Z⁴ and Z⁵ are independently selected from —C(R¹⁰)₂—, —NR¹¹—, —N(C(O)R¹⁰)—, —NS(O₂)R¹¹, —O—, and —S(O)₂—, wherein Z⁵ is additionally selected from a bond.

Embodiment 112. The compound or salt of any one of embodiments 9 to [0313], wherein each of a, b, c, and d are independently selected from 1, 2, and 3.

Embodiment 113. The compound or salt of embodiment [0314], wherein each of a, c, and d are independently selected from 1 and 2.

Embodiment 114. The compound or salt of any one of embodiments 9 to [0315], wherein each R¹⁰ is independently selected from hydrogen, halogen, —OH, optionally substituted C₁₋₃ alkyl, and optionally substituted C₃₋₆ cycloalkyl.

Embodiment 115. The compound or salt of embodiment [0316], wherein each R¹⁰ is independently selected from hydrogen, fluoro, —OH, methyl, and cyclopropyl.

Embodiment 116. The compound or salt of any one of embodiments 9 to [0317], wherein each R¹¹ is independently selected from hydrogen and optionally substituted C₁₋₂ alkyl.

Embodiment 117. The compound or salt of embodiment [0318], wherein each R¹¹ is independently selected from hydrogen, methyl, and ethyl, wherein the ethyl is optionally substituted with —OMe.

Embodiment 118. The compound or salt of any one of embodiments 9 to Error! Reference source not found., wherein the compound is selected from:

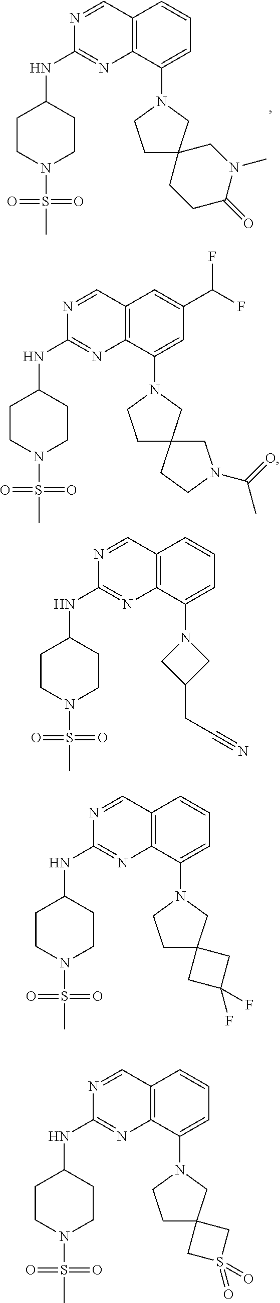

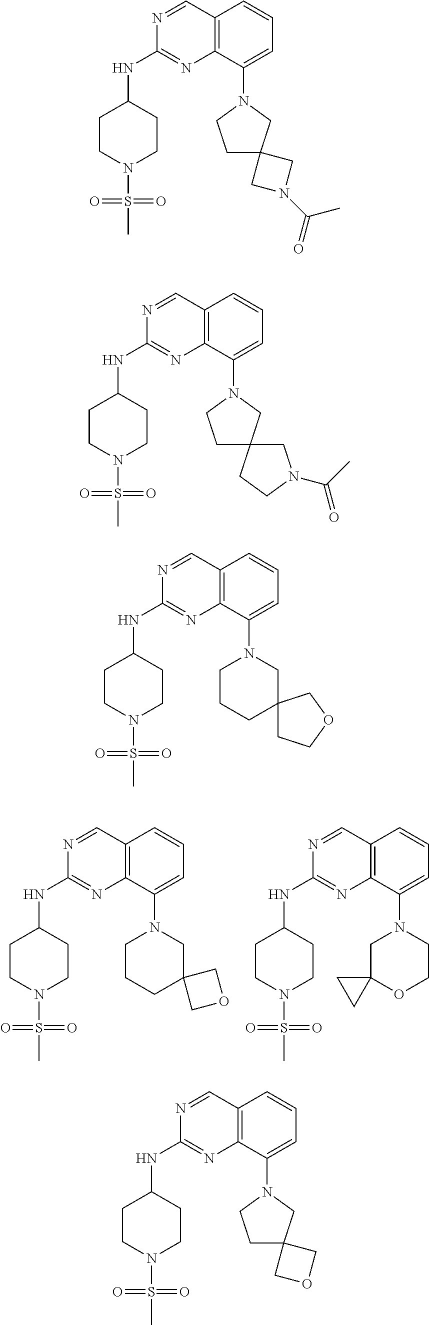

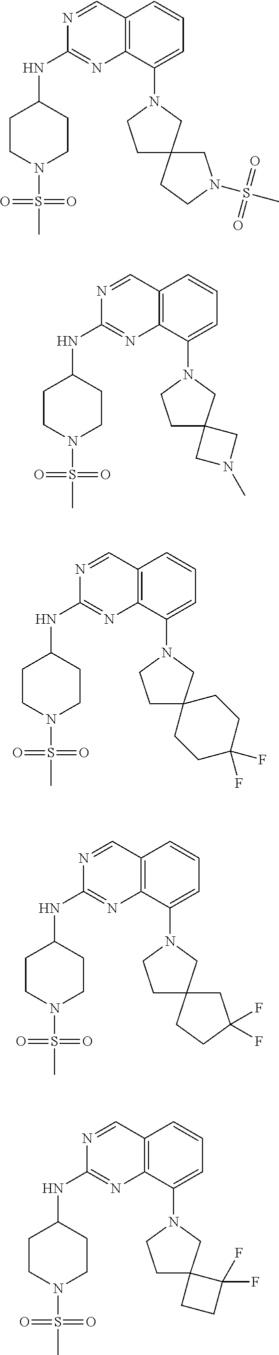 [SMILES: CS(=O)(=O)N1CCC(Nc2ncc3cccc(N4CCC5(CCN(S(C)(=O)=O)C5)C4)c3n2)CC1 ; CN1CC2(CCN(c3cccc4cnc(NC5CCN(S(C)(=O)=O)CC5)nc34)C2)C1 ; CS(=O)(=O)N1CCC(Nc2ncc3cccc(N4CCC5(CCC(F)(F)CC5)C4)c3n2)CC1 ; CS(=O)(=O)N1CCC(Nc2ncc3cccc(N4CCC5(CCC(F)(F)C5)C4)c3n2)CC1 ; CS(=O)(=O)N1CCC(Nc2ncc3cccc(N4CCC5(CCC5(F)F)C4)c3n2)CC1]

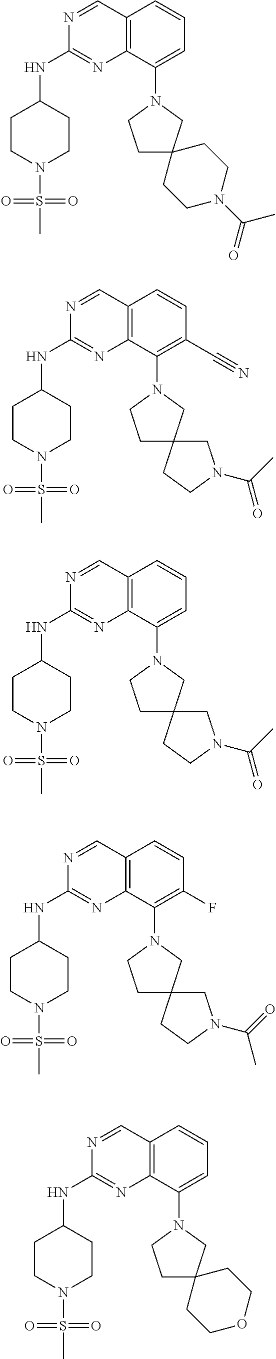 [SMILES: CC(=O)N1CCC2(CC1)CCN(c1cccc3cnc(NC4CCN(S(C)(=O)=O)CC4)nc13)C2 ; CC(=O)N1CCC2(CCN(c3c(C#N)ccc4cnc(NC5CCN(S(C)(=O)=O)CC5)nc34)C2)C1 ; CC(=O)N1CCC2(CCN(c3cccc4cnc(NC5CCN(S(C)(=O)=O)CC5)nc34)C2)C1 ; CC(=O)N1CCC2(CCN(c3c(F)ccc4cnc(NC5CCN(S(C)(=O)=O)CC5)nc34)C2)C1 ; CS(=O)(=O)N1CCC(Nc2ncc3cccc(N4CCC5(CCOCC5)C4)c3n2)CC1]

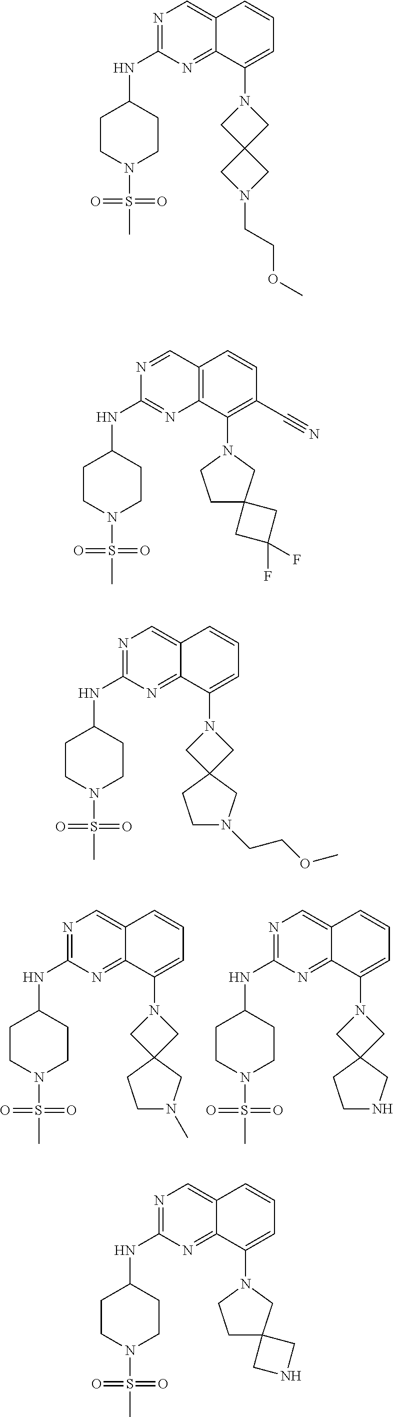

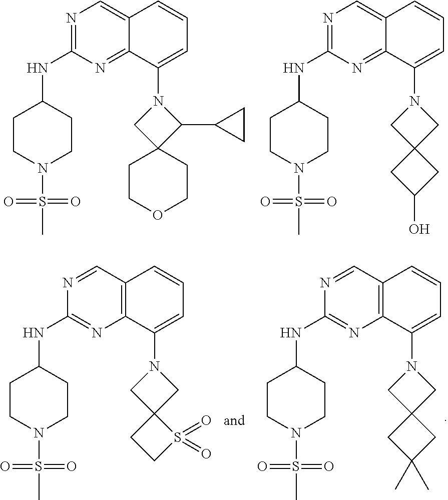

Embodiment 119. The compound or salt of embodiment [0320], wherein the compound is selected from:

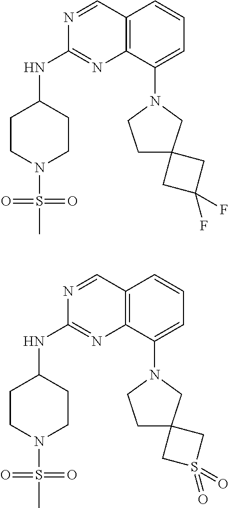 [SMILES: CS(=O)(=O)N1CCC(Nc2ncc3cccc(N4CCC5(C4)CC(F)(F)C5)c3n2)CC1 ; CS(=O)(=O)N1CCC(Nc2ncc3cccc(N4CCC5(C4)CS(=O)(=O)C5)c3n2)CC1]

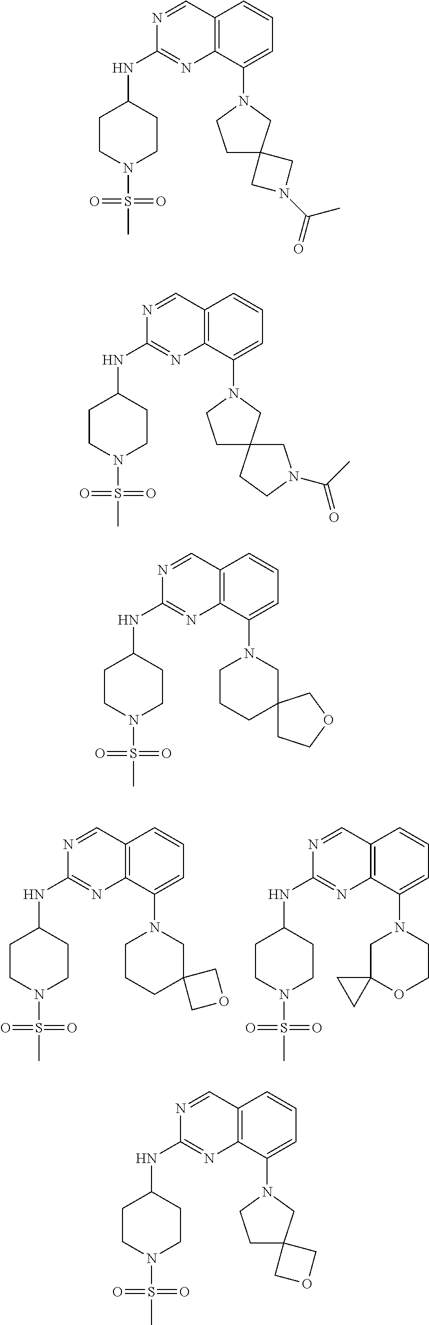

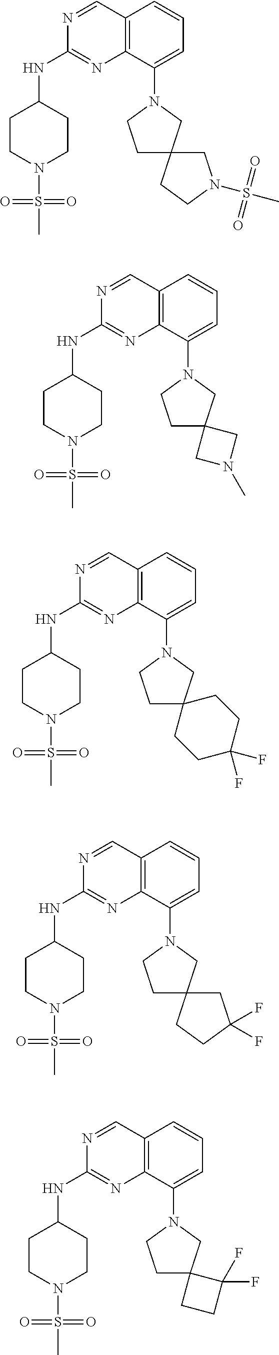 [SMILES: CS(=O)(=O)N1CCC(Nc2ncc3cccc(N4CCC5(CCN(S(C)(=O)=O)C5)C4)c3n2)CC1 ; CN1CC2(CCN(c3cccc4cnc(NC5CCN(S(C)(=O)=O)CC5)nc34)C2)C1 ; CS(=O)(=O)N1CCC(Nc2ncc3cccc(N4CCC5(CCC(F)(F)CC5)C4)c3n2)CC1 ; CS(=O)(=O)N1CCC(Nc2ncc3cccc(N4CCC5(CCC(F)(F)C5)C4)c3n2)CC1 ; CS(=O)(=O)N1CCC(Nc2ncc3cccc(N4CCC5(CCC5(F)F)C4)c3n2)CC1]

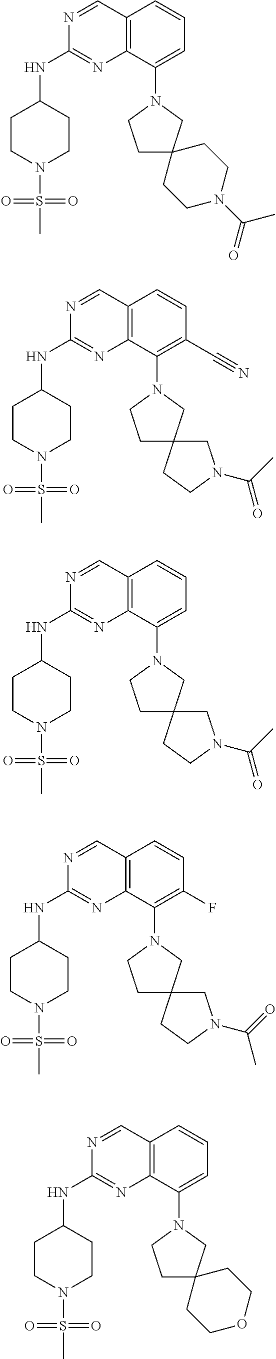 [SMILES: CC(=O)N1CCC2(CC1)CCN(c1cccc3cnc(NC4CCN(S(C)(=O)=O)CC4)nc13)C2 ; CC(=O)N1CCC2(CCN(c3c(C#N)ccc4cnc(NC5CCN(S(C)(=O)=O)CC5)nc34)C2)C1 ; CC(=O)N1CCC2(CCN(c3cccc4cnc(NC5CCN(S(C)(=O)=O)CC5)nc34)C2)C1 ; CC(=O)N1CCC2(CCN(c3c(F)ccc4cnc(NC5CCN(S(C)(=O)=O)CC5)nc34)C2)C1 ; CS(=O)(=O)N1CCC(Nc2ncc3cccc(N4CCC5(CCOCC5)C4)c3n2)CC1]

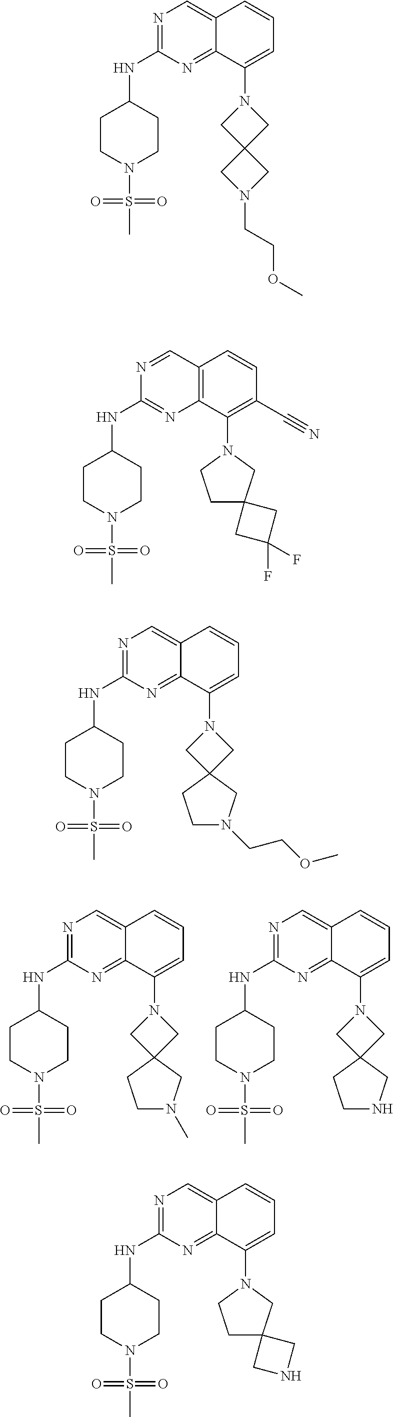

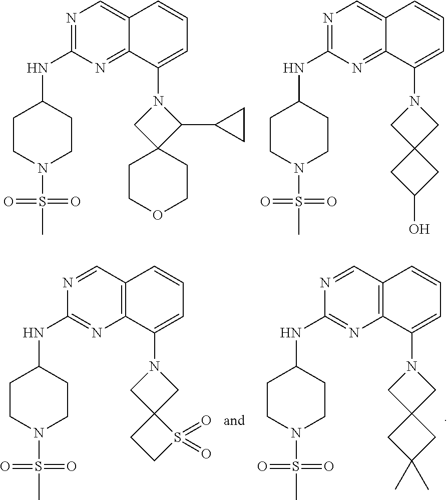

Embodiment 120. The compound or salt of embodiment [0320], wherein the compound is selected from:

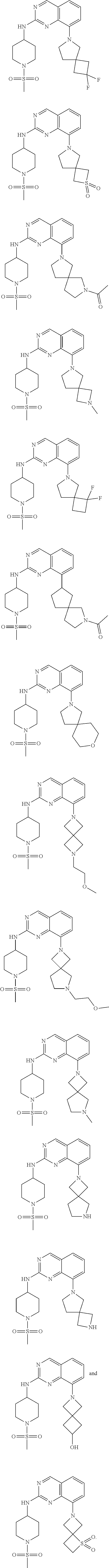

Embodiment 121. The compound, or a pharmaceutically acceptable salt or solvate thereof, of embodiment 1 having the structure of Formula (IB):

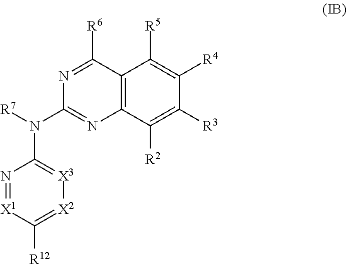

wherein,
each of X¹, X², and X³ is independently selected from N and CR³;
R¹² is selected from hydrogen, halogen, —CN, —NO₂, optionally substituted alkyl, optionally substituted alkenyl, optionally substituted alkynyl, optionally substituted carbocycle, and optionally substituted heterocycle, or R¹² comes together with R¹³ to form an optionally substituted ring; and
each R¹³ is independently selected from hydrogen, halogen, —CN, and optionally substituted C₁₋₄ alkyl.

Embodiment 122. The compound, or a pharmaceutically acceptable salt or solvate thereof, of embodiment [0322] wherein R² is optionally substituted heterocycle.

Embodiment 123. The compound, or a pharmaceutically acceptable salt or solvate thereof, of embodiment [0322] having the structure of Formula (IBB):

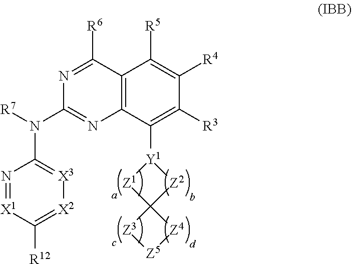

wherein,
Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle; and
each R¹¹ is independently selected from hydrogen and optionally substituted C₁₋₄ alkyl.

Embodiment 124. The compound embodiment [0325], wherein X¹, X², and X³ are each CH.

Embodiment 125. The compound or salt of embodiment [0325] or [0326], wherein each of Z¹, Z², Z³, Z⁴ and Z⁵ are independently selected from —C(R¹⁰)₂—, —NR¹¹—, —N(C(O)R¹⁰)—, —NS(O₂)R¹¹, —O—, and —S(O)₂—, wherein Z⁵ is additionally selected from a bond.

Embodiment 126. The compound or salt of any one of embodiments [0326] to [0327], wherein each of a, b, c, and d are independently selected from 1 and 2.

Embodiment 127. The compound or salt of any one of embodiments [0325] to [0328], wherein each R¹⁰ is independently selected from hydrogen, halogen, —CN, —OH, —O—C₁₋₄ alkyl, and optionally substituted C₁₋₃ alkyl.

Embodiment 128. The compound or salt of embodiment [0329], wherein each R¹⁰ is independently selected from hydrogen, halogen, —CN, —OH, —OMe, methyl, and —CH₂CH₂OCH₃.

Embodiment 129. The compound or salt of any one of embodiments [0325] to [0330], wherein each R¹¹ is independently selected from hydrogen and optionally substituted C₁₋₂ alkyl.

Embodiment 130. The compound or salt of embodiment [0331], wherein each R¹ is independently selected from hydrogen, methyl, and ethyl, wherein the ethyl is optionally substituted with —OMe.

Embodiment 131. The compound or salt of any one of embodiments [0325] to [0332], wherein the compound is selected from:

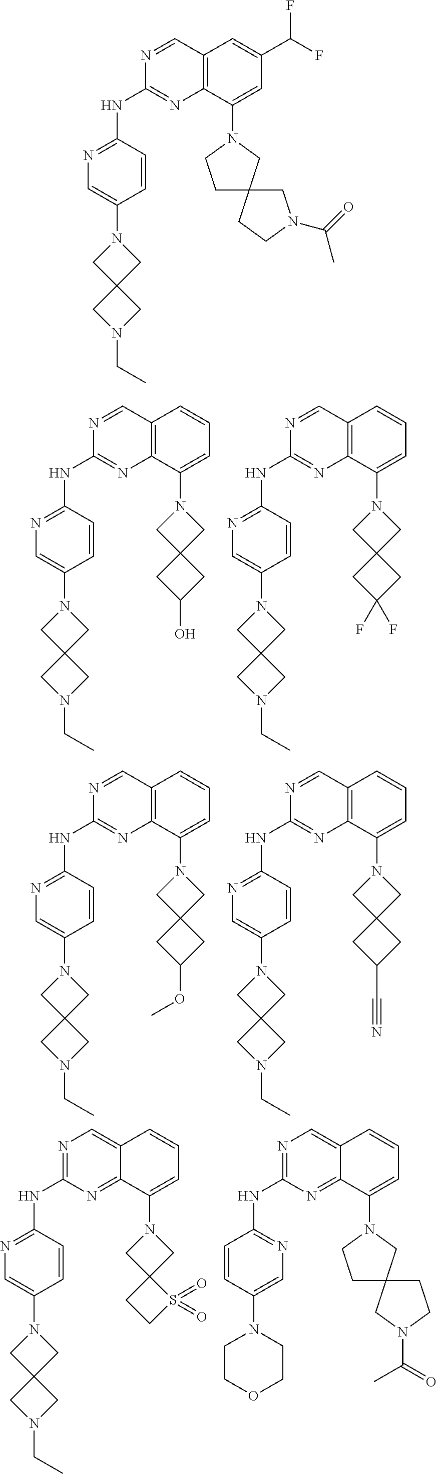

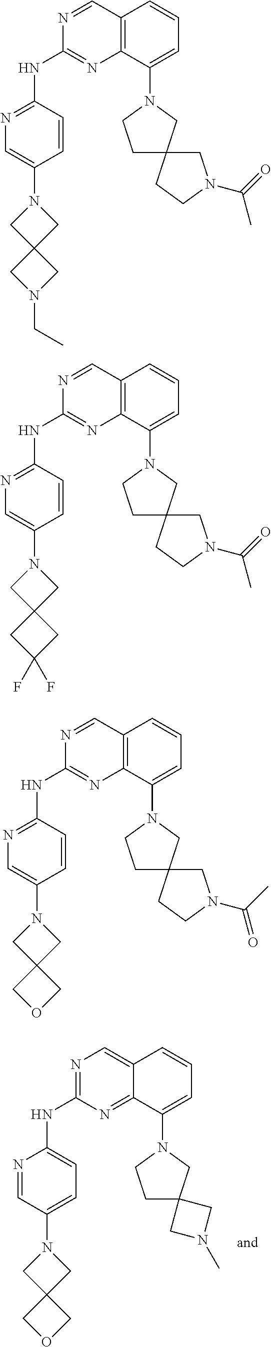

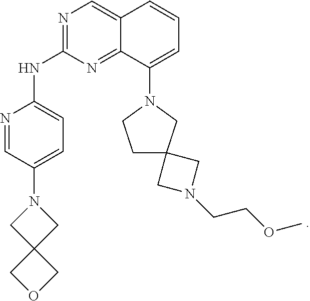 [SMILES: COCCN1CC2(CCN(c3cccc4cnc(Nc5ccc(N6CC7(COC7)C6)cn5)nc34)C2)C1]

Embodiment 132. The compound or salt of embodiment [0333], wherein the compound is selected from:

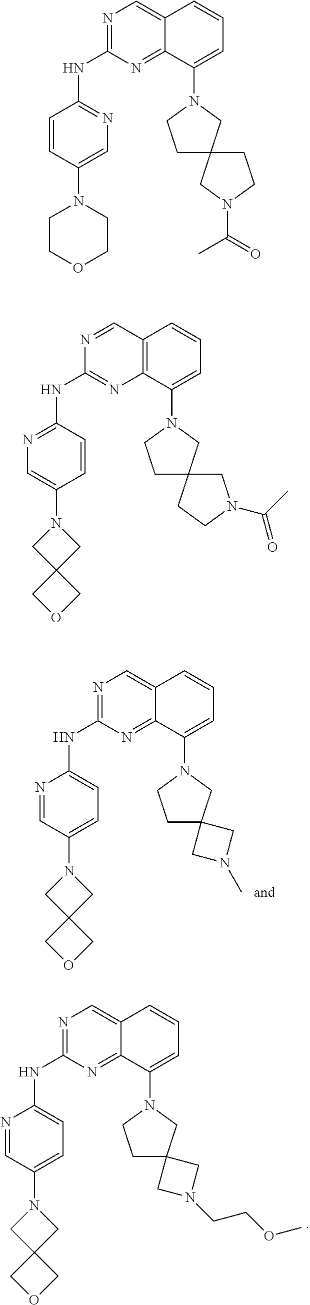

Embodiment 133. The compound, or a pharmaceutically acceptable salt or solvate thereof, of embodiment 1 having the structure of Formula (IC):

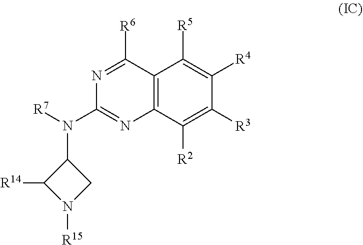

wherein,
   R¹⁴ is selected from hydrogen, halogen, —CN, optionally substituted C₁₋₄ alkyl, optionally substituted C₃₋₆ carbocycle, and optionally substituted 3- to 6-membered heterocycloalkyl, or R⁴ and
   R¹⁵ come together to form an optionally substituted heterocycle; and R⁵ is selected from —S(O)₂R¹⁶—, optionally substituted C₁₋₄ alkyl, optionally substituted C₃₋₆ carbocycle, and optionally substituted 3- to 6-membered heterocycloalkyl.

Embodiment 134. The compound, or a pharmaceutically acceptable salt or solvate thereof, of embodiment [0333] having the structure of Formula (ICC):

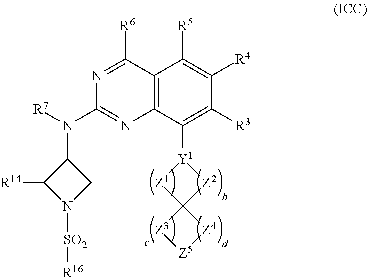

wherein,
Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle;
each R¹¹ is independently selected from hydrogen and optionally substituted C₁₋₄ alkyl; and
R¹⁶ is selected from optionally substituted C₁₋₄ alkyl, optionally substituted C₃₋₆ carbocycle, and optionally substituted 3- to 6-membered heterocycloalkyl.

Embodiment 135. The compound or salt of embodiment 1, wherein R¹ is selected from optionally substituted azabicyclo[3.1.0]hexane, optionally substituted isoindole, and optionally substituted indole.

Embodiment 136. The compound or salt of embodiment 16, wherein R¹ is selected from optionally substituted azabicyclo[3.1.0]hexane, and optionally substituted isoindole.

Embodiment 137. The compound or salt of embodiment 16 or Error! Reference source not found., wherein R¹ is substituted with —SO₂R^{1a} or C₁₋₃ alkyl, wherein R^{1a} is selected from C₁₋₆ alkyl.

Embodiment 138. The compound of any one of embodiments 16 to 17, wherein the compound is selected from:

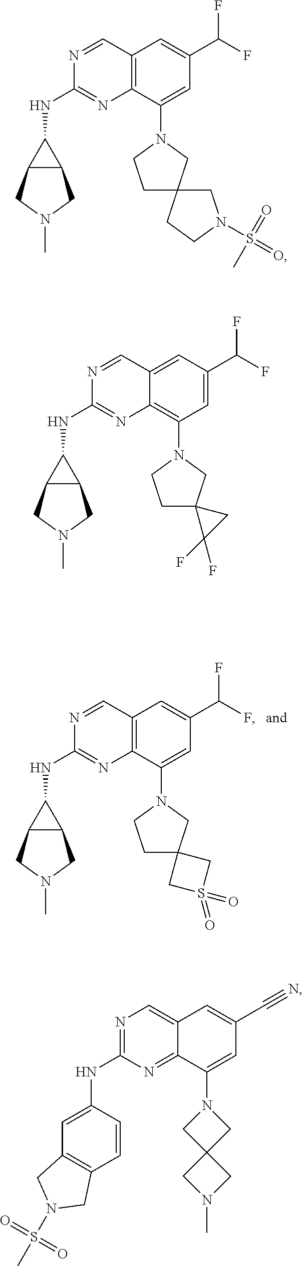 [SMILES: CN1C[C@@H]2[C@H](C1)[C@H]2Nc1ncc2cc(C(F)F)cc(N3CCC4(CCN(S(C)(=O)=O)C4)C3)c2n1 ; CN1C[C@@H]2[C@H](C1)[C@H]2Nc1ncc2cc(C(F)F)cc(N3CCC4(C3)CC4(F)F)c2n1 ; CN1C[C@@H]2[C@H](C1)[C@H]2Nc1ncc2cc(C(F)F)cc(N3CCC4(C3)CS(=O)(=O)C4)c2n1 ; "and" ; CN1CC2(C1)CN(c1cc(C#N)cc3cnc(Nc4ccc5c(c4)CN(S(C)(=O)=O)C5)nc13)C2]

Embodiment 139. The compound or salt of any one of embodiments 1, 2, [0323], or 16 to 17, wherein R² is selected from optionally substituted heterocycle and optionally substituted cycloalkyl.

Embodiment 140. The compound or salt of embodiment 18, wherein R² is selected from optionally substituted heterocycloalkyl.

Embodiment 141. The compound or salt of embodiment 18, wherein R² is selected from optionally substituted 3- to 6-membered heterocycloalkyl.

Embodiment 142. The compound or salt of embodiment Error! Reference source not found., wherein R² is selected from optionally substituted azetidine, optionally substituted pyrrolidine, optionally substituted piperidine, optionally substituted piperazine, and optionally substituted morpholine.

Embodiment 143. The compound or salt of any one of embodiments 18 to 42, wherein R² is substituted with halogen, —SO₂R^{2a}, —NR^{2a}, —C(O)CH₃, —CN, optionally substituted 3- to 6-membered heterocycloalkyl, optionally substituted C₃₋₅ carbocycle, oxo, and optionally substituted C₁₋₃ alkyl, wherein R² is selected from C₁₋₆ alkyl.

Embodiment 144. The compound or salt of embodiment 19, wherein R² is substituted with fluoro, —SO₂Me, oxo, and methyl.

Embodiment 145. The compound or salt of embodiment 19, wherein R² is selected from

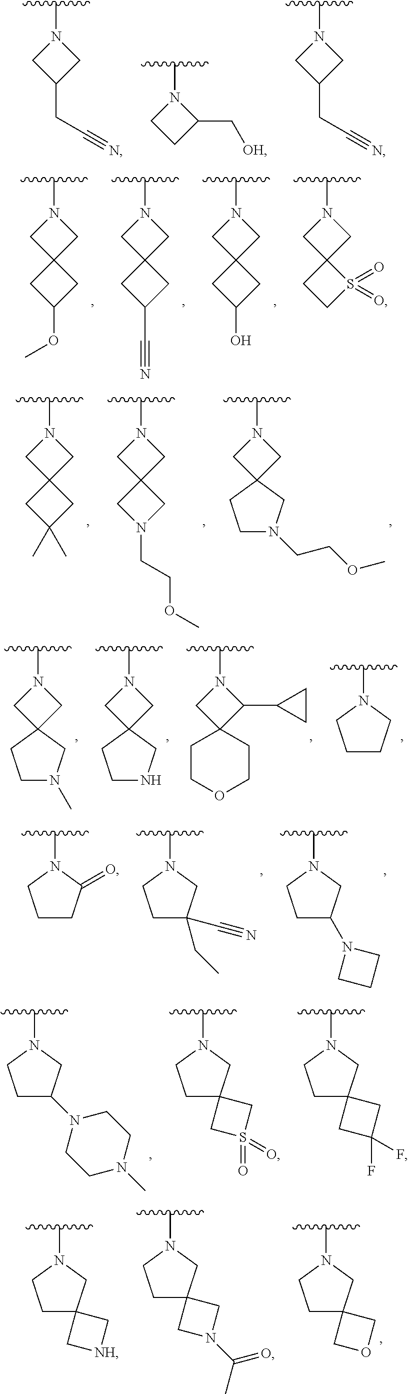

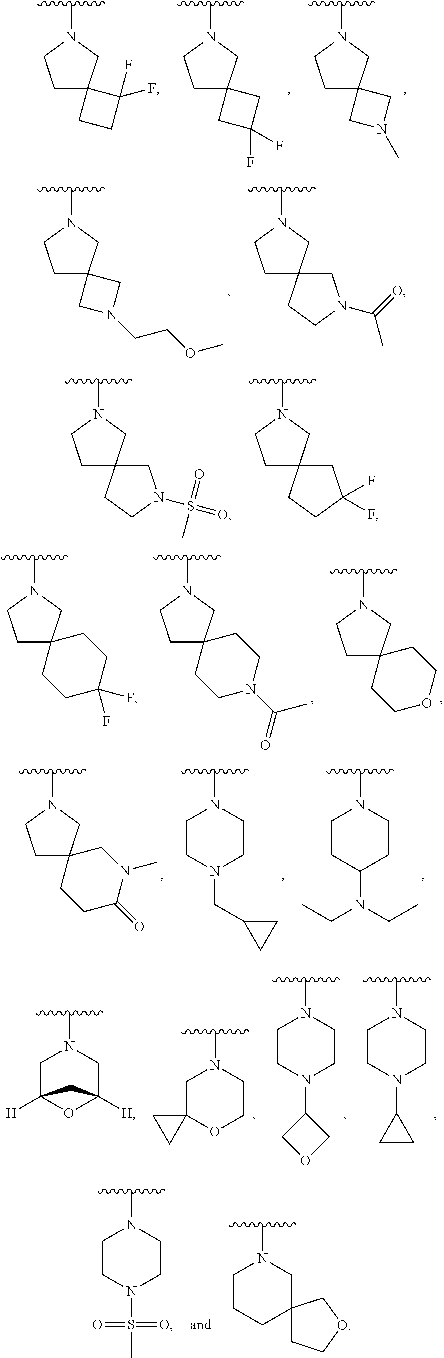

Embodiment 146. The compound or salt of embodiment 18, wherein R² is selected from optionally substituted cycloalkyl.

Embodiment 147. The compound or salt of embodiment 18, wherein R² is selected from optionally substituted cyclopentane.

Embodiment 148. The compound or salt of embodiment 18, wherein R² is selected from

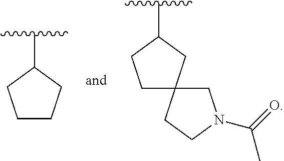 [SMILES: *C1CCCC1 ; "and" ; *C1CCC2(CCN(C(C)=O)C2)C1]

Embodiment 149. The compound or salt of any one of embodiments 1-[0307], 9-Error!Reference source not found., [0322]-[0332], or [0335]-20, wherein R³ is selected from hydrogen, halogen, —CN, optionally substituted C₃₋₄ carbocycle, and optionally substituted 3- to 4 membered heterocycloalkyl.

Embodiment 150. The compound or salt of embodiment 23, wherein R³ is selected from hydrogen, fluoro, —CN, cyclopropyl, cyclobutyl, optionally substituted oxetane, and optionally substituted azetidine.

Embodiment 151. The compound or salt of embodiment 24, wherein R³ is selected from hydrogen, fluoro, and —CN.

Embodiment 152. The compound or salt of embodiment 24, wherein R³ is selected from hydrogen and —CN.

Embodiment 153. The compound or salt of any one of embodiments 1-[0307], 9-Error!Reference source not found., [0322]-[0332], [0335]-20, or 23-Error! Reference source not found., wherein R⁴ is selected from hydrogen, halogen, —CN, optionally substituted C₁ alkyl, optionally substituted C₃₋₄ carbocycle, and optionally substituted 3- to 4 membered heterocycloalkyl.

Embodiment 154. The compound or salt of embodiment 25, wherein R⁴ is selected from hydrogen, —CN, —CHF₂, cyclopropyl, cyclobutyl, optionally substituted oxetane, and optionally substituted azetidine.

Embodiment 155. The compound or salt of embodiment 26, wherein R⁴ is selected from hydrogen, —CN, and —CHF₂.

Embodiment 156. The compound or salt of any one of embodiments 1-[0307], 9-Error!Reference source not found., [0322]-[0332], [0335]-20, or 23-26, wherein R⁵ is selected from hydrogen, halogen, —CN, optionally substituted C₃₋₄ carbocycle, and optionally substituted 3- to 4 membered heterocycloalkyl.

Embodiment 157. The compound or salt of embodiment 27, wherein R⁵ is selected from hydrogen, fluoro, —CN, cyclopropyl, cyclobutyl, optionally substituted oxetane, and optionally substituted azetidine.

Embodiment 158. The compound or salt of embodiment 28, wherein R⁵ is hydrogen.

Embodiment 159. The compound or salt of any one of embodiments 1-[0307], 9-Error!Reference source not found., [0322]-[0332], [0335]-20, or 23-28, wherein R⁶ is selected from hydrogen, halogen, —CN, optionally substituted C₃₋₄ carbocycle, and optionally substituted 3- to 4 membered heterocycloalkyl.

Embodiment 160. The compound or salt of embodiment 29, wherein R⁶ is selected from hydrogen, fluoro, —CN, cyclopropyl, cyclobutyl, optionally substituted oxetane, and optionally substituted azetidine.

Embodiment 161. The compound or salt of embodiment 29, wherein R⁶ is hydrogen.

Embodiment 162. The compound or salt of any one of embodiments 1-[0307], 9-Error!Reference source not found., [0322]-[0332], [0335]-20, or 23-30, wherein R⁷ is hydrogen.

Embodiment 163. The compound or salt of embodiment 1, wherein the compound is selected from:

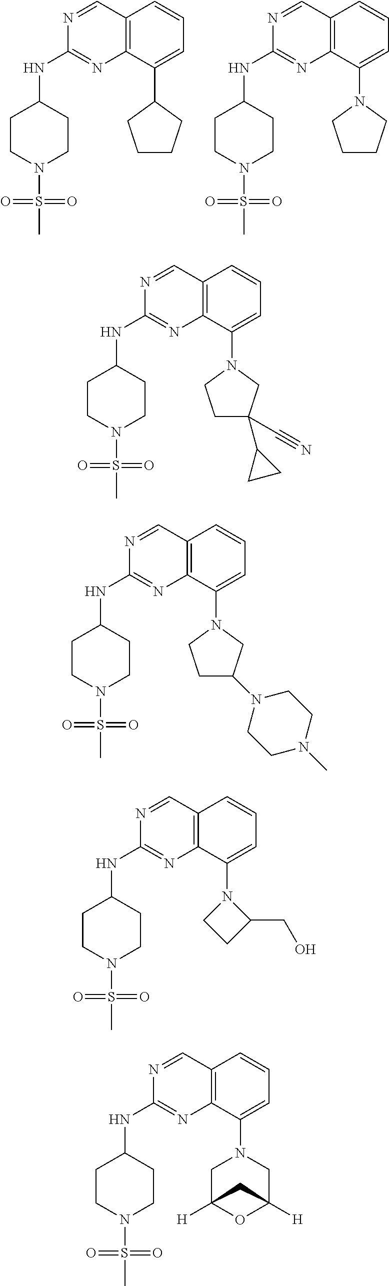

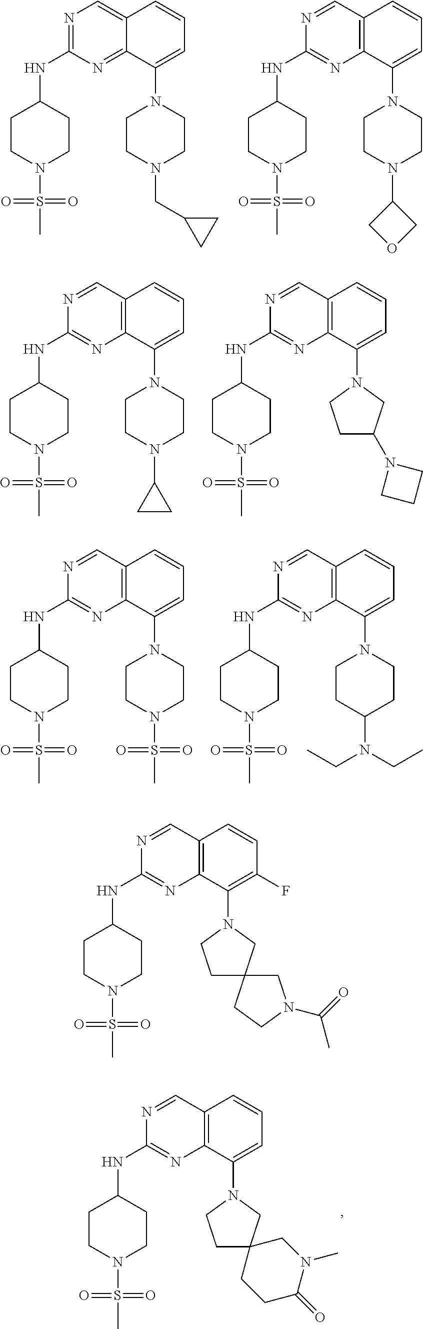

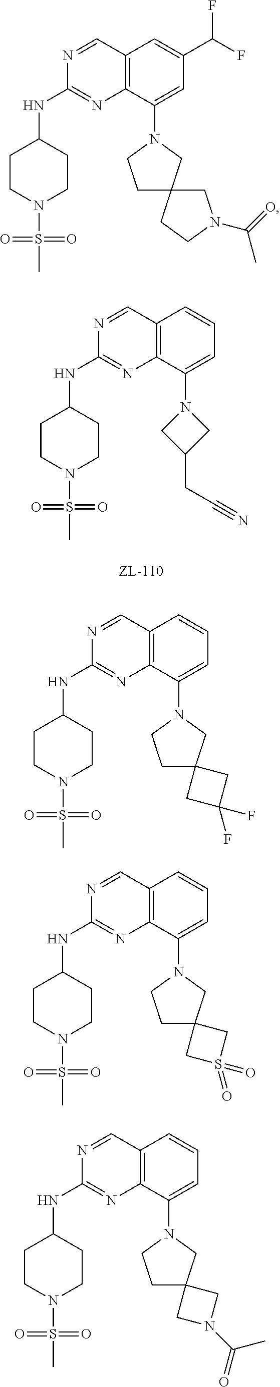 [SMILES: CC(=O)N1CCC2(CCN(c3cc(C(F)F)cc4cnc(NC5CCN(S(C)(=O)=O)CC5)nc34)C2)C1 ; CS(=O)(=O)N1CCC(Nc2ncc3cccc(N4CC(CC#N)C4)c3n2)CC1 ; CS(=O)(=O)N1CCC(Nc2ncc3cccc(N4CCC5(C4)CC(F)(F)C5)c3n2)CC1 ; CS(=O)(=O)N1CCC(Nc2ncc3cccc(N4CCC5(C4)CS(=O)(=O)C5)c3n2)CC1 ; CC(=O)N1CC2(CCN(c3cccc4cnc(NC5CCN(S(C)(=O)=O)CC5)nc34)C2)C1]

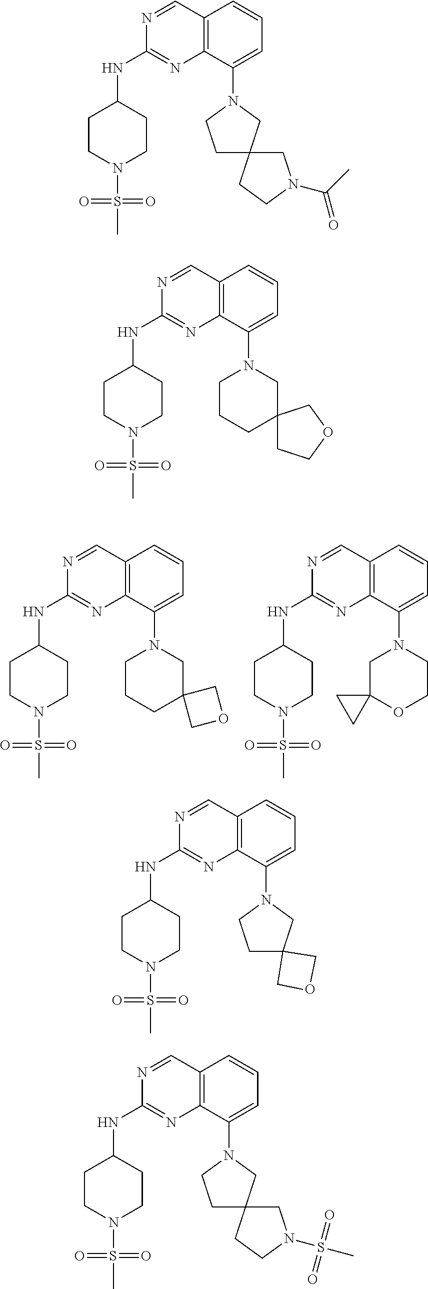

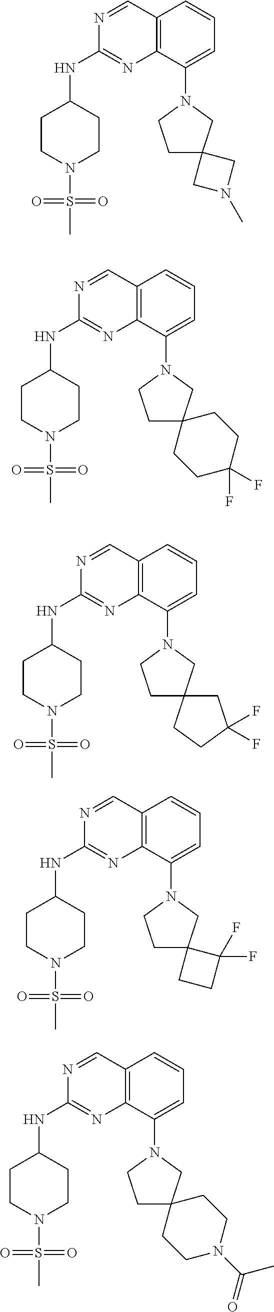

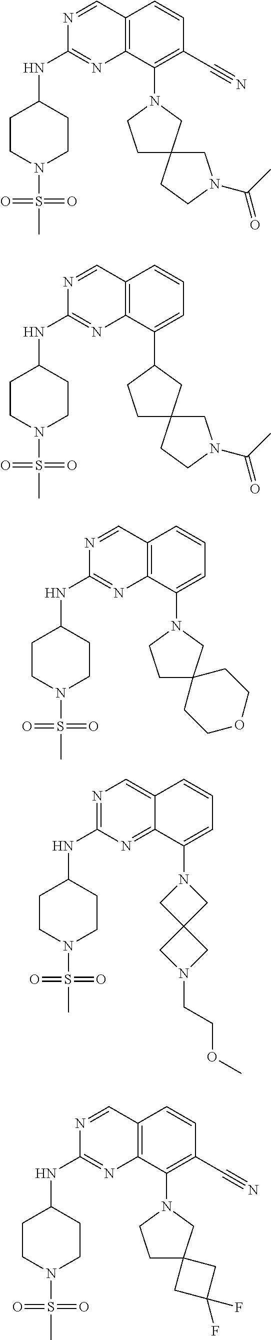 [SMILES: CC(=O)N1CCC2(CCN(c3c(C#N)ccc4cnc(NC5CCN(S(C)(=O)=O)CC5)nc34)C2)C1 ; CC(=O)N1CCC2(CCC(c3cccc4cnc(NC5CCN(S(C)(=O)=O)CC5)nc34)C2)C1 ; CS(=O)(=O)N1CCC(Nc2ncc3cccc(N4CCC5(CCOCC5)C4)c3n2)CC1 ; COCCN1CC2(C1)CN(c1cccc3cnc(NC4CCN(S(C)(=O)=O)CC4)nc13)C2 ; CS(=O)(=O)N1CCC(Nc2ncc3ccc(C#N)c(N4CCC5(C4)CC(F)(F)C5)c3n2)CC1]

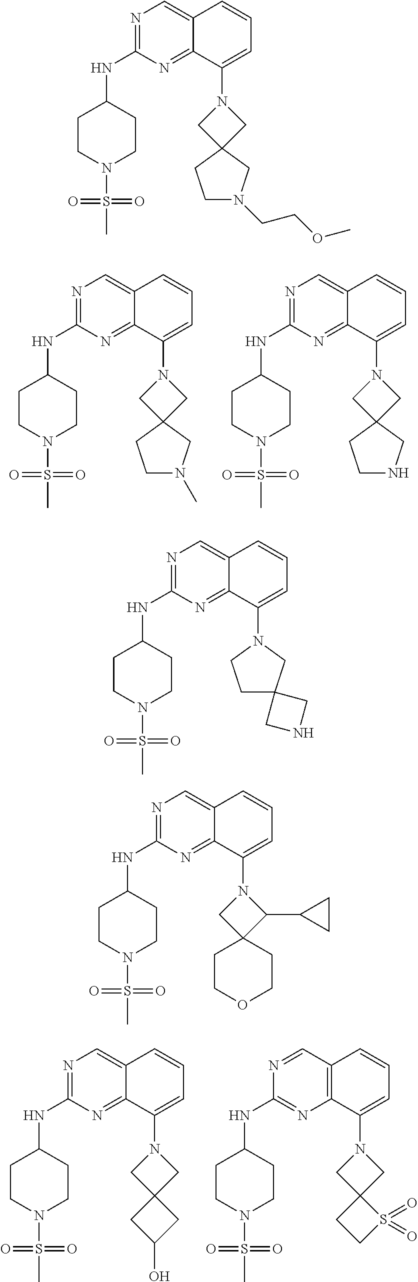

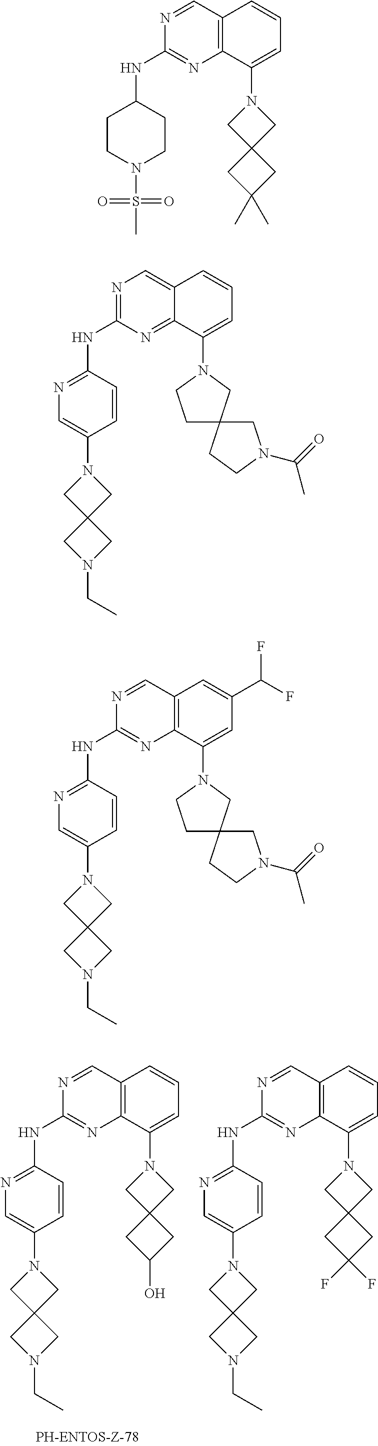

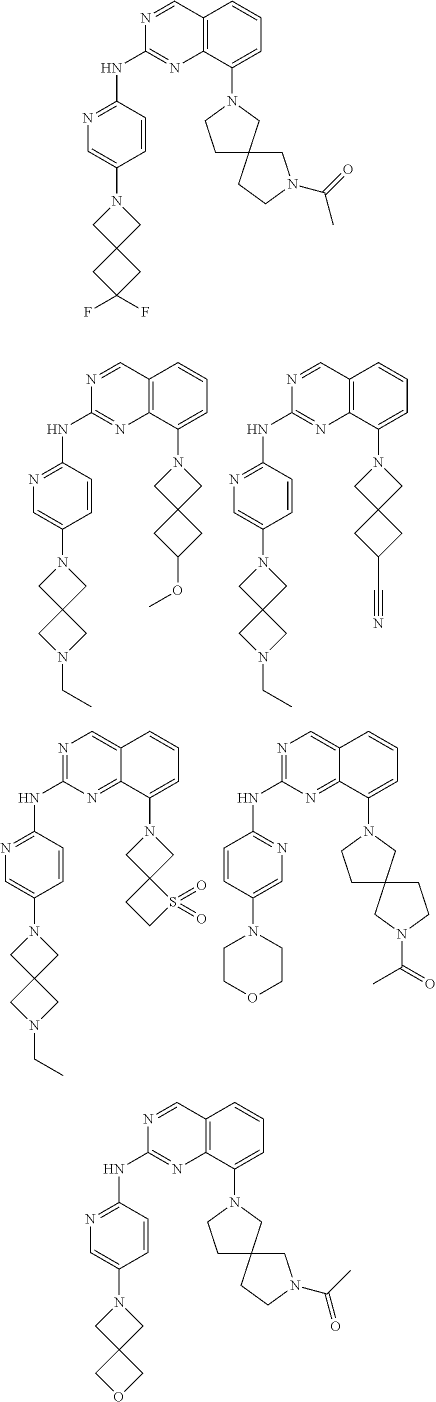

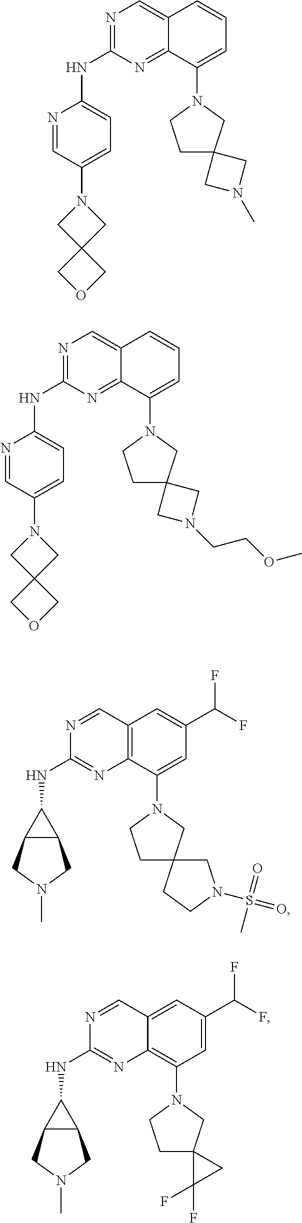

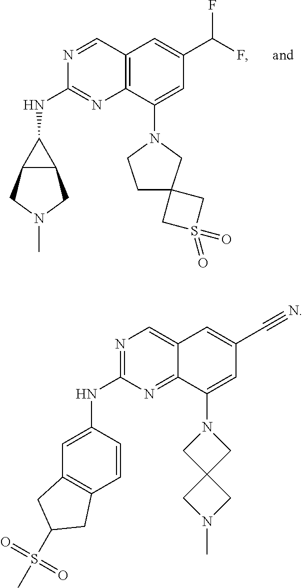 [SMILES: CN1C[C@@H]2[C@H](C1)[C@H]2Nc1ncc2cc(C(F)F)cc(N3CCC4(C3)CS(=O)(=O)C4)c2n1 ; CN1CC2(C1)CN(c1cc(C#N)cc3cnc(Nc4ccc5c(c4)CC(S(C)(=O)=O)C5)nc13)C2]

Embodiment 164. The compound or salt of embodiment 1, wherein the compound is selected from:

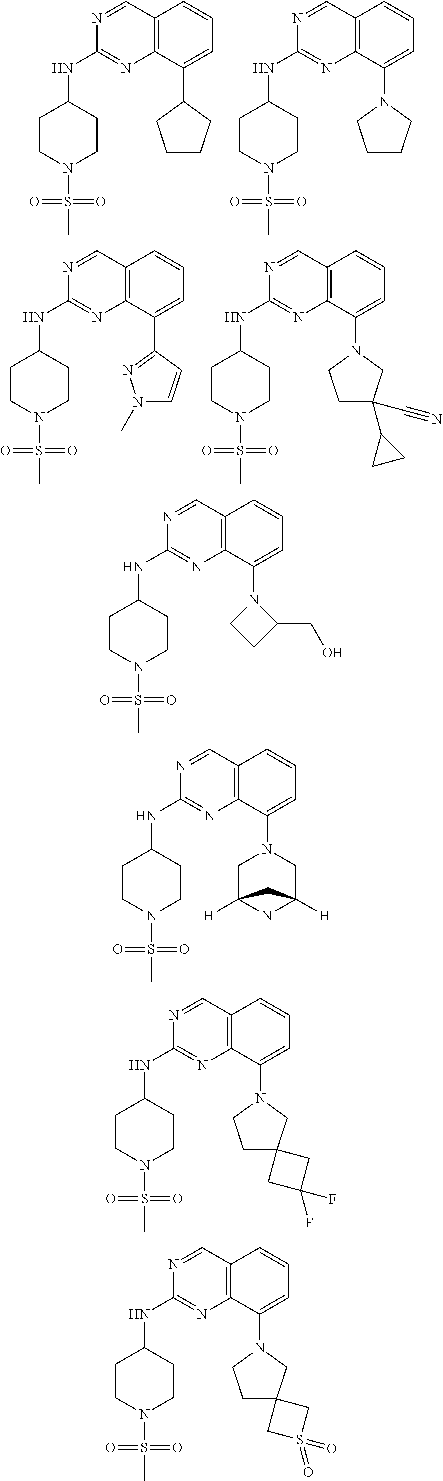

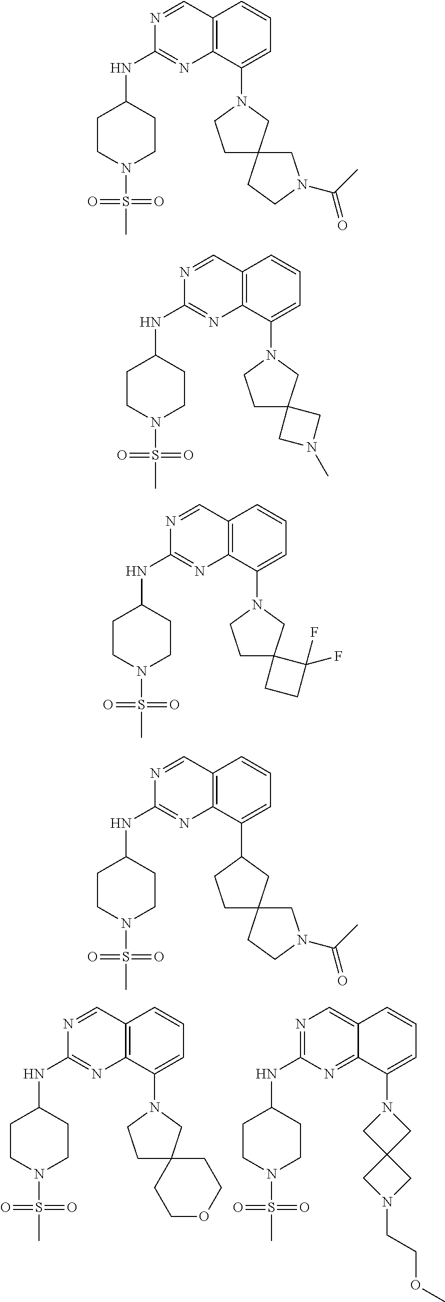

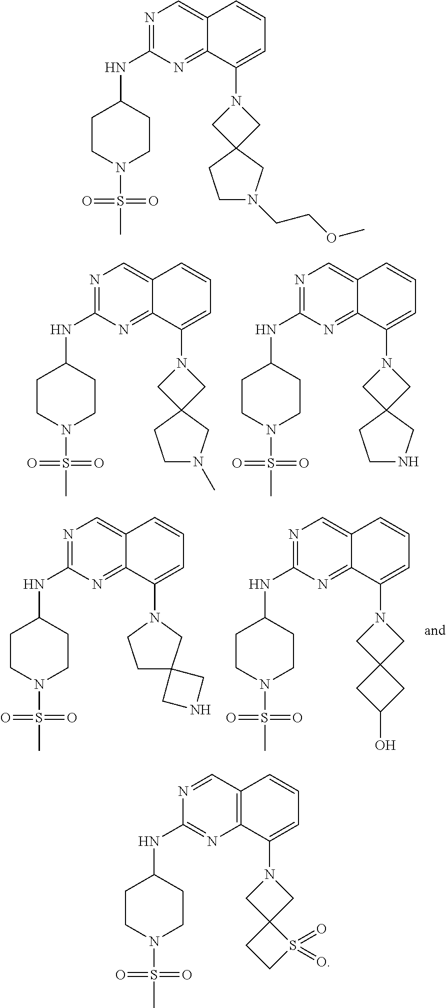

Embodiment 165. A compound, or a pharmaceutically acceptable salt or solvate thereof, having the structure of Formula (II):

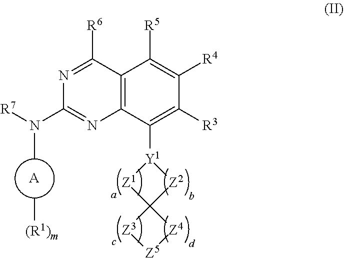

wherein,
A is a ring selected from optionally substituted C₃₋₆ carbocycle and optionally substituted 3- to 12-membered heterocycle;
R¹ is selected from halogen, —CN, —NO₂, optionally substituted alkyl, optionally substituted alkenyl, optionally substituted alkynyl, optionally substituted carbocycle, and optionally substituted heterocycle;
m is selected from 0 to 9;
   each of R³, R⁴, R⁵, R⁶, is independently selected from hydrogen, halogen, —CN, and optionally substituted C₁₋₄ alkyl, optionally substituted C₃₋₄ carbocycle, and optionally substituted 3- to 4-membered heterocyclcoalkyl; and
R⁷ is selected from hydrogen and optionally substituted C₁₋₄ alkyl;
   Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle; and
each R¹¹ is independently selected from hydrogen and optionally substituted C₁₋₄ alkyl.

Embodiment 166. The compound or salt of embodiment 31, wherein A is selected from optionally substituted C₅₋₆ carbocycle and optionally substituted 5- to 10-membered heterocycle.

Embodiment 167. The compound or salt of embodiment [0368], wherein A is selected from phenyl, pyrazole, and tetrahydroisoquinoline.

Embodiment 168. The compound or salt of any one of embodiments 31 to Error! Reference source not found., wherein m is selected from 0 to 4.

Embodiment 169. The compound or salt of embodiment Error! Reference source not found., wherein m is selected from 0 to 2.

Embodiment 170. The compound or salt of any one of embodiments 31 to Error! Reference source not found., wherein R¹ is selected from halogen, —CN, optionally substituted alkyl, optionally substituted carbocycle, and optionally substituted heterocycle.

Embodiment 171. The compound or salt of embodiment Error! Reference source not found., wherein R¹ is selected from optionally substituted C₁₋₃ alkyl, and optionally substituted 5- to 8-membered heterocycle.

Embodiment 172. The compound or salt of embodiment Error! Reference source not found., wherein R¹ is selected from C₁₋₃ alkyl, and substituted 5- to 8-membered heterocycle.

Embodiment 173. The compound, or a pharmaceutically acceptable salt or solvate thereof, of embodiment 31 having the structure of Formula (IIA):

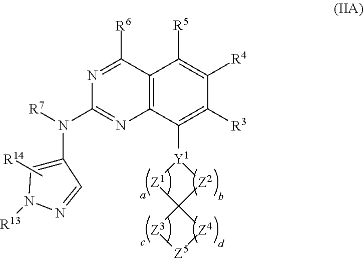

wherein,
   R³ is selected from optionally substituted C₁₋₄ alkyl, optionally substituted C₃₋₆ carbocycle, and optionally substituted 3- to 6-membered heterocycloalkyl, or R³ and R¹⁴ come together to form an optionally substituted heterocycle; and
   R¹⁴ is selected from halogen, —CN, optionally substituted C₁₋₄ alkyl, optionally substituted C₃₋₆ carbocycle, and optionally substituted 3- to 6-membered heterocycloalkyl.

Embodiment 174. The compound or salt of any one of embodiments 31 to [0375], wherein R³ is selected from hydrogen, halogen, —CN, optionally substituted C₃₋₄ carbocycle, and optionally substituted 3- to 4 membered heterocycloalkyl.

Embodiment 175. The compound or salt of embodiment [0376], wherein R³ is selected from hydrogen, fluoro, —CN, cyclopropyl, cyclobutyl, optionally substituted oxetane, and optionally substituted azetidine.

Embodiment 176. The compound or salt of embodiment [0377], wherein R³ is selected from hydrogen, fluoro, and —CN.

Embodiment 177. The compound or salt of any one of embodiments 31 to [0378], wherein R⁴ is selected from hydrogen, halogen, —CN, optionally substituted C₁ alkyl, optionally substituted C₃₋₄ carbocycle, and optionally substituted 3- to 4 membered heterocycloalkyl.

Embodiment 178. The compound or salt of embodiment [0379], wherein R⁴ is selected from hydrogen, —CN, —CHF₂, cyclopropyl, cyclobutyl, optionally substituted oxetane, and optionally substituted azetidine.

Embodiment 179. The compound or salt of embodiment [0380], wherein R⁴ is selected from hydrogen, —CN, and —CHF₂.

Embodiment 180. The compound or salt of any one of embodiments 31 to [0381], wherein R⁵ is selected from hydrogen, halogen, —CN, optionally substituted C₃₋₄ carbocycle, and optionally substituted 3- to 4 membered heterocycloalkyl.

Embodiment 181. The compound or salt of embodiment [0382], wherein R⁵ is selected from hydrogen, fluoro, —CN, cyclopropyl, cyclobutyl, optionally substituted oxetane, and optionally substituted azetidine.

Embodiment 182. The compound or salt of embodiment [0383], wherein R⁵ is hydrogen.

Embodiment 183. The compound or salt of any one of embodiments 31 to [0384], wherein R⁶ is selected from hydrogen, halogen, —CN, optionally substituted C₃₋₄ carbocycle, and optionally substituted 3- to 4 membered heterocycloalkyl.

Embodiment 184. The compound or salt of embodiment [0385], wherein R⁶ is selected from hydrogen, fluoro, —CN, cyclopropyl, cyclobutyl, optionally substituted oxetane, and optionally substituted azetidine.

Embodiment 185. The compound or salt of embodiment [0386], wherein R⁶ is hydrogen.

Embodiment 186. The compound or salt of any one of embodiments 31 to [0387], wherein R⁷ is hydrogen.

Embodiment 187. The compound or salt of any one of embodiments 31 to [0388], wherein each of Z¹, Z², Z³, Z⁴ and Z⁵ are independently selected from —C(R¹⁰)₂—, —NR¹¹—, —N(C(O)R¹⁰)—, —NS(O₂)R¹¹, —O—, and —S(O)₂—, wherein Z⁵ is additionally selected from a bond.

Embodiment 188. The compound or salt of any one of embodiments 31 to [0389], wherein each of a, b, c, and d are independently selected form 1 and 2.

Embodiment 189. The compound or salt of any one of embodiments 31 to [0390], wherein each R¹⁰ is independently selected from hydrogen, halogen, —CN, —OH, —O—C₁₋₄ alkyl, and optionally substituted C₁₋₃ alkyl.

Embodiment 190. The compound or salt of embodiment [0391], wherein each R¹⁰ is independently selected from hydrogen, halogen, —CN, —OH, —OMe, methyl, and —CH₂CH₂OCH₃.

Embodiment 191. The compound or salt of any one of embodiments 31 to [0392], wherein each R¹¹ is independently selected from hydrogen and optionally substituted C₁₋₂ alkyl.

Embodiment 192. The compound or salt of embodiment [0393], wherein each R¹¹ is independently selected from hydrogen, methyl, and ethyl, wherein the ethyl is optionally substituted with —OMe.

Embodiment 193. The compound or salt of any one of embodiments 31 to [0394], wherein the compound is selected from:

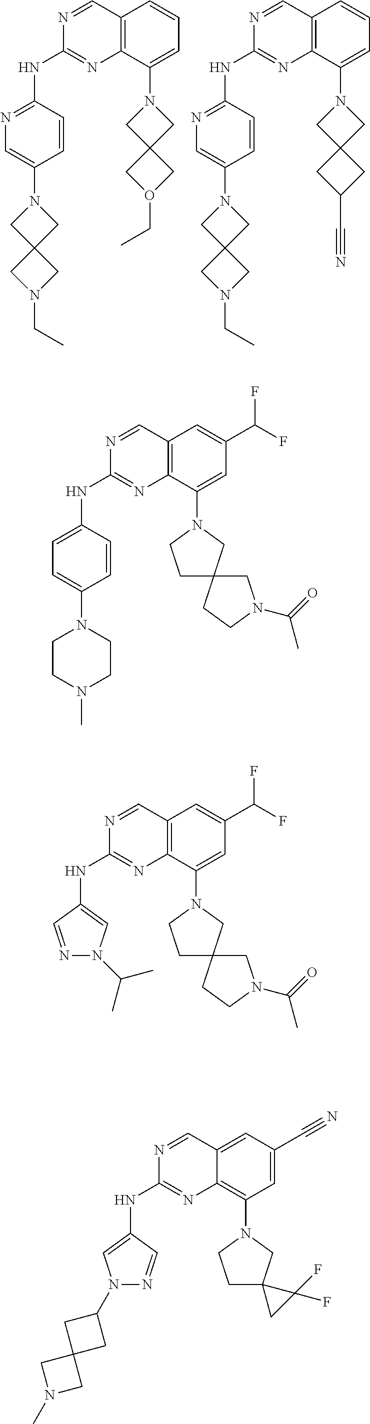

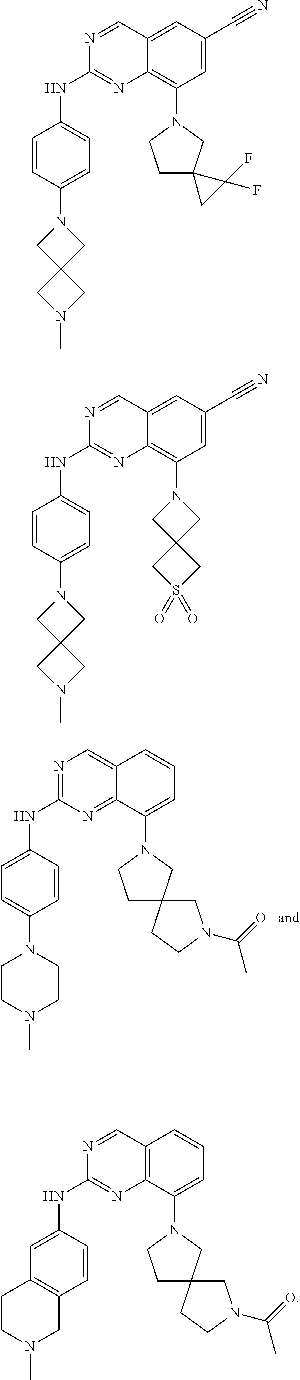

Embodiment 194. The compound or salt of embodiment [0395], wherein the compound is selected from:

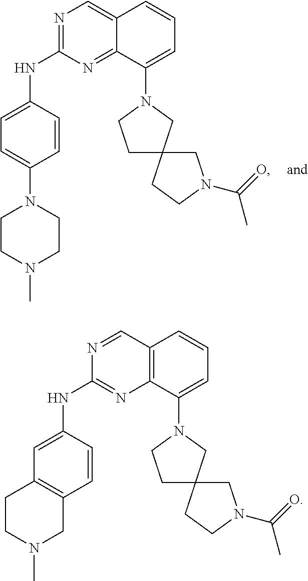 [SMILES: CC(=O)N1CCC2(CCN(c3cccc4cnc(Nc5ccc(N6CCN(C)CC6)cc5)nc34)C2)C1 ; CC(=O)N1CCC2(CCN(c3cccc4cnc(Nc5ccc6c(c5)CCN(C)C6)nc34)C2)C1]

Embodiment 195. A pharmaceutical composition comprising a compound or salt of any one of embodiments 1 to [0396] and a pharmaceutically acceptable excipient.

Embodiment 196. A method of treating cancer, comprising administering to a subject in need thereof the pharmaceutical composition of embodiment 33.

Embodiment 197. The method of embodiment 196, wherein the cancer is a solid tumor.

Embodiment 198. The method of embodiment 196 or 197, wherein the cancer is selected from ovarian cancer, breast cancer, colon cancer, and brain cancer.

Embodiment 199. The method of embodiment 198, wherein the cancer is ovarian cancer or breast cancer.

Embodiment 200. A method of inhibiting a cyclin dependent kinase (CDK) in a cell with a compound or salt of any one of embodiments 1 to [0396] or the pharmaceutical composition of embodiment 33.

Embodiment 201. The method of embodiment 37, wherein the CDK is selected from CDK 2, CDK 4, CD6, or any combination thereof.

Embodiment 202. The method of embodiment 37, wherein the CDK is selected from CDK 2/4, CDK 2/6, CDK 4/6, and CDK 2/4/6.

Embodiment 203. The method of embodiment 202, wherein the CDK is CDK 2/4/6.

### EXAMPLES

The invention now being generally described, it will be more readily understood by reference to the following examples which are included merely for purposes of illustration of certain aspects and embodiments of the present invention, and are not intended to limit the invention in any way.

The following synthetic schemes are provided for purposes of illustration, not limitation. The following examples illustrate the various methods of making compounds described herein. It is understood that one skilled in the art may be able to make these compounds by similar methods or by combining other methods known to one skilled in the art. It is also understood that one skilled in the art would be able to make, in a similar manner as described below by using the appropriate starting materials and modifying the synthetic route as needed. In general, starting materials and reagents can be obtained from commercial vendors or synthesized according to sources known to those skilled in the art or prepared as described herein.

### General Synthetic Schemes 1-4

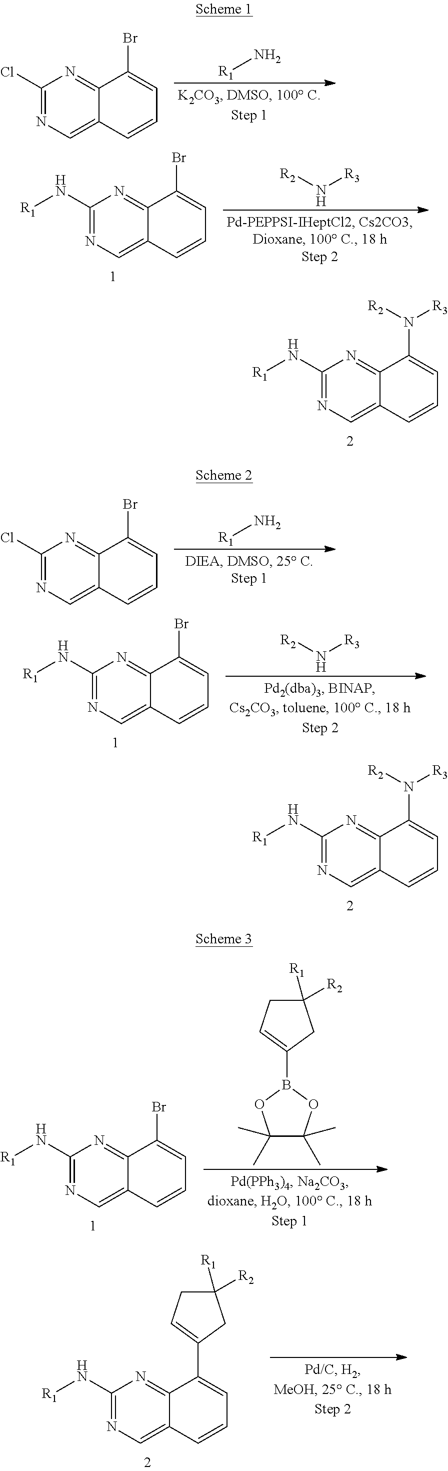

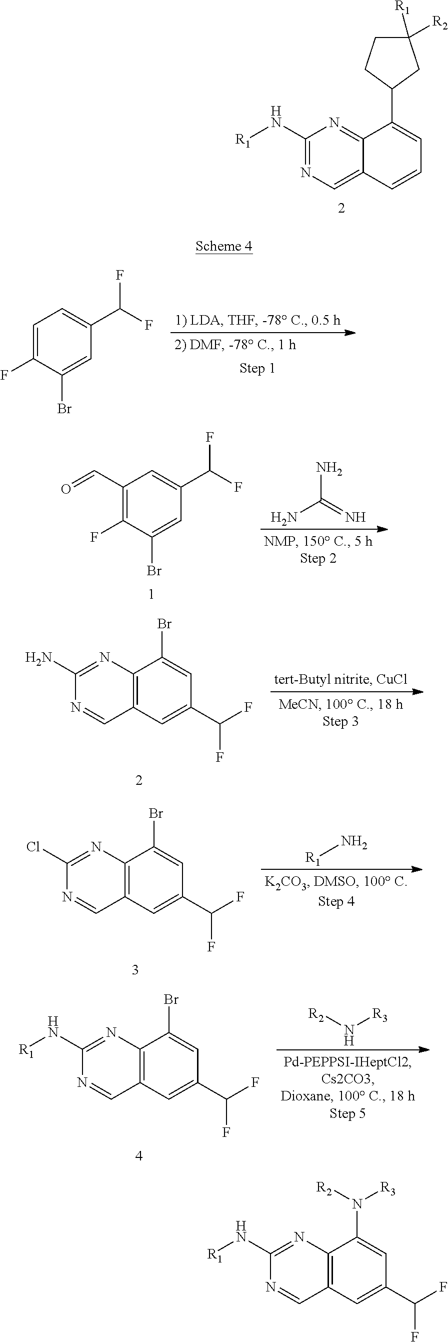

### INTERMEDIATES

### Intermediate 1: 8-bromo-N-(1-(methylsulfonyl)piperidin-4-yl)quinazolin-2-amine

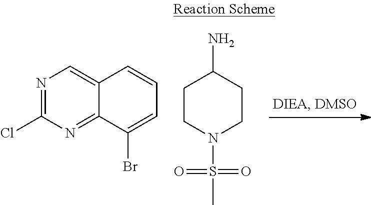

### Detailed Procedure

### Step 1: 8-bromo-N-(1-(methylsulfonyl)piperidin-4-yl)quinazolin-2-amine

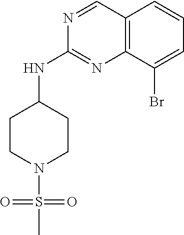 [SMILES: CS(=O)(=O)N1CCC(Nc2ncc3cccc(Br)c3n2)CC1]

To a stirred mixture of 8-bromo-2-chloroquinazoline (20 g, 82.1 mmol) and 1-(methylsulfonyl)piperidin-4-amine (14.64 g, 82.1 mmol) in dimethyl sulfoxide (400 mL) was added N,N-diisopropylethylamine (31.8 g, 246.3 mmol). The resulting mixture was stirred for 3 hours at room temperature, diluted with water (1000 mL) and extracted with ethyl acetate (3×1000 mL). The combined organic layers were washed with brine (1000 mL), dried over anhydrous sodium sulfate and filtered. The filtrate was concentrated under vacuum to afford the crude product. The residue was purified by silica gel column chromatography (1:1 petroleum ether/ethyl acetate) to afford 8-bromo-N-(1-(methylsulfonyl)piperidin-4-yl)quinazolin-2-amine (5.04 g, 13.09 mmol, 15.9% yield).

LCMS (ESI) m/z=385 [M+H]⁺.

¹H NMR (400 MHz, DMSO-d₆) δ 9.13 (s, 1H), 8.04 (d, J=7.6 Hz, 1H), 7.83-7.72 (m, 2H), 7.17-7.13 (m, 1H), 4.03-4.01 (m, 1H), 3.59-3.56 (m, 2H), 2.95-2.90 (m, 5H), 2.13-1.96 (m, 2H), 1.69-1.60 (m, 2H).

### Intermediate 2: 8-(8,8-difluoro-2,6-diazaspiro[3,4]octan-6-yl)-6-methyl-N-(1-((1-methyl-1H-pyrazol-4-yl)sulfonyl)piperidin-4-yl)pyrido[3,4-d]pyrimidin-2-amine

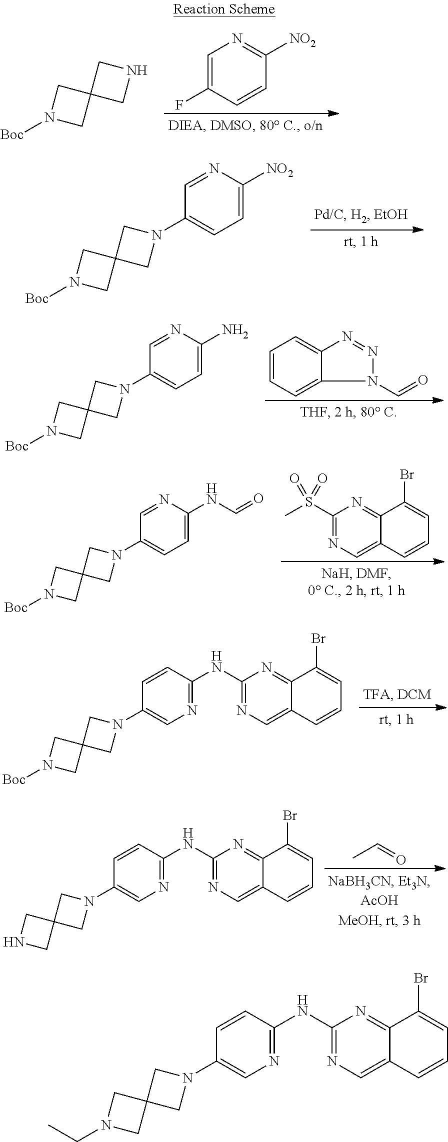

### Detailed Procedure

### Step 1: Tert-butyl 6-(6-nitropyridin-3-yl)-2,6-diazaspiro[3.3]heptane-2-carboxylate

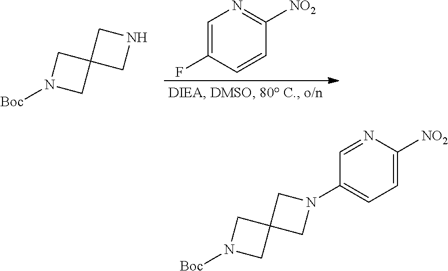 [SMILES: CC(C)(C)OC(=O)N1CC2(CNC2)C1 ; O=[N+]([O-])c1ccc(F)cn1 ; CC(C)(C)OC(=O)N1CC2(C1)CN(c1ccc([N+](=O)[O-])nc1)C2]

DIEA (9.78 g, 75.6 mmol) was added to a stirred mixture of tert-butyl 2,6-diazaspiro[3.3]heptane-2-carboxylate (5.0 g, 25.2 mmol) and 5-fluoro-2-nitropyridine (5.37 g, 37.8 mmol) in DMSO (30 mL). The resulting mixture was heated to 80° C. and stirred overnight. After cooling to room temperature, the reaction mixture was diluted with water (500 mL) and extracted with EA (3×500 mL). The combined organic layers were washed with brine (500 mL), dried over anhydrous sodium sulfate and filtered. The filtrate was concentrated under reduced pressure to afford the crude product. The residue was purified by trituration with 100 mL of 1:5 EA/PE to afford the desired product tert-butyl 6-(6-nitropyridin-3-yl)-2,6-diazaspiro[3.3]heptane-2-carboxylate (6.78 g, 83.7% yield).

LCMS (ESI) m/z=321.1 [M+H]⁺.

### Step 2: Tert-butyl 6-(6-aminopyridin-3-yl)-2,6-diazaspiro[3.3]heptane-2-carboxylate

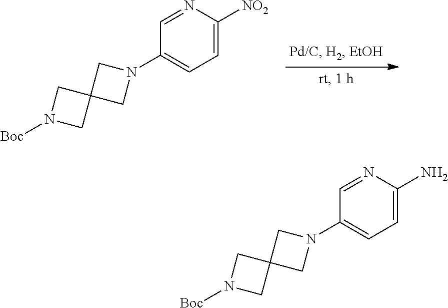 [SMILES: CC(C)(C)OC(=O)N1CC2(C1)CN(c1ccc([N+](=O)[O-])nc1)C2 ; CC(C)(C)OC(=O)N1CC2(C1)CN(c1ccc(N)nc1)C2]

Pd/C (10% on carbon, 200 mg) was added to a mixture of tert-butyl 6-(6-nitropyridin-3-yl)-2,6-diazaspiro[3.3]heptane-2-carboxylate (1 g, 3.12 mmol) in EtOH (25 mL) under a nitrogen atmosphere. The reaction mixture was stirred at room temperature for 1 h under a hydrogen atmosphere. The reaction mixture was filtered and the filtrate was concentrated under reduced pressure to afford crude tert-butyl 6-(6-aminopyridin-3-yl)-2,6-diazaspiro[3.3]heptane-2-carboxylate (800 mg, 83.8% yield).

LCMS (ESI-MS) m/z=291.2 [M+H]⁺.

### Step 3: Tert-butyl 6-formamidopyridin-3-yl)-2,6-diazaspiro[3.3]heptane-2-carboxylate

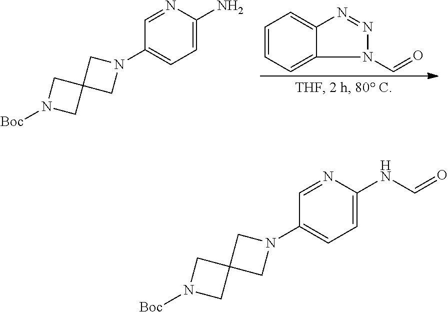 [SMILES: CC(C)(C)OC(=O)N1CC2(C1)CN(c1ccc(N)nc1)C2 ; O=Cn1nnc2ccccc21 ; CC(C)(C)OC(=O)N1CC2(C1)CN(c1ccc(NC=O)nc1)C2]

A mixture of tert-butyl 6-(6-aminopyridin-3-yl)-2,6-diazaspiro[3.3]heptane-2-carboxylate (4.7 g, 16.2 mmol) and 1H-benzo[d][1,2,3]triazole-1-carbaldehyde (2.62 g, 17.8 mmol) in THF (50 mL) was heated to 80° C. and stirred for 2 h. After cooling to room temperature, the reaction mixture was concentrated under reduced pressure to afford the crude product. The residue was purified by silica gel column chromatography (EA/PE, 7:3) to afford the desired product tert-butyl 6-(6-formamidopyridin-3-yl)-2,6-diazaspiro[3.3]heptane-2-carboxylate (5.2 g, 95.8% yield).

LCMS (ESI-MS) m/z=319.2 [M+H]⁺.

### Step 4: Tert-butyl 6-(6-((8-bromoquinazolin-2-yl)amino)pyridin-3-yl)-2,6-diazaspiro[3.3]heptane-2-carboxylate

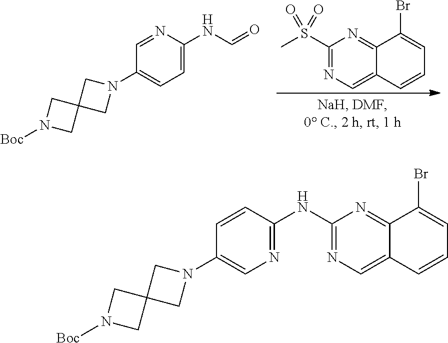

NaH (60% in mineral oil, 0.23 g, 9.42 mmol) was added to a stirred mixture of tert-butyl 6-(6-formamidopyridin-3-yl)-2,6-diazaspiro[3.3]heptane-2-carboxylate (1 g, 3.14 mmol) in DMF (10 mL) at 0° C. The resulting mixture was stirred at 0° C. for 2 h and 8-bromo-2-(methylsulfonyl)quinazoline (0.99 g, 3.45 mmol) was added. The resulting mixture was warmed to room temperature and stirred for another 1 h. The reaction mixture was quenched by addition of water (100 mL) and extracted with EA (3×200 mL). The combined organic layers were washed with brine (200 mL), dried over anhydrous sodium sulfate, filtered and the filtrate was concentrated under reduced pressure to afford the crude product. The residue was purified by trituration with DCM (40 mL) to afford the desired product tert-butyl 6-(6-((8-bromoquinazolin-2-yl)amino)pyridin-3-yl)-2,6-diazaspiro[3.3]heptane-2-carboxylate (800 mg, 46.1% yield).

LCMS (ESI-MS) m/z=497.2 [M+H]⁺.

### Step 5: N-(5-(2,6-diazaspiro[3.3]heptan-2-yl)pyridin-2-yl)-8-bromoquinazolin-2-amine

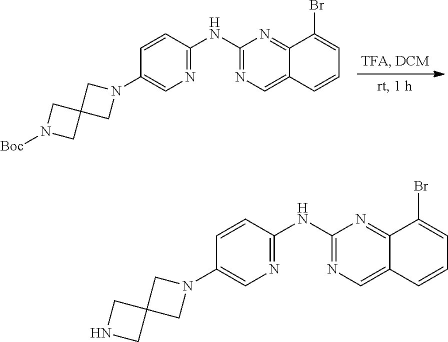 [SMILES: CC(C)(C)OC(=O)N1CC2(C1)CN(c1ccc(Nc3ncc4cccc(Br)c4n3)nc1)C2 ; Brc1cccc2cnc(Nc3ccc(N4CC5(CNC5)C4)cn3)nc12]

TFA (3 mL) was added to a stirred mixture of tert-butyl 6-(6-((8-bromoquinazolin-2-yl)amino)pyridin-3-yl)-2,6-diazaspiro[3.3]heptane-2-carboxylate (800 mg, 1.60 mmol) in DCM (9 mL). The resulting mixture was stirred for 1 h at room temperature and concentrated under reduced pressure to afford crude N-(5-(2,6-diazaspiro[3.3]heptan-2-yl)pyridin-2-yl)-8-bromoquinazolin-2-amine (1 g). The crude product was used for the next step without further purification.

LCMS (ESI-MS) m/z=397.1 [M+H]⁺.

### Step 6: 8-(8,8-difluoro-2,6-diazaspiro[3.4]octan-6-yl)-6-methyl-N-(14(1-methyl-1H-pyrazol-4-yl)sulfonyl)piperidin-4-yl)pyrido[3,4-d]pyrimidin-2-amine

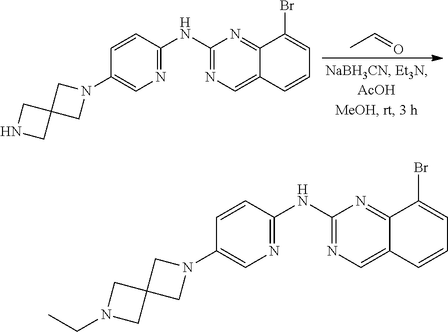

To a solution of N-(5-(2,6-diazaspiro[3.3]heptan-2-yl)pyridin-2-yl)-8-bromoquinazolin-2-amine (1.1 g, 2.76 mmol) in methanol (10 mL) was added triethylamine (0.56 g, 5.53 mmol). The resulting mixture was stirred for 5 minutes and acetaldehyde (0.61 g, 13.8 mmol), AcOH (0.02 g, 0.27 mmol) and NaBH₃CN (1.74 g, 27.7 mmol) were added. The resulting mixture was stirred for 3 h at room temperature and concentrated under reduced pressure. The residue was diluted with water (100 mL) and extracted with ethyl acetate (3×100 mL). The combined organic layers were washed with brine (100 mL), dried over anhydrous sodium sulfate and filtered. The filtrate was concentrated under reduced pressure. The residue was purified by silica gel column chromatography (MeOH/DCM, 1:10) to afford the desired product 8-bromo-N-(5-(6-ethyl-2,6-diazaspiro[3.3]heptan-2-yl)pyridin-2-yl)quinazolin-2-amine (200 mg, 15.3% yield).

LCMS (ESI) m/z=425.1 [M+H]⁺.

### Intermediate 3: N-(5-(6-ethyl-2,6-diazaspiro[3.3]heptan-2-yl) pyridin-2-yl) formamide

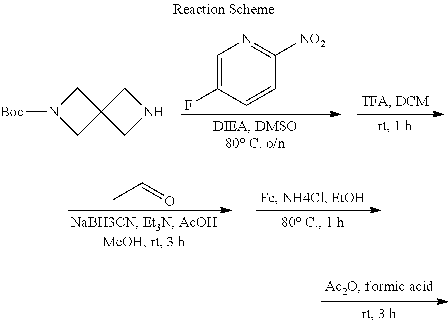

### Detailed Procedure

### Step 1: tert-butyl 6-(6-nitropyridin-3-yl)-2,6-diazaspiro[3.3]heptane-2-carboxylate

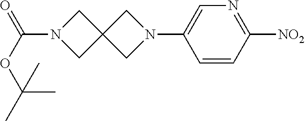 [SMILES: CC(C)(C)OC(=O)N1CC2(C1)CN(c1ccc([N+](=O)[O-])nc1)C2]

To a stirred mixture of tert-butyl 2,6-diazaspiro[3.3]heptane-2-carboxylate (5 g, 25.21 mmol) and 5-fluoro-2-nitropyridine (5.37 g, 37.82 mmol) in dimethyl sulfoxide (30 mL) was N,N-diisopropylethylamine (9.78 g, 75.65 mmol). The resulting mixture was heated to 80° C. and stirred for 3 hours. The reaction mixture was allowed to cool to room temperature, diluted with water (500 mL) and extracted with ethyl acetate (3×500 mL). The combined organic layers were washed with brine (500 mL), dried over anhydrous sodium sulfate, and filtered. The filtrate was concentrated under vacuum to afford the crude product. The residue was purified by trituration with petroleum ether/ethyl acetate (5:1, 100 mL) to afford tert-butyl 6-(6-nitropyridin-3-yl)-2,6-diazaspiro[3.3]heptane-2-carboxylate (6.78 g, 83.7% yield).

LCMS (ESI) m/z=321 [M+H]⁺.

### Step 2: 2-(6-nitropyridin-3-yl)-2,6-diazaspiro[3.3]heptane

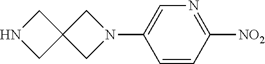 [SMILES: O=[N+]([O-])c1ccc(N2CC3(CNC3)C2)cn1]

To a stirred mixture of tert-butyl 6-(6-nitropyridin-3-yl)-2,6-diazaspiro[3.3]heptane-2-carboxylate (6.78 g, 21.16 mmol) in dichloromethane (80 mL) was added trifluoroacetic acid (16 mL). The resulting mixture was stirred for 1 hour at room temperature and concentrated under vacuum. The residue was diluted with dichloromethane (100 mL) and concentrated under vacuum again to afford crude 2-(6-nitropyridin-3-yl)-2,6-diazaspiro[3.3]heptane trifluoroacetic acid salt (6 g). The crude product was used for the next step without further purification.

LCMS (ESI) m/z=221 [M+H]⁺.

### Step 3: 2-ethyl-6-(6-nitropyridin-3-yl)-2,6-diazaspiro[3.3]heptane

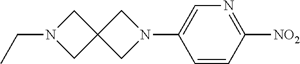 [SMILES: CCN1CC2(C1)CN(c1ccc([N+](=O)[O-])nc1)C2]

A solution of 2-(6-nitropyridin-3-yl)-2,6-diazaspiro[3.3]heptane trifluoroacetic acid salt (6 g, 18.9 mmol) in methanol (100 mL) was treated with triethylamine (5.73 g, 56.7 mmol) for 10 minutes followed by the addition of acetaldehyde (4.16 g, 94.5 mmol), acetic acid (0.23 mL, 4.08 mmol) and sodium cyanoborohydride (2.51 g, 39.8 mmol). The resulting mixture was stirred for 3 hours at room temperature and concentrated under vacuum. The residue was diluted with water (500 mL) and extracted with ethyl acetate (3×500 mL). The combined organic layers were washed with brine (1000 mL), dried over anhydrous sodium sulfate, and filtered. The filtrate was concentrated under reduced pressure to afford the crude product. The residue was purified by trituration with dichloromethane (100 mL) to afford 2-ethyl-6-(6-nitropyridin-3-yl)-2,6-diazaspiro[3.3]heptane (4 g, 58.9% yield).

LCMS (ESI) m/z=249 [M+H]⁺.

### Step 4: 5-(6-ethyl-2,6-diazaspiro[3.3]heptan-2-yl) pyridin-2-amine

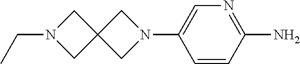 [SMILES: CCN1CC2(C1)CN(c1ccc(N)nc1)C2]

A solution of 2-ethyl-6-(6-nitropyridin-3-yl)-2,6-diazaspiro[3.3]heptane (4 g, 16.11 mmol), ammonium chloride (4.31 g, 80.55 mmol), iron powder (9.00 g, 161.100 mmol) and water (20 mL) in ethanol (60 mL) was stirred for 1 hour at 80° C. The resulting mixture was filtered and the filter cake was washed with ethanol (100 mL). The filtrate was concentrated under vacuum to afford the crude product. The residue was purified by reversed-phase flash chromatography (C18 silica gel, acetonitrile/water (with 10 mmol/L NH₄HCO₃) gradient) to afford 5-(6-ethyl-2,6-diazaspiro[3.3]heptan-2-yl)pyridin-2-amine (2 g, 56.6% yield).

LCMS (ESI) m/z=219 [M+H]⁺.

### Step 5: N-(5(6-ethyl-2,6-diazaspiro[3.3]heptan-2-yl) pyridin-2-yl) formamide

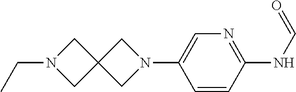 [SMILES: CCN1CC2(C1)CN(c1ccc(NC=O)nc1)C2]

A solution of acetic anhydride (2 mL) in formic acid (4 mL) was stirred for 1 hour at room temperature followed by the addition of 5-(6-ethyl-2,6-diazaspiro[3.3]heptan-2-yl)pyridin-2-amine (400 mg, 1.83 mmol) in portions at room temperature. The resulting mixture was stirred for 3 hours at room temperature. The reaction mixture was neutralized to PH=7 with saturated aqueous sodium bicarbonate (200 mL) and extracted with ethyl acetate (3×100 mL). The combined organic layers were washed with brine, dried over anhydrous sodium sulfate, and filtered. The filtrate was concentrated under vacuum and the residue was purified by preparative reverse phase HPLC (acetonitrile/water (with 10 mM NH₄HCO₃ and 0.1% NH₃·H₂O) gradient) to afford the title compound (70 mg, 15.3% yield).

LCMS (ESI) m/z=247 [M+H]⁺.

### Intermediate 4: 8-bromo-7-fluoro-N-(1-(methylsulfonyl)piperidin-4-yl)quinazolin-2-amine

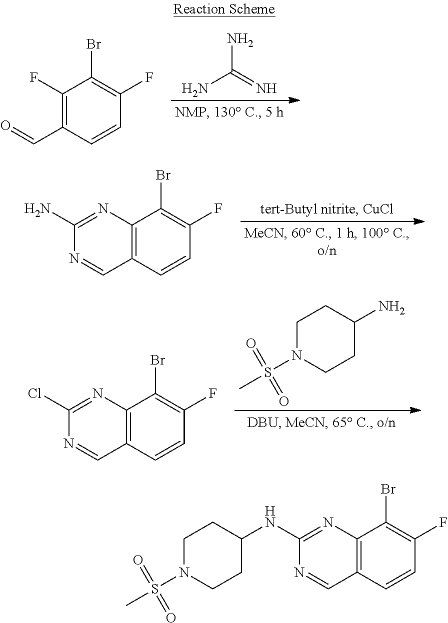

### Detailed Procedure

### Step 1: 8-bromo-7-fluoroquinazolin-2-amine

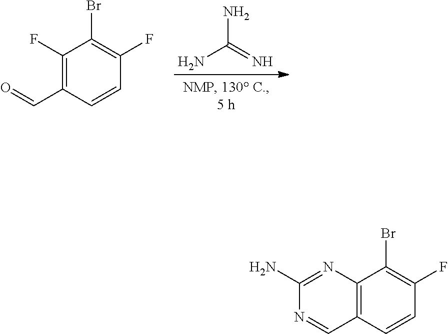

A mixture of 3-bromo-2,4-difluorobenzaldehyde (5 g, 22.6 mmol) and guanidine (4.01 g, 67.8 mmol) in NMP (50 mL) was heated to 130° C. and stirred for 5 h. After cooling to room temperature, the reaction mixture was diluted with water (100 mL) and extracted with EA (3×50 mL). The combined organic layers were washed with brine, dried over anhydrous Na₂SO₄ and concentrated under reduced pressure to afford the crude product. The residue was purified by silica gel column chromatography (EA/PE, 2:1) to afford the desired product 8-bromo-7-fluoroquinazolin-2-amine (800 mg, 14.6% yield).

LCMS (ESI-MS) m/z=242.0 [M+H]⁺.

### Step 2: 8-bromo-2-chloro-7-fluoroquinazoline

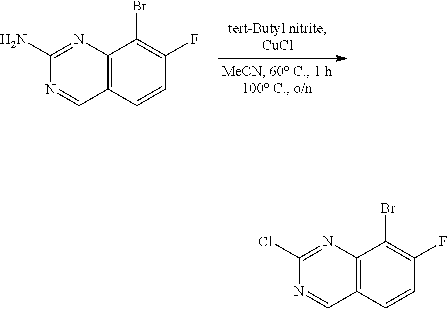 [SMILES: Nc1ncc2ccc(F)c(Br)c2n1 ; Fc1ccc2cnc(Cl)nc2c1Br]

A mixture of tert-butyl nitrite (511 mg, 4.95 mmol) and copper(I) chloride (490 mg, 4.95 mmol) in acetonitrile (10 mL) was stirred for 1 h at 60° C. 8-Bromo-7-fluoroquinazolin-2-amine (800 mg, 3.30 mmol) was added in portions over 1 minute at room temperature. The resulting mixture was stirred overnight at 100° C. After cooling to room temperature, the reaction mixture was concentrated under reduced pressure. The residue was purified by Prep-TLC (PE/EA, 1:1) to afford 8-bromo-2-chloro-7-fluoroquinazoline (300 mg, 34.7% yield).

LCMS (ESI-MS) m/z=261.0 [M+H]⁺.

### Step 3: 8-bromo-7-fluoro-N-(1-(methylsulfonyl)piperidin-4-yl)quinazolin-2-amine

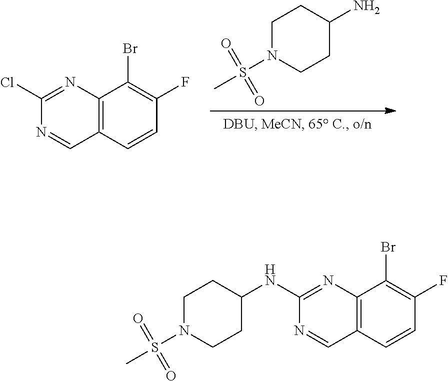

1,8-diazabicyclo[5.4.0]undec-7-ene (349 mg, 2.29 mmol) was added to a mixture of 8-bromo-2-chloro-7-fluoroquinazoline (300 mg, 1.15 mmol) and 1-(methylsulfonyl)piperidin-4-amine (206 mg, 1.15 mmol) in McCN (3 mL). The resulting mixture was stirred overnight at 65° C. After cooling to room temperature, the reaction mixture was concentrated under reduced pressure. The residue was purified by Prep-TLC (ethyl acetate) to afford the desired product 8-bromo-7-fluoro-N-(1-(methylsulfonyl)piperidin-4-yl)quinazolin-2-amine (120 mg, 25.8% yield).

LCMS (ESI-MS) m/z=403.1 [M+H]⁺.

### Intermediate 5: 8-bromo-2-chloro-6-(difluoromethyl)quinazoline

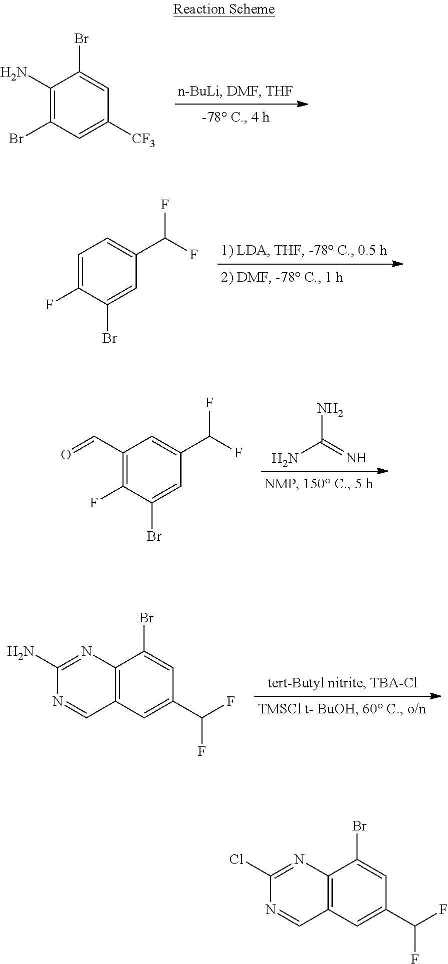

### Detailed Procedure

### Step 1: 2-bromo-4-(difluoromethyl)-1-fluorobenzene

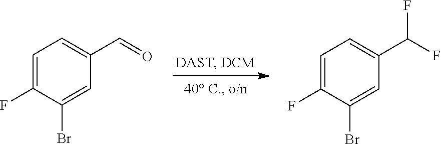 [SMILES: O=Cc1ccc(F)c(Br)c1 ; Fc1ccc(C(F)F)cc1Br]

DAST (47.6 g, 296 mmol) was added to a stirred mixture of 3-bromo-4-fluorobenzaldehyde (30 g, 148 mmol) in DCM (300 mL) dropwise at 0° C. The resulting mixture was stirred overnight at 40° C. and quenched by the addition of saturated aqueous NaHCO₃(500 ml) at 0° C. The mixture was extracted with DCM (3×500 mL) and the combined organic layers were dried over anhydrous Na₂SO₄. After filtration, the filtrate was concentrated under reduced pressure to afford crude 2-bromo-4-(difluoromethyl)-1-fluorobenzene (32.2 g, 96.8% yield).

LCMS (ESI-MS) m/z=225.0 [M+H]⁺.

### Step 2: 3-bromo-5-(difluoromethyl)-2-fluorobenzaldehyde

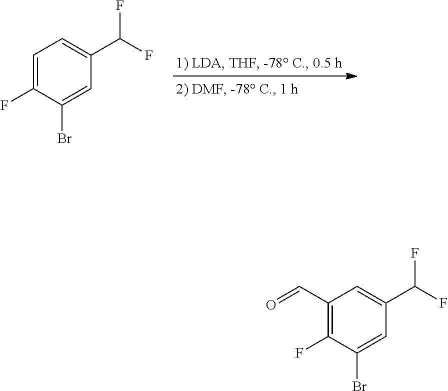 [SMILES: Fc1ccc(C(F)F)cc1Br ; O=Cc1cc(C(F)F)cc(Br)c1F]

To a cooled to −78° C. mixture of 2-bromo-4-(difluoromethyl)-1-fluorobenzene (10 g, 44.4 mmol) in 100 mL of THF was added LDA (2 M in THF, 24.4 mL, 48.8 mmol) dropwise under a nitrogen atmosphere. The mixture was stirred at −78° C. for 0.5 h. DMF (3.89 g, 53.3 mmol) was added dropwise and the resulting mixture was stirred at −78° C. for another 1 h. The reaction mixture was slowly poured into 200 mL of saturated aqueous NH₄Cl at 0° C. and stirred for 1 h. The solution was diluted with H₂O (200 mL) and extracted with EA (400 mL×3). The organic phase was dried over Na₂SO₄, filtered and concentrated under reduced pressure to afford crude 3-bromo-5-(difluoromethyl)-2-fluorobenzaldehyde (15 g). The crude product was used for the next step without further purification.

### Step 3: 8-bromo-6-(difluoromethyl)quinazolin-2-amine

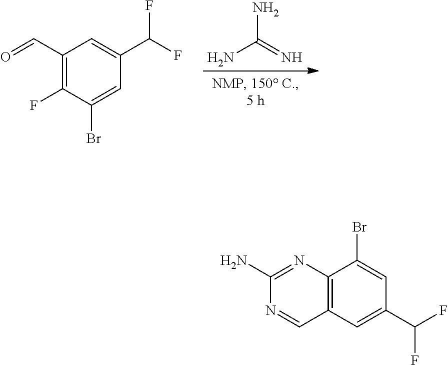

Guanidine (3.85 g, 65.21 mmol) was added to a mixture of 3-bromo-5-(difluoromethyl)-2-fluorobenzaldehyde (15 g, 59.28 mmol) in NMP (13 mL). The mixture was stirred at 150° C. for 5 h. After cooling to room temperature, the reaction mixture was diluted with water (200 mL) and extracted with EA (200 mL). The organic layers were washed with brine (2×200 mL), dried over anhydrous sodium sulfate and concentrated under reduced pressure. The residue was purified by silica gel column chromatography (EA/PE, 45:55) to afford 8-bromo-6-(difluoromethyl)quinazolin-2-amine (0.9 g, 5.54% yield).

LCMS (ESI-MS) m/z=274.0 [M+H]⁺.

### Step 4: 8-bromo-2-chloro-6-(difluoromethyl)quinazoline

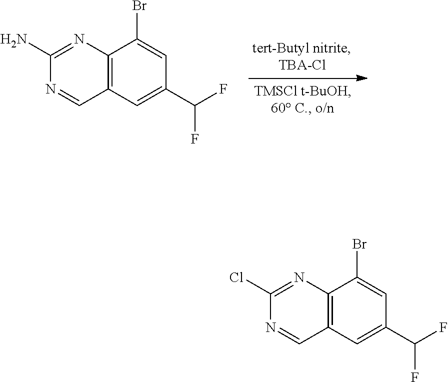 [SMILES: Nc1ncc2cc(C(F)F)cc(Br)c2n1 ; FC(F)c1cc(Br)c2nc(Cl)ncc2c1]

Tert-Butyl nitrite (3.9 mL) was added to a mixture of 8-bromo-6-(difluoromethyl)quinazolin-2-amine (3 g, 10.94 mmol), TBA-Cl (5.4 mL) in TMSCl and t-BuOH (15 mL). The mixture was stirred overnight at 60° C. The reaction mixture was diluted with water (30 mL) and extracted with DCM (2×200 mL). The combined organic layers were washed with brine (2×200 mL), dried over anhydrous Na₂SO₄. After filtration, the filtrate was concentrated under reduced pressure. The residue was purified by silica gel column chromatography (EA/PE, 6:94) to afford 8-bromo-2-chloro-6-(difluoromethyl)quinazoline (1.03 g, 31.9% yield).

¹H NMR (400 MHz, DMSO-d₆) δ 9.79-9.76 (m, 1H), 8.57-8.54 (m, 2H), 7.43-7.16 (m, 1H). LCMS (ESI-MS) m/z=292.9 [M+H]⁺.

### Intermediate 6: 8-Bromo-6-(difluoromethyl)-N-(2-methylisoindolin-5-yl)quinazolin-2-amin

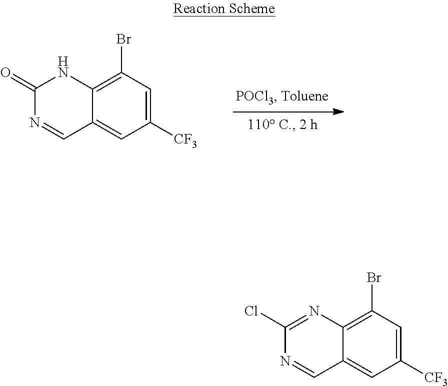 [SMILES: O=c1ncc2cc(C(F)(F)F)cc(Br)c2[nH]1 ; FC(F)(F)c1cc(Br)c2nc(Cl)ncc2c1]

### Detailed Procedure

### Step 1: 2-amino-3-bromo-5-(trifluoromethyl)benzaldehyde

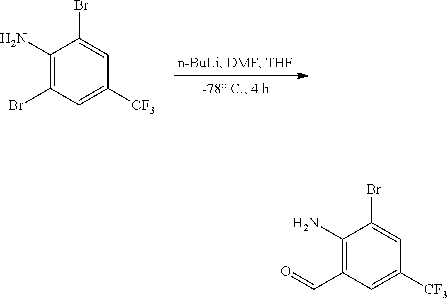 [SMILES: Nc1c(Br)cc(C(F)(F)F)cc1Br ; Nc1c(Br)cc(C(F)(F)F)cc1C=O]

A solution of n-BuLi (2.5 M in hexane, 52.6 mL, 131.5 mmol) was added to a stirred mixture of 2,6-dibromo-4-(trifluoromethyl)aniline (20 g, 62.7 mmol) in THF (250 mL) at −78° C. under a nitrogen atmosphere. A solution of DMF (6.42 g, 87.8 mmol) in THF (10 mL) was slowly added and the resulting mixture was stirred at −78° C. for 3 h, quenched by addition of water (500 mL) and extracted with EA (2×500 mL). The combined organic layers were washed with brine (2×1000 ml), dried over anhydrous sodium sulfate, filtered and concentrated under reduced pressure to afford crude 2-amino-3-bromo-5-(trifluoromethyl)benzaldehyde (10.2 g, 60.7% yield).

LCMS (ESI-MS) m/z=268.1 [M+H]⁺.

### Step 2: 8-bromo-6-(trifluoromethyl)quinazolin-2(1H)-one

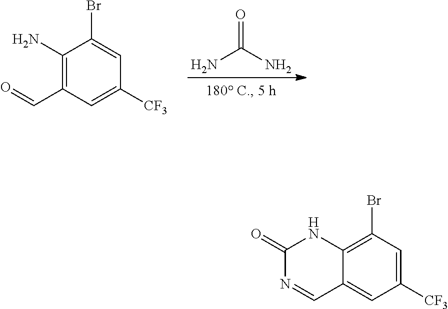

A mixture of 2-amino-3-bromo-5-(trifluoromethyl)benzaldehyde (10.2 g, 38.2 mmol) and urea (34.4 g, 57.3 mmol) was heated to 180° C. and stirred for 5 h. After cooling to room temperature, the reaction mixture was filtered and the filter cake was washed with water (2×200 mL) and EA (2×200 mL). The collected solid was dried under high vacuum to afford crude 8-bromo-6-(trifluoromethyl)quinazolin-2(11)-one (7.3 g). The crude product was used for the next step without further purification.

LCMS (ESI-MS) m/z=293.2 [M+H]⁺.

### Step 3: 8-bromo-2-chloro-6-(trifluoromethyl)quinazoline

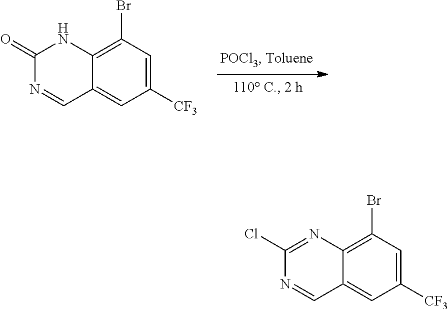 [SMILES: O=c1ncc2cc(C(F)(F)F)cc(Br)c2[nH]1 ; FC(F)(F)c1cc(Br)c2nc(Cl)ncc2c1]

POCl₃ (60 mL, 643 mmol) was added to a mixture of 8-bromo-6-(trifluoromethyl)quinazolin-2(1H)-one (7.3 g, 24.9 mmol) in toluene (60 mL). The resulting mixture was stirred at 110° C. for 2 h and concentrated under reduced pressure. The residue was slowly quenched by addition of saturated anhydrous NaHCO₃ at 0° C. until no bubbles appeared. The resulting mixture was extracted with EA (3×200 mL). The combined organic layers were washed with brine (2×300 mL), dried over anhydrous Na₂SO₄, filtered and the filtrate was concentrated under reduced pressure. The residue was purified by silica gel column chromatography (EA/PE, 1:20) to afford 8-bromo-2-chloro-6-(trifluoromethyl)quinazoline (1.06 g, 13.6% yield).

¹H NMR (400 MHz, DMSO-d₆) δ 9.80 (s, 1H), 8.80 (s, 1H), 8.74 (s, 1H).

LCMS (ESI-MS) m/z=312.7 [M+H]⁺.

### Intermediate 7: 2-Methylisoindolin-5-amine

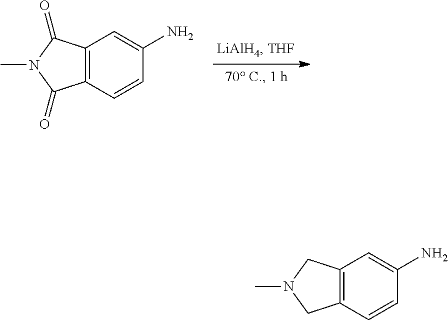

A solution of LiAlH₄ (2 M in THF, 4.25 mL, 8.50 mmol) was added to 5-amino-2-methylisoindoline-1,3-dione (500 mg, 2.83 mmol) in THF (10 mL) at 0° C. The reaction mixture was heated to 70° C. and stirred for 1 h. After cooling to 0° C., the reaction was quenched by the addition of ethanol and water. The resulting slurry is filtered through a pad of diatomaceous earth, and the filtrate was concentrated under reduced pressure to afford crude 2-methylisoindolin-5-amine (300 mg). The crude product was used for the next step without further purification.

LCMS (ESI-MS) m/z=149.1 [M+H]⁺.

### Intermediate 8: 8-Bromo-6-(difluoromethyl)-N-(2-methylisoindolin-5-yl)quinazolin-2-amine

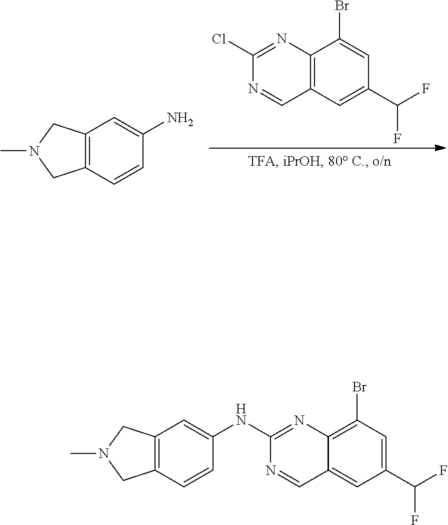 [SMILES: CN1Cc2ccc(N)cc2C1 ; FC(F)c1cc(Br)c2nc(Cl)ncc2c1 ; CN1Cc2ccc(Nc3ncc4cc(C(F)F)cc(Br)c4n3)cc2C1]

TFA (385 mg, 3.37 mmol) was added to a mixture of 2-methylisoindolin-5-amine (250 mg, 1.68 mmol) and 8-bromo-2-chloro-6-(difluoromethyl) quinazoline (495 mg, 1.68 mmol) in propan-2-ol (5 mL). The resulting mixture was heated to 80° C. and stirred overnight. After cooling to room temperature, the reaction mixture was concentrated under reduced pressure. The residue was purified by silica gel column chromatography(MeOH/DCM, 10:90) to afford 8-bromo-6-(difluoromethyl)-N-(2-methylisoindolin-5-yl)quinazolin-2-amine (180 mg, 26.3% yield).

LCMS (ESI-MS) m/z=405.0 [M+H]⁺.

### Intermediate 9: 1-(1-methylazetidin-3-yl)-1H-pyrazol-4-amine

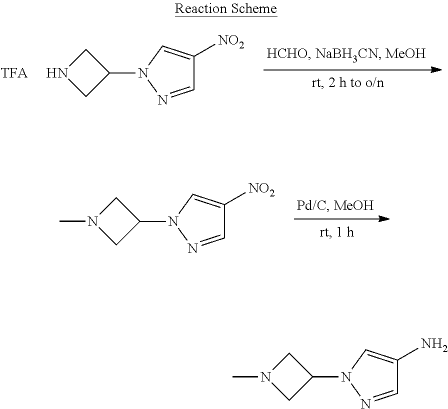

### Detailed Procedure

### Step 1: 1-(1-methylazetidin-3-yl)-4-nitro-1H-pyrazole-(1-methylazetidin-3-yl)-1H-pyrazol-4-amine

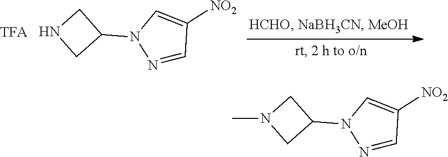

A solution of 1-(azetidin-3-yl)-4-nitro-1H-pyrazole (2 g, 7.08 mmol) and HCHO (319 mg, 10.6 mmol) in MeOH (30 mL) was stirred for 2 h at room temperature. NaBH₃CN (891 mg, 14.2 mmol) was added. The resulting mixture was stirred overnight at room temperature, filtered and the filter cake was washed with MeOH (20 mL). The filtrate was concentrated under reduced pressure. The residue was purified by silica gel column chromatography (MeOH/DCM, 10:90) to afford 1-(1-methylazetidin-3-yl)-4-nitro-1H-pyrazole (500 mg, 33.3% yield).

LCMS (ESI-MS) m/z=183.1 [M+H]⁺.

### Step 2: 1-(1-methylazetidin-3-yl)-1H-pyrazol-4-amine

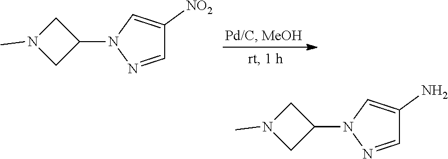

Pd/C (10% on carbon, 99.3 mg) was added to a solution of 1-(1-methylazetidin-3-yl)-4-nitro-1H-pyrazole (680 mg, 3.73 mmol) in MeOH (10 mL) under a nitrogen atmosphere. The reaction mixture was stirred for 1 h at room temperature under a hydrogen atmosphere, filtered and the filter cake was washed with MeOH (30 mL). The filtrate was concentrated under reduced pressure to afford the crude title product (700 mg). The crude product was used for the next step without further purification.

LCMS (ESI-MS) m/z=153.1 [M+H]⁺.

### Intermediate 10: 1-(5-cyclopropylpyrimidin-2-yl)piperidin-4-amine

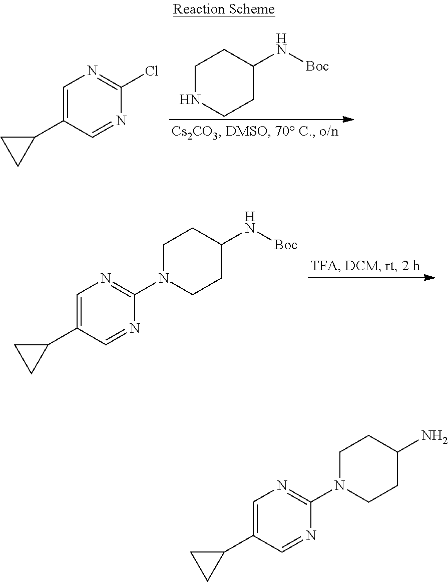

### Step 1: Tert-butyl (1-(5-cyclopropylpyrimidin-2-yl)piperidin-4-yl)carbamate

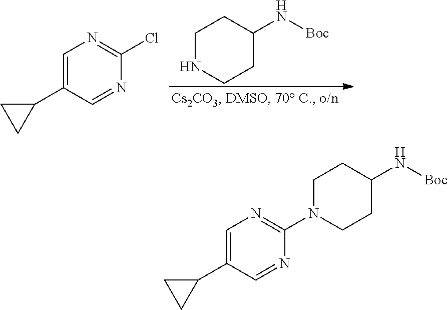

Cs₂CO₃ (4.21 g, 12.9 mmol) was added to a mixture of 2-chloro-5-cyclopropylpyrimidine (1 g, 6.46 mmol) and tert-butyl N-(piperidin-4-yl)carbamate (1.30 g, 6.46 mmol) in DMSO (20 mL). The resulting mixture was stirred overnight at 70° C., diluted with water (30 mL) and extracted with EA (3×50 mL). The combined organic layers were washed with brine, dried over anhydrous Na₂SO₄, filtered and concentrated under reduced pressure to afford the crude product. The residue was purified by column chromatography(EA/PE, 0:100 to 20:80) to afford the desired product tert-butyl N-[1-(5-cyclopropylpyrimidin-2-yl)piperidin-4-yl]carbamate (1.5 g, 70.3% yield).

LCMS (ESI-MS) m/z=319.2 [M+H]⁺.

### Step 2: 1-(5-cyclopropylpyrimidin-2-yl)piperidin-4-amine

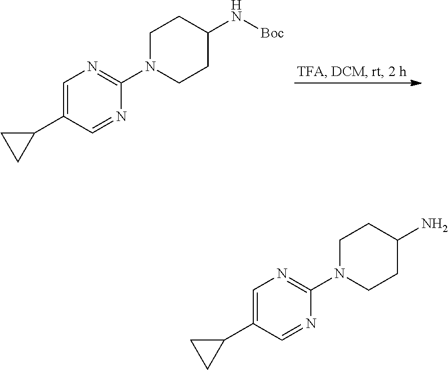 [SMILES: CC(C)(C)OC(=O)NC1CCN(c2ncc(C3CC3)cn2)CC1 ; NC1CCN(c2ncc(C3CC3)cn2)CC1]

TFA (3 mL) was added to a stirred mixture of tert-butyl N-[1-(5-cyclopropylpyrimidin-2-yl)piperidin-4-yl]carbamate (1.5 g, 4.71 mmol) in DCM (10 mL). The resulting mixture was stirred at room temperature for 2 h and concentrated under high vacuum to afford crude 1-(5-cyclopropylpyrimidin-2-yl)piperidin-4-amine (1.5 g crude). The crude product was used for the next step without further purification.

LCMS (ESI-MS) m/z=219.1 [M+H]⁺.

### Intermediate 11: 1-(7-cyclopropyl-7H-pyrrolo[2,3-d]pyrimidin-2-yl)piperidin-4-amine

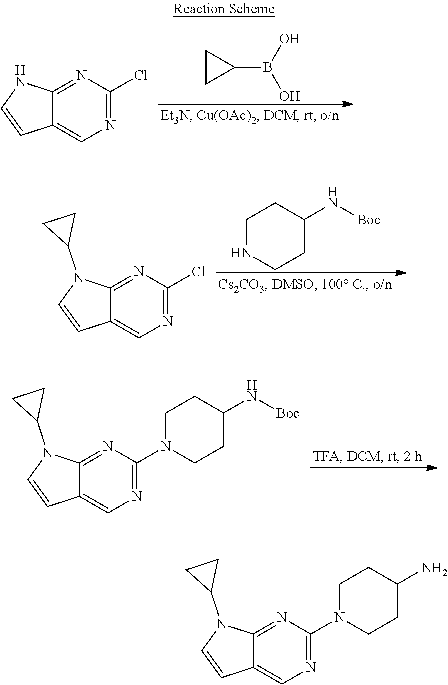 [SMILES: Clc1ncc2cc[nH]c2n1 ; OB(O)C1CC1 ; Clc1ncc2ccn(C3CC3)c2n1 ; CC(C)(C)OC(=O)NC1CCNCC1 ; CC(C)(C)OC(=O)NC1CCN(c2ncc3ccn(C4CC4)c3n2)CC1 ; NC1CCN(c2ncc3ccn(C4CC4)c3n2)CC1]

### Step 1: 2-chloro-7-cyclopropyl-7H-pyrrolo[2,3-d]pyrimidine

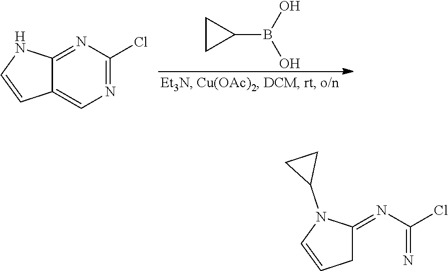 [SMILES: Clc1ncc2cc[nH]c2n1 ; OB(O)C1CC1 ; N=C(Cl)/N=C1\CC=CN1C1CC1]

A mixture of 2-chloro-7H-pyrrolo[2,3-d]pyrimidine (2 g, 13.0 mmol), cyclopropylboronic acid (1.12 g, 13.0 mmol), Cu(OAc)₂ (4.73 g, 26.0 mmol) and Et₃N (2.64 g, 26.0 mmol) in DCM (20 mL) was stirred overnight at room temperature. The reaction mixture was diluted with water (50 mL) and extracted with DCM (3×50 mL). The combined organic layers were washed with brine (50 mL), dried over anhydrous Na₂SO₄, filtered and concentrated under reduced pressure. The residue was purified by column chromatography (EA/PE, from 0:100 to 20:80) to afford the desired product 2-chloro-7-cyclopropyl-7H-pyrrolo[2,3-d]pyrimidine (1.5 g, 59.4% yield).

LCMS (ESI-MS) m/z=194.0 [M+H]⁺.

### Step 2: Tert-butyl (1-(7-cyclopropyl-7H-pyrrolo[2,3-d]pyrimidin-2-yl)piperidin-4-yl)carbamate

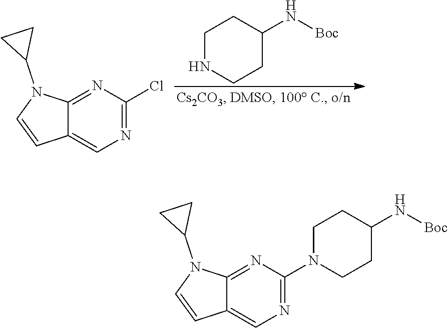 [SMILES: Clc1ncc2ccn(C3CC3)c2n1 ; CC(C)(C)OC(=O)NC1CCNCC1 ; CC(C)(C)OC(=O)NC1CCN(c2ncc3ccn(C4CC4)c3n2)CC1]

Cs₂CO₃ (4.71 g, 14.4 mmol) was added to a mixture of 2-chloro-7-cyclopropylpyrrolo[2,3-d]pyrimidine (1.4 g, 7.23 mmol) and tert-butyl N-(piperidin-4-yl)carbamate (1.45 g, 7.23 mmol) in DMSO (15 mL). The resulting mixture was stirred overnight at 100° C. After cooling to room temperature, the reaction mixture was diluted with water (50 mL) and extracted with DCM (3×50 mL). The combined organic layers were washed with brine (50 mL), dried over anhydrous Na₂SO₄, filtered and concentrated under reduced pressure. The residue was purified by column chromatography (EA/PE, from 0:100 to 20:80) to afford the desired product tert-butyl (1-(7-cyclopropyl-7H-pyrrolo[2,3-d]pyrimidin-2-yl)piperidin-4-yl)carbamate (1.5 g, 58.0% yield).

LCMS (ESI-MS) m/z=358.2 [M+H]⁺.

### Step 3: 1-(7-cyclopropyl-7H-pyrrolo[2,3-d]pyrimidin-2-yl)piperidin-4-amine

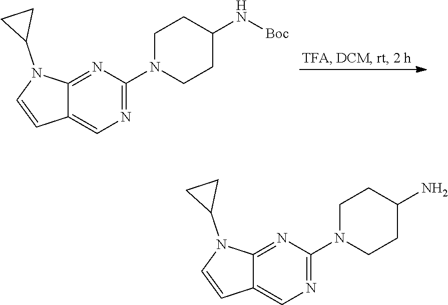 [SMILES: CC(C)(C)OC(=O)NC1CCN(c2ncc3ccn(C4CC4)c3n2)CC1 ; NC1CCN(c2ncc3ccn(C4CC4)c3n2)CC1]

TFA (3 mL) was added to a stirred mixture of tert-butyl (1-(7-cyclopropyl-7H-pyrrolo[2,3-d]pyrimidin-2-yl)piperidin-4-yl)carbamate (1.5 g, 4.19 mmol) in DCM (10 mL). The resulting mixture was stirred at room temperature for 2 h and concentrated under high vacuum to afford crude 1-(7-cyclopropyl-7H-pyrrolo[2,3-d]pyrimidin-2-yl)piperidin-4-amine (1.5 g). The crude product was used for the next step without further purification.

LCMS (ESI-MS) m/z=258.2 [M+H]⁺.

### Intermediate 12: 2-(2-methoxyethyl)-2,6-diazaspiro[3.3]heptane

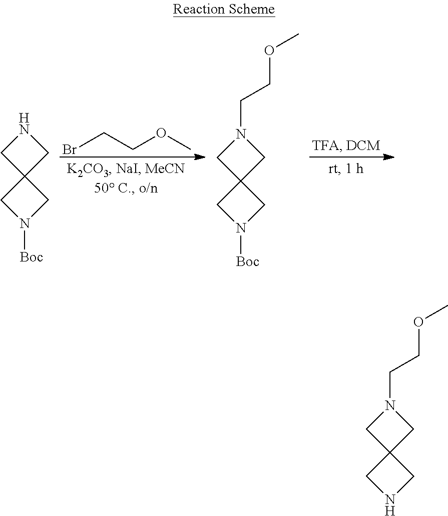

### Detailed Procedure

### Step 1: Tert-butyl 6-(2-methoxyethyl)-2,6-diazaspiro[3.3]heptane-2-carboxylate

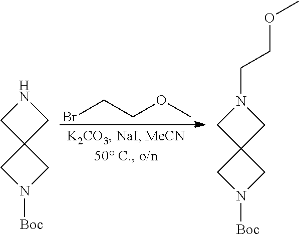

NaI (29.5 mg, 0.19 mmol) was added to a mixture of tert-butyl 2,6-diazaspiro[3.3]heptane-2-carboxylate (300 mg, 1.51 mmol), 2-bromoethyl methyl ether (326 mg, 2.34 mmol) and K₂CO₃ (544 mg, 3.93 mmol) in MCCN (15 mL). The reaction mixture was heated at 50° C. overnight, quenched by addition of water (50 mL) and extracted with EA (3×50 mL). The combined organic layers were dried over anhydrous Na₂SO₄, filtered and concentrated under reduced pressure to afford the crude title product (150 mg). The crude product was used for the next step without further purification.

LCMS (ESI-MS) m/z=257.2 [M+H]⁺.

### Step 2: 2-(2-methoxyethyl)-2,6-diazaspiro[3.3]heptane

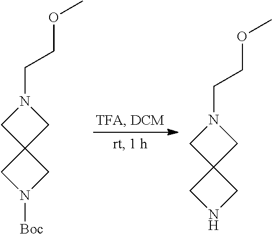 [SMILES: COCCN1CC2(C1)CN(C(=O)OC(C)(C)C)C2 ; COCCN1CC2(CNC2)C1]

TFA (0.5 mL) was added to a stirred mixture of tert-butyl 6-(2-methoxyethyl)-2,6-diazaspiro[3.3]heptane-2-carboxylate (150 mg, 0.58 mmol) in DCM (1.5 mL). The resulting mixture was stirred for 1 h at room temperature and concentrated under high vacuum to afford the crude title product (150 mg). The crude product was used for the next step without further purification.

LCMS (ESI-MS) m/z=157.1 [M+H]⁺.

### Intermediate 13: 1-((1-methylcyclopropyl)sulfonyl)piperidin-4-amine

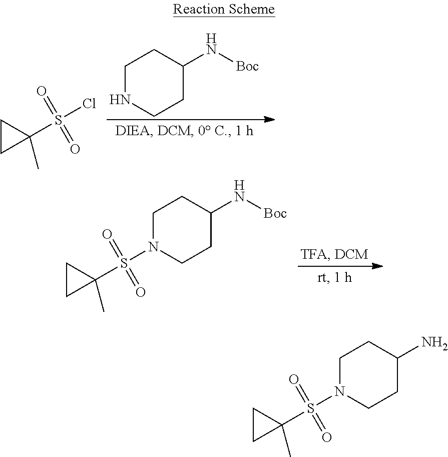

### Detailed Procedure

### Step 1: Tert-butyl (1-((1-methylcyclopropyl)sulfonyl)piperidin-4-yl)carbamate

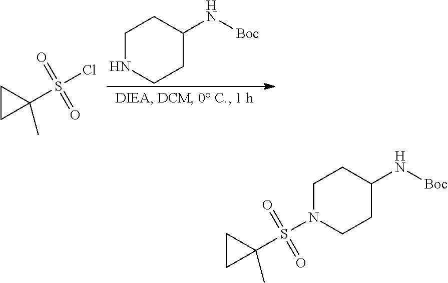

1-methylcyclopropane-1-sulfonyl chloride (463 mg, 2.99 mmol) was added dropwise to a cooled to 0° C. solution of tert-butyl piperidin-4-ylcarbamate (400 mg, 1.99 mmol) and DIEA (774 mg, 5.99 mmol) in DCM (10 mL). The resulting mixture was stirred for 1 h at room temperature, diluted with water (10 mL) and extracted with DCM (3×250 mL). The combined organic layers were dried over anhydrous Na₂SO₄, filtered and concentrated under reduced pressure. The residue was purified by silica gel column chromatography (EA/PE, 1:5) to afford tert-butyl (1-((1-methylcyclopropyl)sulfonyl)piperidin-4-yl)carbamate (580 mg, 82.1% yield).

LCMS (ESI-MS) m/z=263.2 [M+H-56]⁺.

### Step 2: 1-((1-methylcyclopropyl)sulfonyl)piperidin-4-amine

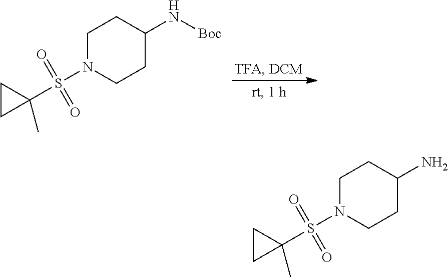 [SMILES: CC(C)(C)OC(=O)NC1CCN(S(=O)(=O)C2(C)CC2)CC1 ; CC1(S(=O)(=O)N2CCC(N)CC2)CC1]

TFA (2 mL) was added to a stirred mixture of tert-butyl (1-((1-methylcyclopropyl)sulfonyl)piperidin-4-yl)carbamate (500 mg, 1.57 mmol) in DCM (6 mL). The resulting mixture was stirred for 2 h at room temperature and concentrated under reduced pressure to afford the TFA salt of 1-((1-methylcyclopropyl)sulfonyl)piperidin-4-amine (255 mg, 74.3% yield).

¹H NMR (400 MHz, DMSO-d₆) δ 8.09 (s, 3H), 3.69 (d, J=12.5 Hz, 2H), 3.20 (s, 1H), 3.07-2.94 (m, 2H), 1.95 (d, J=12.4 Hz, 2H), 1.55-1.43 (m, 2H), 1.39 (s, 3H), 1.18-1.11 (m, 1H), 0.86-0.73 (m, 3H). LCMS (ESI-MS) m/z=219.1 [M+H]⁺.

### Intermediate 14: 1-((1-methyl-1H-pyrazol-4-yl)sulfonyl)piperidin-4-amine 2,2,2-trifluoroacetate

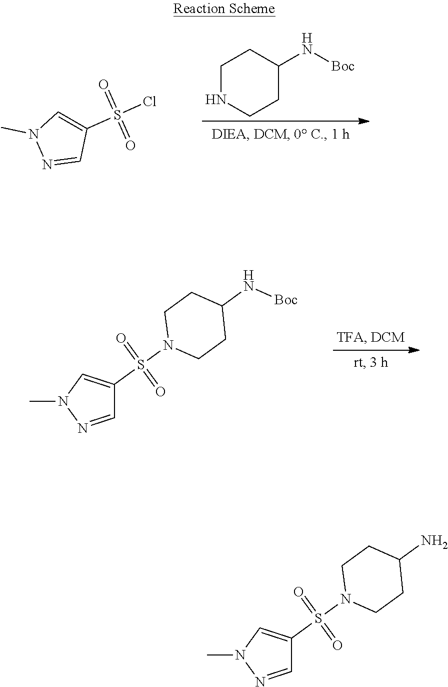

### Detailed Procedure

### Step 1: Tert-butyl (1-((1-methyl-1H-pyrazol-4-yl)sulfonyl)piperidin-4-yl)carbamate

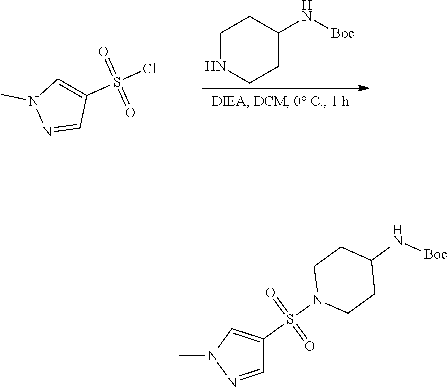

1-methyl-1H-pyrazole-4-sulfonyl chloride (1.8 g, 9.96 mmol) was added to a cooled to 0° C. solution of tert-butyl piperidin-4-ylcarbamate (2.00 g, 9.96 mmol) and DIEA (3.22 g, 24.9 mmol) in DCM (40 mL). The resulting mixture was stirred at 0° C. for 1 h, quenched by addition of water (20 mL) and extracted with DCM (3×20 mL). The combined organic layers were concentrated under reduced pressure and the residue was purified by silica gel column chromatography (MeOH/DCM, 0:100 to 5:95) to afford tert-butyl (1-((1-methyl-1H-pyrazol-4-yl)sulfonyl)piperidin-4-yl)carbamate (2.2 g, 64.1% yield).

LCMS (ESI-MS) m/z=367.1 [M+Na+H]⁺.

### Step 2: 1-((1-methyl-1H-pyrazol-4-yl)sulfonyl)piperidin-4-amine 2,2,2-trifluoroacetate

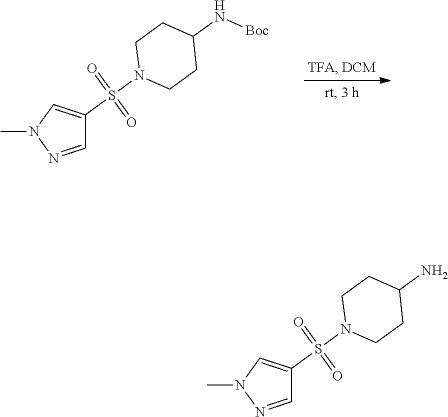 [SMILES: Cn1cc(S(=O)(=O)N2CCC(NC(=O)OC(C)(C)C)CC2)cn1 ; Cn1cc(S(=O)(=O)N2CCC(N)CC2)cn1]

TFA (5 mL) was added to a stirred mixture of tert-butyl (1-((1-methyl-1H-pyrazol-4-yl)sulfonyl)piperidin-4-yl)carbamate (2 g, 5.80 mmol) in DCM (30 mL). The resulting mixture was stirred at room temperature for 3 h and concentrated under reduced pressure. The residue was purified by silica gel column chromatography (MeOH/DCM, from 0:100 to 18:82) to afford 1-((1-methyl-1H-pyrazol-4-yl)sulfonyl)piperidin-4-amine 2,2,2-trifluoroacetate (250.7 mg, 11.6% yield).

¹H NMR (400 MHz, DMSO-d₆) δ 8.32 (s, 1H), 8.21 (s, 3H), 3.89 (s, 3H), 3.61-3.48 (m, 2H), 3.13-3.00 (m, 1H), 2.42-2.25 (m, 2H), 2.07-1.89 (m, 2H), 1.67-1.50 (m, 2H). LCMS (ESI-MS) m/z=245.0 [M+H]⁺.

### Intermediate 15: 1-(cyclopropylsulfonyl)piperidin-4-amine

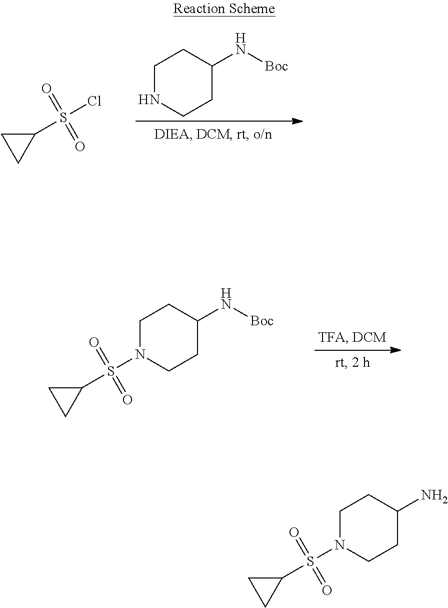 [SMILES: O=S(=O)(Cl)C1CC1 ; CC(C)(C)OC(=O)NC1CCNCC1 ; CC(C)(C)OC(=O)NC1CCN(S(=O)(=O)C2CC2)CC1 ; NC1CCN(S(=O)(=O)C2CC2)CC1]

### Detailed Procedure

### Step 1: Tert-butyl (1-(cyclopropylsulfonyl)piperidin-4-yl)carbamate

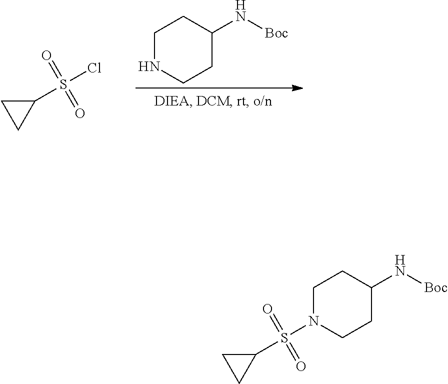 [SMILES: O=S(=O)(Cl)C1CC1 ; CC(C)(C)OC(=O)NC1CCNCC1 ; CC(C)(C)OC(=O)NC1CCN(S(=O)(=O)C2CC2)CC1]

To a solution of cyclopropanesulfonyl chloride (7.02 g, 49.93 mmol) and DIEA (19.36 g, 149.79 mmol) in DCM (100 mL) was added tert-butyl N-(piperidin-4-yl)carbamate (10 g, 49.93 mmol) dropwise at 0° C. The resulting mixture was stirred overnight at room temperature. The resulting mixture was diluted with water (500 mL). The aqueous solution was extracted with CH₂Cl₂ (3×500 mL). The combined organic layers were dried over anhydrous Na₂SO₄. After filtration, the filtrate was concentrated under reduced pressure. The residue was purified by trituration with EA (100 mL) to afford tert-butyl (1-(cyclopropylsulfonyl)piperidin-4-yl)carbamate (10 g, 59.2% yield).

LCMS (ESI-MS) m/z=249.1 [M+H-56]⁺.

### Step 2: 1-(cyclopropylsulfonyl)piperidin-4-amine

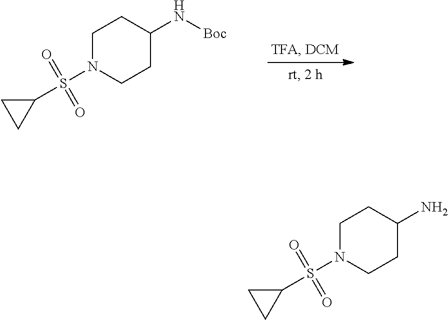 [SMILES: CC(C)(C)OC(=O)NC1CCN(S(=O)(=O)C2CC2)CC1 ; NC1CCN(S(=O)(=O)C2CC2)CC1]

A solution of tert-butyl (1-(cyclopropylsulfonyl)piperidin-4-yl)carbamate (10 g, 32.87 mmol) in TFA (15 mL) and DCM (45 mL) was stirred for 2 h at room temperature. The resulting mixture was concentrated under reduced pressure to afford 1-(cyclopropylsulfonyl)piperidin-4-amine (8 g). The crude product was used for the next step without further purification.

LCMS (ESI-MS) m/z=205.1 [M+H]⁺.

### Intermediate 16: Tert-butyl 8,8-difluoro-2-hydroxy-6-azaspiro[3.4]octane-6-carboxylate

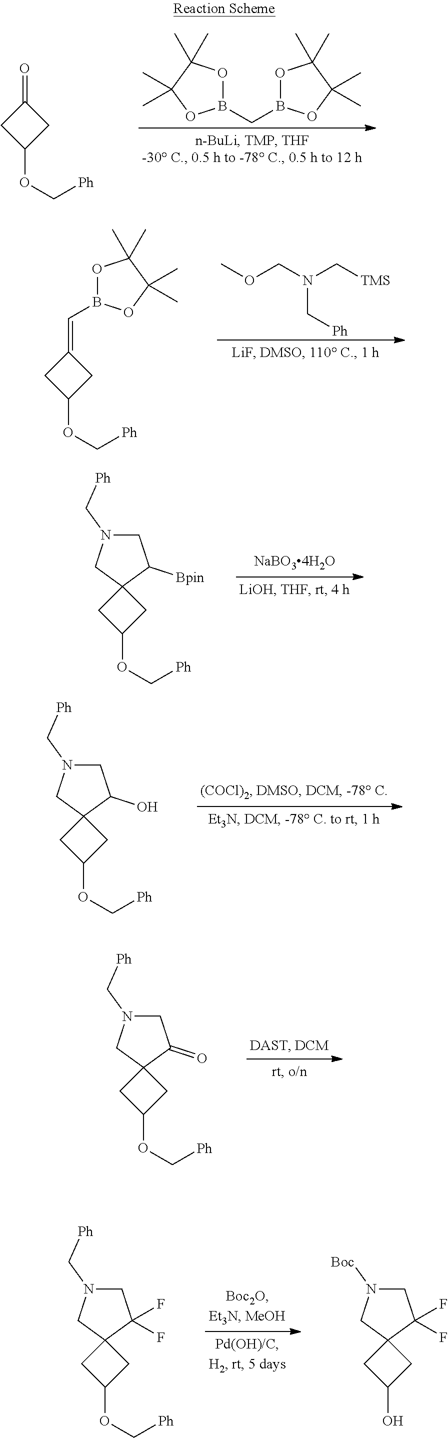

### Detailed Procedure

### Step 1: 2-((3-(benzyloxy)cyclobutylidene)methyl)-4,4,5,5-tetramethyl-1,3,2-dioxaborolane

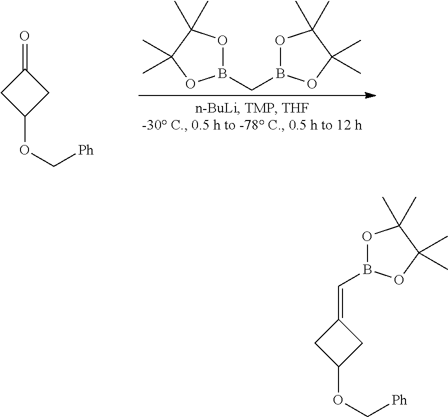

To a cooled to −30° C. solution of 2,2,6,6-tetramethylpiperidine (9.62 g, 68.09 mmol) in dry THF (100 mL) was added n-BuLi (2.5 M, 27.2 mL) dropwise under an N₂ atmosphere. The mixture was stirred at −30° C. for 0.5 h. The reaction was then cooled to −78° C. and a solution of bis(4,4,5,5-tetramethyl-1,3,2-dioxaborolan-2-yl)methane (15.21 g, 56.74 mmol) in 50 mL of dry THF was added dropwise. The reaction mixture was stirred at −78° C. for 0.5 h and a solution of 3-(benzyloxy)cyclobutan-1-one (10 g, 56.74 mmol) in 50 mL of dry THF was added dropwise. The reaction mixture was then warmed to 20° C. and stirred for an additional 12 h. The reaction mixture was slowly poured into 20 mL of saturated aqueous NH₄Cl at 0° C. and after stirring for 1 h, the solution was diluted with H₂O (200 mL) and extracted with EtOAc (400 mL×3). The organic phase was dried over Na₂SO₄, filtered and concentrated under reduced pressure to afford crude 2-((3-(benzyloxy)cyclobutylidene)methyl)-4,4,5,5-tetramethyl-1,3,2-dioxaborolane (13.7 g) which was used without further purification.

LCMS (ESI-MS) m/z=301.1 [M+H]⁺.

### Step 2: 6-benzyl-2-(benzyloxy)-8-(4,4,5,5-tetramethyl-1,3,2-dioxaborolan-2-yl)-6-azaspiro[3.4]octane

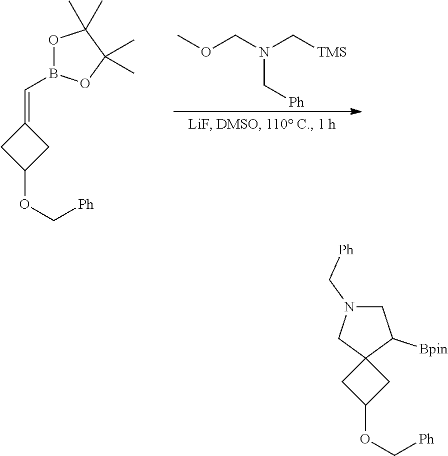

A solution of 2-((3-(benzyloxy)cyclobutylidene)methyl)-4,4,5,5-tetramethyl-1,3,2-dioxaborolane (13.7 g crude), N-benzyl-1-methoxy-N-((trimethylsilyl)methyl)methanamine (13.00 g, 54.76 mmol) and LiF (3.55 g, 136.90 mmol) in DMSO (200 mL) was stirred at 110° C. for 1 h. The reaction mixture was diluted with H₂O (200 mL) and extracted with EA (1000 mL). The combined organic layers were washed with brine, dried over anhydrous Na₂SO₄, filtered and concentrated under reduced pressure to afford crude 6-benzyl-2-(benzyloxy)-8-(4,4,5,5-tetramethyl-1,3,2-dioxaborolan-2-yl)-6-azaspiro[3.4]octane (20 g) as a colorless oil. The crude product was used for the next step without further purification.

LCMS (ESI-MS) m/z=434.2 [M+H]⁺.

### Step 3: 6-benzyl-2-(benzyloxy)-6-azaspiro[3.4]octan-8-ol

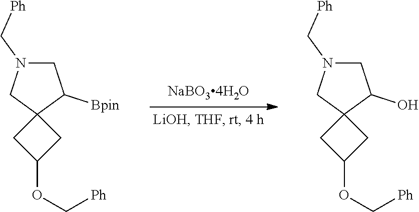

A solution of 6-benzyl-2-(benzyloxy)-8-(4,4,5,5-tetramethyl-1,3,2-dioxaborolan-2-yl)-6-azaspiro[3.4]octane (20 g crude), sodium perborate (4.53 g, 55.37 mmol) and LiOH (3.32 g, 138.44 mmol) in THF (50 mL) and H₂O (200 mL) was stirred at room temperature for 4 h. The reaction mixture was diluted with H₂O (200 mL) and extracted with EA (3×500 mL). The combined organic layers were washed with brine, dried over anhydrous Na₂SO₄, filtered and concentrated under reduced pressure. The residue was purified by silica gel column chromatography (MeOH/DCM, 3:97) to obtain 6-benzyl-2-(benzyloxy)-6-azaspiro[3.4]octan-8-ol (10 g, 67.0%) as colorless oil.

LCMS (ESI-MS) m/z=324.1 [M+H]⁺.

### Step 4: 6-benzyl-2-(benzyloxy)-6-azaspiro[3.4]octan-8-one

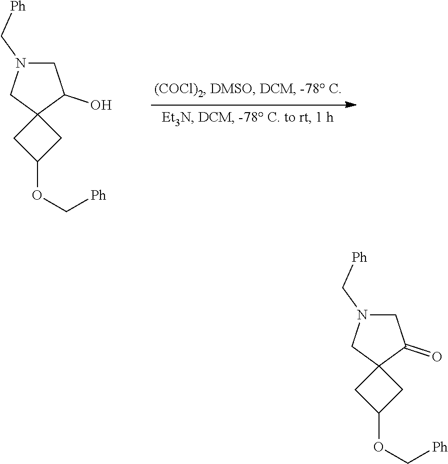 [SMILES: OC1CN(Cc2ccccc2)CC12CC(OCc1ccccc1)C2 ; O=C1CN(Cc2ccccc2)CC12CC(OCc1ccccc1)C2]

To a cooled to −78° C. solution of oxalyl chloride (7.85 g, 61.8 mmol) in DCM (100 mL) was added dropwise a solution of DMSO (4.83 g, 61.8 mmol) in DCM (20 mL) under a nitrogen atmosphere. The mixture was stirred at −78° C. for 20 min. A solution of 6-benzyl-2-(benzyloxy)-6-azaspiro[3.4]octan-8-ol (10 g, 30.9 mmol) in DCM (20 mL) was then added dropwise and the mixture stirred for 20 min. Et₃N (12.5 g, 123 mmol) was added dropwise and the mixture stirred for 20 min. The reaction mixture was diluted with water (200 mL) and extracted with DCM (3×200 mL). The combined organic layers were washed with brine, dried over anhydrous Na₂SO₄, filtered and concentrated under reduced pressure. The residue was purified by silica gel column chromatography (EA in PE, 0 to 10%). The fractions with the desired mass signal were combined and concentrated under reduced pressure to afford 6-benzyl-2-(benzyloxy)-6-azaspiro[3.4]octan-8-one (5.8 g, 58.4% yield).

LCMS (ESI-MS) m/z=322.2 [M+H]⁺.

### Step 5: 6-benzyl-2-(benzyloxy)-8,8-difluoro-6-azaspiro[3.4]octane

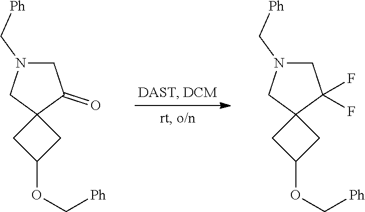 [SMILES: O=C1CN(Cc2ccccc2)CC12CC(OCc1ccccc1)C2 ; FC1(F)CN(Cc2ccccc2)CC12CC(OCc1ccccc1)C2]

DAST (8.73 g, 54.1 mmol) was added to a solution of 6-benzyl-2-(benzyloxy)-6-azaspiro[3.4]octan-8-one (5.8 g, 18.0 mmol) in DCM (60 mL) at 0° C. The resulting mixture was stirred overnight at room temperature. The reaction mixture was concentrated under reduced pressure. The residue was purified by silica gel column chromatography (EA in PE, 0% to 10%). The fractions with the desired mass signal were combined and concentrated under reduced pressure to afford 6-benzyl-2-(benzyloxy)-8,8-difluoro-6-azaspiro[3.4]octane (1.2 g, 19.4% yield).

LCMS (ESI-MS) m/z=344.2 [M+H]⁺.

### Step 6: Tert-butyl 8,8-difluoro-2-hydroxy-6-azaspiro[3.4]octane-6-carboxylate

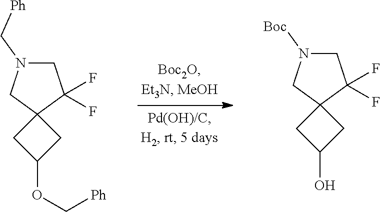 [SMILES: FC1(F)CN(Cc2ccccc2)CC12CC(OCc1ccccc1)C2 ; CC(C)(C)OC(=O)N1CC(F)(F)C2(CC(O)C2)C1]

Pd(OH)₂/C (0.49 g, 3.49 mmol) was added to a solution of 6-benzyl-2-(benzyloxy)-8,8-difluoro-6-azaspiro[3.4]octane (1.2 g, 3.49 mmol), Boc₂O (0.92 g, 4.19 mmol) and Et₃N (1.06 g, 10.48 mmol) in MeOH (120 mL). The resulting mixture was stirred 5 days at room temperature under a H₂ atmosphere. The reaction mixture was filtered and the filtrate was concentrated under reduced pressure. The residue was purified by silica gel column chromatography (MeOH in DCM, 0% to 5%). The fractions with the desired mass signal were combined, concentrated under reduced pressure and lyophilized to afford the tert-butyl 8,8-difluoro-2-hydroxy-6-azaspiro[3.4]octane-6-carboxylate (500 mg, 54.4% yield).

¹H NMR (400 MHz, DMSO-d₆) δ 5.3-5.06 (m, 1H), 4.20-4.00 (m, 1H), 3.69-3.50 (m, 3H), 3.47-3.39 (m, 2H), 2.14-2.10 (m, 1H), 2.04-1.96 (m, 1H), 1.93-1.85 (m, 1H), 1.40 (s, 9H).

### Intermediate 17: 3-(((tert-butyldiphenylsilyl)oxy)methyl)-3-(difluoromethyl)cyclobutan-1-ol

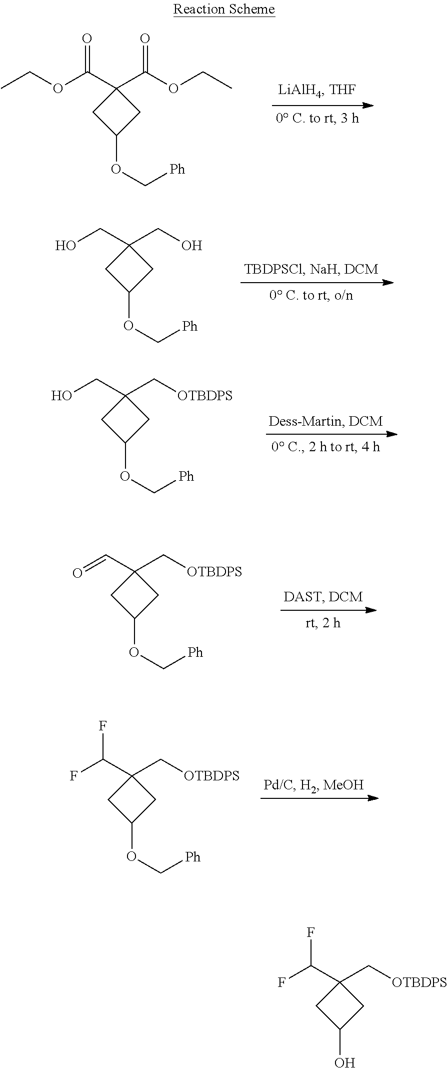

### Detailed Procedure

### Step 1: (3-(benzyloxy)cyclobutane-1,1-diyl)dimethanol

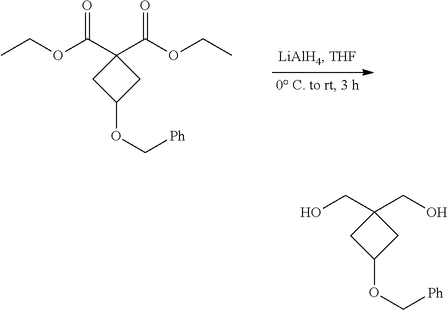 [SMILES: CCOC(=O)C1(C(=O)OCC)CC(OCc2ccccc2)C1 ; OCC1(CO)CC(OCc2ccccc2)C1]

A solution of LiAlH₄ in THF (2 M, 32.6 mL, 65.2 mmol) was added to a stirred mixture of 1,1-diethyl 3-(benzyloxy)cyclobutane-1,1-dicarboxylate (5 g, 16.3 mmol) in THF (50 mL) dropwise at 0° C. The resulting mixture was stirred at room temperature for 3 h, carefully quenched by addition of water (50 mL) and extracted with DCM (3×50 mL). The combined organic layers were washed with brine, dried over anhydrous Na₂SO₄, filtered and concentrated under reduced pressure to afford crude [3-(benzyloxy)-1-(hydroxymethyl)cyclobutyl] methanol (3.2 g) as colorless oil. The crude product was used for the next step without further purification.

LCMS (ESI-MS) m/z=223.3 [M+H]⁺.

### Step 2: (3-(benzyloxy)-1-(((tert-butyldiphenylsilyl)oxy)methyl)cyclobutyl)methanol

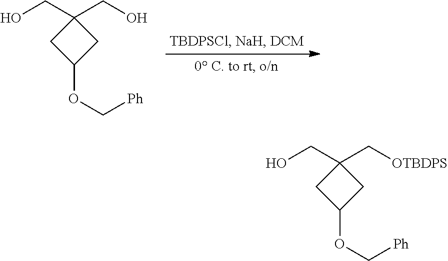

NaH (60% in mineral oil, 0.99 g, 24.8 mmol) was added to a cooled to 0° C. solution of [3-(benzyloxy)-1-(hydroxymethyl)cyclobutyl]methanol (3.2 g, 14.3 mmol) in THF (40 mL). The resulting suspension was stirred at 0° C. for 1 h and TBDPSCl (3.96 g, 14.3 mmol) was slowly added. The resulting mixture was stirred at room temperature overnight, diluted with saturated aqueous NH₄Cl (100 mL) and extracted with EA (3×100 mL). The combined organic layers were washed with brine (100 mL), dried over Na₂SO₄, filtered and concentrated under reduced pressure to afford crude (3-(benzyloxy)-1-(((tert-butyldiphenylsilyl)oxy) methyl)cyclobutyl)methanol (6.5 g). The crude product was used for the next step without further purification.

LCMS (ESI-MS) m/z=461.4 [M+H]⁺.

### Step 3: 3-(benzyloxy)-1-(((tert-butyldiphenylsilyl)oxy)methyl)cyclobutane-1-carbaldehyde

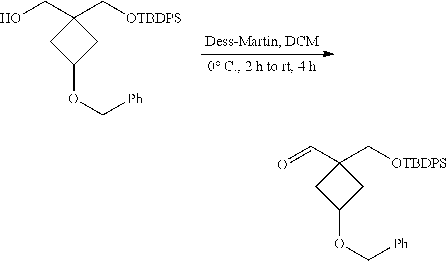

Dess-Martin periodinate (4.14 g, 9.76 mmol) was slowly added to a cooled to 0° C. solution of (3-(benzyloxy)-1-(((tert-butyldiphenylsilyl)oxy)methyl)cyclobutyl)methanol (3 g crude, around 6.51 mmol) in DCM (30 mL) under a nitrogen atmosphere. After stirring for 2 h at 0° C., the reaction was warmed to room temperature and stirred for 4 h. The reaction was quenched by addition of saturated aqueous NaHCO₃(50 mL) and extracted with DCM (3×50 mL). The combined organic layers were dried over anhydrous Na₂SO₄, filtered and concentrated under reduced pressure. The residue was purified by silica gel flash column (EA/PE, 10:90) to afford 3-(benzyloxy)-1-(((tert-butyldiphenylsilyl)oxy)methyl) cyclobutane-1-carbaldehyde (1.8 g, 60.2% yield).

LCMS (ESI-MS) m/z=459.2 [M+H]⁺.

### Step 4: ((3-(benzyloxy)-1-(difluoromethyl)cyclobutyl)methoxy)(tert-butyl)diphenylsilane

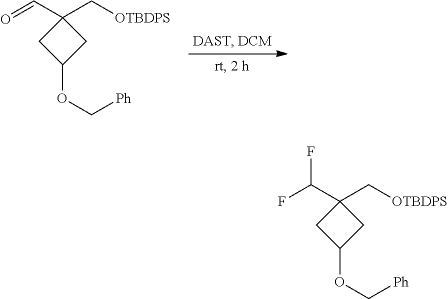

DAST (948 mg, 5.88 mmol) was added to a stirred mixture of 3-(benzyloxy)-1-(((tert-butyldiphenylsilyl)oxy)methyl)cyclobutane-1-carbaldehyde (1.8 g, 3.92 mmol) in DCM (20 mL). The resulting mixture was stirred at room temperature for 2 h, quenched by addition of water (50 mL) and extracted with DCM (3×50 mL). The combined organic layers were washed with brine, dried over anhydrous Na₂SO₄, filtered and concentrated under reduced pressure to give ((3-(benzyloxy)-1-(difluoromethyl) cyclobutyl)methoxy)(tert-butyl)diphenylsilane (1 g, 53.0% yield).

LCMS (ESI-MS) m/z=481.4 [M+H]⁺.

### Step 5: 3-(((tert-butyldiphenylsiyl)oxy)methyl)-3-(difluoromethyl)cyclobutan-1-ol

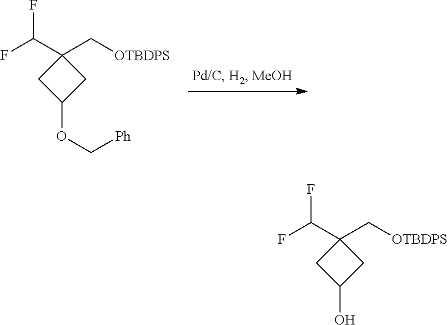

Pd/C (10% on carbon, 1.11 g) was added to a mixture of (3-(benzyloxy)-1-(difluoromethyl)cyclobutyl)methoxy)(tert-butyl)diphenylsilane (1 g, 2.08 mmol) in MeOH (15 mL) under a nitrogen atmosphere. The reaction mixture was stirred overnight at room temperature under a hydrogen atmosphere, filtered and the filtrate was concentrated under reduced pressure. The residue was purified by preparative RP-HPLC to give 3-(((tert-butyldiphenylsilyl)oxy)methyl)-3-(difluoromethyl)cyclobutan-1-ol (100 mg, 12.3% yield).

LCMS (ESI-MS) m/z=391.2 [M+H]⁺.

### Intermediate 18: Racemic (3R,4R)-4-amino-1-(methylsulfonyl)piperidin-3-ol

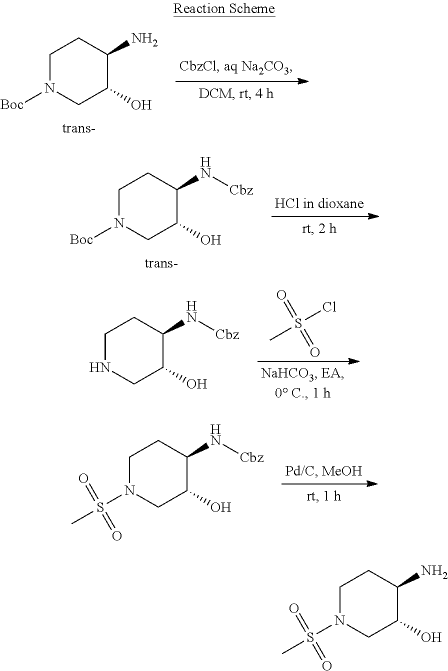

### Step 1: Racemic tert-butyl (3R,4R)-(((benzyloxy)carbonyl)amino)-3-hydroxypiperidine-1-carboxylate

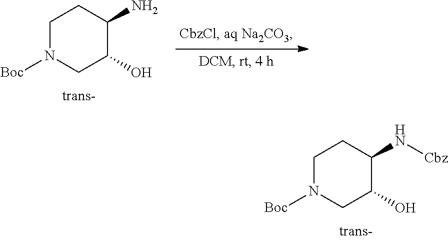 [SMILES: CC(C)(C)OC(=O)N1CC[C@@H](N)[C@H](O)C1 ; CC(C)(C)OC(=O)N1CC[C@@H](NC(=O)OCc2ccccc2)[C@H](O)C1]

To a mixture of racemic tert-butyl (3R,4R)-4-amino-3-hydroxypiperidine-1-carboxylate (500 mg, 2.31 mmol) in DCM (3.8 mL) and saturated aqueous Na₂CO₃ (3.8 mL) was added a solution of Cb₂Cl (50% in toluene, 946 mg, 2.77 mmol) dropwise at 0° C. The resulting mixture was stirred for 4 h at room temperature, diluted with water (20 mL) and extracted with DCM (2×20 mL). The combined organic layers were washed with brine (20 mL), dried over anhydrous Na₂SO₄, filtered and concentrated under reduced pressure. The residue was purified by Prep-TLC (EA/PE, 2:1) to afford racemic tert-butyl (3R,4R)-4-(((benzyloxy)carbonyl)amino)-3-hydroxypiperidine-1-carboxylate (800 mg, 98.7% yield).

LCMS (ESI-MS) m/z=351.2 [M+H]⁺.

### Step 2: Racemic benzyl ((3R,4R)-3-hydroxypiperidin-4-yl)carbamate

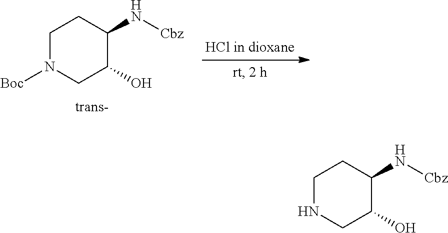 [SMILES: CC(C)(C)OC(=O)N1CC[C@@H](NC(=O)OCc2ccccc2)[C@H](O)C1 ; O=C(N[C@@H]1CCNC[C@H]1O)OCc1ccccc1]

A solution of racemic tert-butyl (3R,4R)-4-(((benzyloxy)carbonyl)amino)-3-hydroxypiperidine-1-carboxylate (750 mg, 2.14 mmol) was added to a solution of HCl in 1,4-dioxane (4 M, 5 mL, 20 mmol). The resulting mixture was stirred for 2 h at room temperature and concentrated under reduced pressure. The crude product was used for the next step without further purification.

LCMS (ESI-MS) m/z=251.2 [M+H]⁺.

### Step 3: Racemic benzyl ((3R,4R)-3-hydroxy-1-(methylsulfonyl)piperidin-4-yl)carbamate

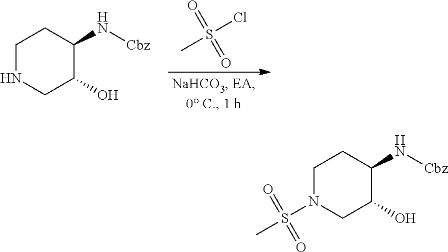

Methanesulfonyl chloride (549 mg, 4.79 mmol) was added to a stirred mixture of crude racemic benzyl ((3R,4R)-3-hydroxypiperidin-4-yl)carbamate (750 mg, 2.99 mmol) and NaHCO₃(420 mg, 5 mmol) in EA (4.5 mL) at 0° C. The resulting mixture was stirred for 1 h, quenched by addition of water (20 mL) and extracted with EA (2×20 mL). The combined organic layers were dried over anhydrous Na₂SO₄, filtered and concentrated under reduced pressure. The residue was purified by Prep-TLC (EA) to afford racemic benzyl ((3R,4R)-3-hydroxy-1-(methylsulfonyl)piperidin-4-yl)carbamate (500 mg, 50.8% yield).

LCMS (ESI-MS) m/z=329.1 [M+H]⁺.

### Step 4: Racemic (3R,4R)-4-amino-1-(methylsulfonyl)piperidin-3-ol

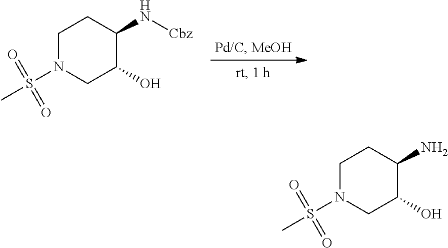 [SMILES: CS(=O)(=O)N1CC[C@@H](NC(=O)OCc2ccccc2)[C@H](O)C1 ; CS(=O)(=O)N1CC[C@@H](N)[C@H](O)C1]

Pd/C (10% on carbon, 25 mg) was added to a mixture of racemic benzyl ((3R,4R)-3-hydroxy-1-(methylsulfonyl)piperidin-4-yl)carbamate (100 mg, 0.30 mmol) in MeOH (5 mL) under a nitrogen atmosphere. The resulting mixture was stirred for 1 h at room temperature under a hydrogen atmosphere and filtered. The filter cake was washed with MeOH (50 mL). The filtrate was concentrated under reduced pressure to afford crude racemic (3R,4R)-4-amino-1-(methylsulfonyl)piperidin-3-ol (75.6 mg). The crude product was used for the next step without further purification.

LCMS (ESI-MS) m/z=195.1 [M+H]⁺.

### Example 31: N-(1-(methylsulfonyl)piperidin-4-yl)-8-(2,6-diazaspiro[3.4]octan-2-yl)quinazolin-2-amine

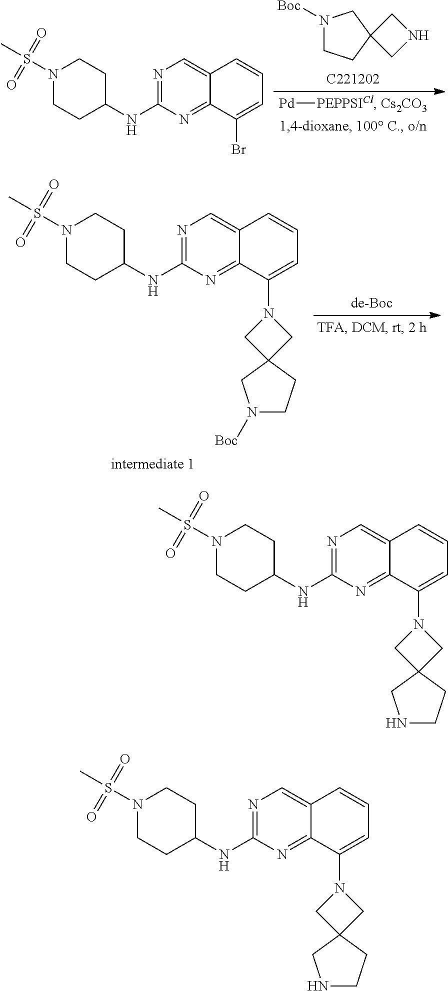

### Step 1: tert-butyl 2-(2-((1-(methyl sulfonyl) piperidin-4-yl) amino) quinazolin-8-yl)-2,6-diazaspiro[3.4]octane-6-carboxylate

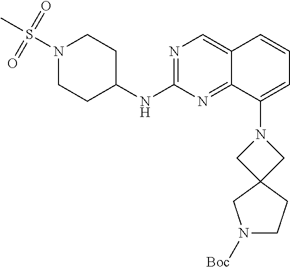 [SMILES: CC(C)(C)OC(=O)N1CCC2(C1)CN(c1cccc3cnc(NC4CCN(S(C)(=O)=O)CC4)nc13)C2]

To a mixture of 8-bromo-N-(1-(methyl sulfonyl) piperidin-4-yl) quinazolin-2-amine (80 mg, 0.21 mmol), tert-butyl 2,6-diazaspiro[3.4]octane-6-carboxylate (53 mg, 0.25 mmol) and cesium carbonate (205 mg, 0.63 mmol) in 1,4-dioxane (3.0 mL) was added Pd-PEPPSI^{Cl} (10.18 mg, 0.01 mmol) under a nitrogen atmosphere. The resulting mixture was heated to 100° C. and stirred overnight under a nitrogen atmosphere. H₂O (0.1 mL) was added to the mixture and the residue was purified by preparative reverse phase HPLC to provide the title compound (50 mg, 38% yield) as a yellow solid.

LCMS (ESI) [M+H]⁺=517.1

### Step 2: N-(1-(methyl sulfonyl) piperidin-4-yl)-8-(2,6-diazaspiro[3.4]octan-2-yl) quinazolin-2-amine

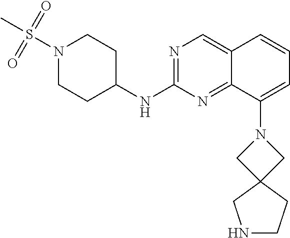 [SMILES: CS(=O)(=O)N1CCC(Nc2ncc3cccc(N4CC5(CCNC5)C4)c3n2)CC1]

To a mixture of tert-butyl 2-(2-((1-(methyl sulfonyl) piperidin-4-yl) amino) quinazolin-8-yl)-2,6-diazaspiro[3.4]octane-6-carboxylate (50 mg, 0.10 mmol) in DCM (2.5 mL) was added trifluoroacetic acid (0.25 mL, 3.98 mmol). The mixture was stirred at room temperature for 2 h. The solvent was removed under reduced pressure and dissolved in DMSO/MeOH/Et₃N (1:1:0.05, 3 mL). The mixture was purified by preparative reverse phase HPLC to give the title compound (12.9 mg, 32% yield) as a yellow solid. LCMS (ESI) [M+H]⁺=417.1.

### Example Compounds

### Example 24: 8-(2-methyl-2,6-diazaspiro[3.4]octan-6-yl)-N-(1-(methylsulfonyl)piperidin-4-yl)quinazolin-2-amine

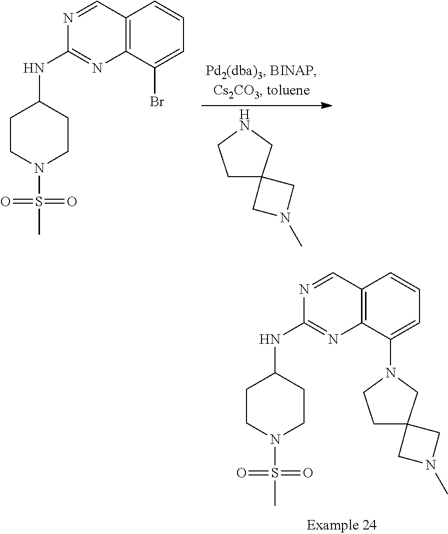

To a mixture of 8-bromo-N-(1-(methylsulfonyl)piperidin-4-yl)quinazolin-2-amine (200 mg, 0.52 mmol), 2-methyl-2,6-diazaspiro[3.4]octane (72.06 mg, 0.57 mmol) and cesium carbonate (507.40 mg, 1.55 mmol) in toluene (2 mL) was added Pd₂(dba)₃ (47.6 mg, 0.052 mmol) and BINAP (32.3 mg, 0.052 mmol) under a nitrogen atmosphere. The resulting mixture was heated to 100° C. and stirred overnight under a nitrogen atmosphere. The reaction mixture was allowed to cool to room temperature and then filtered. The collected solid was washed with dichloromethane (5 mL) and the combined filtrate was concentrated under reduced pressure. The residue was purified by Prep-TLC (dichloromethane/methanol, 10:1). The product was further purified by preparative reverse phase HPLC (acetonitrile/water (with 10 mM NH₄HCO₃ and 0.1% NH₃·H₂O) gradient) to afford the title compound (59.6 mg, 25.4% yield).

LCMS (ESI) m/z=431 [M+H]⁺.

¹H NMR (400 MHz, CDCl₃) δ 8.90 (s, 1H), 7.15-7.09 (m, 2H), 6.83-6.79 (m, 1H), 5.31 (s, 1H), 4.11-4.05 (m, 3H), 3.80-3.75 (m, 2H), 3.67-3.43 (m, 6H), 3.05-2.98 (m, 2H), 2.84 (s, 3H), 2.48 (s, 3H), 2.26-2.19 (m, 4H), 1.81-1.69 (m, 2H).

### Example 32: N-(1-(methyl sulfonyl) piperidin-4-yl)-8-(2,6-diazaspiro[3.4]octan-2-yl) quinazolin-2-amine

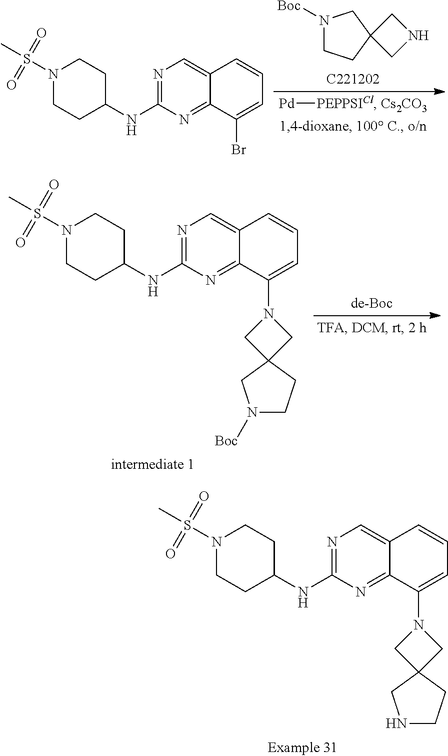

### Step 1: Tert-butyl 2-(2-((1-(methyl sulfonyl) piperidin-4-yl) amino) quinazolin-8-yl)-2,6-diazaspiro[3.4]octane-6-carboxylate

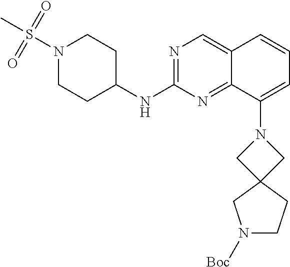 [SMILES: CC(C)(C)OC(=O)N1CCC2(C1)CN(c1cccc3cnc(NC4CCN(S(C)(=O)=O)CC4)nc13)C2]

To a mixture of 8-bromo-N-(1-(methyl sulfonyl) piperidin-4-yl) quinazolin-2-amine (80 mg, 0.21 mmol), tert-butyl 2,6-diazaspiro[3.4]octane-6-carboxylate (53 mg, 0.25 mmol) and cesium carbonate (205 mg, 0.63 mmol) in 1,4-dioxane (3.0 mL) was added Pd-PEPPSI^{Cl} (10.18 mg, 0.01 mmol) under a nitrogen atmosphere. The resulting mixture was heated to 100° C. and stirred overnight under a nitrogen atmosphere. H₂O (0.1 mL) was added to a mixture and the residue was purified by preparative reverse phase HPLC to provide the title compound (50 mg, 38% yield). LCMS (ESI) [M+H]⁺=517.1

### Step 2: N-(1-(methyl sulfonyl) piperidin-4-yl)-8-(2,6-diazaspiro[3.4]octan-2-yl) quinazolin-2-amine (Example 31)

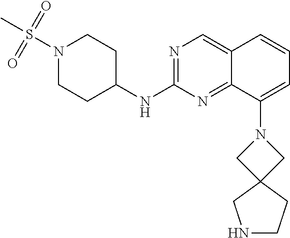 [SMILES: CS(=O)(=O)N1CCC(Nc2ncc3cccc(N4CC5(CCNC5)C4)c3n2)CC1]

To a mixture of tert-butyl 2-(2-((1-(methyl sulfonyl) piperidin-4-yl) amino) quinazolin-8-yl)-2,6-diazaspiro[3.4]octane-6-carboxylate (50 mg, 0.10 mmol) in DCM (2.5 mL) was added trifluoroacetic acid (0.25 mL, 3.98 mmol). The mixture was stirred at room temperature for 2 h. The solvent was removed under reduced pressure and the residue dissolved in DMSO/MeOH/Et₃N (1:1:0.05, 3 mL). The mixture was purified by preparative reverse phase HPLC to give the title compound (12.9 mg, 32% yield). LCMS (ESI) [M+H]⁺=417.1.

### Example 35: 1-(7-(2-((1-(methylsulfonylpiperidin-4-yl)amino)quinazolin-8-yl)-2-azaspiro[4.4]nonan-2-yl)ethan-1-one

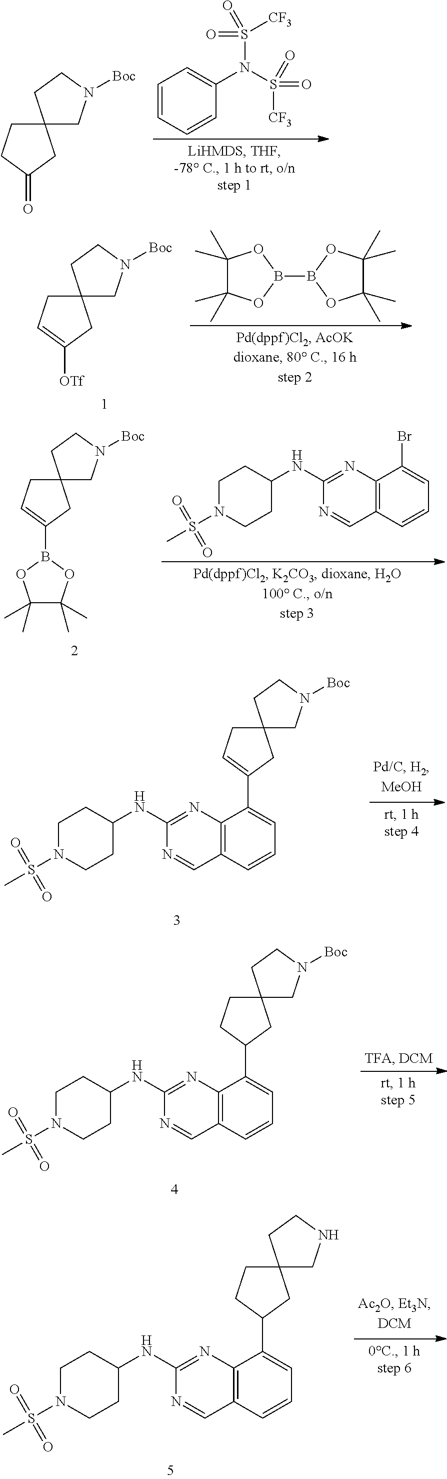

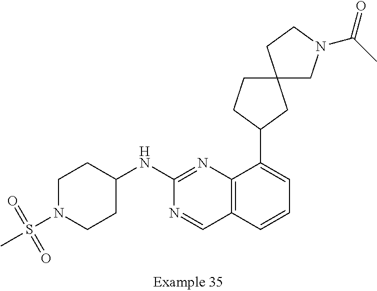 [SMILES: CC(=O)N1CCC2(CCC(c3cccc4cnc(NC5CCN(S(C)(=O)=O)CC5)nc34)C2)C1]

### Detailed Procedure

### Step 1: Tert-butyl 7-(((trifluoromethyl)sulfonyl)oxy)-2-azaspiro[4.4]non-7-ene-2-carboxylate

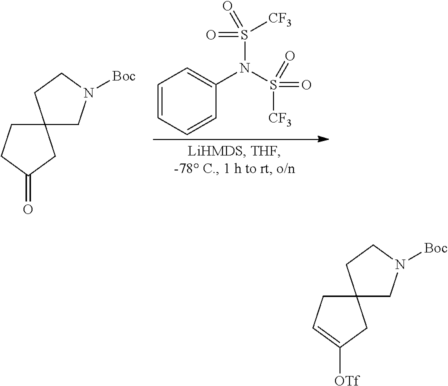

A solution of LiHMDS (1 M, 4.10 mL, 4.10 mmol) in THF was added to a stirred mixture of tert-butyl 7-oxo-2-azaspiro[4.4]nonane-2-carboxylate (500 mg, 2.04 mmol) in THF (6 mL) at −78° C. under a nitrogen atmosphere. The resulting mixture was stirred for 1 h at −78° C. under a nitrogen atmosphere and a solution of 1,1,1-trifluoro-N-phenyl-N-((trifluoromethyl)sulfonyl)methanesulfonamide (1.10 g, 3.07 mmol) in THF (6 mL) was added. The resulting mixture was stirred overnight at room temperature under a nitrogen atmosphere, quenched by addition of saturated aqueous NH₄Cl (50 mL) and extracted with EA (3×50 mL). The combined organic layers were washed with brine (50 mL), dried over anhydrous Na₂SO₄, filtered and the filtrate was concentrated under reduced pressure. The residue was purified by silica gel column chromatography (EA/PE, from 0:100 to 10:90) to afford tert-butyl 7-(((trifluoromethyl)sulfonyl)oxy)-2-azaspiro[4.4]non-7-ene-2-carboxylate (700 mg, 73.6% yield).

LCMS (ESI-MS) m/z=372.1 [M+H]⁺.

### Step 2: Tert-butyl 7-(4,4,5,5-tetramethyl-1,3,2-dioxaborolan-2-yl)-2-azaspiro[4.4]non-7-ene-2-carboxylate

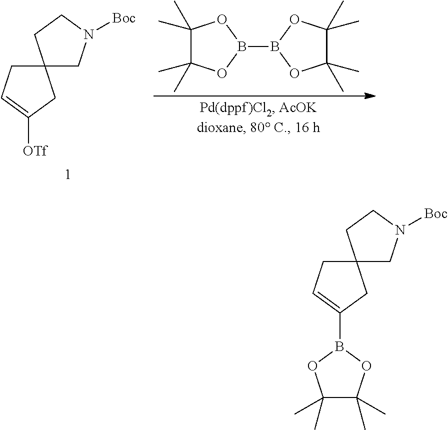

A mixture of tert-butyl 7-(((trifluoromethyl)sulfonyl)oxy)-2-azaspiro[4.4]non-7-ene-2-carboxylate (650 mg, 1.58 mmol), bis(pinacolato)diboron (606 mg, 2.36 mmol), Pd(dppf)Cl₂·CH₂Cl₂ (64.8 mg, 0.08 mmol) and KOAc (312 mg, 3.15 mmol) in 1,4-dioxane (8 mL) was stirred for 16 h at 80° C. under a nitrogen atmosphere. After cooled to room temperature, the reaction mixture was concentrated under reduced pressure directly. The residue was purified by silica-gel column chromatography (EA/PE, 0:100 to 50:50) to afford the desired product tert-butyl 7-(4,4,5,5-tetramethyl-1,3,2-dioxaborolan-2-yl)-2-azaspiro[4.4]non-7-ene-2-carboxylate (850 mg, 84.3% yield).

LCMS (ESI-MS) m/z=350.2 [M+H]⁺.

### Step 3: Tert-butyl 7-(2-((1-(methylsulfonyl)piperidin-4-yl)amino)quinazolin-8-yl)-2-azaspiro[4.4]non-7-ene-2-carboxylate

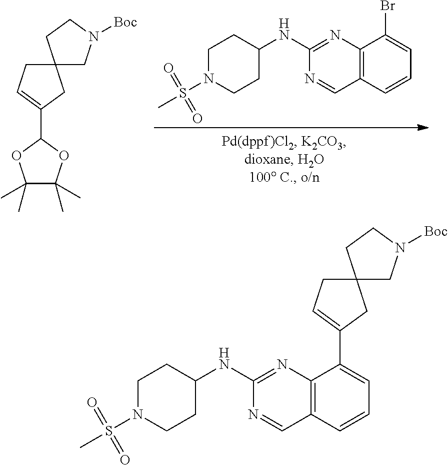 [SMILES: CC(C)(C)OC(=O)N1CCC2(CC=C(C3OC(C)(C)C(C)(C)O3)C2)C1 ; CS(=O)(=O)N1CCC(Nc2ncc3cccc(Br)c3n2)CC1 ; CC(C)(C)OC(=O)N1CCC2(CC=C(c3cccc4cnc(NC5CCN(S(C)(=O)=O)CC5)nc34)C2)C1]

A mixture of tert-butyl 7-(4,4,5,5-tetramethyl-1,3,2-dioxaborolan-2-yl)-2-azaspiro[4.4]non-7-ene-2-carboxylate (850 mg, 1.33 mmol), 8-bromo-N-(1-(methylsulfonyl)piperidin-4-yl)quinazolin-2-amine (517 mg, 1.33 mmol), Pd(dppf)Cl₂·CH₂Cl₂ (54.7 mg, 0.06 mmol) and K₂CO₃ (371 mg, 2.66 mmol) in 1,4-dioxane (4 mL) and water (2 mL) was stirred overnight at 100° C. under a nitrogen atmosphere. After cooling to room temperature, the reaction mixture was concentrated under reduced pressure. The residue was purified by silica-gel column chromatography(EA/PE, 0:100 to 70:30) to afford the desired product tert-butyl 7-(2-((1-(methylsulfonyl)piperidin-4-yl)amino)quinazolin-8-yl)-2-azaspiro[4.4]non-7-ene-2-carboxylate (750 mg, 69.6% yield).

LCMS (ESI-MS) m/z=528.3 [M+H]⁺.

### Step 3: Tert-butyl 7-(2-((1-(methylsulfonyl)piperidin-4-yl)amino)quinazolin-8-yl)-2-azaspiro[4.4]nonane-2-carboxylate

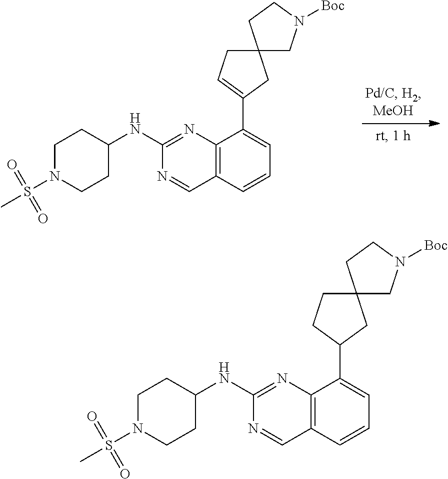 [SMILES: CC(C)(C)OC(=O)N1CCC2(CC=C(c3cccc4cnc(NC5CCN(S(C)(=O)=O)CC5)nc34)C2)C1 ; CC(C)(C)OC(=O)N1CCC2(CCC(c3cccc4cnc(NC5CCN(S(C)(=O)=O)CC5)nc34)C2)C1]

Pd/C (10% on carbon, 25.8 mg) was added to a mixture of tert-butyl 7-(2-((1-(methylsulfonyl)piperidin-4-yl)amino)quinazolin-8-yl)-2-azaspiro[4.4]non-7-ene-2-carboxylate (50 mg, 0.06 mmol) in MeOH (3 mL). The resulting mixture was stirred for 1 h at room temperature under a hydrogen atmosphere. The reaction mixture was filtered and the filtrate was concentrated under reduced pressure to afford crude product tert-butyl 7-(2-((1-(methylsulfonyl)piperidin-4-yl)amino)quinazolin-8-yl)-2-azaspiro[4.4]nonane-2-carboxylate (180 mg crude).

LCMS (ESI-MS) m/z=530.3 [M+H]⁺.

### Step 4: N-(1-(methylsulfonyl)piperidin-4-yl)-8-(2-azaspiro[4.4]nonan-7-yl)quinazolin-2-amine

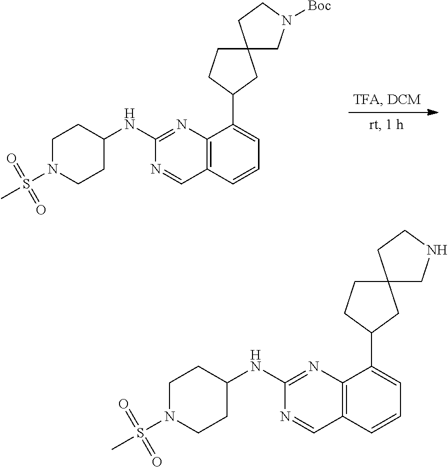 [SMILES: CC(C)(C)OC(=O)N1CCC2(CCC(c3cccc4cnc(NC5CCN(S(C)(=O)=O)CC5)nc34)C2)C1 ; CS(=O)(=O)N1CCC(Nc2ncc3cccc(C4CCC5(CCNC5)C4)c3n2)CC1]

TFA (0.2 mL) was added to a stirred mixture of tert-butyl 7-(2-((1-(methylsulfonyl)piperidin-4-yl)amino)quinazolin-8-yl)-2-azaspiro[4.4]nonane-2-carboxylate (50 mg, 0.06 mmol) in DCM (0.6 mL). The resulting mixture was stirred for 1 h at room temperature and concentrated under reduced pressure to afford crude N-(1-(methylsulfonyl)piperidin-4-yl)-8-(2-azaspiro[4.4]nonan-7-yl)quinazolin-2-amine (180 mg crude).

LCMS (ESI-MS) m/z=430.2 [M+H]⁺.

### Step 5: 1-(7-(2-((1-(methylsulfonyl)piperidin-4-yl)amino)quinazolin-8-yl)-2-azaspiro[4.4]nonan-2-yl)ethan-1-one

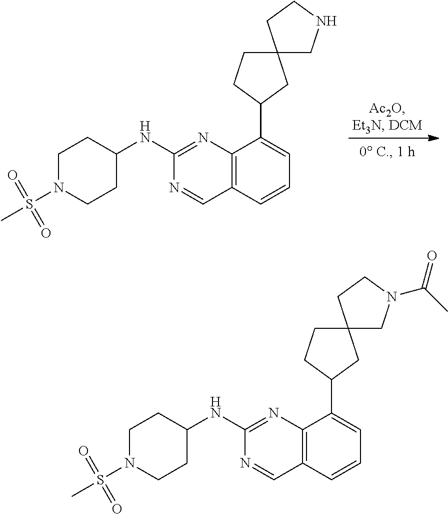 [SMILES: CS(=O)(=O)N1CCC(Nc2ncc3cccc(C4CCC5(CCNC5)C4)c3n2)CC1 ; CC(=O)N1CCC2(CCC(c3cccc4cnc(NC5CCN(S(C)(=O)=O)CC5)nc34)C2)C1]

Ac₂O (6.31 mg, 0.06 mmol) was added to a stirred mixture of N-(1-(methylsulfonyl)piperidin-4-yl)-8-(2-azaspiro[4.4]nonan-7-yl)quinazolin-2-amine (35 mg, 0.06 mmol) and Et₃N (24.7 mg, 0.2 mmol) in DCM (0.5 mL). The resulting mixture was stirred at 0° C. for 1 h and concentrated under reduced pressure. The residue was purified by preparative RP-HPLC to afford the desired product 1-(7-(2-((1-(methylsulfonyl)piperidin-4-yl)amino)quinazolin-8-yl)-2-azaspiro[4.4]nonan-2-yl)ethan-1-one (29.0 mg, 99.4% yield).

¹H NMR (300 MHz, DMSO-d₆) δ 9.09 (s, 1H), 7.53-7.65 (m, 2H), 7.45-7.52 (m, 1H), 7.14-7.21 (m, 1H), 3.96-4.13 (m, 2H), 3.61-3.85 (m, 3H), 3.42-3.57 (m, 2H), 3.22-3.39 (m, 1H), 2.80-3.07 (m, 5H), 2.08-2.29 (m, 4H), 1.51-2.07 (in, 11H).

LCMS (ESI-MS) m/z=472.2 [M+H]⁺, 98.9% purity.

### Example 33: 1-(7-(2-((4-(4-methylpiperazin-1-yl)phenyl)amino) quinazolin-8-yl)-2,7-diazaspiro[4.4]nonan-2-yl)ethan-1-one

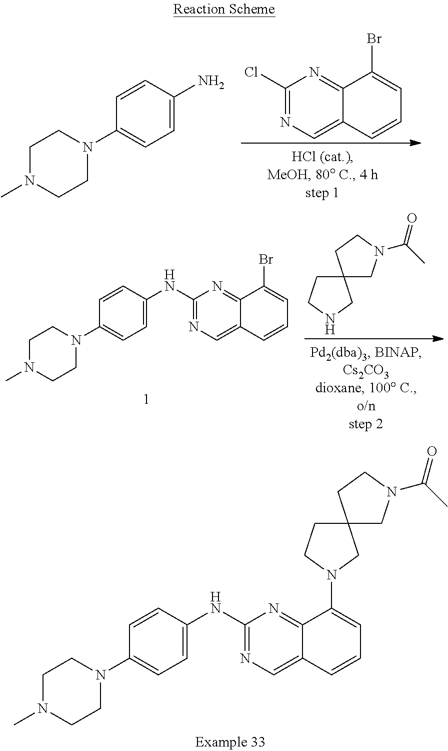

### Detailed Procedure

### Step 1: 8-bromo-N-(4-(4-methylpiperazin-1-yl)phenyl)quinazolin-2-amine

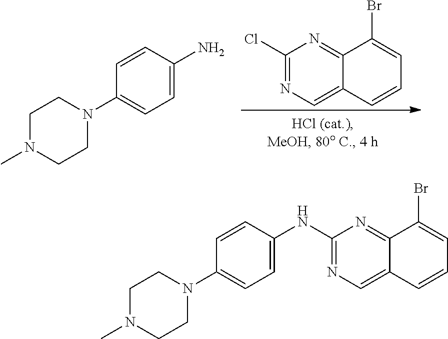 [SMILES: CN1CCN(c2ccc(N)cc2)CC1 ; Clc1ncc2cccc(Br)c2n1 ; CN1CCN(c2ccc(Nc3ncc4cccc(Br)c4n3)cc2)CC1]

A drop of conc. HCl was added to a mixture of 4-(4-methylpiperazin-1-yl)aniline (191 mg, 1.00 mmol) and 8-bromo-2-chloroquinazoline (243 mg, 1.00 mmol) in MeOH (4 mL). The resulting mixture was stirred at 80° C. for 4 h and concentrated under reduced pressure. The residue was purified by silica gel column chromatography (MeOH/DCM, 0:100 to 10:90) to afford the desired product 8-bromo-N-(4-(4-methylpiperazin-1-yl)phenyl)quinazolin-2-amine (180 mg, 42.1% yield).

LCMS (ESI-MS) m/z=398.1 [M+H]⁺.

### Step 2: 1-(7-(2-((4-(4-methylpiperazin-1-yl)phenyl)amino)quinazolin-8-yl)-2,7-diazaspiro[4.4]nonan-2-yl)ethan-1-one

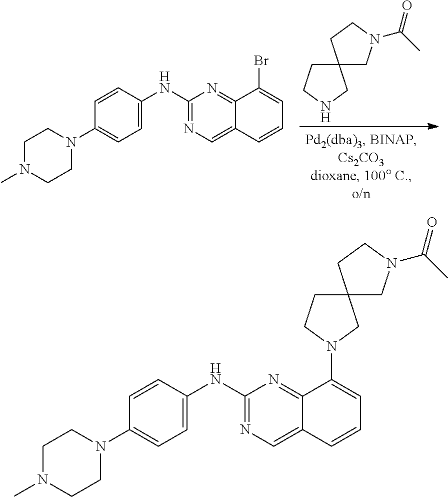

Pd₂(dba)₃ (18.4 mg, 0.02 mmol) was added to a mixture of 8-bromo-N-[4-(4-methylpiperazin-1-yl)phenyl]quinazolin-2-amine (80 mg, 0.21 mmol), 1-(2,7-diazaspiro[4.4]nonan-2-yl)ethan-1-one (33.8 mg, 0.20 mmol), BINAP (12.5 mg, 0.02 mmol) and Cs₂CO₃ (196 mg, 0.60 mmol) in 1,4-dioxane (4 mL) under a nitrogen atmosphere. The resulting mixture was heated to 100° C. and stirred overnight under a nitrogen atmosphere. After cooling to room temperature, the reaction mixture was filtered and the filter cake was washed with DCM (50 mL). The filtrate was concentrated under reduced pressure. The residue was purified by preparative RP-HPLC to afford the desired product 1-(7-(2-((4-(4-methylpiperazin-1-yl)phenyl)amino) quinazolin-8-yl)-2,7-diazaspiro[4.4]nonan-2-yl)ethan-1-one (40.7 mg, 41% yield) (Example 33).

¹H NMR (400 MHz, DMSO-d₆) δ 9.32 (s, 1H), 9.10 (s, 1H), 7.55-7.51 (m, 2H), 7.18-7.11 (m, 2H), 7.02-6.82 (m, 3H), 3.67-3.40 (m, 7H), 3.38-3.21 (m, 2H), 3.17-2.97 (m, 4H), 2.50-2.49 (m, 4H), 2.22 (s, 3H), 1.95-1.82 (m, 6H).

LCMS (ESI-MS) m/z=486.2 [M+H]⁺, 0.3% purity.

### Example 46: 1-(7-(2-((5-(6-ethyl-2,6-diazaspiro[3.3]heptan-2-yl)pyridin-2-yl)amino)quinazolin-8-yl)-2,7-diazaspiro[4.4]nonan-2-yl)ethan-1-one

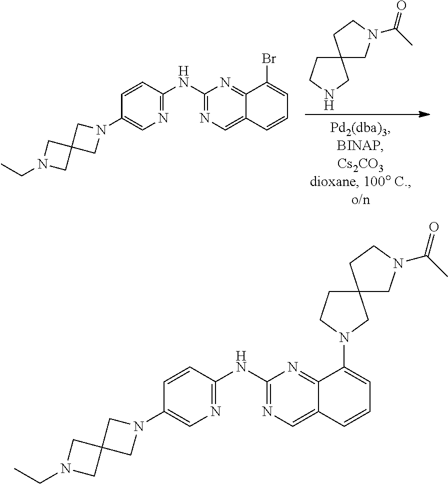

A mixture of 8-bromo-N-(5-(6-ethyl-2,6-diazaspiro[3.3]heptan-2-yl)pyridin-2-yl)quinazolin-2-amine (80 mg, 0.18 mmol), 1-(2,7-diazaspiro[4.4]nonan-2-yl)ethan-1-one (28.7 mg, 0.17 mmol), Cs₂CO₃ (176 mg, 0.54 mmol), BINAP (10.6 mg, 0.017 mmol) and Pd₂(dba)₃ (15.7 mg, 0.017 mmol) in 1,4-dioxane (1 mL) was heated to 100° C. and stirred overnight under a nitrogen atmosphere. After cooling to room temperature, the reaction mixture was filtered and the filter cake was washed with DCM (2×5 mL). The filtrate was concentrated under reduced pressure and the residue was purified by Prep-TLC (MeOH/DCM, 1:10). The residue was further purified purified by preparative RP-HPLC to afford the desired product 1-(7-(2-((5-(6-ethyl-2,6-diazaspiro[3.3]heptan-2-yl)pyridin-2-yl)amino)quinazolin-8-yl)-2,7-diazaspiro[4.4]nonan-2-yl)ethan-1-one (19.6 mg, 22.3% yield).

¹H NMR (300 MHz, Chloroform-d) δ 9.07 (d, J=4.2 Hz, 1H), 8.15 (dd, J=8.8, 4.1 Hz, 1H), 7.81 (s, 1H), 7.63 (t, J=2.7 Hz, 1H), 7.26-7.15 (m, 2H), 6.95-6.82 (m, 2H), 4.07-3.97 (m, 4H), 3.79-3.53 (m, 7H), 3.51-3.39 (m, 5H), 2.51 (dd, J=7.2, 4.7 Hz, 2H), 2.14-2.01 (m, 7H), 1.08-0.93 (m, 3H).

LCMS (ESI-MS) m/z=513.3 [M+H]⁺, 99.6% purity.

### Example 45: 1-(7-(7-fluoro-2-((1-(methylsulfonyl)piperidin-4-yl)amino)quinazolin-8-yl)-2,7-diazaspiro[4.4]nonan-2-yl)ethan-1-one

A mixture of 8-bromo-7-fluoro-N-(1-methanesulfonylpiperidin-4-yl)quinazolin-2-amine (80 mg, 0.19 mmol), 1-(2,7-diazaspiro[4.4]nonan-2-yl)ethan-1-one (33.4 mg, 0.19 mmol), tetrakis(triphenylphosphine)palladium (18.2 mg, 0.02 mmol), 1.1′-Binaphthyl-2.2′-diphemyl phosphine (24.7 mg, 0.04 mmol) and sodium t-butoxide (38.1 mg, 0.39 mmol) in 1,4-dioxane (3 mL) was heated to 100° C. and stirred overnight under a nitrogen atmosphere. After cooling to room temperature, the reaction mixture was diluted with water (10 mL) and extracted with EA (3×30 mL). The combined organic layers were washed with brine, dried over anhydrous Na₂SO₄ and concentrated under reduced pressure. The residue (100 mg) was purified by preparative RP-HPLC to afford the desired product 1-(7-(7-fluoro-2-((1-(methylsulfonyl)piperidin-4-yl)amino)quinazolin-8-yl)-2,7-diazaspiro[4.4]nonan-2-yl)ethan-1-one (28.5 mg, 29.2% yield).

¹H NMR (400 MHz, DMSO-d₆) δ 9.01 (s, 1H), 7.48-7.32 (m, 2H), 7.05-7.00 (m, 1H), 3.83-3.42 (m, 11H), 2.92-2.82 (m, 5H), 2.07-1.90 (m, 9H), 1.61-1.54 (m, 2H).

LCMS (ESI-MS) m/z=491.1 [M+H]⁺.

### Example 58: 6-(difluoromethyl)-N-(2-methylisoindolin-5-yl)-8-(6-(methylsulfonyl)-2,6-diazaspiro[3.3]heptan-2-yl)quinazolin-2-amine

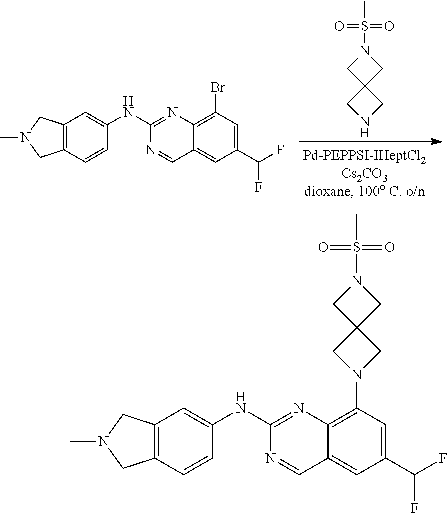 [SMILES: CN1Cc2ccc(Nc3ncc4cc(C(F)F)cc(Br)c4n3)cc2C1 ; CS(=O)(=O)N1CC2(CNC2)C1 ; CN1Cc2ccc(Nc3ncc4cc(C(F)F)cc(N5CC6(C5)CN(S(C)(=O)=O)C6)c4n3)cc2C1]

Pd-PEPPSI-IHeptCl₃-chloropyridine (20.4 mg, 0.02 mmol) was added to a mixture of 8-bromo-6-(difluoromethyl)-N-(2-methylisoindolin-5-yl)quinazolin-2-amine (85 mg, 0.21 mmol), 2-(methylsulfonyl)-2,6-diazaspiro[3.3]heptane (36.9 mg, 0.21 mmol) and Cs₂CO₃ (205 mg, 0.63 mmol) in 1,4-dioxane (2 mL) under a nitrogen atmosphere. The resulting mixture was heated to 100° C. and stirred overnight under a nitrogen atmosphere. After cooling to room temperature, the reaction mixture was filtered and the filter cake was washed with DCM (20 mL). The filtrate was concentrated under reduced pressure. The residue was purified by Prep-TLC (MeOH/DCM, 1:10). The product was further purified by preparative RP-HPLC to afford the desired product 6-(difluoromethyl)-N-(2-methylisoindolin-5-yl)-8-(6-(methylsulfonyl)-2,6-diazaspiro[3.3]heptan-2-yl)quinazolin-2-amine (23.2 mg, 21.7% yield) (Example 58).

¹H NMR (300 MHz, Chloroform-d) δ 9.06 (s, 1H), 7.60 (s, 1H), 7.38 (d, J=8.4 Hz, 1H), 7.29 (s, 2H), 7.24 (d, J=8.1 Hz, 1H), 6.69 (t, J=54.3 Hz, 2H), 4.35 (s, 4H), 4.15 (s, 4H), 4.04 (d, J=10.9 Hz, 4H), 2.92 (s, 3H), 2.69 (s, 3H).

LCMS (ESI-MS) m/z=501.0 [M+H]⁺.

### Example 67: 6-(difluoromethyl)-8-(2-methyl-2-azaspiro[3.3]heptan-6-yl)-N-(1-(methylsulfonyl)piperidin-4-yl)quinazolin-2-amine

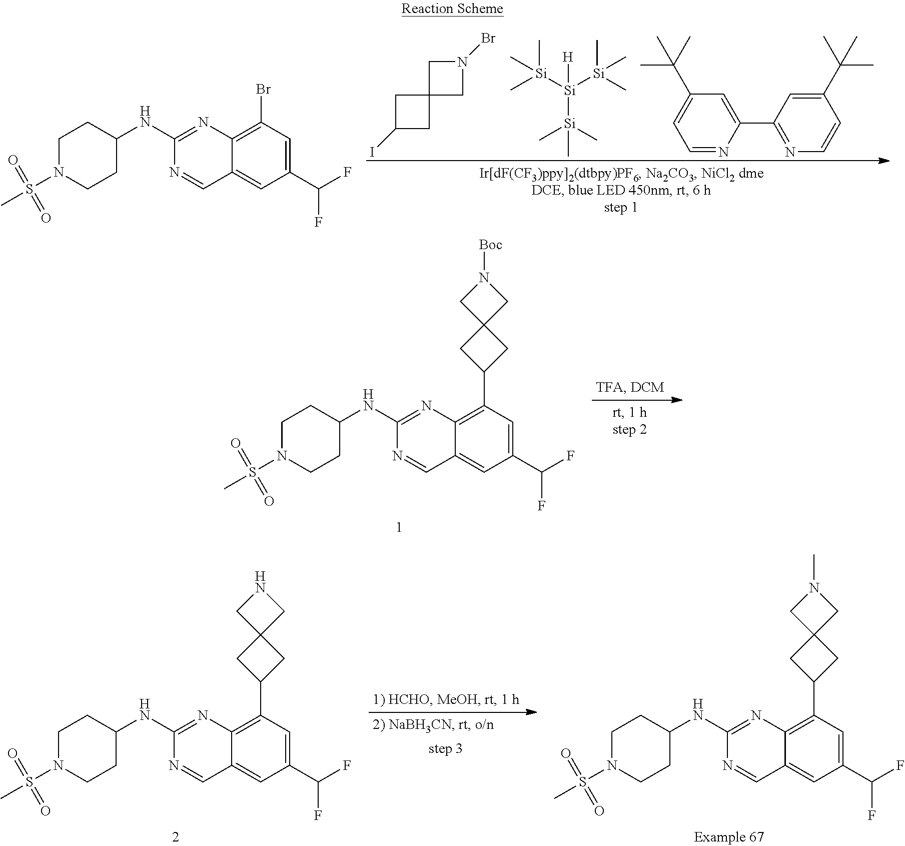

### Step 1: tert-butyl 6-(6-(difluoromethyl)-2-((1-(methylsulfonyl)piperidin-4-yl)amino)quinazolin-8-yl)-2-azaspiro[3.3]heptane-2-carboxylate

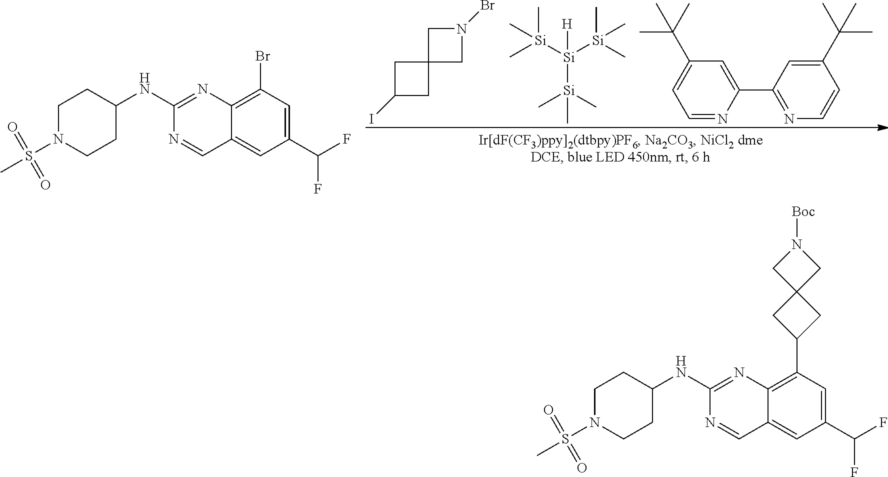

An oven-dried 20 mL vial was charged with 4,4′-di-tert-butyl-2,2′-bipyridine (30.8 mg, 0.11 mmol) and NiCl₂·dme (25.2 mg, 0.11 mmol). DCE (3 mL) was added under a nitrogen atmosphere and the reaction mixture was stirred at 60° C. for 10 minutes (mixture A). Another oven-dried 40 mL vial was charged with 8-bromo-6-(difluoromethyl)-N-(1-(methylsulfonyl)piperidin-4-yl)quinazolin-2-amine (500 mg, 1.15 mmol), tert-butyl 6-iodo-2-azaspiro[3.3]heptane-2-carboxylate (744 mg, 2.30 mmol), 1,1,1,3,3,3-hexamethyl-2-(trimethylsilyl)trisilane (314 mg, 1.26 mmol), Na₂CO₃ (243 mg, 2.30 mmol) and Ir[dF(CF₃)ppy]₂(dtbbpy)PF₆ (16.6 mg, 0.11 mmol). DCE (5 mL) was added under a nitrogen atmosphere (mixture B). The mixture A was added to the mixture B under a nitrogen atmosphere and the resulting mixture was stirred and irradiated with a 450 nm LED lamp under a fan for 6 h. The reaction mixture was quenched with water (20 mL) and extracted with EA (3×20 mL). The combined organic layers were dried over anhydrous Na₂SO₄, filtered and concentrated under reduced pressure. The residue was purified by Prep-TLC (EA/PE, 2:1) to afford tert-butyl 6-(6-(difluoromethyl)-2-((1-(methylsulfonyl)piperidin-4-yl)amino)quinazolin-8-yl)-2-azaspiro[3.3]heptane-2-carboxylate (230 mg).

LCMS (ESI-MS) m/z=552.2 [M+H]⁺.

### Step 2: 6-(difluoromethyl)-N-(1-(methylsulfonyl)piperidin-4-yl)-8-(2-azaspiro[3.3]heptan-6-yl)quinazolin-2-amine

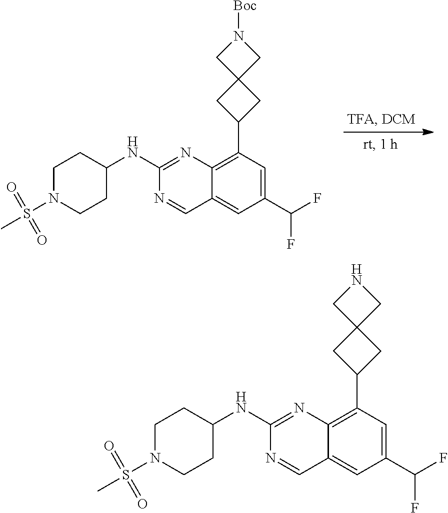 [SMILES: CC(C)(C)OC(=O)N1CC2(CC(c3cc(C(F)F)cc4cnc(NC5CCN(S(C)(=O)=O)CC5)nc34)C2)C1 ; CS(=O)(=O)N1CCC(Nc2ncc3cc(C(F)F)cc(C4CC5(CNC5)C4)c3n2)CC1]

TFA (1 mL) was added to a stirred mixture of tert-butyl 6-(6-(difluoromethyl)-2-((1-(methylsulfonyl) piperidin-4-yl)amino)quinazolin-8-yl)-2-azaspiro[3.3]heptane-2-carboxylate (230 mg, 0.41 mmol) in DCM (3 mL). The resulting mixture was stirred for 1 h at room temperature and concentrated under reduced pressure. The residue was purified by silica gel column chromatography (MeOH/DCM, 10:90) to afford 6-(difluoromethyl)-N-(1-(methylsulfonyl)piperidin-4-yl)-8-(2-azaspiro[3.3]heptan-6-yl)quinazolin-2-amine (100 mg, 47.8% yield).

LCMS (ESI-MS) m/z=452.1 [M+H]⁺.

### Step 3: 6-(difluoromethyl)-8-(2-methyl-2-azaspiro[3.3]heptan-6-yl)-N-(1-(methylsulfonyl)piperidin-4-yl)quinazolin-2-amine

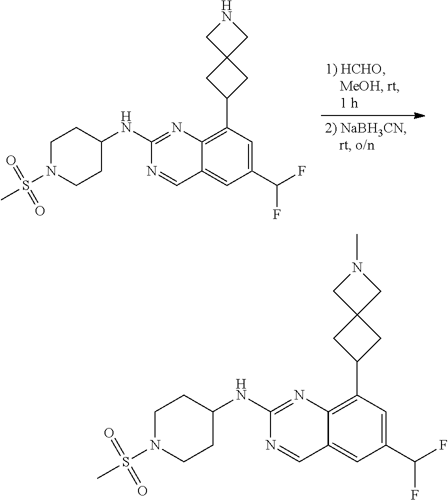 [SMILES: CS(=O)(=O)N1CCC(Nc2ncc3cc(C(F)F)cc(C4CC5(CNC5)C4)c3n2)CC1 ; CN1CC2(CC(c3cc(C(F)F)cc4cnc(NC5CCN(S(C)(=O)=O)CC5)nc34)C2)C1]

A solution of 6-(difluoromethyl)-N-(1-(methylsulfonyl)piperidin-4-yl)-8-(2-azaspiro[3.3]heptan-6-yl) quinazolin-2-amine (70 mg, 0.15 mmol) and HCHO (23.3 mg, 0.77 mmol) in MeOH (1 mL) was stirred for 1 h at room temperature. NaBH₃CN (97.4 mg, 1.55 mmol) was added and the resulting mixture was stirred overnight at room temperature and concentrated under reduced pressure. The residue was purified by Prep-TLC (MeOH/DCM, 1:7). The product was further purified by preparative RP-HPLC to afford 6-(difluoromethyl)-8-(2-methyl-2-azaspiro[3.3]heptan-6-yl)-N-(1-(methylsulfonyl)piperidin-4-yl)quinazolin-2-amine (7.9 mg, 10.7% yield).

¹H NMR (400 MHz, Chloroform-d) δ 8.97 (s, 1H), 7.70-7.54 (m, 2H), 6.71 (t, J=56.4 Hz, 1H), 5.30 (d, J=7.3 Hz, 1H), 4.14-3.95 (m, 2H), 3.80 (d, J=12.4 Hz, 2H), 3.47 (s, 2H), 3.20 (s, 2H), 3.08-2.97 (m, 2H), 2.86 (s, 3H), 2.72-2.63 (m, 2H), 2.39-2.27 (m, 7H), 1.75 (s, 1H), 1.25 (s, 1H). LCMS (ESI-MS) m/z=466.1 [M+H]⁺.

### Example 74: N-(1-(cyclopropylsulfonyl)piperidin-4-yl)-6-(difluoromethyl)-8-(2,6-diazaspiro[3.4]octan-2-yl)quinazolin-2-amine

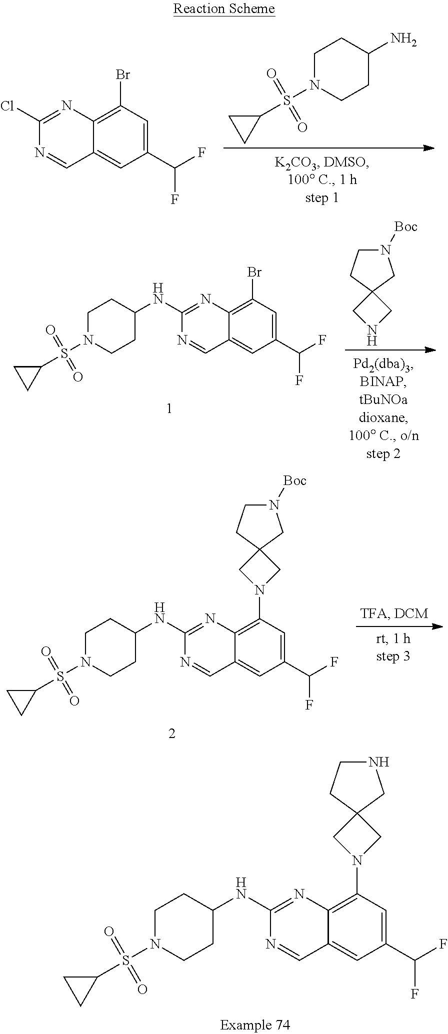 [SMILES: FC(F)c1cc(Br)c2nc(Cl)ncc2c1 ; NC1CCN(S(=O)(=O)C2CC2)CC1 ; O=S(=O)(C1CC1)N1CCC(Nc2ncc3cc(C(F)F)cc(Br)c3n2)CC1 ; CC(C)(C)OC(=O)N1CCC2(CNC2)C1 ; CC(C)(C)OC(=O)N1CCC2(C1)CN(c1cc(C(F)F)cc3cnc(NC4CCN(S(=O)(=O)C5CC5)CC4)nc13)C2 ; O=S(=O)(C1CC1)N1CCC(Nc2ncc3cc(C(F)F)cc(N4CC5(CCNC5)C4)c3n2)CC1]

### Detailed Procedure

### Step 1: 8-bromo-N-(1-(cyclopropylsulfonyl)piperidin-4-yl)-6-(difluoromethyl)quinazolin-2-amine

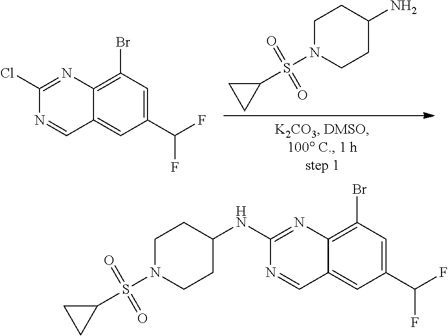

K₂CO₃ (1.84 g, 13.3 mmol) was added to a mixture of 1-(cyclopropylsulfonyl)piperidin-4-amine (0.90 g, 4.42 mmol) and 8-bromo-2-chloro-6-(difluoromethyl)quinazoline (1.3 g, 4.42 mmol) in DMSO (10 mL). The resulting mixture was stirred at 100° C. for 1 h, diluted with water (100 mL) and extracted with DCM (3×100 mL). The combined organic layers were dried over anhydrous Na₂SO₄, filtered and concentrated under reduced pressure. The residue was purified by silica gel column chromatography(EA/PE, 4:1) to afford 8-bromo-N-(1-(cyclopropylsulfonyl)piperidin-4-yl)-6-(difluoromethyl) quinazolin-2-amine (1.2 g, 55.8% yield).

LCMS (ESI-MS) m/z=461.2 [M+H]⁺.

### Step 2: tert-butyl 2-(2-((1-(cyclopropylsulfonyl)piperidin-4-yl)amino)-6-(difluoromethyl)quinazolin-8-yl)-2,6-diazaspiro[3.4]octane-6-carboxylate

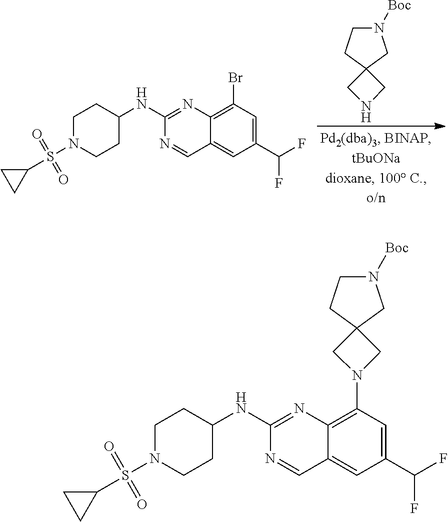 [SMILES: O=S(=O)(C1CC1)N1CCC(Nc2ncc3cc(C(F)F)cc(Br)c3n2)CC1 ; CC(C)(C)OC(=O)N1CCC2(CNC2)C1 ; CC(C)(C)OC(=O)N1CCC2(C1)CN(c1cc(C(F)F)cc3cnc(NC4CCN(S(=O)(=O)C5CC5)CC4)nc13)C2]

A solution of 8-bromo-N-(1-(cyclopropylsulfonyl)piperidin-4-yl)-6-(difluoromethyl) quinazolin-2-amine (150 mg, 0.32 mmol), tert-butyl 2,6-diazaspiro[3.4]octane-6-carboxylate (69.0 mg, 0.32 mmol), sodium 2-methylpropan-2-olate (93.7 mg, 0.97 mmol), BINAP (20.3 mg, 0.03 mmol) and Pd₂(dba)₃ (29.8 mg, 0.03 mmol) in 1,4-dioxane (1 mL) was stirred overnight at 100° C. under a nitrogen atmosphere. After cooling to room temperature, the reaction mixture was filtered and the filter cake was washed with DCM (2×5 mL). The filtrate was concentrated under reduced pressure. The residue was purified by Prep-TLC (MeOH/DCM, 1:10) to afford tert-butyl 2-(2-((1-(cyclopropylsulfonyl)piperidin-4-yl)amino)-6-(difluoromethyl)quinazolin-8-yl)-2,6-diazaspiro[3.4]octane-6-carboxylate (90 mg, 42.0% yield).

LCMS (ESI-MS) m/z=593.4 [M+H]⁺.

### Step 3: N-(1-(cyclopropylsulfonyl)piperidin-4-yl)-6-(difluoromethyl)-8-(2,6-diazaspiro[3.4]octan-2-yl)quinazolin-2-amine

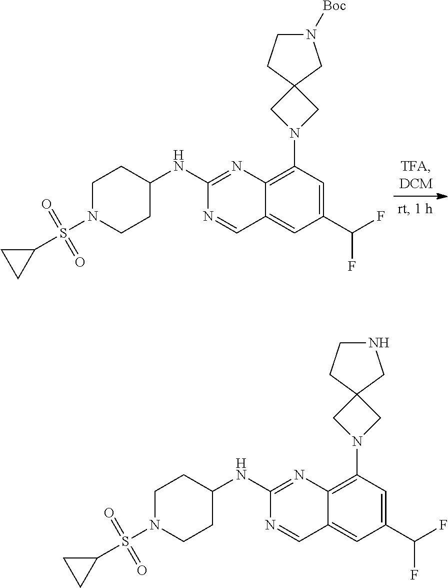 [SMILES: CC(C)(C)OC(=O)N1CCC2(C1)CN(c1cc(C(F)F)cc3cnc(NC4CCN(S(=O)(=O)C5CC5)CC4)nc13)C2 ; O=S(=O)(C1CC1)N1CCC(Nc2ncc3cc(C(F)F)cc(N4CC5(CCNC5)C4)c3n2)CC1]

TFA (0.3 mL) was added to a stirred mixture of tert-butyl 2-(2-((1-(cyclopropylsulfonyl)piperidin-4-yl)amino)-6-(difluoromethyl)quinazolin-8-yl)-2,6-diazaspiro[3.4]octane-6-carboxylate (80 mg, 0.13 mmol) in DCM (1 mL). The resulting mixture was stirred for 1 h at room temperature and concentrated under reduced pressure. The residue was purified by Prep-TLC (MeOH/DCM, 1:10). The product was further purified by preparative RP-HPLC to afford N-(1-(cyclopropylsulfonyl)piperidin-4-yl)-6-(difluoromethyl)-8-(2,6-diazaspiro[3.4]octan-2-yl) quinazolin-2-amine (18.3 mg, 27.2% yield) (Example 74).

¹H NMR (400 MHz, DMSO-d₆) δ 9.09 (s, 1H), 7.64-7.49 (m, 1H), 7.31 (s, 1H), 6.99 (t, J=56.1 Hz, 1H), 6.56 (d, J=7.0 Hz, 1H), 4.14 (d, J=9.8 Hz, 3H), 3.87 (s, 1H), 3.65 (d, J=12.8 Hz, 2H), 3.06-2.93 (m, 4H), 2.91-2.81 (m, 2H), 2.67-2.57 (m, 2H), 2.09-1.92 (m, 4H), 1.66-1.51 (m, 2H), 1.24 (s, 1H), 1.05-0.91 (in, 4H).

LCMS (ESI-MS) m/z=493.1 [M+H]⁺, 98.6% purity.

### Example 89

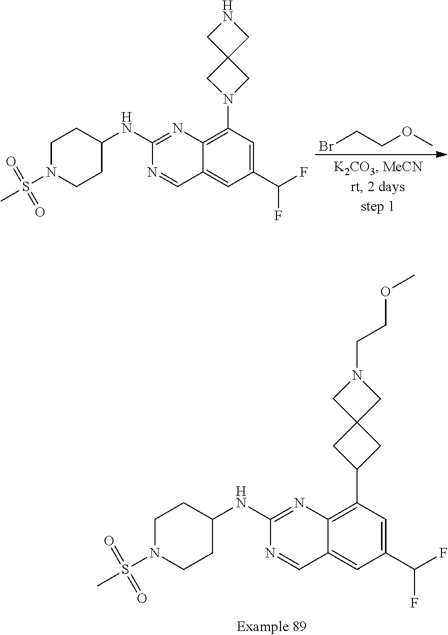

K₂CO₃ (61.21 mg, 0.44 mmol) was added to a mixture of 6-(difluoromethyl)-N-(1-(methylsulfonyl) piperidin-4-yl)-8-(2-azaspiro[3.3]heptan-6-yl)quinazolin-2-amine (100 mg, 0.22 mmol) and 2-bromoethyl methyl ether (30.8 mg, 0.22 mmol) in McCN (4 mL). The resulting mixture was stirred for 2 days at room temperature, diluted with water (20 mL) and extracted with EA (3×20 mL). The combined organic layers were dried over anhydrous Na₂SO₄, filtered and the filtrate was concentrated under reduced pressure. The residue was purified by Prep-TLC (MeOH/DCM, 1:10) to afford 6-(difluoromethyl)-8-(2-(2-methoxyethyl)-2-azaspiro[3.3]heptan-6-yl)-N-(1-(methylsulfonyl) piperidin-4-yl)quinazolin-2-amine (7.3 mg, 6.33% yield) (Example 89).

¹H NMR (400 MHz, Chloroform-d) δ 8.98 (s, 1H), 7.66 (d, J=2.0 Hz, 1H), 7.58 (s, 1H), 6.72 (t, J=56.4 Hz, 1H), 5.39 (d, J=7.3 Hz, 1H), 4.16-3.94 (m, 2H), 3.87-3.76 (m, 2H), 3.66-3.60 (m, 2H), 3.46 (t, J=5.4 Hz, 2H), 3.36 (s, 5H), 3.13-3.01 (m, 2H), 2.89 (s, 3H), 2.79-2.64 (m, 4H), 2.42-2.24 (m, 4H), 1.83-1.67 (m, 2H).

LCMS (ESI-MS) m/z=510.3 [M+H]⁺.

### Example 101

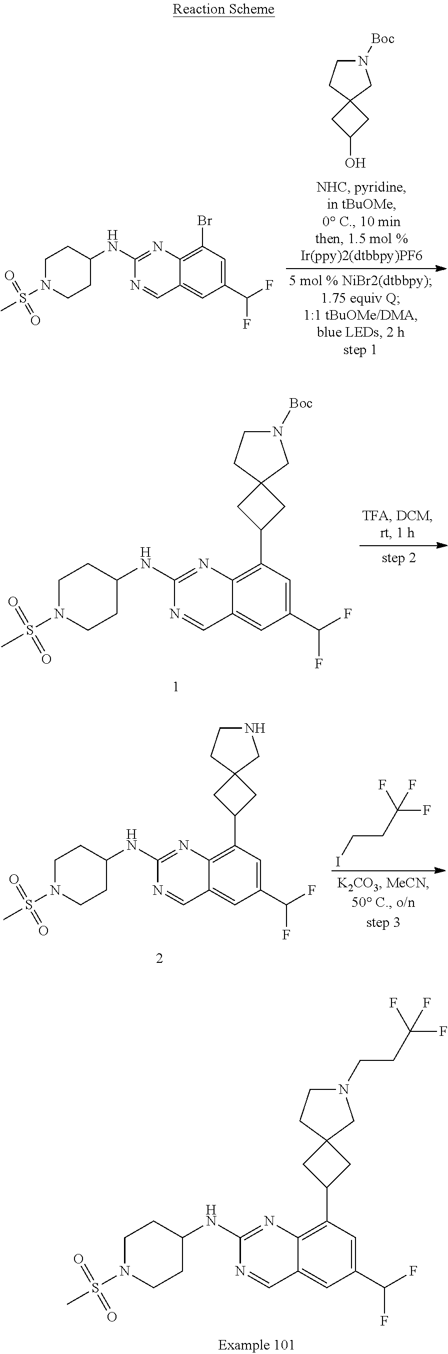

### Detailed Procedure

### Step 1: Tert-butyl 2-(6-(difluoromethyl)-2-((1-(methylsulfonyl)piperidin-4-yl)amino)quinazolin-8-yl)-6-azaspiro[3.4]octane-6-carboxylate

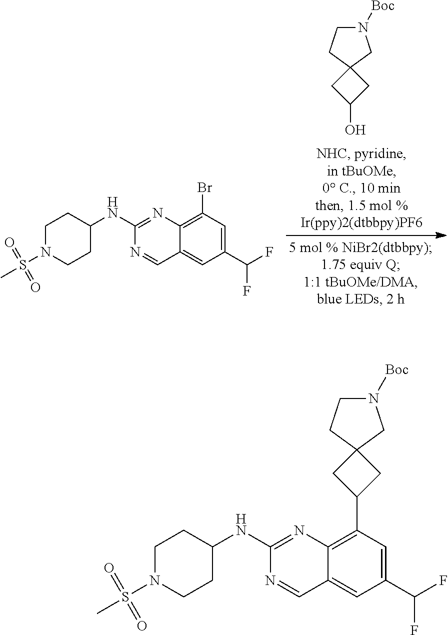 [SMILES: CC(C)(C)OC(=O)N1CCC2(CC(O)C2)C1 ; CS(=O)(=O)N1CCC(Nc2ncc3cc(C(F)F)cc(Br)c3n2)CC1 ; CC(C)(C)OC(=O)N1CCC2(CC(c3cc(C(F)F)cc4cnc(NC5CCN(S(C)(=O)=O)CC5)nc34)C2)C1]

An oven-dried 20 mL vial was charged with tert-butyl 2-hydroxy-6-azaspiro[3.4]octane-6-carboxylate (548 mg, 2.41 mmol) and 5,7-Di-tert-butyl-3-phenylbenzo[d]oxazol-3-ium tetrafluoroborate (NHC) (871 mg, 2.21 mmol). Under nitrogen, tert-butyl methyl ether (24 mL) was added and the reaction stirred at room temperature for 5 minutes. A mixture of pyridine (401 mg, 5.10 mmol) in tert-butyl methyl ether (6 mL) was added and the mixture stirred at room temperature for 10 minutes (mixture A). Another oven-dried 40 mL vial was charged with 8-bromo-6-(difluoromethyl)-N-(1-(methylsulfonyl)piperidin-4-yl)quinazolin-2-amine (600 mg, 1.37 mmol), NiBr₂(dtbbpy) (33.56 mg, 0.06 mmol), Ir(ppy)₂(dtbbpy)PF₆ (18.9 mg, 0.021 mmol) and 1-azabicyclo[2.2.2]octane (Q, 268.2 mg, 2.41 mmol). DMA (30 mL) was added under nitrogen (mixture B). The mixture A was added to the mixture B under a nitrogen atmosphere and the resulting mixture was stirred and irradiated with a 450 nm LED lamp under a fan for 2 h. The reaction mixture was diluted with water (100 mL) and extracted with EA (3×200 mL). The combined organic layer was dried over anhydrous Na₂SO₄, filtered and concentrated under reduced pressure. The residue was purified by Prep-TLC (EA/PE, 2:1) to afford methyl 1-methyl-3-(6-methyl-2-((1-(methylsulfonyl)piperidin-4-yl)amino)pyrido[3,4-d]pyrimidin-8-yl) cyclobutane-1-carboxylate (700 mg).

LCMS (ESI-MS) m/z=566.2 [M+H]⁺.

### Step 2: 6-(difluoromethyl)-N-(1-(methylsulfonyl)piperidin-4-yl)-8-(6-azaspiro[3.4]octan-2-yl)quinazolin-2-amine

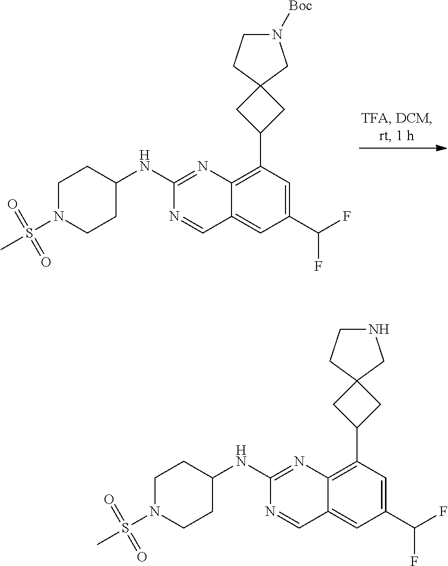 [SMILES: CC(C)(C)OC(=O)N1CCC2(CC(c3cc(C(F)F)cc4cnc(NC5CCN(S(C)(=O)=O)CC5)nc34)C2)C1 ; CS(=O)(=O)N1CCC(Nc2ncc3cc(C(F)F)cc(C4CC5(CCNC5)C4)c3n2)CC1]

TFA (2 mL) was added to a stirred mixture of tert-butyl 2-(6-(difluoromethyl)-2-((1-(methylsulfonyl)piperidin-4-yl)amino)quinazolin-8-yl)-6-azaspiro[3.4]octane-6-carboxylate (700 mg, 1.23 mmol) in DCM (6 mL). The resulting mixture was stirred for 1 h at room temperature and concentrated under reduced pressure. The residue was purified by Prep-TLC (MeOH/DCM, 1:10) to afford 6-(difluoromethyl)-N-(1-(methylsulfonyl)piperidin-4-yl)-8-(6-azaspiro[3.4]octan-2-yl)quinazolin-2-amine (500 mg, 86.8% yield).

LCMS (ESI-MS) m/z=466.2 [M+H]⁺.

### Step 3: 6-(difluoromethyl)-N-(1-(methylsulfonyl)piperidin-4-yl)-8-(6-(3,3,3-trifluoropropyl)-6-azaspiro[3.4]octan-2-yl)quinazolin-2-amine

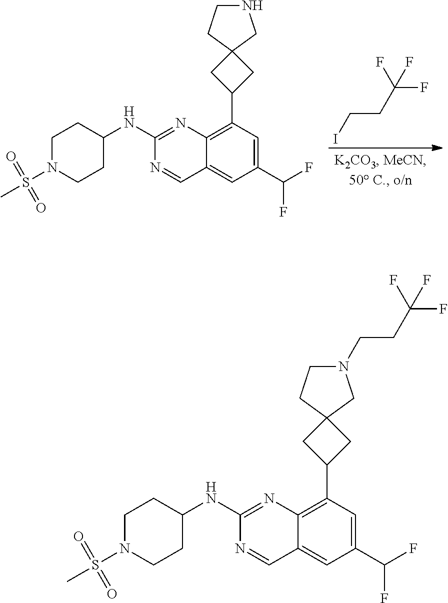

K₂CO₃ (89.1 mg, 0.64 mmol) was added to a mixture of 1,1,1-trifluoro-3-iodopropane (48.1 mg, 0.21 mmol) and 6-(difluoromethyl)-N-(1-(methylsulfonyl)piperidin-4-yl)-8-(6-azaspiro[3.4]octan-2-yl)quinazolin-2-amine (100 mg, 0.21 mmol) in MCCN (1 mL). The resulting mixture was stirred overnight at 50° C., filtered and the filtrate was concentrated under reduced pressure. The residue was purified by preparative RP-HPLC to afford 6-(difluoromethyl)-N-(1-(methylsulfonyl)piperidin-4-yl)-8-(6-(3,3,3-trifluoropropyl)-6-azaspiro[3.4]octan-2-yl)quinazolin-2-amine (9.8 mg, 8.1% yield).

¹H NMR (400 MHz, Chloroform-d) δ 8.98 (s, 1H), 7.72-7.53 (m, 2H), 6.72 (t, J=56.4 Hz, 1H), 5.30 (d, J=7.1 Hz, 1H), 4.2-4.02 (m, 2H), 3.78 (dd, J=10.9, 6.0 Hz, 2H), 3.09-2.94 (m, 2H), 2.85 (s, 3H), 2.76-2.65 (m, 4H), 2.62 (s, 2H), 2.57-2.47 (m, 2H), 2.40-2.23 (m, 6H), 2.22-2.15 (m, 2H), 1.81-1.69 (m, 2H). LCMS (ESI-MS) m/z=562.3 [M+H]⁺.

### Example 102:6-(difluoromethyl)-N-(1-(methylsulfonyl)piperidin-4-yl)-8-(6-(oxetan-3-yl)-6-azaspiro[3.4]octan-2-yl)quinazolin-2-amine

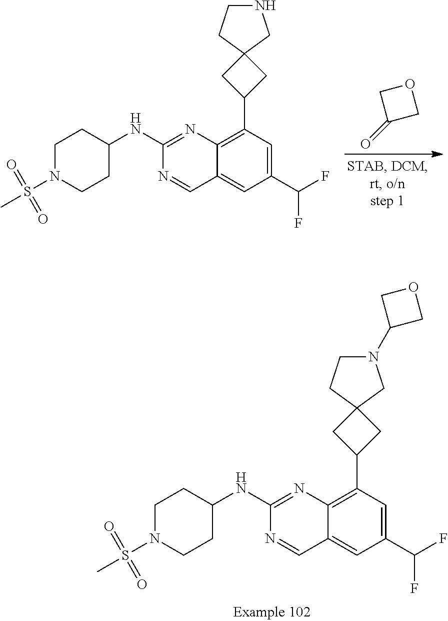 [SMILES: CS(=O)(=O)N1CCC(Nc2ncc3cc(C(F)F)cc(C4CC5(CCNC5)C4)c3n2)CC1 ; O=C1COC1 ; CS(=O)(=O)N1CCC(Nc2ncc3cc(C(F)F)cc(C4CC5(CCN(C6COC6)C5)C4)c3n2)CC1]

STAB (91.1 mg, 0.43 mmol) was added to a mixture of oxetan-3-one (15.5 mg, 0.21 mmol) and 6-(difluoromethyl)-N-(1-(methylsulfonyl)piperidin-4-yl)-8-(6-azaspiro[3.4]octan-2-yl)quinazolin-2-amine (100 mg, 0.21 mmol) in DCM (1 mL). The resulting mixture was stirred overnight at room temperature, diluted with water (10 mL) and extracted with DCM (3×10 mL). The combined organic layers were dried over anhydrous Na₂SO₄, filtered and the filtrate was concentrated under reduced pressure. The residue was purified by preparative RP-HPLC to afford 6-(difluoromethyl)-N-(1-(methylsulfonyl)piperidin-4-yl)-8-(6-(oxetan-3-yl)-6-azaspiro[3.4]octan-2-yl)quinazolin-2-amine (6.2 mg, 5.5% yield) (Example 102).

¹H NMR (400 MHz, Chloroform-d) δ 9.04-8.91 (m, 1H), 7.71-7.50 (m, 2H), 6.71 (t, J=59.2 Hz, 1H), 5.35 (s, 1H), 4.75-4.57 (m, 3H), 4.21-3.93 (m, 2H), 3.85-3.62 (m, 2H), 3.08-2.76 (m, 4H), 2.74-2.44 (m, 5H), 2.36-1.95 (m, 6H), 1.84-1.54 (m, 2H), 1.26 (s, 3H), 0.88 (s, 1H). LCMS (ESI-MS) m/z=522.4 [M+H]⁺.

### Example 106: 3-(2-(6-(difluoromethyl)-2-((1-(methylsulfonylpiperidin-4-yl)amino)quinazolin-8-yl)-6-azaspiro[3.4]octan-6-yl)-2,2-difluoropropan-1-ol

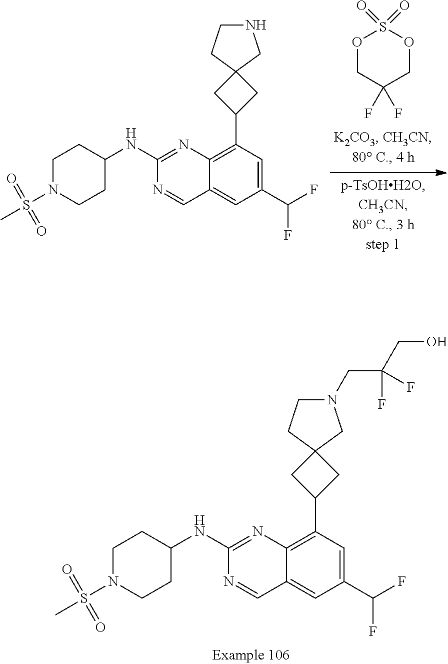 [SMILES: CS(=O)(=O)N1CCC(Nc2ncc3cc(C(F)F)cc(C4CC5(CCNC5)C4)c3n2)CC1 ; O=S1(=O)OCC(F)(F)CO1 ; CS(=O)(=O)N1CCC(Nc2ncc3cc(C(F)F)cc(C4CC5(CCN(CC(F)(F)CO)C5)C4)c3n2)CC1]

A solution of 6-(difluoromethyl)-N-(1-(methylsulfonyl)piperidin-4-yl)-8-(6-azaspiro[3.4]octan-2-yl)quinazolin-2-amine (120 mg, 0.25 mmol), K₂CO₃ (24.9 mg, 0.18 mmol) and 5,5-difluoro-1,3,2-dioxathiane 2,2-dioxide (45.8 mg, 0.26 mmol) in ACN (1 mL) was stirred for 4 h at 80° C. After cooling to room temperature, the reaction mixture was filtered and the filter cake was washed with MCCN (2×0.5 ml). To the combined filtrate was added 4-methylbenzene-1-sulfonic acid hydrate (53.9 mg, 0.28 mmol) and H₂O (51.1 mg, 2.83 mmol). The resulting biphasic mixture was heated to 80° C. and stirred for 3 h. The resulting mixture was filtered and the filter cake was washed with DCM (2×1 mL). The filtrate was concentrated under reduced pressure. The residue was purified by Prep-TLC (MeOH/DCM, 1:10) to afford 3-(2-(6-(difluoromethyl)-2-((1-(methylsulfonyl)piperidin-4-yl)amino)quinazolin-8-yl)-6-azaspiro[3.4]octan-6-yl)-2,2-difluoropropan-1-ol (2.7 mg, 1.8% yield) (Example 106).

¹H NMR (400 MHz, Methanol-d₄) δ 9.05 (d, J=2.1 Hz, 1H), 7.78 (s, 1H), 7.66 (s, 1H), 6.85 (t, J=56.3 Hz, 1H), 5.36 (t, J=4.8 Hz, 1H), 4.18-4.04 (m, 2H), 3.83-3.70 (m, 4H), 3.08-2.97 (m, 4H), 2.91 (s, 3H), 2.85 (t, J=6.8 Hz, 1H), 2.75 (d, J=3.6 Hz, 2H), 2.64-2.50 (m, 2H), 2.28-2.18 (m, 5H), 2.04 (d, J=6.1 Hz, 1H), 1.90 (d, J=2.6 Hz, 1H), 1.80-1.72 (m, 2H), 1.62 (s, 1H). LCMS (ESI-MS) m/z=560.4 [M+H]⁺, 95.1% purity.

### Example 120

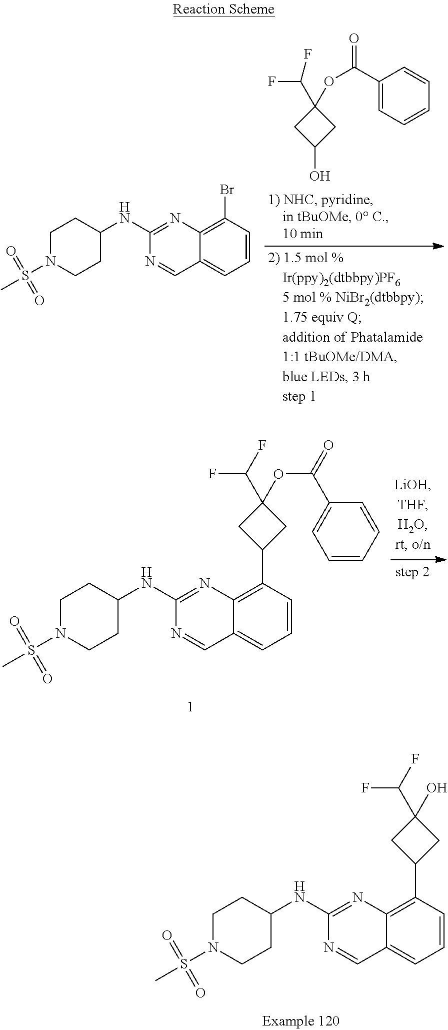 [SMILES: O=C(OC1(C(F)F)CC(O)C1)c1ccccc1 ; CS(=O)(=O)N1CCC(Nc2ncc3cccc(Br)c3n2)CC1 ; CS(=O)(=O)N1CCC(Nc2ncc3cccc(C4CC(OC(=O)c5ccccc5)(C(F)F)C4)c3n2)CC1 ; CS(=O)(=O)N1CCC(Nc2ncc3cccc(C4CC(O)(C(F)F)C4)c3n2)CC1]

### Detailed Procedure

### Step 1: 1-(difluoromethyl)-3-(2-((1-(methylsulfonyl)piperidin-4-yl)amino)quinazolin-8-yl)cyclobutyl benzoate

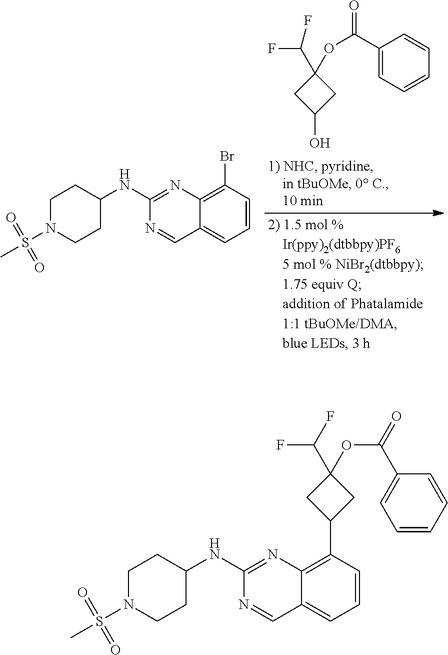 [SMILES: O=C(OC1(C(F)F)CC(O)C1)c1ccccc1 ; CS(=O)(=O)N1CCC(Nc2ncc3cccc(Br)c3n2)CC1 ; CS(=O)(=O)N1CCC(Nc2ncc3cccc(C4CC(OC(=O)c5ccccc5)(C(F)F)C4)c3n2)CC1]

An oven-dried 20 mL vial was charged with 1-(difluoromethyl)-3-hydroxycyclobutyl benzoate (110 mg, 0.45 mmol) and 5,7-Di-tert-butyl-3-phenylbenzo[d]oxazol-3-ium tetrafluoroborate (NHC) (164 mg, 0.42 mmol). Under nitrogen, tert-butyl methyl ether (4 mL) was added and the reaction stirred at room temperature for 5 minutes. A mixture of pyridine (32.8 mg, 0.42 mmol) in tert-butyl methyl ether (1 mL) was added and the mixture stirred at room temperature for 10 minutes (mixture A). Another oven-dried 40 mL vial was charged with 8-bromo-N-(1-(methylsulfonyl)piperidin-4-yl)quinazolin-2-amine (100 mg, 0.26 mmol), Ir(ppy)₂(dtbbpy)PF₆ (3.56 mg, 0.004 mmol), NiBr₂(dtbbpy) (9.48 mg, 0.02 mmol) and 1-azabicyclo[2.2.2]octane (50.5 mg, 0.46 mmol). DMA (5 mL) was added under nitrogen (mixture B). The mixture A was added to the mixture B under a nitrogen atmosphere and the resulting mixture was stirred and irradiated with a 450 nm LED lamp under a fan for 3 h. The reaction mixture was diluted with water (20 mL) and extracted with EA (3×20 mL). The combined organic layers were dried over anhydrous Na₂SO₄, filtered and concentrated under reduced pressure. The residue was purified by Prep-TLC (EA/PE, 2:1) to afford 1-(difluoromethyl)-3-(2-((1-(methylsulfonyl)piperidin-4-yl)amino)quinazolin-8-yl)cyclobutyl benzoate (150 mg).

LCMS (ESI-MS) m/z=531.2 [M+H]⁺.

### Step 2: 1-(difluoromethyl)-3-(2-((1-(methylsulfonyl)piperidin-4-yl)amino)quinazolin-8-yl)cyclobutan-1-ol

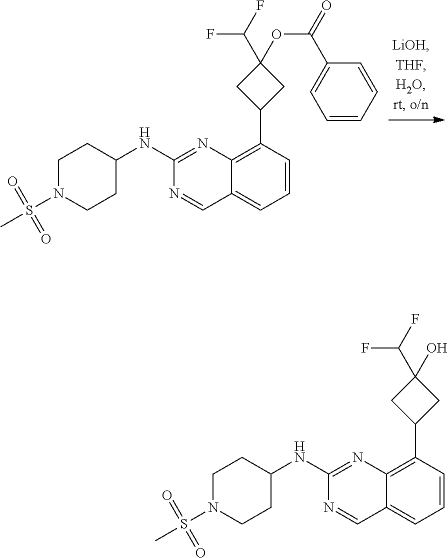 [SMILES: CS(=O)(=O)N1CCC(Nc2ncc3cccc(C4CC(OC(=O)c5ccccc5)(C(F)F)C4)c3n2)CC1 ; CS(=O)(=O)N1CCC(Nc2ncc3cccc(C4CC(O)(C(F)F)C4)c3n2)CC1]

LiOH (33.6 mg, 1.41 mmol) was added to a mixture of 1-(difluoromethyl)-3-(2-((1-(methylsulfonyl) piperidin-4-yl)amino)quinazolin-8-yl)cyclobutyl benzoate (150 mg, 0.28 mmol) in THF (3 mL) and H₂O (1 mL). The resulting mixture was stirred overnight at room temperature, neutralized by addition of aqueous HCl (1 N) until the pH was adjusted to 5-6 and extracted with EA (3×5 mL). The combined organic layers were dried over anhydrous Na₂SO₄ and concentrated under reduced pressure. The residue was purified by preparative RP-HPLC to afford 1-(difluoromethyl)-3-(2-((1-(methylsulfonyl)piperidin-4-yl)amino)quinazolin-8-yl)cyclobutan-1-ol (14.5 mg, 11.6% yield) (Example 120).

¹H NMR (400 MHz, DMSO-d₆) δ 9.09 (s, 1H), 7.67-7.59 (m, 2H), 7.44 (s, 1H), 7.26-7.17 (m, 1H), 6.27-5.81 (m, 2H), 4.03-3.88 (m, 1H), 3.82-3.71 (m, 1H), 3.67-3.54 (m, 2H), 2.97-2.86 (m, 5H), 2.83-2.74 (m, 2H), 2.40-2.33 (m, 1H), 2.29-2.19 (m, 1H), 2.12-2.00 (m, 2H), 1.67-1.55 (m, 2H).

LCMS (ESI-MS) m/z=427.1[M+H]⁺.

### Example 121: (1-(difluoromethyl)-3-(2-((1-methanesulfonylpiperidin-4-yl)amino)quinazolin-8-yl)cyclobutyl]methanol

### Reaction Scheme

### Step 1: 8-(3-(((tert-butyldiphenylsilyl)oxy)methyl)-3-(difluoromethyl)cyclobutyl)-N-(1-(methylsulfonyl) piperidin-4-yl)quinazolin-2-amine

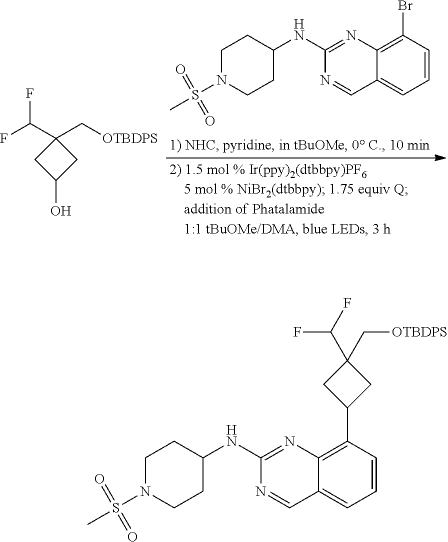

An oven-dried 20 mL vial was charged with methyl 3-(((tert-butyldiphenylsilyl)oxy)methyl)-3-(difluoromethyl)cyclobutan-1-ol (80 mg, 0.20 mmol) and 5,7-di-tert-butyl-3-phenylbenzo[d]oxazol-3-ium tetrafluoroborate (NHC) (70 mg, 0.17 mmol). Under nitrogen, tert-butyl methyl ether (4 mL) was added and the reaction stirred at room temperature for 5 minutes. A mixture of pyridine (13.9 mg, 0.17 mmol) in tert-butyl methyl ether (1 mL) was added and the mixture stirred at room temperature for 10 minutes (mixture A). Another oven-dried 40 mL vial was charged with 8-bromo-N-(1-(methylsulfonyl)piperidin-4-yl)quinazolin-2-amine (43.1 mg, 0.11 mmol), NiBr₂(dtbbpy) (4 mg, 0.01 mmol), Ir(ppy)₂(dtbbpy)PF₆ (5.43 mg, 0.006 mmol), phatalamide (9.65 mg, 0.02 mmol) and 1-azabicyclo[2.2.2]octane (77.1 mg, 0.69 mmol). DMA (5 mL) was added under nitrogen (mixture B). The mixture A was added to the mixture B under a nitrogen atmosphere and the resulting mixture was stirred and irradiated with a 450 nm LED lamp under a fan for 3 h. The reaction mixture was diluted with water (20 mL) and extracted with EA (3×20 mL). The combined organic layers were dried over anhydrous Na₂SO₄, filtered, and concentrated under reduced pressure. The residue was purified by Prep-TLC (EA/PE, 2:1) to afford 8-(3-(((tert-butyldiphenylsilyl)oxy)methyl)-3-(difluoromethyl) cyclobutyl)-N-(1-(methylsulfonyl)piperidin-4-yl)quinazolin-2-amine (40 mg).

LCMS (ESI-MS) m/z=679.3 [M+H]⁺.

### Step 2: (1-(difluoromethyl)-3-(2-((1-(methylsulfonyl)piperidin-4-yl)amino)quinazolin-8-yl)cyclobutyl)methanol

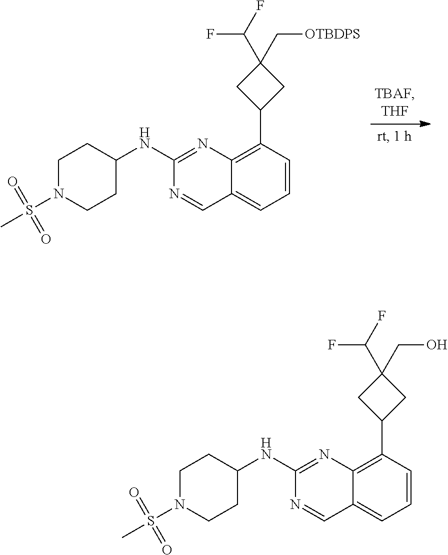

A solution of TBAF in THF (1 M, 0.06 mL, 0.06 mmol) was added to a stirred mixture of 8-(3-(((tert-butyldiphenylsilyl)oxy)methyl)-3-(difluoromethyl)cyclobutyl)-N-(1-methanesulfonylpiperidin-4-yl)quinazolin-2-amine (40 mg, 0.059 mmol) in THF (1 mL). The resulting mixture was stirred at room temperature for 1 h, quenched by addition of water (5 mL) and extracted with EA (3×5 mL). The combined organic layers were dried over Na₂SO₄, filtered and concentrated under reduced pressure. The residue was purified via Prep-TLC (EA) to afford (1-(difluoromethyl)-3-(2-((1-methanesulfonylpiperidin-4-yl)amino)quinazolin-8-yl)cyclobutyl]methanol (1.0 mg, 3.9% yield) (Example 121).

¹H NMR (400 MHz, Chloroform-d) δ 8.99 (s, 1H), 7.65 (s, 1H), 7.60 (s, 2H), 5.43-5.15 (m, 2H), 4.35 (s, 1H), 4.19-3.94 (m, 2H), 3.82 (s, 2H), 3.61-3.45 (s, 1H), 3.00 (t, J=11.1 Hz, 1H), 2.85 (s, 3H), 2.68-2.44 (m, 1H), 2.27 (d, J=11.0 Hz, 2H), 2.06-1.90 (m, 1H), 1.75 (s, 2H), 1.56 (s, 2H), 1.39-1.23 (in, 2H). LCMS (ESI-MS) m/z=441.2 [M+H]⁺.

### Example 116

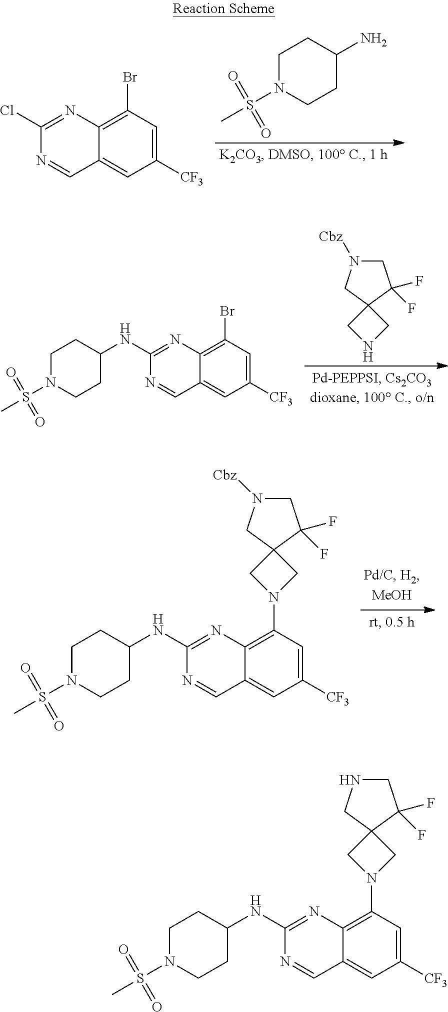 [SMILES: FC(F)(F)c1cc(Br)c2nc(Cl)ncc2c1 ; CS(=O)(=O)N1CCC(N)CC1 ; CS(=O)(=O)N1CCC(Nc2ncc3cc(C(F)(F)F)cc(Br)c3n2)CC1 ; O=C(OCc1ccccc1)N1CC(F)(F)C2(CNC2)C1 ; CS(=O)(=O)N1CCC(Nc2ncc3cc(C(F)(F)F)cc(N4CC5(CN(C(=O)OCc6ccccc6)CC5(F)F)C4)c3n2)CC1 ; CS(=O)(=O)N1CCC(Nc2ncc3cc(C(F)(F)F)cc(N4CC5(CNCC5(F)F)C4)c3n2)CC1]

### Detailed Procedure

### Step 1: S-bromo-N-(1-(methylsulfonyl)piperidin-4-yl)-6-(trifluoromethyl)quinazolin-2-amine

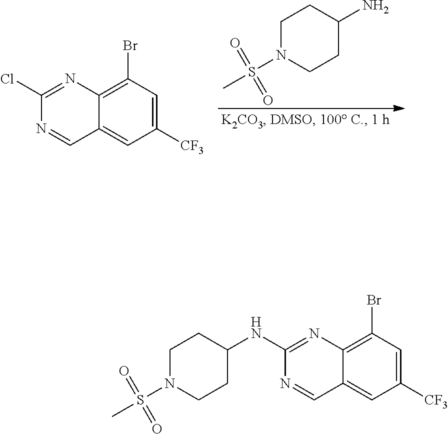 [SMILES: FC(F)(F)c1cc(Br)c2nc(Cl)ncc2c1 ; CS(=O)(=O)N1CCC(N)CC1 ; CS(=O)(=O)N1CCC(Nc2ncc3cc(C(F)(F)F)cc(Br)c3n2)CC1]

K₂CO₃ (399 mg, 2.88 mmol) was added to a mixture of 8-bromo-2-chloro-6-(trifluoromethyl) quinazoline (300 mg, 0.96 mmol) and 1-methanesulfonylpiperidin-4-amine (171 mg, 0.96 mmol) in DMSO (8 mL). The resulting mixture was heated to 100° C. and stirred for 1 h. After cooling to room temperature, the reaction mixture was quenched by addition of water (30 mL) and extracted with EA (3×30 mL). The combined organic layers were dried over anhydrous Na₂SO₄, filtered and concentrated under reduced pressure. The residue was purified by Prep-TLC (EA/PE, 1:1) to afford 8-bromo-N-(1-(methylsulfonyl) piperidin-4-yl)-6-(trifluoromethyl)quinazolin-2-amine (400 mg, 91.6% yield).

LCMS (ESI-MS) m/z=453.0 [M+H]⁺.

### Step 2: Benzyl 8,8-difluoro-2-(2-((1-(methylsulfonyl)piperidin-4-yl)amino)-6-(trifluoromethyl)quinazolin-8-yl)-2,6-diazaspiro[3.4]octane-6-carboxylate

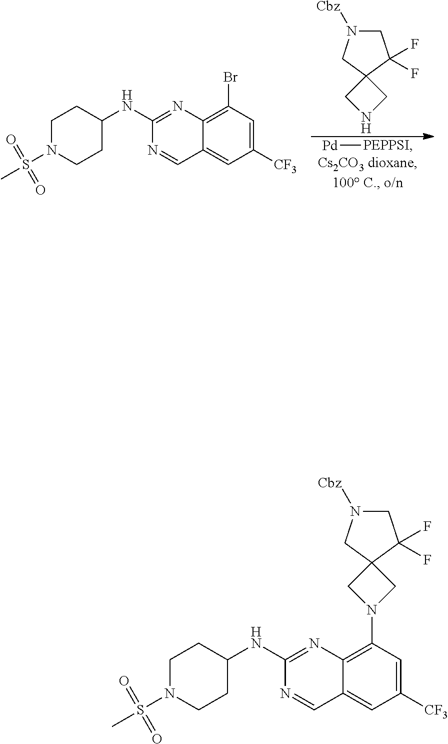

Pd-PEPPSI-IHeptCl 3-chloropyridine (23.6 mg, 0.02 mmol) was added to a mixture of 8-bromo-N-(1-(methylsulfonyl) piperidin-4-yl)-6-(trifluoromethyl)quinazolin-2-amine (110 mg, 0.24 mmol), benzyl 8,8-difluoro-2,6-diazaspiro[3.4]octane-6-carboxylate (68.5 mg, 0.24 mmol) and Cs₂CO₃ (158 mg, 0.48 mmol) in 1,4-dioxane (1 mL) under a nitrogen atmosphere. The resulting mixture was heated to 100° C. and stirred overnight under a nitrogen atmosphere. After cooling to room temperature, the reaction mixture was filtered and the filter cake was washed with DCM (20 mL). The filtrate was concentrated under reduced pressure. The residue was purified by Prep-TLC (EA/PE, 2:1) to afford benzyl 8,8-difluoro-2-(2-((1-(methylsulfonyl)piperidin-4-yl)amino)-6-(trifluoromethyl)quinazolin-8-yl)-2,6-diazaspiro[3.4]octane-6-carboxylate (110 mg, 73.0% yield).

LCMS (ESI-MS) m/z=655.2 [M+H]⁺.

### Step 3: 8-(8,8-difluoro-2,6-diazaspiro[3.4]octan-2-yl)-N-(1-(methylsulfonyl)piperidin-4-yl)-6-(trifluoromethyl)quinazolin-2-amine

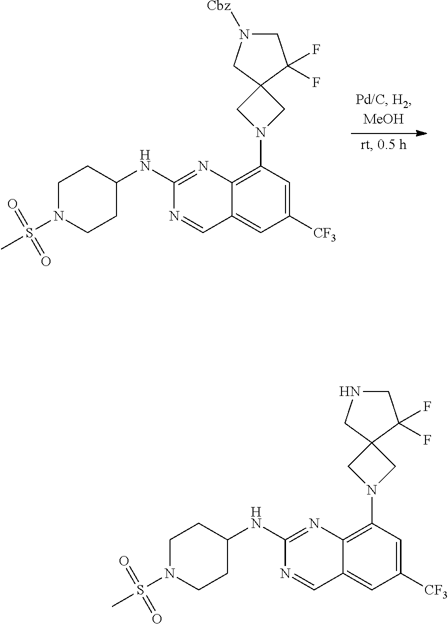 [SMILES: CS(=O)(=O)N1CCC(Nc2ncc3cc(C(F)(F)F)cc(N4CC5(CN(C(=O)OCc6ccccc6)CC5(F)F)C4)c3n2)CC1 ; CS(=O)(=O)N1CCC(Nc2ncc3cc(C(F)(F)F)cc(N4CC5(CNCC5(F)F)C4)c3n2)CC1]

Pd/C (10% on carbon, 10 mg) was added to a mixture of benzyl 8,8-difluoro-2-(2-((1-(methylsulfonyl)piperidin-4-yl)amino)-6-(trifluoromethyl)quinazolin-8-yl)-2,6-diazaspiro[3.4]octane-6-carboxylate (100 mg, 0.15 mmol) in MeOH (8 mL) under a nitrogen atmosphere. The resulting mixture was stirred for 0.5 h at room temperature under a hydrogen atmosphere, filtered and the filter cake was washed with MeOH (20 mL). The filtrate was concentrated under reduced pressure. The residue was purified by Prep-TLC (MeOH/DCM, 1:10). The product was further purified by preparative RP-HPLC to afford 8-(8,8-difluoro-2,6-diazaspiro[3.4]octan-2-yl)-N-(1-(methylsulfonyl)piperidin-4-yl)-6-(trifluoromethyl)quinazolin-2-amine (9.8 mg, 12.2% yield) (Example 116).

¹H NMR (400 MHz, DMSO-d₆) δ 9.15 (s, 1H), 7.77 (s, 1H), 7.54 (d, J=1.9 Hz, 1H), 6.63 (s, 1H), 4.34 (d, J=8.7 Hz, 2H), 4.11 (d, J=8.7 Hz, 2H), 3.86 (s, 1H), 3.68-3.52 (m, 3H), 3.26 (s, 2H), 3.17 (t, J=14.2 Hz, 2H), 2.97-2.81 (m, 5H), 2.12-1.95 (m, 2H), 1.70-1.53 (m, 2H).

LCMS (ESI-MS) m/z=521.2 [M+H]⁺.

### Example 114: 1-cyclopropyl-3-(2-((1-(methylsulfonyl)piperidin-4-yl)amino)-6-(trifluoromethyl)quinazolin-8-yl)cyclobutan-1-ol

### Reaction Scheme

### Detailed Procedure

### Step 1: 3-(2-((1-(methylsulfonyl)piperidin-4-yl)amino)-6-(trifluoromethyl)quinazolin-8-yl)cyclobutan-1-one

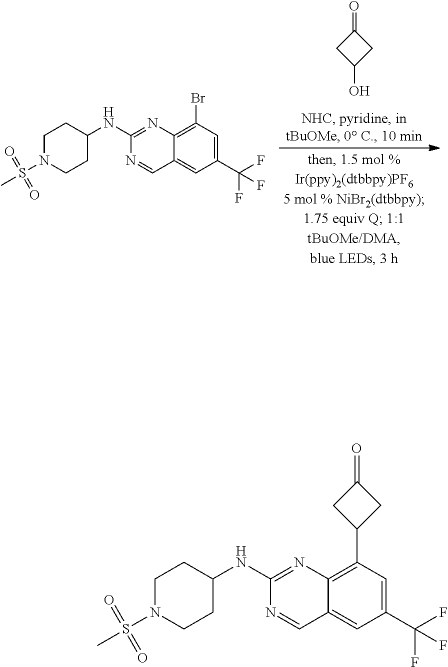 [SMILES: O=C1CC(O)C1 ; CS(=O)(=O)N1CCC(Nc2ncc3cc(C(F)(F)F)cc(Br)c3n2)CC1 ; CS(=O)(=O)N1CCC(Nc2ncc3cc(C(F)(F)F)cc(C4CC(=O)C4)c3n2)CC1]

An over-dried 20 mL vial was charged with 3-hydroxycyclobutan-1-one (43 mg, 0.49 mmol) and 5,7-di-tert-butyl-3-phenylbenzo[d]oxazol-3-ium tetrafluoroborate (NHC) (177 mg, 0.45 mmol). Under nitrogen, tert-butyl methyl ether (4 mL) was added and the reaction stirred at room temperature for 5 minutes. A mixture of pyridine (81 mg, 1.03 mmol) in tert-butyl methyl ether (1 mL) was added and the mixture stirred at room temperature for 10 minutes (mixture A). Another over-dried 40 mL vial was charged with 8-chloro-6-methyl-N-(1-(methylsulfonyl)piperidin-4-yl)pyrido[3,4-d]pyrimidin-2-amine (200 mg, 0.56 mmol), 5,7-di-tert-butyl-3-phenylbenzo[d]oxazol-3-ium tetrafluoroborate (NHC) (177.72 mg, 0.45 mmol), Ir(ppy)₂(dtbbpy)PF₆ (3.85 mg, 0.004 mmol), NiBr₂(dtbbpy) (6.84 mg, 0.014 mmol) and 1-azabicyclo[2.2.2]octane (54.68 mg, 0.49 mmol). DMA (5 mL) was added under nitrogen (mixture B). The mixture A was added to the mixture B under a nitrogen atmosphere and the resulting mixture was stirred and irradiated with a 450 nm LED lamp under a fan for 3 h. The reaction mixture was quenched with water (20 mL) and extracted with EA (3×20 mL). The combined organic layers were dried over anhydrous Na₂SO₄, filtered and concentrated under reduced pressure. The residue was purified by preparative RP-HPLC to afford 3-(2-((1-(methylsulfonyl)piperidin-4-yl)amino)-6-(trifluoromethyl)quinazolin-8-yl)cyclobutan-1-one (50 mg).

LCMS (ESI-MS) m/z=443.1 [M+H]⁺.

### Step 2: 1-cyclopropyl-3-(2-((1-(methylsulfonyl)piperidin-4-yl)amino)-6-(trifluoromethyl)quinazolin-8-yl)cyclobutan-1-ol

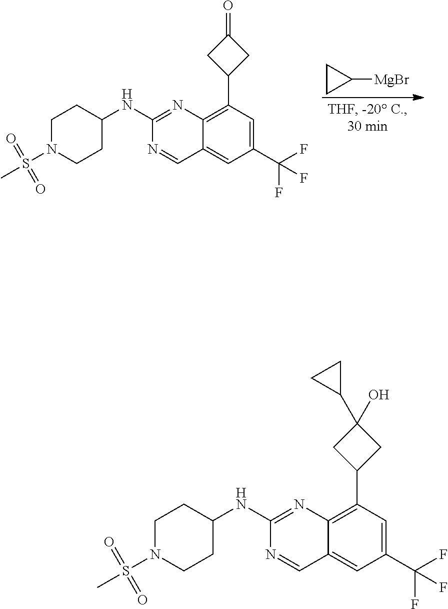

A solution of cyclopropylmagnesium bromide in THF (1 M, 0.2 mL, 0.2 mmol) was added dropwise to a cooled to −20° C. solution of 1-cyclopropyl-3-(2-((1-(methylsulfonyl)piperidin-4-yl)amino)-6-(trifluoro-methyl)quinazolin-8-yl) cyclobutan-1-ol (50 mg, 0.1 mmol) in THF (30 mL) under a nitrogen atmosphere. The resulting mixture was stirred at −20° C. for 30 minutes, quenched by the addition of aqueous NH₄Cl (30 mL) at 0° C. and extracted with EA (3×30 mL). The combined organic layers were dried over anhydrous Na₂SO₄, filtered and concentrated under reduced pressure. The residue was purified by silica gel column chromatography (EA/PE, 1:3) to afford 1-cyclopropyl-3-(2-((1-(methylsulfonyl)piperidin-4-yl)amino)-6-(trifluoromethyl)quinazolin-8-yl)cyclobutan-1-ol (2 mg, 3.6% yield) (Example 114).

¹H NMR (400 MHz, Chloroform-d) δ 9.02 (s, 1H), 7.83 (s, 1H), 7.71 (s, 1H), 5.44 (s, 1H), 4.13 (s, 1H), 3.83 (d, J=11.9 Hz, 2H), 3.64-3.60 (m, 1H), 3.02 (t, J=11.8 Hz, 1H), 2.86 (d, J=3.2 Hz, 3H), 2.61-2.48 (m, 2H), 2.44-2.22 (m, 3H), 1.86-1.71 (m, 2H), 1.59 (s, 2H), 1.39-1.19 (m, 2H), 0.67-0.60 (m, 2H), 0.53-0.50 (m, 2H).

LCMS (ESI-MS) m/z=485.2 [M+H]⁺.

### Example 117: (3S,4R)-4-((8-(8,8-difluoro-2,6-diazaspiro[3.4]octan-6-yl)-6-(difluoromethyl) quinazolin-2-yl)amino)tetrahydro-2H-pyran-3-ol

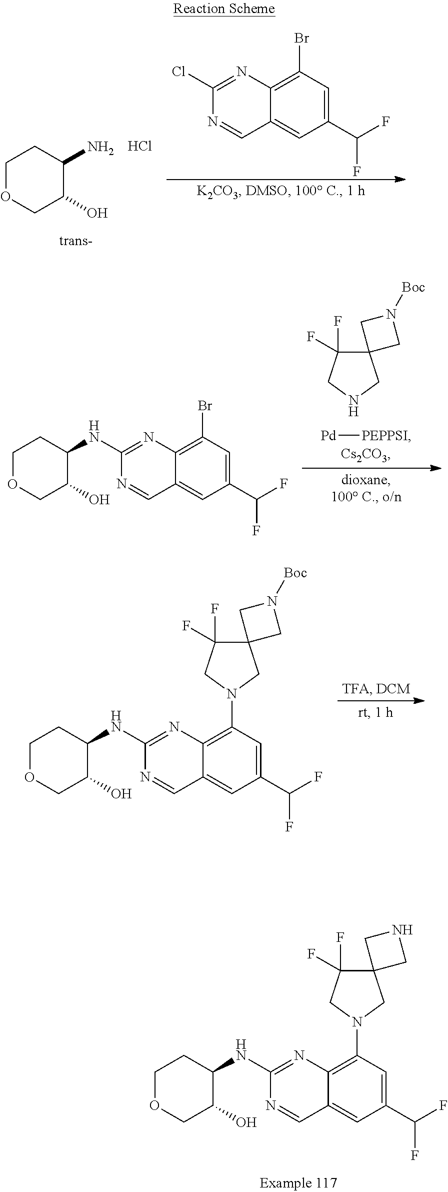

### Detailed Procedure

### Step 1: Racemic (3S,4R)-4-((8-bromo-6-(difluoromethyl)quinazolin-2-yl)amino)tetrahydro-2H-pyran-3-ol

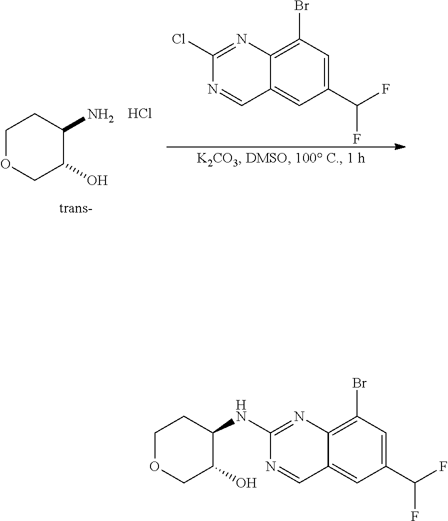 [SMILES: N[C@@H]1CCOC[C@H]1O ; Cl ; FC(F)c1cc(Br)c2nc(Cl)ncc2c1 ; O[C@@H]1COCC[C@H]1Nc1ncc2cc(C(F)F)cc(Br)c2n1]

K₂CO₃ (360 mg, 2.60 mmol) was added to a mixture of racemic (3S,4R)-4-aminotetrahydro-2H-pyran-3-ol hydrochloride (100 mg, 0.65 mmol) and 8-bromo-2-chloro-6-(difluoromethyl)quinazoline (191 mg, 0.65 mmol) in DMSO (5 mL). The resulting mixture was stirred for 1 h at 100° C. After cooling to room temperature, the reaction mixture was quenched by addition of water (30 mL) and extracted with EA (2×50 mL). The combined organic layers were washed with brine (2×50 mL), dried over anhydrous sodium sulfate, filtered and concentrated under reduced pressure. The residue was purified by silica gel column chromatography (EA/PE, from 80:20 to 100:0) to afford racemic (3S,4R)-4-((8-bromo-6-(difluoromethyl)quinazolin-2-yl)amino)tetrahydro-2H-pyran-3-ol (130 mg, 48.03% yield).

LCMS (ESI-MS) m/z=374.1 [M+H]⁺.

### Step 2: Racemic tert-butyl6-(6-(difluoromethyl)-2-(((3S,4R)-3-hydroxytetrahydro-2H-pyran-4-yl)amino)quinazolin-8-yl)-8,8-difluoro-2,6-diazaspiro[3.4]octane-2-carboxylate

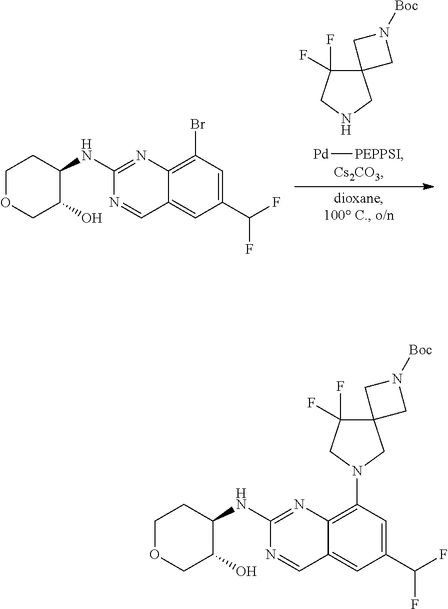

Pd-PEPPSI-IPentCl (23.0 mg, 0.02 mmol) was added to a mixture of racemic (3S,4R)-4-((8-bromo-6-(difluoromethyl)quinazolin-2-yl)amino)tetrahydro-2H-pyran-3-ol (100 mg, 0.26 mmol), tert-butyl 8,8-difluoro-2,6-diazaspiro[3.4]octane-2-carboxylate (66.4 mg, 0.26 mmol) and Cs₂CO₃ (174 mg, 0.53 mmol) in 1,4-dioxane (1 mL) under a nitrogen atmosphere. The resulting mixture was heated to 100° C. and stirred overnight under a nitrogen atmosphere. After cooling to room temperature, the resulting mixture was filtered, the filter cake was washed with DCM (50 mL). The filtrate was concentrated under reduced pressure. The residue was purified by silica gel column chromatography (EA/PE, from 80:20 to 100:0) to afford racemic tert-butyl 6-(6-(difluoromethyl)-2-(((3S,4R)-3-hydroxytetrahydro-2H-pyran-4-yl)amino)quinazolin-8-yl)-8,8-difluoro-2,6-diazaspiro[3.4]octane-2-carboxylate (80 mg, 49.7% yield).

LCMS (ESI-MS) m/z=542.5 [M+H]⁺.

### Step 3: Racemic (3S,4R)-4-((8-(8,8-difluoro-2,6-diazaspiro[3.4]octan-6-yl)-6-(difluoromethyl)quinazolin-2-yl)amino)tetrahydro-2H-pyran-3-ol

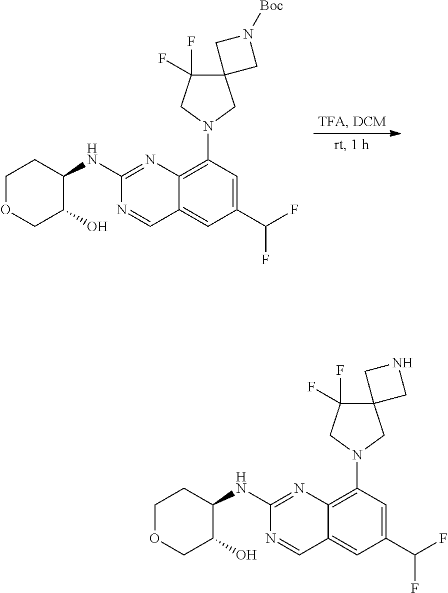 [SMILES: CC(C)(C)OC(=O)N1CC2(C1)CN(c1cc(C(F)F)cc3cnc(N[C@@H]4CCOC[C@H]4O)nc13)CC2(F)F ; O[C@@H]1COCC[C@H]1Nc1ncc2cc(C(F)F)cc(N3CC(F)(F)C4(CNC4)C3)c2n1]

TFA (0.3 mL) was added to a stirred mixture of racemic tert-butyl 6-(6-(difluoromethyl)-2-(((3S,4R)-3-hydroxytetrahydro-2H-pyran-4-yl)amino)quinazolin-8-yl)-8,8-difluoro-2,6-diazaspiro[3.4]octane-2-carboxylate (80 mg, 0.14 mmol) in DCM (1 mL). The resulting mixture was stirred for 1 h at room temperature and concentrated under reduced pressure. The residue was purified by preparative RP-HPLC to afford racemic (3S,4R)-4-((8-(8,8-difluoro-2,6-diazaspiro[3.4]octan-6-yl)-6-(difluoromethyl) quinazolin-2-yl)amino)tetrahydro-2H-pyran-3-ol (13.8 mg, 21.1% yield) (Example 117).

¹H NMR (400 MHz, DMSO-d₆) δ 8.97 (s, 1H), 7.34-7.27 (m, 1H), 6.94-6.88 (m, 1H), 6.82-6.53 (m, 1H), 5.58 (s, 1H), 4.56-4.42 (m, 1H), 4.35-4.21 (m, 3H), 4.17-4.11 (m, 3H), 4.06-4.03 (m, 1H), 3.94-3.92 (m, 2H), 3.67-3.46 (m, 5H), 2.42-2.25 (m, 1H), 1.78-1.60 (m, 1H), 1.35-1.22 (in, 1H).

LCMS (ESI-MS) m/z=442.1 [M+H]⁺.

### Example 118

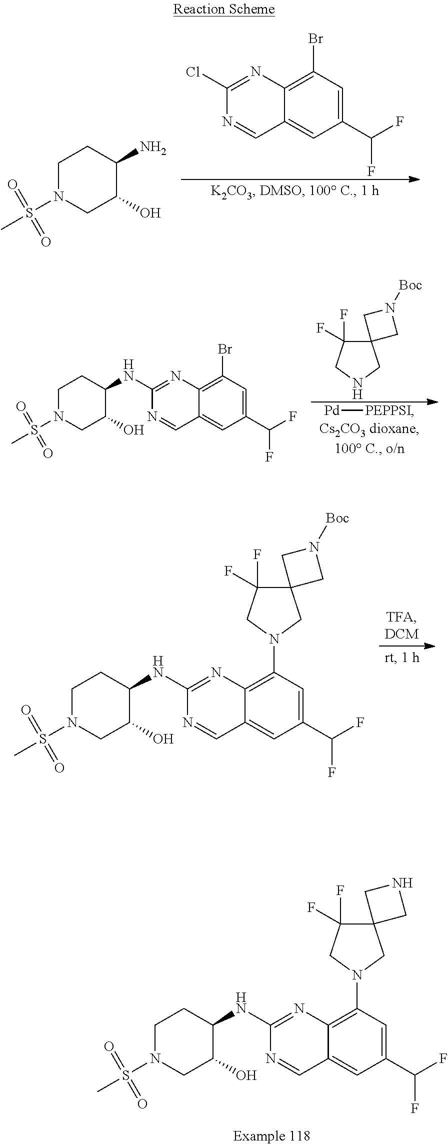

### Detailed Procedure

### Step 1: Racemic (3R,4R)-4-((S-bromo-6-(difluoromethyl)quinazolin-2-yl)amino)-1-(methylsulfonyl)piperidin-3-ol

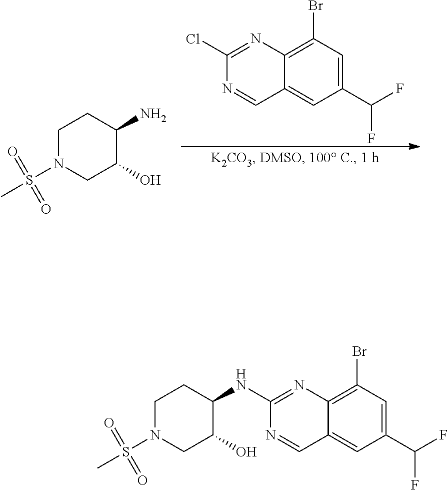 [SMILES: CS(=O)(=O)N1CC[C@@H](N)[C@H](O)C1 ; FC(F)c1cc(Br)c2nc(Cl)ncc2c1 ; CS(=O)(=O)N1CC[C@@H](Nc2ncc3cc(C(F)F)cc(Br)c3n2)[C@H](O)C1]

K₂CO₃ (107 mg, 0.77 mmol) was added to a mixture of racemic (3R,4R)-4-amino-1-(methylsulfonyl)piperidin-3-ol (50 mg, 0.25 mmol) and 8-bromo-2-chloro-6-(difluoromethyl)quinazoline (75.6 mg, 0.25 mmol) in DMSO (2 mL). The resulting mixture was heated to 100° C. and stirred for 1 h. After cooling to room temperature, the reaction mixture was quenched by addition of water (15 mL) and extracted with EA (3×15 mL). The combined organic layers were washed with brine (15 mL), dried over anhydrous Na₂SO₄, filtered and concentrated under reduced pressure. The residue was purified by Prep-TLC (EA) to afford racemic (3R,4R)-4-((8-bromo-6-(difluoromethyl)quinazolin-2-yl)amino)-1-(methylsulfonyl)piperidin-3-ol (100 mg, 86.1% yield).

LCMS (ESI-MS) m/z=451.0 [M+H]⁺.

### Step 2: Racemic tert-butyl 6-(6-(difluoromethyl)-2-(((3R,4R)-3-hydroxy-1-(methylsulfonyl)piperidin-4-yl)amino) quinazolin-8-yl)-8,8-difluoro-2,6-diazaspiro[3.4]octane-2-carboxylate

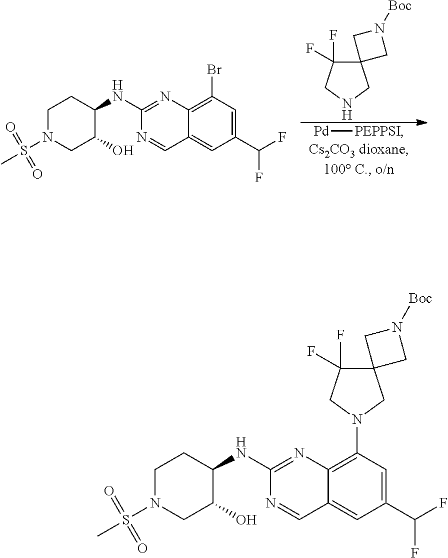

Pd-PEPPSI-IHeptCl 3-chloropyridine (19.4 mg, 0.02 mmol) was added to a mixture of racemic (3R,4R)-4-((8-bromo-6-(difluoromethyl)quinazolin-2-yl)amino)-1-(methylsulfonyl)piperidin-3-ol (90 mg, 0.20 mmol), tert-butyl 8,8-difluoro-2,6-diazaspiro[3.4]octane-2-carboxylate (49.5 mg, 0.20 mmol) and Cs₂CO₃ (130 mg, 0.40 mmol) in 1,4-dioxane (2 mL) under a nitrogen atmosphere. The resulting mixture was heated to 100° C. and stirred overnight under a nitrogen atmosphere. After cooling to room temperature, the reaction mixture was filtered and the filter cake was washed with DCM (20 mL). The filtrate was concentrated under reduced pressure. The residue was purified by Prep-TLC (EA) to afford racemic tert-butyl 6-(6-(difluoromethyl)-2-(((3R,4R)-3-hydroxy-1-(methylsulfonyl)piperidin-4-yl)amino)quinazolin-8-yl)-8,8-difluoro-2,6-diazaspiro[3.4]octane-2-carboxylate (40 mg, 32.4% yield).

LCMS (ESI-MS) m/z=619.2 [M+H]⁺.

### Step 3: Racemic (3R,4R)-4-((8-(8,8-difluoro-2,6-diazaspiro[3.4]octan-6-yl)-6-(difluoromethyl)quinazolin-2-yl)amino)-1-(methylsulfonyl)piperidin-3-ol

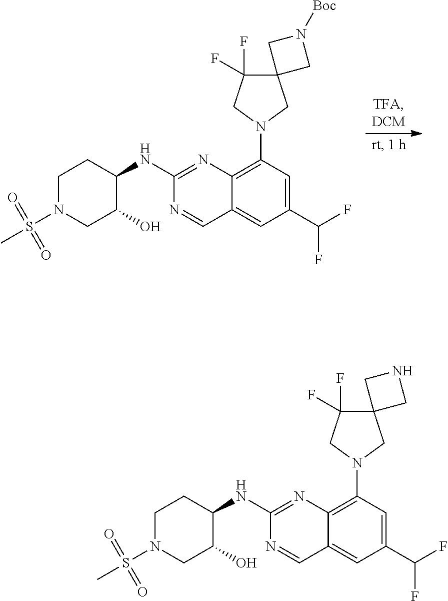 [SMILES: CC(C)(C)OC(=O)N1CC2(C1)CN(c1cc(C(F)F)cc3cnc(N[C@@H]4CCN(S(C)(=O)=O)C[C@H]4O)nc13)CC2(F)F ; CS(=O)(=O)N1CC[C@@H](Nc2ncc3cc(C(F)F)cc(N4CC(F)(F)C5(CNC5)C4)c3n2)[C@H](O)C1]

TFA (0.3 mL) was added to a stirred mixture of racemic tert-butyl 6-(6-(difluoromethyl)-2-(((3R,4R)-3-hydroxy-1-(methylsulfonyl)piperidin-4-yl)amino)quinazolin-8-yl)-8,8-difluoro-2,6-diazaspiro[3.4]octane-2-carboxylate (75 mg, 0.12 mmol) in DCM (0.9 mL). The resulting mixture was stirred for 1 h at room temperature and concentrated under reduced pressure. The residue was purified by Prep-TLC (MeOH/DCM, 1:10) to afford racemic (3R,4R)-4-((8-(8,8-difluoro-2,6-diazaspiro[3.4]octan-6-yl)-6-(difluoromethyl) quinazolin-2-yl) amino)-1-(methylsulfonyl)piperidin-3-ol (41.7 mg, 66.0% yield) (Example 118).

¹H NMR (400 MHz, DMSO-d₆) δ 9.14 (s, 1H), 7.59 (s, 1H), 7.48 (s, 1H), 7.20-6.83 (m, 2H), 4.37-3.91 (m, 7H), 3.90-3.48 (m, 7H), 3.00-2.86 (m, 4H), 2.80-2.68 (m, 1H), 2.29-2.13 (m, 1H), 1.65-1.44 (m, 1H).

LCMS (ESI-MS) m/z=519.1[M+H]⁺.

### Example 122: 8-(8,8-difluoro-6-azaspiro[3.4]octan-2-yl)-N-(1-(methylsulfonyl)piperidin-4-yl)quinazolin-2-amine

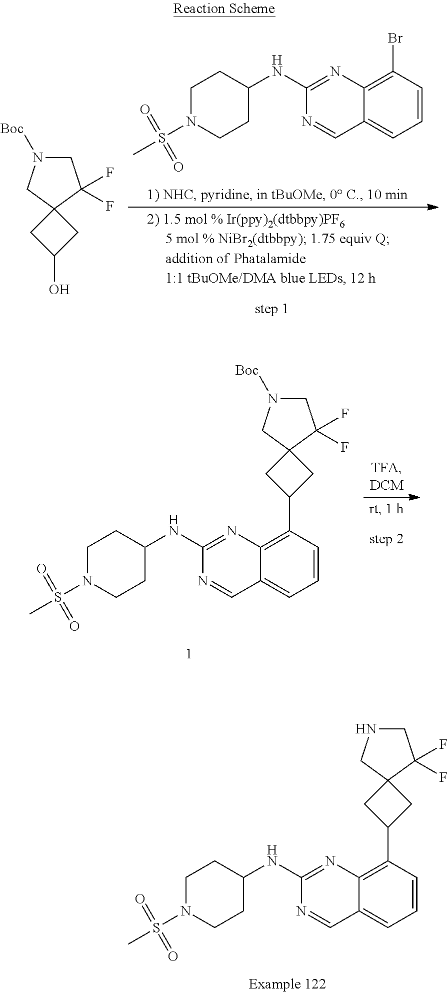

### Detailed Procedure

### Step 1: Tert-butyl8,8-difluoro-2-(2-((1-(methylsulfonyl)piperidin-4-yl)amino)quinazolin-8-yl)-6-azaspiro[3.4]octane-6-carboxylate

An oven-dried 20 mL vial was charged with tert-butyl 8,8-difluoro-2-hydroxy-6-azaspiro[3.4]octane-6-carboxylate (240 mg, 0.91 mmol) and 5,7-di-tert-butyl-3-phenylbenzo[d]oxazol-3-ium tetrafluoroborate (NHC) (328 mg, 0.83 mmol). Under nitrogen, tert-butyl methyl ether (4 mL) was added and the reaction stirred at room temperature for 5 minutes. A mixture of pyridine (65.7 mg, 0.83 mmol) in tert-butyl methyl ether (1 mL) was added and the mixture stirred at room temperature for 5 minutes (mixture A). Another oven-dried 40 mL vial was charged with 8-bromo-N-(1-(methylsulfonyl)piperidin-4-yl)quinazolin-2-amine (200 mg, 0.52 mmol), Ir(ppy)₂(dtbbpy)PF₆ (7.3 mg, 0.008 mmol), NiBr₂(dtbbpy) (12.7 mg, 0.026 mmol), phthalimide (30.9 mg, 0.21 mmol) and 1-azabicyclo[2.2.2]octane (101 mg, 0.91 mmol). DMA (5 mL) was added under nitrogen (mixture B). The mixture A was added to the mixture B under a nitrogen atmosphere and the resulting mixture was stirred and irradiated with a 450 nm LED lamp under a fan for 12 h. The reaction mixture was quenched with water (20 mL) and extracted with EA (3×20 mL). The combined organic layers were dried over anhydrous Na₂SO₄, filtered and concentrated under reduced pressure. The residue was purified by Prep-TLC (EA/PE, 2:1) to afford tert-butyl 8,8-difluoro-2-(2-((1-(methylsulfonyl)piperidin-4-yl)amino)quinazolin-8-yl)-6-azaspiro[3.4]octane-6-carboxylate (50 mg, 17.4% yield).

LCMS (ESI-MS) m/z=552.2 [M+H]⁺.

### Step 2: 8-(8,8-difluoro-6-azaspiro[3.4]octan-2-yl)-N-(1-(methylsulfonyl)piperidin-4-yl)quinazolin-2-amine

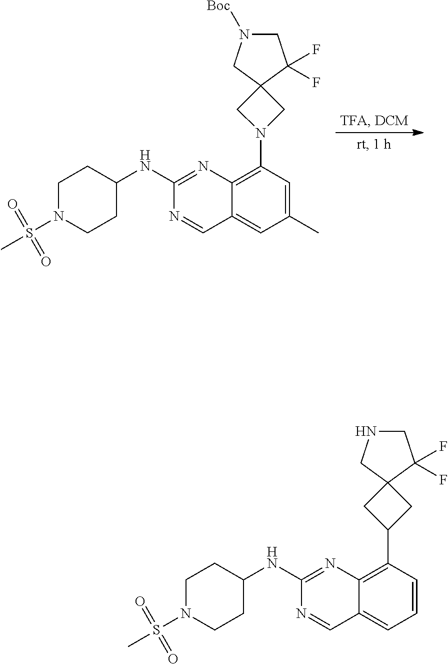

TFA (0.3 mL) was added to a stirred mixture of tert-butyl 8,8-difluoro-2-(2-((1-(methylsulfonyl)piperidin-4-yl)amino)quinazolin-8-yl)-6-azaspiro[3.4]octane-6-carboxylate (50 mg, 0.09 mmol) in DCM (1 mL). The resulting mixture was stirred for 1 h at room temperature and concentrated under reduced pressure. The residue was purified by Prep-TLC (MeOH/DCM, 1:10) to afford 8-(8,8-difluoro-6-azaspiro[3.4]octan-2-yl)-N-(1-(methylsulfonyl)piperidin-4-yl)quinazolin-2-amine (4.3 mg, 10.6% yield).

¹H NMR (400 MHz, DMSO-d₆) δ 9.10 (s, 1H), 7.68-7.55 (m, 2H), 7.47 (s, 1H), 7.25-7.18 (m, 1H), 4.16-3.85 (m, 2H), 3.65-3.55 (m, 2H), 3.25-3.04 (m, 3H), 2.96-2.84 (m, 6H), 2.80-2.71 (m, 2H), 2.39-2.25 (m, 2H), 2.19-2.05 (m, 3H), 1.70-1.56 (m, 2H).

LCMS (ESI-MS) m/z=452.2 [M+H]⁺.

In some embodiments, compounds of the disclosure are below in Table 1.

TABLE 1

| | | | Mass | |
|---|---|---|---|---|
| Cmpd | | | Spec. | |
| # | Structure | Name | M + H/1 | 1H NMR (ppm |
| 1 | 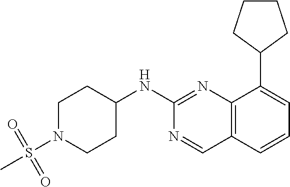 [SMILES: CS(=O)(=O)N1CCC(Nc2ncc3cccc(C4CCCC4)c3n2)CC1] | 8-cyclopentyl-N-(1- (methylsulfonyl)piperidin- 4-yl)quinazolin-2-amine | 375.2 | ¹H NMR (400 MHz, DMSO-d₆) δ (ppm) 9.08 (s, 1H), 7.63-7.58 (m, 2H), 7.41 (s, 1H), 7.18-7.15 (m, 1H), 3.95-3.81 (m, 2H), 3.59-3.56 (m, 2H), 3.35-3.32 (m, 5H), 2.06 (s, 4H), 1.69-1.59 (m, 8H). |
| 2 | | N-(1- | 376.2 | ¹H NMR (400 MHz, |
| | | (methylsulfonyl)piperidin- | | DMSO-d₆) δ (ppm) 8.98 |
| | | 4-yl)-8-(pyrrolidin-1- | | (s, 1H), 7.21 (d, J = 4.4 Hz, |
| | | yl)quinazolin-2-amine | | 1H), 7.08-7.03 (m, 2H), |
| | | | | 6.75-6.72 (m, 1H), |
| | | | | 3.89-3.87 (m, 1H), |
| | | | | 3.64-3.56 (m, 6H), |
| | | | | 2.90-2.85 (m, 5H), |
| | | | | 2.08-1.94 (m, 2H), |
| | | | | 1.92-1.91 (m, 4H), |
| | | | | 1.64-1.95 (m, 2H). |
| 3 | | 8-(2,2-difluoro-6- | 452.2 | ¹H NMR (400 MHz, |
| | | azaspiro[3.4]octan-6-yl)- | | DMSO-d₆) δ (ppm) 8.99 |
| | | N-(1- | | (s, 1H), 7.24 (s, 1H), |
| | | (methylsulfonyl)piperidin- | | 7.09-7.04 (m, 2H), |
| | | 4-yl)quinazolin-2-amine | | 6.73-6.70 (m, 1H), 3.91 (s, |
| | | | | 3H), 3.60-3.57 (m, 4H), |
| | | | | 2.90-2.85 (m, 5H), |
| | | | | 2.68-2.61 (m, 4H), |
| | | | | 2.08-2.02 (m, 4H), |
| | | | | 1.66-1.57 (m, 2H) |
| 4 | | 6-(2-((1- | 466.2 | ¹H NMR (400 MHz, |
| | | (methylsulfonyl)piperidin- | | DMSO-d₆) δ (ppm) 8.99 |
| | | 4-yl)amino)quinazolin-8- | | (s, 1H), 7.27 (s, 1H), |
| | | yl)-2-thia-6- | | 7.12-7.04 (m, 2H), |
| | | azaspiro[3.4]octane 2,2- | | 6.75-6.73 (m, 1H), |
| | | dioxide | | 4.28-4.13 (m, 6H), 4.13 (s, |
| | | | | 1H), 3.59-3.56 (m, 4H), |
| | | | | 2.95-2.89 (m, 5H), |
| | | | | 2.28-2.23 (m, 2H), |
| | | | | 2.07-2.02 (m, 2H), |
| | | | | 1.60-1.57 (m, 2H) |
| 5 | 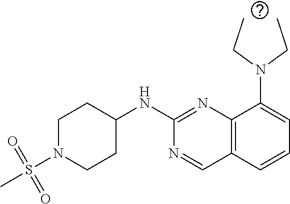 [SMILES: CCN(CC)c1cccc2cnc(NC3CCN(S(C)(=O)=O)CC3)nc12] | 1-(6-(2-((1- (methylsulfonyl)piperidin- 4-yl)amino)quinazolin-8- yl)-2,6- diazaspiro[3.4]octan-2- yl)ethan-1-one | 459.2 | ¹H NMR (400 MHz, CDCl₃) δ (ppm) 8.95 (s, 1H), 7.21-7.14 (m, 2H), 6.91-6.83 (m, 1H), 5.10 (s, 1H), 4.24-4.03 (m, 6H), 3.85-3.58 (m, 5H), 2.96 (s, 2H), 2.86 (s, 3H), 2.26-2.21 (m, 4H), 1.90 (s, 3H), 1.75-1.67 (m, 2H) |
| 6 | | 1-(7-(2-((1- | 473.2 | ¹H NMR (400 MHz, |
| | | (methylsulfonyl)piperidin- | | DMSO-d₆) δ (ppm) 8.98 |
| | | 4-yl)amino)quinazolin-8- | | (s, 1H), 7.24 (s, 1H), |
| | | yl)-2,7- | | 7.08-7.04 (m, 2H), |
| | | diazaspiro[4.4]nonan-2- | | 6.75-6.73 (m, 1H), |
| | | yl)ethan-1-one | | 3.86-3.77 (m, 3H), |
| | | | | 3.65-3.57 (m, 5H), |
| | | | | 3.51-3.27 (m, 3H), |
| | | | | 2.91-2.81 (m, 3H), 2.74 (s, |
| | | | | 2H), 2.04-1.97 (m, 9H), |
| | | | | 1.54 (s, 2H). |
| 7 | | N-(1- | 446.2 | ¹H NMR (400 MHz, |
| | | (methylsulfonyl)piperidin- | | DMSO-d₆) δ (ppm) 9.04 |
| | | 4-yl)-8-(2-oxa-7- | | (s, 1H), 7.34-7.20 (m, 2H), |
| | | azaspiro[4.5]decan-7- | | 7.18-7.06 (m, 2H), |
| | | yl)quinazolin-2-amine | | 4.18-3.99 (m, 2H), |
| | | | | 3.80-3.71 (m, 2H), |
| | | | | 3.63-3.53 (m, 2H), |
| | | | | 3.52-3.46 (m, 2H), |
| | | | | 3.30-3.22 (m, 1H), |
| | | | | 3.08-2.91 (m, 3H), 2.89 (s, |
| | | | | 3H), 2.75-2.66 (m, 1H), |
| | | | | 2.12-1.98 (m, 2H), |
| | | | | 1.88-1.76 (m, 2H), |
| | | | | 1.72-1.60 (m, 6H). |
| 8 | 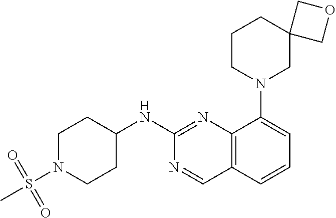 [SMILES: CS(=O)(=O)N1CCC(Nc2ncc3cccc(N4CCCC5(COC5)C4)c3n2)CC1] | N-(1- (methylsulfonyl)piperidin- 4-yl)-8-(2-oxa-6- azaspiro[3.5]nonan-6- yl)quinazolin-2-amine | 432.2 | ¹H NMR (400 MHz, DMSO-d₆) δ (ppm) 9.05 (s, 1H), 7.40-7.34 (m, 2H), 7.16-7.11 (m, 2H), 4.56-4.55 (m, 2H), 4.38-4.37 (m, 2H) 4.19-4.12 (m, 1H), 3.58-3.55 (m, 4H), 3.02 (s, 2H), 2.89-2.84 (m, 5H), 2.08-2.00 (m, 2H), 1.80 (s, 2H) 1.67-1.59 (m, 4H). |
| 9 | | N-(1- | 418.2 | ¹H NMR (400 MHz, |
| | | (methylsulfonyl)piperidin- | | DMSO-d₆) δ (ppm) 9.06 |
| | | 4-yl)-8-(4-oxa-7- | | (s, 1H), 7.42-7.35 (m, 2H), |
| | | azaspiro[2.5]octan-7- | | 7.15-7.06 (m, 2H), |
| | | yl)quinazolin-2-amine | | 3.91-3.89 (m, 3H), |
| | | | | 3.60-3.57 (m, 2H), 3.41 (s, |
| | | | | 2H), 3.33-3.29 (m, 2H), |
| | | | | 2.91-2.82 (m, 5H), |
| | | | | 2.02-1.98 (m, 2H), |
| | | | | 1.68-1.59 (m, 2H), |
| | | | | 0.79-0.75 (m, 2H), |
| | | | | 0.68-0.64 (m, 2H). |
| 10 | | (1-(2-((1- | 392 | |
| | | (methylsulfonyl)piperidin- | | |
| | | 4-yl)amino)quinazolin-8- | | |
| | | yl)azetidin-2-yl)methanol | | |
| 11 | | N-(1- | 447.1 | |
| | | (methylsulfonyl)piperidin- | | |
| | | 4-yl)-8-(4-(oxetan-3- | | |
| | | yl)piperazin-1- | | |
| | | yl)quinazolin-2-amine | | |
| 12 | 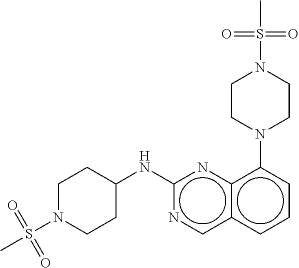 | 8-(4- (methylsulfonyl)piperazin- 1-yl)-N-(1- (methylsulfonyl)piperidin- 4-yl)quinazolin-2-amine | 469 | |
| 13 | | 8-(4- | 431.3 | |
| | | cyclopropylpiperazin-1- | | |
| | | yl)-N-(1- | | |
| | | (methylsulfonyl)piperidin- | | |
| | | 4-yl)quinazolin-2-amine | | |
| 14 | | 2-(1-(2-((1- | 401.1 | |
| | | (methylsulfonyl)piperidin- | | |
| | | 4-yl)amino)quinazolin-8- | | |
| | | yl)azetidin-3- | | |
| | | yl)acetonitrile | | |
| 15 | | N-(1- | 417.1 | ¹H NMR (300 MHz, |
| | | (methylsulfonyl)piperidin- | | DMSO-d₆) δ 8.99 (s, 1H), |
| | | 4-yl)-8-(2,6- | | 7.23 (m, 1H), 7.07 (m, |
| | | diazaspiro[3.4]octan-6- | | 2H), 6.72 (m, 1H), |
| | | yl)quinazolin-2-amine | | 3.95-3.36 (m, 12H), 2.89 |
| | | | | (m, 5H), 2.19-2.00 (m, |
| | | | | 4H), 1.63 (m, 2H). |
| 16 | 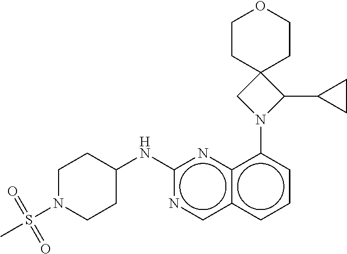 | 8-(1-cyclopropyl-7-oxa-2- azaspiro[3.5]nonan-2-yl)- N-(1- (methylsulfonyl)piperidin- 4-yl)quinazolin-2-amine | 472.3 | |
| 17 | | 8-(4-(cyclopropylmethyl) | 445.2 | |
| | | piperazin-1-yl)-N-(1- | | |
| | | (methylsulfonyl)piperidin- | | |
| | | 4-yl)quinazolin-2-amine | | |
| 18 | | 2-(2-((1- | 418.1 | |
| | | (methylsulfonyl)piperidin- | | |
| | | 4-yl)amino)quinazolin-8- | | |
| | | yl)-2-azaspiro[3.3]heptan- | | |
| | | 6-ol | | |
| 19 | | 6-(2-((1- | 452.2 | |
| | | (methylsulfonyl)piperidin- | | |
| | | 4-yl)amino)quinazolin-8- | | |
| | | yl)-1-thia-6- | | |
| | | azaspiro[3.3]heptane 1,1- | | |
| | | dioxide | | |
| 20 | 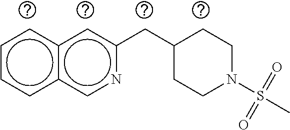 | 8-(4- (diethylamino)piperidin-1- yl)-N-(1- (methylsulfonyl)piperidin- 4-yl)quinazolin-2-amine | 461.3 | |
| 21 | | 8-(6,6-dimethyl-2- | 430.1 | |
| | | azaspiro[3.3]heptan-2-yl)- | | |
| | | N-(1- | | |
| | | (methylsulfonyl)piperidin- | | |
| | | 4-yl)quinazolin-2-amine | | |
| 22 | | N-(1- | 418.2 | ¹H NMR (400 MHz, |
| | | (methylsulfonyl)piperidin- | | CDCl₃) δ (ppm) 8.92 (s, |
| | | 4-yl)-8-(2-oxa-6- | | 1H), 7.15 (s, 2H), 6.99 (s, |
| | | azaspiro[3.4]octan-6- | | 1H) 5.13-5.06 (m, 1H), |
| | | yl)quinazolin-2-amine | | 4.93-4.72 (m, 4H), 4.07 (s, |
| | | | | 3H), 3.79-3.57 (m, 2H), |
| | | | | 3.48 (s, 2H), 3.16 (s, 2H), |
| | | | | 2.83 (s, 3H), 2.46-2.22 (m, |
| | | | | 4H), 1.85-1.73 (m, 2H). |
| 23 | 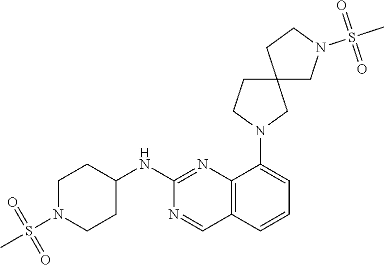 | 8-(7-(methylsulfonyl)-2,7- diazaspiro[4.4]nonan-2- yl)-N-(1- (methylsulfonyl)piperidin- 4-yl)quinazolin-2-amine | 509.2 | ¹H NMR (400 MHz, DMSO-d₆) δ (ppm) 8.99 (s, 1H), 7.24 (s, 1H), 7.11-7.04 (m, 2H), 6.76-6.73 (m, 1H), 3.90-3.87 (m, 2H), 3.76-3.72 (m, 1H), 3.62-3.58 (m, 4H), 3.45-3.31 (m, 4H), 2.98-2.94 (s, 3H), 2.88-2.83 (m, 5H), 2.07-1.89 (m, 6H), 1.65-1.55 (m, 2H). |
| 24 | | 8-(2-methyl-2,6- | 431 | ¹H NMR (400 MHz, |
| | | diazaspiro[3.4]octan-6-yl)- | | CDCl₃) δ 8.90 (s, 1H), |
| | | N-(1- | | 7.15-7.09 (m, 2H), |
| | | (methylsulfonyl)piperidin- | | 6.83-6.79 (m, 1H), 5.31 (s, |
| | | 4-yl)quinazolin-2-amine | | 1H), 4.11-4.05 (m, 3H), |
| | | | | 3.80-3.75 (m, 2H), |
| | | | | 3.67-3.43 (m, 6H), |
| | | | | 3.05-2.98 (m, 2H), 2.84 (s, |
| | | | | 3H), 2.48 (s, 3H), |
| | | | | 2.26-2.19 (m, 4H), |
| | | | | 1.81-1.69 (m, 2H). |
| 25 | | 1-(2-(2-((1- | 487.2 | ¹H NMR (400 MHz, |
| | | (methylsulfonyl)piperidin- | | DMSO-d₆) δ (ppm) |
| | | 4-yl)amino)quinazolin-8- | | 9.00-8.92 (m, 1H), 7.22 (s, |
| | | yl)-2,8- | | 1H), 7.12-6.97 (m, 2H), |
| | | diazaspiro[4.5]decan-8- | | 6.80-6.66 (m, 1H), |
| | | yl)ethan-1-one | | 3.95-3.80 (m, 1H), |
| | | | | 3.71-3.53 (m, 6H), |
| | | | | 3.49-3.37 (m, 4H), |
| | | | | 2.93-2.76 (m, 5H), |
| | | | | 2.10-1.94 (m, 5H), |
| | | | | 1.90-1.76 (m, 2H), |
| | | | | 1.70-1.43 (m, 6H). |
| 26 | 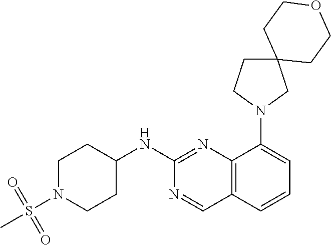 [SMILES: CS(=O)(=O)N1CCC(Nc2ncc3cccc(N4CCC5(CCOCC5)C4)c3n2)CC1] | N-(1- (methylsulfonyl)piperidin- 4-yl)-8-(8-oxa-2- azaspiro[4.5]decan-2- yl)quinazolin-2-amine | 446.2 | ¹H NMR (400 MHz, DMSO-d₆) δ (ppm) 8.98 (s, 1H), 7.23 (s, 1H), 7.08-7.03 (m, 2H), 6.75-6.72 (m, 1H), 3.90-3.88 (m, 1H), 3.65-3.55 (m, 10H), 2.89-2.83 (m, 5H), 2.08-2.03 (m, 2H), 1.88-1.84 (m, 2H), 1.67-1.58 (m, 6H). |
| 27 | | 3-cyclopropyl-1-(2-((1- | 441.2 | ¹H NMR (400 MHz, |
| | | (methylsulfonyl)piperidin- | | CDCl₃) δ (ppm) 8.94 (s, |
| | | 4-yl)amino)quinazolin-8- | | 1H), 7.19 (s, 2H), |
| | | yl)pyrrolidine-3- | | 6.98-6.91 (m, 1H), |
| | | carbonitrile | | 5.24-5.22 (m, 1H), |
| | | | | 4.67-4.58 (m, 1H), |
| | | | | 4.13-4.04 (m, 1H), |
| | | | | 3.85-3.81 (m, 3H), |
| | | | | 3.78-3.66 (m, 1H), 3.01 (s, |
| | | | | 2H), 2.82 (s, 3H), |
| | | | | 2.57-2.48 (m, 1H), |
| | | | | 2.35-2.20 (m, 3H), |
| | | | | 1.75-1.47 (m, 2H), 1.25 (s, |
| | | | | 1H), 1.22-1.13 (m, 1H), |
| | | | | 0.82-0.53 (m, 4H). |
| 28 | | 8-(8,8-difluoro-2- | 480.2 | ¹H NMR (400 MHz, |
| | | azaspiro[4.5]decan-2-yl)- | | CDCl₃) δ (ppm) 8.92 (s, |
| | | N-(1- | | 1H), 7.16-7.14 (m, 2H), |
| | | (methylsulfonyl)piperidin- | | 6.91 (s, 1H), 5.13 (s, 1H), |
| | | 4-yl)quinazolin-2-amine | | 4.07 (s, 1H), 3.75-3.53 (m, |
| | | | | 5H), 2.99 (s, 2H), 2.83 (s, |
| | | | | 3H), 2.24-2.20 (m, 2H), |
| | | | | 2.07-1.86 (m, 7H), |
| | | | | 1.80-1.60 (m, 6H). |
| 29 | 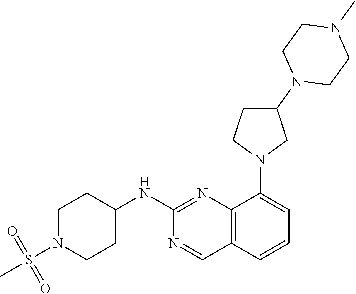 | 8-(3-(4-methylpiperazin- 1-yl)pyrrolidin-1-yl)-N-(1- (methylsulfonyl)piperidin- 4-yl)quinazolin-2-amine | 474.3 | ¹H NMR (400 MHz, CDCl₃) δ (ppm) 8.90 (s, 1H), 7.16-7.09 (m, 2H), 6.84-6.81 (m, 1H), 5.15 (s, 1H), 4.14-4.08 (m, 1H), 3.81-3.66 (m, 5H), 3.63-3.57 (m, 1H), 3.11-2.97 (m, 3H), 2.85 (s, 3H), 2.71-2.62 (m, 6H), 2.47 (s, 4H), 2.24-2.01 (m, 4H), 1.98-1.95 (m, 1H), 1.78-1.73 (m, 2H). |
| 30 | | 8-(6-oxa-3- azabicyclo[3.1.1]heptan-3- yl)-N-(1- (methylsulfonyl)piperidin- 4-yl)quinazolin-2-amine | 404.2 | |
| 31 | | N-(1- | 417.1 | ¹H NMR (300 MHz, |
| | | (methylsulfonyl)piperidin- | | Methanol-d₄) δ (ppm) 8.92 |
| | | 4-yl)-8-(2,6- | | (s, 1H), 7.21-7.06 (m, 2H), |
| | | diazaspiro[3.4]octan-2- | | 6.70 (dd, J = 7.0, 1.9 Hz, |
| | | yl)quinazolin-2-amine | | 1H), 4.15 (d, J = 1.7 Hz, |
| | | | | 4H), 4.07-3.92 (m, 1H), |
| | | | | 3.74 (d, J = 12.6 Hz, 2H), |
| | | | | 3.16 (s, 2H), 3.09-2.94 (m, |
| | | | | 4H), 2.90 (s, 3H), |
| | | | | 2.24-2.08 (m, 4H), |
| | | | | 1.85-1.66 (m, 2H). |
| 32 | 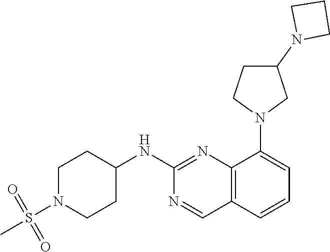 [SMILES: CS(=O)(=O)N1CCC(Nc2ncc3cccc(N4CCC(N5CCC5)C4)c3n2)CC1] | 8-(3-(azetidin-1- yl)pyrrolidin-1-yl)-N-(1- (methylsulfonyl)piperidin- 4-yl)quinazolin-2-amine | 431.2 | |
| 33 | | 1-(7-(2-((4-(4- | 486.3 | ¹H NMR (400 MHz, |
| | | methylpiperazin-1- | | DMSO-d₆) δ (ppm) 9.32 |
| | | yl)phenyl)amino) | | (s, 1H) 9.10 (s, 1H) |
| | | quinazolin-8-yl)-2,7- | | 7.51-7.55 (m, 2H) |
| | | diazaspiro[4.4]nonan-2- | | 7.02-7.18 (m, 2H) |
| | | yl)ethan-1-one | | 6.82-6.92 (m, 3H) |
| | | | | 3.42-3.67 (m, 7H) |
| | | | | 3.29-3.40 (m, 2H) |
| | | | | 2.97-3.38 (m, 4H) |
| | | | | 2.45-2.72 (m, 4H) |
| | | | | 2.18-2.26 (m, 3H) |
| | | | | 2.10-2.13 (m, 6H). |
| 34 | | 8-(7,7-difluoro-2- | 466.2 | ¹H NMR (400 MHz, |
| | | azaspiro[4.4]nonan-2-yl)- | | DMSO-d₆) δ (ppm) 8.97 |
| | | N-(1- | | (s, 1H), 7.25 (s, 1H), |
| | | (methylsulfonyl)piperidin- | | 7.06-7.02 (m, 2H), |
| | | 4-yl)quinazolin-2-amine | | 6.72-6.69 (m, 1H), |
| | | | | 3.86-3.61 (m, 3H), |
| | | | | 3.56-3.34 (m, 4H), |
| | | | | 2.88-2.82 (m, 5H), |
| | | | | 2.30-2.17 (m, 4H), |
| | | | | 2.15-1.97 (m, 4H), |
| | | | | 1.95-1.89 (m, 2H), |
| | | | | 1.81-1.62 (m, 2H). |
| 35 | | 1-(7-(2-((1- | 472.2 | ¹H NMR (300 MHz, |
| | | (methylsulfonyl)piperidin- | | DMSO-6) δ (ppm) 9.09 (s, |
| | | 4-yl)amino)quinazolin-8- | | 1H), 7.53-7.65 (m, 2H), |
| | | yl)-2-azaspiro[4.4]nonan- | | 7.45-7.52 (m, 1H), |
| | | 2-yl)ethan-1-one | | 7.14-7.21 (m, 1H), |
| | | | | 3.96-4.13 (m, 2H), |
| | | | | 3.61-3.85 (m, 3H), |
| | | | | 3.42-3.57 (m, 2H), |
| | | | | 3.22-3.39 (m, 1H), |
| | | | | 2.80-3.07 (m, 5H), |
| | | | | 2.08-2.29 (m, 4H), |
| | | | | 1.51-2.07 (m, 11H). |
| 36 | 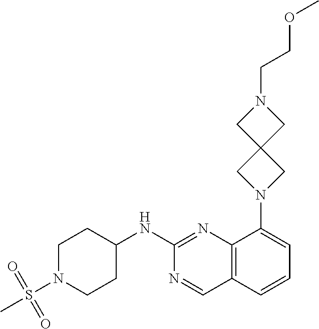 | 8-(6-(2-methoxyethyl)- 2,6-diazaspiro[3.3]heptan- 2-yl)-N-(1- (methylsulfonyl)piperidin- 4-yl)quinazolin-2-amine | 461.2 | ¹H NMR (300 MHz, DMSO-d₆) δ (ppm) 8.98 (s, 1H), 7.26 (s, 1H), 7.10-7.01 (m, 2H), 6.52-6.50 (m, 1H), 4.17 (s, 4H), 3.91-3.82 (m, 1H), 3.72-3.61 (m, 2H), 3.59-3.36 (m, 2H), 3.31-3.28 (m, 3H), 3.26-3.21 (m, 3H), 2.99-2.91 (m, 5H), 2.53-2.51 (m, 3H), 2.14-1.93 (m, 2H), 1.72-1.57 (m, 2H). |
| 37 | | 8-(1,1-difluoro-6- | 452.2 | ¹H NMR (400 MHz, |
| | | azaspiro[3.4]octan-6-yl)- | | CDCl₃) δ (ppm) 8.94 (s, |
| | | N-(1- | | 1H), 7.25-7.03 (m, 2H), |
| | | (methylsulfonyl)piperidin- | | 6.97-6.75 (m, 1H), |
| | | 4-yl)quinazolin-2-amine | | 5.39-4.99 (m, 1H), |
| | | | | 4.60-4.30 (m, 1H), |
| | | | | 4.23-4.04 (m, 1H), |
| | | | | 3.86-3.69 (m, 3H), |
| | | | | 3.64-3.49 (m, 2H), |
| | | | | 3.15-2.93 (m, 2H), 2.84 (s, |
| | | | | 3H), 2.65-2.44 (m, 3H), |
| | | | | 2.34-2.15 (m, 2H), |
| | | | | 2.09-1.97 (m, 1H), |
| | | | | 1.93-1.82 (m, 2H), |
| | | | | 1.81-1.62 (m, 2H). |
| 38 | | 1-(7-(2-((2-methyl- | 457.3 | ¹H NMR (400 MHz, |
| | | 1,2,3,4- | | DMSO-d₆) δ (ppm) 9.46 |
| | | tetrahydroisoquinolin-6- | | (s, 1H), 9.15 (s, 1H), 7.55 |
| | | yl)amino)quinazolin-8-yl)- | | (s, 1H), 7.48-7.42 (m, 1H), |
| | | 2,7-diazaspiro[4.4]nonan- | | 7.23-7.16 (m, 2H), |
| | | 2-yl)ethan-1-one | | 6.99-6.96 (m, 1H), |
| | | | | 6.89-6.87 (m, 1H), |
| | | | | 3.81-3.73 (m, 2H), |
| | | | | 3.71-3.20 (m, 8H), 2.83 (s, |
| | | | | 2H), 2.66 (s, 2H), 2.59 (s, |
| | | | | 3H), 2.01-1.87 (m, 7H). |
| 39 | 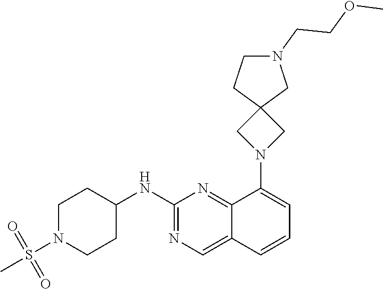 | 8-(6-(2-methoxyethyl)- 2,6-diazaspiro[3.4]octan- 2-yl)-N-(1- (methylsulfonyl)piperidin- 4-yl)quinazolin-2-amine | 475.2 | ¹H NMR (400 MHz, DMSO-d₆) δ (ppm) 8.98 (s, 1H), 7.25 (s, 1H), 7.09-7.01 (m, 2H), 6.53-6.50 (m, 1H), 4.09-4.02 (m, 4H), 3.99 (s, 1H), 3.59-3.44 (m, 2H), 3.42-3.33 (m, 2H), 3.24-3.17 (m, 3H), 2.93-2.86 (m, 5H), 2.77 (s, 2H), 2.59-2.49 (m, 4H), 2.05-2.00 (m, 4H), 1.66-1.53 (m, 2H) |
| 40 | | 8-(6-methyl-2,6- | 431.2 | ¹H NMR (400 MHz, |
| | | diazaspiro[3.4]octan-2-yl)- | | DMSO-d₆) δ (ppm) 8.99 |
| | | N-(1- | | (s, 1H), 7.26 (s, 1H), |
| | | (methylsulfonyl)piperidin- | | 7.10-7.02 (m, 2H), |
| | | 4-yl)quinazolin-2-amine | | 6.52-6.51 (m, 1H), |
| | | | | 4.10-4.01 (m, 4H), 3.87 (s, |
| | | | | 1H), 3.58-3.55 (m, 2H), |
| | | | | 2.93-2.88 (m, 5H), 2.72 (s, |
| | | | | 2H), 2.51-2.46 (m, 2H), |
| | | | | 2.25 (s, 3H), 2.08-2.02 (m, |
| | | | | 4H), 1.65-1.57 (m, 2H) |
| 41 | | 1-(7-(2-((5-(2-oxa-6- | 486.3 | ¹H NMR (400 MHz, |
| | | azaspiro[3.3]heptan-6- | | CDCl₃) δ (ppm) 9.07 (d, |
| | | yl)pyridin-2- | | J = 5.0 Hz, 1H), 8.20 (t, |
| | | yl)amino)quinazolin-8-yl)- | | J = 8.8 Hz, 1H), 7.89 (s, |
| | | 2,7-diazaspiro[4.4]nonan- | | 1H), 7.65-7.58 (m, 1H), |
| | | 2-yl)ethan-1-one | | 7.26-7.14 (m, 2H), |
| | | | | 6.98-6.84 (m, 2H), 4.89 (d, |
| | | | | J = 1.5 Hz, 4H), 4.14-4.05 |
| | | | | (m, 4H), 3.89-3.69 (m, |
| | | | | 4H), 3.67-3.54 (m, 3H), |
| | | | | 3.50 (d, J = 3.4 Hz, 1H), |
| | | | | 2.13-1.96 (m, 7H). |
| 42 | 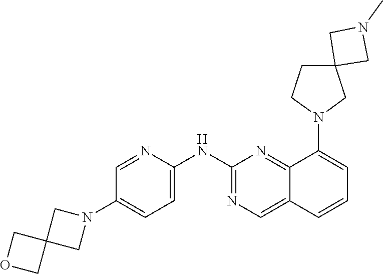 [SMILES: CN1CC2(CCN(c3cccc4cnc(Nc5ccc(N6CC7(COC7)C6)cn5)nc34)C2)C1] | N-(5-(2-oxa-6- azaspiro[3.3]heptan-6- yl)pyridin-2-yl)-8-(2- methyl-2,6- diazaspiro[3.4]octan-6- yl)quinazolin-2-amine | 444.2 | ¹H NMR (400 MHz, CDCl₃) δ (ppm) 9.07 (s, 1H), 8.22 (d, J = 8.9 Hz, 1H), 7.90 (s, 1H), 7.65 (d, J = 2.9 Hz, 1H), 7.27-7.13 (m, 2H), 6.96-6.82 (m, 2H), 4.89 (s, 4H), 4.08 (s, 4H), 3.84 (s, 2H), 3.65 (t, J = 6.8 Hz, 2H), 3.36 (d, J = 7.0 Hz, 2H), 3.26 (d, J = 7.1 Hz, 2H), 2.40 (s, 3H), 2.26 (t, J = 6.8 Hz, 2H). |
| 43 | | N-(5-(2-oxa-6- | 488.3 | ¹H NMR (300 MHz, |
| | | azaspiro[3.3]heptan-6- | | DMSO-d₆) δ (ppm) 9.46 |
| | | yl)pyridin-2-yl)-8-(2-(2- | | (s, 1H), 9.14-9.12 (m, 1H), |
| | | methoxyethyl)-2,6- | | 7.89-7.86 (m, 1H), |
| | | diazaspiro[3.4]octan-6- | | 7.64-7.60 (m, 1H), |
| | | yl)quinazolin-2-amine | | 7.24-7.17 (m, 2H), |
| | | | | 6.98-6.95 (m, 1H), |
| | | | | 6.80-6.78 (m, 1H), 4.72 (s, |
| | | | | 4H), 4.00 (s, 4H), 3.77 (s, |
| | | | | 2H), 3.46-3.44 (m, 2H), |
| | | | | 3.31-3.29 (m, 2H), |
| | | | | 3.27-3.17 (m, 5H), |
| | | | | 3.11-3.09 (m, 2H), |
| | | | | 2.56-2.54 (m, 2H), |
| | | | | 2.10-2.06 (m, 2H). |
| 44 | | 6-(2-((5-(6-ethyl-2,6- | 492.2 | ¹H NMR (400 MHz, |
| | | diazaspiro[3.3]heptan-2- | | CDCl₃) δ (ppm) 9.07 (s, |
| | | yl)pyridin-2- | | 1H), 8.24-8.22 (m, 1H), |
| | | yl)amino)quinazolin-8-yl)- | | 7.93 (s, 1H), 7.64-7.63 (m, |
| | | 1-thia-6- | | 1H), 7.28-7.23 (m, 2H), |
| | | azaspiro[3.3]heptane 1,1- | | 7.08-7.06 (m, 1H), |
| | | dioxide | | 6.74-6.72 (m, 1H), |
| | | | | 5.02-4.99 (m, 2H), |
| | | | | 4.20-4.13 (m, 2H), |
| | | | | 4.09-4.02 (m, 6H), 3.51 (s, |
| | | | | 4H), 2.55-2.49 (m, 2H), |
| | | | | 2.39-2.30 (m, 2H), |
| | | | | 1.27-1.03 (m, 3H). |
| 45 | | 1-(7-(7-fluoro-2-((1- | 491.2 | ¹H NMR (400 MHz, |
| | | (methylsulfonyl)piperidin- | | DMSO-d₆) δ (ppm) 9.01 |
| | | 4-yl)amino)quinazolin-8- | | (s, 1H), 7.48-7.32 (m, 2H), |
| | | yl)-2,7- | | 7.05-7.00 (m, 1H), |
| | | diazaspiro[4.4]nonan-2- | | 3.83-3.42 (m, 11H), |
| | | yl)ethan-1-one | | 2.92-2.82 (m, 5H), |
| | | | | 2.07-1.90 (m, 9H), |
| | | | | 1.61-1.54 (m, 2H). |
| 46 | | 1-(7-(2-((5-(6-ethyl-2,6- | 513.3 | ¹H NMR (400 MHz, |
| | | diazaspiro[3.3]heptan-2- | | CDCl₃) δ (ppm) 9.05 (s, |
| | | yl)pyridin-2- | | 1H), 8.15-8.11 (m, 1H), |
| | | yl)amino)quinazolin-8-yl)- | | 7.79 (s, 1H), 7.61-7.59 (m, |
| | | 2,7-diazaspiro[4.4]nonan- | | 1H), 7.26-7.15 (m, 2H), |
| | | 2-yl)ethan-1-one | | 6.91-6.80 (m, 2H), |
| | | | | 4.02-3.97 (m, 4H), |
| | | | | 3.87-3.69 (m, 7H), |
| | | | | 3.65-3.53 (m, 5H), |
| | | | | 2.53-2.44 (m, 2H), |
| | | | | 2.15-2.02 (m, 7H), |
| | | | | 1.02-0.96 (m, 3H). |
| 47 | | 1-(7-(2-((5-(6,6-difluoro- | 520.3 | ¹H NMR (400 MHz, |
| | | 2-azaspiro[3.3]heptan-2- | | DMSO-d₆) δ (ppm) 9.42 |
| | | yl)pyridin-2- | | (s, 1H), 9.16-9.15 (s, 1H), |
| | | yl)amino)quinazolin-8-yl)- | | 7.87 (m, 1H), 7.61-7.59 |
| | | 2,7-diazaspiro[4.4]nonan- | | (m, 1H), 7.21-7.19 (m, |
| | | 2-yl)ethan-1-one | | 2H), 7.00-6.95 (m, 1H), |
| | | | | 6.85-6.80 (s, 1H), |
| | | | | 3.94-3.93 (m, 4H), |
| | | | | 3.93-3.32 (m, 8H), |
| | | | | 2.90-2.83 (m, 4H), |
| | | | | 2.50-1.87 (m, 7H) |
| 48 | 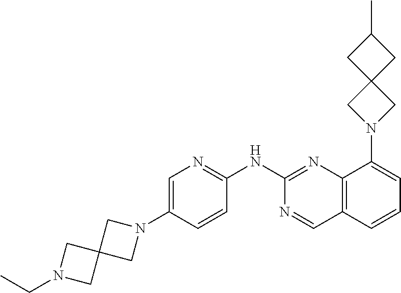 [SMILES: CCN1CC2(C1)CN(c1ccc(Nc3ncc4cccc(N5CC6(CC(C)C6)C5)c4n3)nc1)C2] | 2-(2-((5-(6-ethyl-2,6- diazaspiro[3.3]heptan-2- yl)pyridin-2- yl)amino)quinazolin-8-yl)- 2-azaspiro[3.3]heptan-6-ol | 458.2 | ¹H NMR (400 MHz, Chloroform-d) δ (ppm) 9.05 (s, 1H), 8.22 (d, J = 8.9 Hz, 1H), 7.79 (s, 1H), 7.63 (d, J = 2.9 Hz, 1H), 7.22-7.13 (m, 2H), 6.92 (dd, J = 8.8, 2.9 Hz, 1H), 6.65 (dd, J = 7.5, 1.4 Hz, 1H), 4.36-4.24 (m, 1H), 4.19 (d, J = 5.7 Hz, 4H), 4.02 (s, 4H), 3.45 (s, 4H), 2.72-2.62 (m, 2H), 2.57-2.46 (m, 2H), 2.24-2.13 (m, 3H), 1.06-0.98 (m, 3H). |
| 49 | | 8-(6,6-difluoro-2- | 478.1 | ¹H NMR (400 MHz, |
| | | azaspiro[3.3]heptan-2-yl)- | | Chloroform-d) δ (ppm) |
| | | N-(5-(6-ethyl-2,6- | | 9.06 (s, 1H), 8.15 (d, |
| | | diazaspiro[3.3]heptan-2- | | J = 8.9 Hz, 1H), 7.79 (s, |
| | | yl)pyridin-2- | | 1H), 7.64 (d, J = 2.8 Hz, |
| | | yl)quinazolin-2-amine | | 1H), 7.27-7.15 (m, 2H), |
| | | | | 6.90 (dd, J = 8.9, 2.9 Hz, |
| | | | | 1H), 6.67 (dd, J = 7.2, 1.7 |
| | | | | Hz, 1H), 4.28 (s, 4H), 4.02 |
| | | | | (s, 4H), 3.46 (s, 4H), |
| | | | | 2.91-2.79 (m, 4H), 2.53 (d, |
| | | | | J = 7.3 Hz, 2H), 1.03 (t, |
| | | | | J = 7.2 Hz, 3H). |
| 50 | | 1-(7-(6-(difluoromethyl)- | 523.2 | ¹H NMR (400 MHz, |
| | | 2-((1- | | DMSO-d₆) δ (ppm) 9.08 |
| | | (methylsulfonyl)piperidin- | | (s, 1H), 7.54 (s, 1H), 7.30 |
| | | 4-yl)amino)quinazolin-8- | | (s, 1H), 7.15-6.56 (m, 2H), |
| | | yl)-2,7- | | 3.93-3.78 (m, 3H), |
| | | diazaspiro[4.4]nonan-2- | | 3.73-3.53 (m, 5H), |
| | | yl)ethan-1-one | | 3.52-3.37 (m, 2H), |
| | | | | 3.32-3.26 (m, 1H), 2.91 (d, |
| | | | | J = 4.4 Hz, 3H), 2.89-2.66 |
| | | | | (m, 2H), 2.08-1.85 (m, |
| | | | | 9H), 1.67-1.52 (m, 2H). |
| 51 | | 1-(7-(6-(difluoromethyl)- | 536.2 | ¹H NMR (400 MHz, |
| | | 2-((4-(4-methylpiperazin- | | DMSO-d₆) δ (ppm) 9.51 |
| | | 1-yl)phenyl)amino) | | (s, 1H), 9.20 (d, J = 1.3 Hz, |
| | | quinazolin-8-yl)-2,7- | | 1H), 7.50 (dd, J = 8.9, 4.8 |
| | | diazaspiro[4.4]nonan-2- | | Hz, 2H), 7.38 (d, J = 1.6 |
| | | yl)ethan-1-one | | Hz, 1H), 7.06-6.82 (m, |
| | | | | 4H), 3.72-3.28 (m, 6H), |
| | | | | 3.33-3.25 (m, 2H), |
| | | | | 3.11-3.06 (m, 4H), |
| | | | | 2.48-2.43 (m, 4H), 2.23 (s, |
| | | | | 3H), 1.97-1.82 (m, 7H). |
| 52 | | 1-(7-(6-(difluoromethyl)- | 470.2 | ¹H NMR (400 MHz, |
| | | 2-((1-isopropyl-1H- | | DMSO-d6) δ (ppm) 9.48 |
| | | pyrazol-4- | | (s, 1H), 9.19 (s, 1H), 7.90 |
| | | yl)amino)quinazolin-8-yl)- | | (d, J = 3.2 Hz, 1H), 7.53 |
| | | 2,7-diazaspiro[4.4]nonan- | | (d, J = 6.8 Hz, 1H), 7.41 (s, |
| | | 2-yl)ethan-1-one | | 1H), 7.19-6.85 (m. 2H), |
| | | | | 4.56-4.40 (m, 1H), 3.75 (s, |
| | | | | 2H), 3.60-3.49 (m, 3H), |
| | | | | 3.48-3.44 (m, 1H), |
| | | | | 3.43-3.36 (m, 1H), |
| | | | | 3.32-3.24 (m, 1H), |
| | | | | 2.04-1.82 (m, 7H), 1.43 |
| | | | | (dd, J = 6.7, 1.4 Hz, 6H). |
| 53 | | 8-(1,1-difluoro-5- | 422.1 | ¹H NMR (300 MHz, |
| | | azaspiro[2.4]heptan-5-yl)- | | Chloroform-d) δ (ppm) |
| | | 6-(difluoromethyl)-N- | | 8.93 (s, 1H), 7.25 (d, |
| | | ((1R,5S,6s)-3- | | J = 2.1 Hz, 1H), 6.88 (s, |
| | | azabicyclo[3.1.0]hexan-6- | | 1H), 6.68 (t, J = 56.5 Hz, |
| | | yl)quinazolin-2-amine | | 1H), 5.37 (s, 1H), |
| | | | | 4.12-3.85 (m, 4H), |
| | | | | 3.25-3.13 (m, 3H), 2.50 (d, |
| | | | | J = 9.1 Hz, 2H), 2.36 (s, |
| | | | | 4H), 2.20-2.06 (m, 1H), |
| | | | | 1.66 (s, 2H), 1.54-1.39 (m, |
| | | | | 2H). |
| 54 | | 6-(6-(difluoromethyl)-2- | 450.1 | ¹H NMR (300 MHz, |
| | | (((1R,5S,6s)-3-methyl-3- | | Chloroform-d) δ (ppm) |
| | | azabicyclo[3.1.0]hexan-6- | | 8.91 (s, 1H), 7.22 (d, |
| | | yl)amino)quinazolin-8-yl)- | | J = 1.7 Hz, 1H), 6.89-6.46 |
| | | 2-thia-6- | | (m, 2H), 5.35 (s, 1H), |
| | | azaspiro[3.4]octane 2,2- | | 4.33-4.20 (m, 4H), 4.14 (d, |
| | | dioxide | | J = 13.7 Hz, 2H), 3.95 (t, |
| | | | | J = 6.8 Hz, 2H), 3.30-3.18 |
| | | | | (m, 2H), 2.53 (d, J = 9.1 |
| | | | | Hz, 2H), 2.44-2.33 (m, |
| | | | | 5H), 1.67 (s, 3H). |
| 55 | | 7-methyl-2-(2-((1- | 473.1 | ¹H NMR (400 MHz, |
| | | (methylsulfonyl)piperidin- | | DMSO-d₆) δ (ppm) 9.00 |
| | | 4-yl)amino)quinazolin-8- | | (s, 1H), 7.26 (s, 1H), |
| | | yl)-2,7- | | 7.14-7.05 (m, 2H), |
| | | diazaspiro[4.5]decan-8- | | 6.78-6.75 (m, 1H), |
| | | one | | 3.93-3.52 (m, 7H), 3.28 (s, |
| | | | | 2H), 2.96-2.80 (m, 8H), |
| | | | | 2.40-2.24 (m, 2H), |
| | | | | 2.10-1.97 (m, 2H), |
| | | | | 1.96-1.80 (m, 4H), |
| | | | | 1.69-1.51 (m, 2H). |
| 56 | 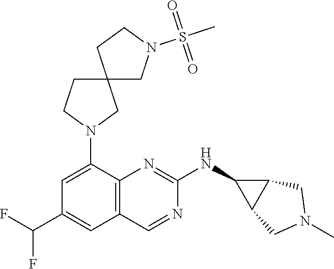 [SMILES: CN1C[C@@H]2[C@H](C1)[C@H]2Nc1ncc2cc(C(F)F)cc(N3CCC4(CCN(S(C)(=O)=O)C4)C3)c2n1] | 6-(difluoromethyl)-N- ((1R,5S,6s)-3-methyl-3- azabicyclo[3.1.0]hexan-6- yl)-8-(7-(methylsulfonyl)- 2,7-diazaspiro[4.4]nonan- 2-yl)quinazolin-2-amine | 493.1 | ¹H NMR (300 MHz, Chloroform-d) δ (ppm) 8.91 (s, 1H), 7.20 (d, J = 1.9 Hz, 1H), 6.88-6.46 (m, 2H), 5.37 (s, 1H), 4.03-3.76 (m, 4H), 3.62-3.37 (m, 5H), 3.24 (s, 3H), 2.92 (s, 4H), 2.56 (s, 2H), 2.42 (s, 3H), 2.17-2.00 (m, 4H). |
| 57 | | 6-(difluoromethyl)-8-(6- | 437.2 | ¹H NMR (300 MHz, |
| | | methyl-2,6- | | Chloroform-d) δ (ppm) |
| | | diazaspiro[3.3]heptan-2- | | 9.04 (s, 1H), 7.65 (s, 1H), |
| | | yl)-N-(2-methylisoindolin- | | 7.35 (d, J = 6.8 Hz, 1H), |
| | | 5-yl)quinazolin-2-amine | | 7.23 (d, J = 11.0 Hz, 3H), |
| | | | | 6.88-6.48 (m, 2H), 4.31 (s, |
| | | | | 4H), 4.03 (d, J = 13.9 Hz, |
| | | | | 4H), 3.49 (s, 4H), 2.68 (s, |
| | | | | 3H), 2.39 (s, 3H). |
| 58 | | 6-(difluoromethyl)-N-(2- | 501.0 | ¹H NMR (300 MHz, |
| | | methylisoindolin-5-yl)-8- | | Chloroform-d) δ 9.06 (s, |
| | | (6-(methylsulfonyl)-2,6- | | 1H), 7.60 (s, 1H), 7.38 (d, |
| | | diazaspiro[3.3]heptan-2- | | J = 8.4 Hz, 1H), 7.29 (s, |
| | | yl)quinazolin-2-amine | | 2H), 7.24 (d, J = 8.1 Hz, |
| | | | | 1H), 6.69 (t, J = 54.3 Hz, |
| | | | | 2H), 4.35 (s, 4H), 4.15 (s, |
| | | | | 4H), 4.04 (d, J = 10.9 Hz, |
| | | | | 4H), 2.92 (s, 3H), 2.69 (s, |
| | | | | 3H) |
| 59 | | 6-(difluoromethyl)-8-(6-6- | 441.2 | ¹H-NMR (400 MHz, |
| | | methyl-2,6- | | Chloroform-d) δ (ppm) |
| | | diazaspiro[3.3]heptan-2- | | 9.03 (s, 1H), 7.96 (s, 1H), |
| | | yl)-N-(1-(1- | | 7.66 (s, 1H), 7.26-7.17 (m, |
| | | methylazetidin-3-yl)-1H- | | 1H), 6.96 (s, 1H), |
| | | pyrazol-4-yl)quinazolin-2- | | 6.84-6.49 (m, 2H), |
| | | amine | | 5.04-4.88 (m, 1H), |
| | | | | 4.36-4.17 (m, 4H), |
| | | | | 3.94-3.81 (m, 2H), |
| | | | | 3.70-3.56 (m, 2H), |
| | | | | 3.53-3.32 (m, 4H), |
| | | | | 2.59-2.45 (m, 3H), |
| | | | | 2.37-2.28 (m, 3H). |
| 60 | | 3-cyclopropyl-1-(6- | 505.2 | ¹H NMR (400 MHz, |
| | | (difluoromethyl)-2-((1-(2- | | Chloroform-d) δ (ppm) |
| | | methyl-2- | | 9.03 (s, 1H), 7.81 (s, 1H), |
| | | azaspiro[3.3]heptan-6-yl)- | | 7.60 (d, J = 3.8 Hz, 1H), |
| | | 1H-pyrazol-4- | | 7.34-7.29 (m, 1H), 7.10 (s, |
| | | yl)amino)quinazolin-8- | | 1H), 6.92 (s, 1H), 6.69 (t, |
| | | yl)pyrrolidine-3- | | J = 56.4 Hz, 1H), |
| | | carbonitrile | | 4.75-4.61 (m, 1H), 4.19 (d, |
| | | | | J = 10.7 Hz, 1H), |
| | | | | 4.00-3.92 (m, 1H), 3.81 (d, |
| | | | | J = 10.6 Hz, 1H), 3.68 (s, |
| | | | | 1H), 3.36 (t, J = 3.3 Hz, |
| | | | | 2H), 3.29 (s, 2H), |
| | | | | 2.80-2.64 (m, 4H), |
| | | | | 2.53-2.40 (m, 1H), |
| | | | | 2.25-2.12 (m, 1H), |
| | | | | 2.09-1.99 (m, 3H), |
| | | | | 1.16-1.06 (m, 1H), |
| | | | | 0.74-0.57 (m, 4H). |
| 61 | 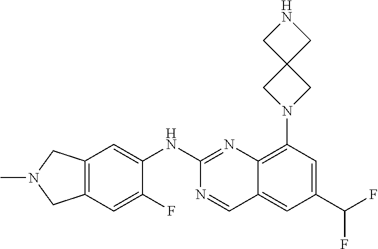 [SMILES: CN1Cc2cc(F)c(Nc3ncc4cc(C(F)F)cc(N5CC6(CNC6)C5)c4n3)cc2C1] | 6-(difluoromethyl)-N-(6- fluoro-2-methylisoindolin- 5-yl)-8-(2,6- diazaspiro[3.3]heptan-2- yl)quinazolin-2-amine | 441.2 | ¹H NMR (400 MHz, DMSO-d₆) δ (ppm) 9.28 (s, 1H), 9.24 (s, 1H), 7.62-7.59 (m, 1H), 7.39 (s, 1H), 7.21-6.88 (m, 2H), 6.59 (s, 1H), 4.09 (s, 4H), 3.87 (s, 5H), 3.75-3.51 (m, 4H), 2.49 (s, 3H). |
| 62 | | 6-(difluoromethyl)-8-(2- | 481.1 | ¹H NMR (300 MHz, |
| | | methyl-2,6- | | DMSO-d₆) δ (ppm) 9.06 |
| | | diazaspiro[3.4]octan-6-yl)- | | (s, 1H), 7.58-7.42 (m, 1H), |
| | | N-(1- | | 7.28 (s, 1H), 6.97 (t, |
| | | (methylsulfonyl)piperidin- | | J = 56.1 Hz, 1H), 6.72 (s, |
| | | 4-yl)quinazolin-2-amine | | 1H), 3.91 (s, 3H), 3.60 (d, |
| | | | | J = 13.3 Hz, 4H), 3.12 (d, |
| | | | | J = 1.7 Hz, 4H), 2.88 (s, |
| | | | | 5H), 2.22 (s, 3H), |
| | | | | 2.10-2.01 (m, 4H), 1.60 (d, |
| | | | | J = 12.3 Hz, 2H). |
| 63 | | 6-(difluoromethyl)-N-(1- | 467.2 | ¹H-NMR (300 MHz, |
| | | (methylsulfonyl)piperidin- | | DMSO-d₆) δ (ppm) 9.09 |
| | | 4-yl)-8-(2,6- | | (s, 1H), 7.56 (s, 1H), |
| | | diazaspiro[3.4]octan-2- | | 7.37-7.27 (m, 1H), |
| | | yl)quinazolin-2-amine | | 7.22-6.80 (m, 1H), 6.55 (s, |
| | | | | 1H), 4.20-4.05 (m, 4H), |
| | | | | 3.97-3.76 (m, 1H), |
| | | | | 3.71-3.55 (m, 3H), |
| | | | | 3.40-3.25 (m, 1H), |
| | | | | 3.02-2.80 (m, 8H), |
| | | | | 2.16-1.92 (m, 4H), |
| | | | | 1.71-1.50 (m, 2H). |
| 64 | 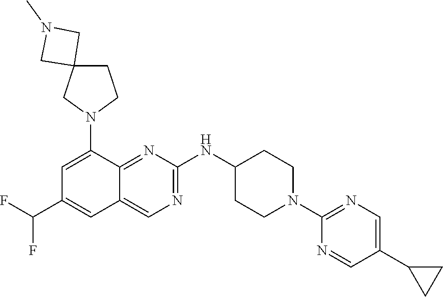 [SMILES: CN1CC2(CCN(c3cc(C(F)F)cc4cnc(NC5CCN(c6ncc(C7CC7)cn6)CC5)nc34)C2)C1] | N-(1-(5- cyclopropylpyrimidin-2- yl)piperidin-4-yl)-6- (difluoromethyl)-8-(2- methyl-2,6- diazaspiro[3.4]octan-6- yl)quinazolin-2-amine | 521.1 | ¹H NMR (400 MHz, Chloroform-d) δ (ppm) 8.94 (s, 1H), 8.16 (s, 2H), 7.28 (s, 1H), 7.21 (s, 1H), 6.82-6.53 (m, 2H), 4.69 (d, J = 13.7 Hz, 2H), 4.21-4.02 (m, 3H), 3.71 (s, 2H), 3.47 (s, 3H), 3.24-3.15 (m, 2H), 2.49 (s, 2H), 2.31-2.18 (m, 4H), 1.80-1.47 (m, 5H), 0.97-0.88 (m, 2H), 0.65-0.56 (m, 2H). |
| 65 | | 8-(6-cyclopropyl-2,6- | 463.1 | ¹H NMR (400 MHz, |
| | | diazaspiro[3.3]heptan-2- | | DMSO-d₆) δ (ppm) 9.81 |
| | | yl)-6-(difluoromethyl)-N- | | (s, 1H), 9.25 (s, 1H), 7.82 |
| | | (2-methylisoindolin-5- | | (s, 1H), 7.41 (d, J = 1.7 Hz, |
| | | yl)quinazolin-2-amine | | 1H), 7.35 (dd, J = 7.9, 1.9 |
| | | | | Hz, 1H), 7.20-7.16 (m, |
| | | | | 1H), 7.04 (s, 1H), 6.65 (s, |
| | | | | 1H), 4.19 (s, 4H), 3.87 (s, |
| | | | | 2H), 3.80 (s, 2H), |
| | | | | 3.40-3.34 (m, 4H), |
| | | | | 3.33-3.30 (m, 3H), |
| | | | | 1.83-1.80 (m, 1H), |
| | | | | 0.33-0.30 (m, 2H), |
| | | | | 0.22-0.21 (m, 2H). |
| 66 | | 6-(difluoromethyl)-8-(2- | 451.1 | ¹H NMR (400 MHz, |
| | | methyl-2,6- | | DMSO-d₆) δ (ppm) 9.76 |
| | | diazaspiro[3.4]octan-6-yl)- | | (s, 1H), 9.26 (s, 1H), 7.66 |
| | | N-(2-methylisoindolin-5- | | (d, J = 1.9 Hz, 1H), 7.46 |
| | | yl)quinazolin-2-amine | | (dd, J = 8.2, 2.0 Hz, 1H), |
| | | | | 7.42 (d, J = 1.8 Hz, 1H), |
| | | | | 7.18 (d, J = 7.8 Hz, 1H), |
| | | | | 7.17-7.15 (m, 1H) 6.86 (d, |
| | | | | J = 1.7 Hz, 1H), 3.81-3.79 |
| | | | | (m, 4H), 3.74 (s, 2H), |
| | | | | 3.70-3.66 (m, 2H) |
| | | | | 3.60-3.55 (m, 3H), |
| | | | | 3.24-3.22 (m, 2H), 3.18 (s, |
| | | | | 2H), 2.29 (s, 3H), |
| | | | | 2.11-2.10 (m, 2H). |
| 67 | | 6-(difluoromethyl)-8-(2- | 466.1 | ¹H NMR (400 MHz, |
| | | methyl-2- | | Chloroform-d) δ 8.97 (s, |
| | | azaspiro[3.3]heptan-6-yl)- | | 1H), 7.70-7.54 (m, 2H), |
| | | N-(1- | | 6.71 (t, J = 56.4 Hz, 1H), |
| | | (methylsulfonyl)piperidin- | | 5.30 (d, J = 7.3 Hz, 1H), |
| | | 4-yl)quinazolin-2-amine | | 4.14-3.95 (m, 2H), 3.80 (d, |
| | | | | J = 12.4 Hz, 2H), 3.47 (s, |
| | | | | 2H), 3.20 (s, 2H), |
| | | | | 3.08-2.97 (m, 2H), 2.86 (s, |
| | | | | 3H), 2.72-2.63 (m, 2H), |
| | | | | 2.39-2.27 (m, 7H), 1.75 (s, |
| | | | | 1H), 1.25 (s, 1H). |
| 68 | | N-(1-(5- | 507.1 | ¹H NMR (400 MHz, |
| | | cyclopropylpyrimidin-2- | | DMSO-d6) δ (ppm) 9.07 |
| | | yl)piperidin-4-yl)-6- | | (s, 1H), 8.18 (s, 2H), 7.53 |
| | | (difluoromethyl)-8-(2,6- | | (s, 1H), 7.30 (d, J = 1.8 Hz, |
| | | diazaspiro[3.4]octan-2- | | 1H), 7.14-6.85 (m, 1H), |
| | | yl)quinazolin-2-amine | | 6.56 (d, J = 7.8 Hz, 1H), |
| | | | | 4.59 (d, J = 13.5 Hz, 2H), |
| | | | | 4.14 (s, 4H), 4.00 (s, 1H), |
| | | | | 3.48 (s, 1H), 3.12-2.99 (m, |
| | | | | 3H), 2.85 (t, J = 8.0 Hz, |
| | | | | 2H), 2.16-1.94 (m, 5H), |
| | | | | 1.80-1.73 (m, 1H), |
| | | | | 1.50-1.37 (m, 2H), |
| | | | | 0.90-0.83 (m, 2H), |
| | | | | 0.67-0.60 (m, 2H). |
| 69 | 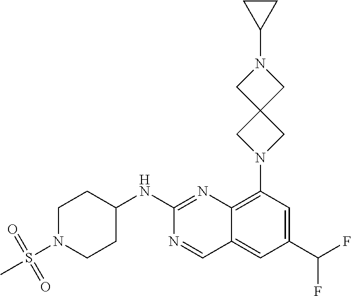 [SMILES: CS(=O)(=O)N1CCC(Nc2ncc3cc(C(F)F)cc(N4CC5(C4)CN(C4CC4)C5)c3n2)CC1] | 8-(6-cyclopropyl-2,6- diazaspiro[3.3]heptan-2- yl)-6-(difluoromethyl)-N- (1- (methylsulfonyl)piperidin- 4-yl)quinazolin-2-amine | 493 | ¹H NMR (300 MHz, Chloroform-d) δ (ppm) 8.94 (s, 1H), 7.21 (s, 1H), 6.86-6.45 (m, 2H), 5.23 (d, J = 7.5 Hz, 1H), 4.34 (s, 4H), 4.08 (d, J = 9.7 Hz, 1H), 3.87-3.71 (m, 2H), 3.59 (s, 4H), 3.10 (s, 2H), 2.88 (s, 3H), 2.26 (d, J = 12.8 Hz, 2H), 1.85-1.68 (m, 3H), 0.45 (s, 4H). |
| 70 | | 6-(difluoromethyl)-N-(1- | 466 | ¹H NMR (400 MHz, |
| | | (methylsulfonyl)piperidin- | | DMSO-d₆) δ (ppm) 9.19 |
| | | 4-yl)-8-(6- | | (s, 1H), 7.89 (s, 1H), |
| | | azaspiro[3.4]octan-2- | | 7.80-7.73 (m, 1H), 7.65 (d, |
| | | yl)quinazolin-2-amine | | J = 9.1 Hz, 1H), 7.12 (t, |
| | | | | J = 56.0 Hz, 1H), 413-3.89 |
| | | | | (m, 2H), 3.60 (d, J = 11.9 |
| | | | | Hz, 3H), 2.99-2.78 (m, |
| | | | | 8H), 2.46-2.34 (m, 2H), |
| | | | | 2.26-1.97 (m, 5H), |
| | | | | 1.91-1.51 (m, 4H). |
| 71 | | N-(1-(7-cyclopropyl-7H- | 560.2 | ¹H NMR (400 MHz, |
| | | pyrrolo[2,3-d]pyrimidin-2- | | DMSO-d6) δ (ppm) 9.07 |
| | | yl)piperidin-4-yl)-6- | | (s, 1H), 8.57 (s, 1H), 7.51 |
| | | (difluoromethyl)-8-(2- | | (s, 1H), 7.31 (s, 1H), |
| | | methyl-2,6- | | 7.15-6.86 (m, 2H), 6.75 (s, |
| | | diazaspiro[3.4]octan-6- | | 1H), 6.31 (d, J = 3.6 Hz, |
| | | yl)quinazolin-2-amine | | 1H), 4.80 (d, J = 13.1 Hz, |
| | | | | 2H), 4.16-3.95 (m, 3H), |
| | | | | 3.60 (t, J = 6.7 Hz, 2H), |
| | | | | 3.51-3.41 (m, 5H), 3.08 (t, |
| | | | | J = 12.4 Hz, 2H), 2.43 (s, |
| | | | | 3H), 2.19-2.14 (m, 2H), |
| | | | | 2.05 (d, J = 12.2 Hz, 2H), |
| | | | | 1.57-1.47 (m, 2H), 1.00 (d, |
| | | | | J = 5.5 Hz, 4H). |
| 72 | 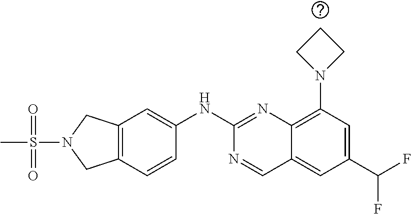 [SMILES: CS(=O)(=O)N1Cc2ccc(Nc3ncc4cc(C(F)F)cc(N5CCC5)c4n3)cc2C1] | 8-(6-cyclopropyl-2,6- diazaspiro[3.3]heptan-2- yl)-6-(difluoromethyl)-N- (2-(methylsulfonyl) isoindolin-5- yl)quinazolin-2-amine | 527.2 | ¹H NMR (400 MHz, DMSO-d₆) δ (ppm) 9.96 (s, 1H), 9.28 (s, 1H), 7.93 (d, J = 2.0 Hz, 1H), 7.56-7.49 (m, 1H), 7.45-7.41 (m, 1H), 7.31 (d, J = 8 Hz, 1H), 7.05 (s, 1H), 6.67 (d, J = 1.8 Hz, 1H), 4.73-7.57 (m, 4H), 4.22 (s, 4H), 3.41-3.34 (m, 4H), 2.99 (s, 3H), 1.88-1.81 (m, 1H), 0.36-0.19 (m, 4H). |
| 73 | | 6-(difluoromethyl)-8-(2- | 515.2 | ¹H NMR (400 MHz, |
| | | methyl-2,6- | | DMSO-d₆) δ (ppm) 9.89 |
| | | diazaspiro[3.4]octan-6-yl)- | | (s, 1H), 9.28 (s, 1H), 7.75 |
| | | (methylsulfonyl)N-(2- | | (s, 1H), 7.62 (d, J = 8.4 Hz, |
| | | isoindolin-5- | | 1H), 7.43 (s, 1H), 7.30 (d, |
| | | yl)quinazolin-2amine | | J = 8.2 Hz, 1H), 7.05 (s, |
| | | | | 1H), 6.89 (d, J = 15.0 Hz, |
| | | | | 1H), 4.63 (d, J = 13.2 Hz, |
| | | | | 4H), 3.75 (s, 2H), |
| | | | | 3.66-3.55 (m, 2H), |
| | | | | 3.20-3.06 (m, 4H), 2.99 (s, |
| | | | | 3H), 2.23 (s, 3H), |
| | | | | 2.15-2.07 (m, 2H). |
| 74 | | N-(1- | 493.1 | ¹H NMR (400 MHz, |
| | | (cyclopropylsulfonyl) | | DMSO-d₆) δ 9.09 (s, 1H), |
| | | piperidin-4-yl)-6- | | 7.64-7.49 (m, 1H), 7.31 (s, |
| | | (difluoromethyl)-8-(2,6- | | 1H), 6.99 (t, J = 56.1 Hz, |
| | | diazaspiro[3.4]octan-2- | | 1H), 6.56 (d, J = 7.0 Hz, |
| | | yl)quinazolin-2-amine | | 1H), 4.14 (d, J = 9.8 Hz, |
| | | | | 3H), 3.87 (s, 1H), 3.65 (d, |
| | | | | J = 12.8 Hz, 2H), |
| | | | | 3.06-2.93 (m, 4H), |
| | | | | 2.91-2.81 (m, 2H), |
| | | | | 2.67-2.57 (m, 2H), |
| | | | | 2.09-1.92 (m, 4H), |
| | | | | 1.66-1.51 (m, 2H), 1.24 (s, |
| | | | | 1H), 1.05-0.91 (m, 4H). |
| 75 | | 6-(difluoromethyl)-N-(1- | 436.1 | ¹H NMR (300 MHz, |
| | | methyl-1H-indazol-5-yl)- | | DMSO-d₆) δ (ppm) 9.87 |
| | | 8-(2,6- | | (s, 1H), 9.27 (s, 1H), 8.27 |
| | | diazaspiro[3.4]octan-2- | | (s, 1H), 7.98 (d, J = 18.0 |
| | | yl)quinazolin-2-amine | | Hz, 1H), 7.67-7.57 (m, |
| | | | | 2H), 7.45 (d, J = 2.5 Hz, |
| | | | | 1H), 7.06 (t, J = 56.0 Hz, |
| | | | | 1H), 6.69 (d, J = 1.8 Hz, |
| | | | | 1H), 4.12 (s, 3H), 4.05 (s, |
| | | | | 3H), 3.44 (s, 1H), 3.16 (s, |
| | | | | 2H), 2.99 (t, J = 7.0 Hz, |
| | | | | 2H), 2.11 (t, J = 7.0 Hz, |
| | | | | 2H), 1.24 (s, 1H). |
| 76 | | N-(1- | 537.1 | ¹H-NMR (400 MHz, |
| | | (cyclopropylsulfonyl) | | Chloroform-d) δ (ppm) |
| | | piperidin-4-yl)-6- | | 9.09-8.84 (m, 1H), |
| | | (difluoromethyl)-8-(6-(2- | | 7.28-7.17 (m, 1H), |
| | | methoxyethyl)-2,6- | | 6.89-6.44 (m, 2H), |
| | | diazaspiro[3.3]heptan-2- | | 5.56-5.18 (m, 1H), |
| | | yl)quinazolin-2-amine | | 4.54-4.24 (m, 3H), |
| | | | | 4.20-4.03 (m, 1H), |
| | | | | 3.96-3.11 (m, 12H), |
| | | | | 2.98-2.64 (m, 2H), |
| | | | | 2.51-2.12 (m, 3H), |
| | | | | 1.43-1.16 (m, 5H), |
| | | | | 1.13-0.85 (m, 3H). |
| 77 | 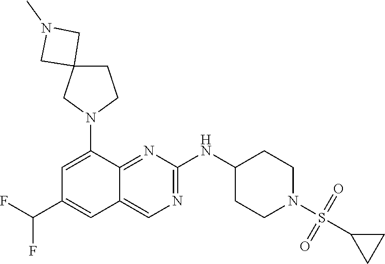 [SMILES: CN1CC2(CCN(c3cc(C(F)F)cc4cnc(NC5CCN(S(=O)(=O)C6CC6)CC5)nc34)C2)C1] | N-(1- (cyclopropylsulfonyl) piperidin-4-yl)-6- (difluoromethyl)-8-(2- methyl-2,6- diazaspiro[3.4]octan-6- yl)quinazolin-2-amine | 507.1 | ¹H NMR (400 MHz, DMSO-d₆) δ (ppm) 9.08 (s, 1H), 7.54 (s, 1H), 7.30 (t, J = 1.8 Hz, 1H), 6.99 (t, J = 56.1 Hz, 1H), 6.73 (d, J = 1.8 Hz, 1H), 4.02-3.85 (m, 3H), 3.72-3.62 (m, 2H), 3.53 (s, 2H), 3.18-3.08 (m, 4H), 3.04-2.93 (m, 2H), 2.63-2.54 (m, 1H), 2.23 (s, 3H), 2.13-2.02 (m, 4H), 1.68-1.55 (m, 2H), 1.04-0.91 (m, 4H). |
| 78 | | 8-(6-cyclopropyl-2,6- | 519.1 | ¹H NMR (300 MHz, |
| | | diazaspiro[3.3]heptan-2- | | DMSO-d₆) δ (ppm) 9.08 |
| | | yl)-N-(1- | | (s, 1H), 7.61 (s, 1H), 7.31 |
| | | (cyclopropylsulfonyl) | | (s, 1H), 6.99 (t, J = 56.0 |
| | | piperidin-4-yl)-6- | | Hz, 1H), 6.55 (s, 1H), 4.23 |
| | | (difluoromethyl) | | (s, 4H), 3.91 (s, 1H), 3.65 |
| | | quinazolin-2-amine | | (d, J = 11.6 Hz, 2H), 3.40 |
| | | | | (s, 3H), 3.05 (t, J = 11.5 |
| | | | | Hz, 2H), 2.68-2.58 (m, |
| | | | | 1H), 2.11-1.97 (m, 2H), |
| | | | | 1.87-1.80 (m, 1H), |
| | | | | 1.70-1.51 (m, 2H), 1.24 (s, |
| | | | | 1H), 1.08-0.92 (m, 4H), |
| | | | | 0.32 (d, J = 5.4 Hz, 2H), |
| | | | | 0.21 (d, J = 3.5 Hz, 2H). |
| 79 | | 8-(6-cyclopropyl-2,6- | 477.3 | ¹H NMR (300 MHz, |
| | | diazaspiro[3.3]heptan-2- | | DMSO-d₆) δ (ppm) 9.71 |
| | | yl)-6-(difluoromethyl)-N- | | (s, 1H), 9.24 (s, 1H), 7.68 |
| | | (2-methyl-1,2,3,4- | | (s, 1H), 7.40 (s, 1H), |
| | | tetrahydroisoquinolin-6- | | 7.33-7.19 (m, 1H), |
| | | yl)quinazolin-2-amine | | 7.07-6.97 (m, 2H), 6.64 (s, |
| | | | | 1H), 4.21 (s, 4H), 3.48 (s, |
| | | | | 2H), 3.38-3.36 (m, 4H), |
| | | | | 2.91-2.84 (m, 2H), |
| | | | | 2.67-2.58 (m, 2H), 2.36 (s, |
| | | | | 3H), 1.87-1.76 (m, 1H), |
| | | | | 0.38-0.30 (m, 2H), |
| | | | | 0.25-0.16 (m, 2H). |
| 80 | | N-(1- | 507.1 | ¹H NMR (400 MHz, |
| | | (cyclopropylsulfonyl) | | DMSO-d₆) δ (ppm) 9.08 |
| | | piperidin-4-yl)-6- | | (s, 1H), 7.55 (s, 1H), 7.26 |
| | | (difluoromethyl)-8-(2,6- | | (d, J = 1.8 Hz, 1H), 6.98 (t, |
| | | diazaspiro[3.5]nonan-2- | | J = 56.1 Hz, 1H), 6.51 (d, |
| | | yl)quinazolin-2-amine | | J = 1.8 Hz, 1H), 4.07-3.79 |
| | | | | (m, 5H), 3.66 (dd, J = 9.9, |
| | | | | 6.7 Hz, 2H), 3.08-2.95 (m, |
| | | | | 2H), 2.82 (s, 2H), |
| | | | | 2.68-2.55 (m, 4H), 2.07 (d, |
| | | | | J = 9.7 Hz, 2H), 1.72 (t, |
| | | | | J = 5.9 Hz, 2H), 1.66-1.54 |
| | | | | (m, 2H), 1.44 (d, J = 6.6 |
| | | | | Hz, 2H), 1.05-0.92 (m, |
| | | | | 4H). |
| 81 | | N-(1- | 507.1 | ¹H-NMR (400 MHz, |
| | | (cyclopropylsulfonyl) | | Chloroform-d) δ (ppm) |
| | | piperidin-4-yl)-6- | | 8.94 (s, 1H), 6.87-6.37 (m, |
| | | (difluoromethyl)-8-(6- | | 2H), 5.29 (s, 1H), |
| | | methyl-2,6- | | 4.43-3.98 (m, 5H), 3.78 (s, |
| | | diazaspiro[3.4]octan-2- | | 2H), 3.16 (s, 2H), 2.88 (s, |
| | | yl)quinazolin-2-amine | | 2H), 2.66 (s, 2H), 2.39 (d, |
| | | | | J = 34.1 Hz, 4H), 2.22 (s, |
| | | | | 4H), 1.73 (s, 2H), 1.22 (s, |
| | | | | 2H), 1.04 (s, 2H). |
| 82 | 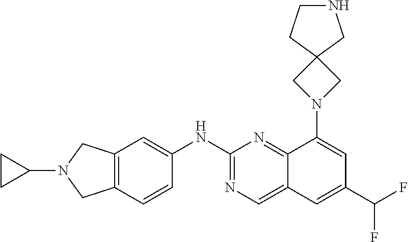 [SMILES: FC(F)c1cc(N2CC3(CCNC3)C2)c2nc(Nc3ccc4c(c3)CN(C3CC3)C4)ncc2c1] | N-(2- cyclopropylisoindolin-5- yl)-6-(difluoromethyl)-8- (2,6-diazaspiro[3.4]octan- 2-yl)quinazolin-2-amine | 463.3 | ¹H NMR (400 MHz, DMSO-d₆) δ (ppm) 9.85 (s, 1H), 9.28 (s, 1H), 7.73 (s, 1H), 7.51-7.46 (m, 2H), 7.22-7.18 (m, 1H), 7.08-6.92 (m, 1H), 6.71 (d, J = 1.6 Hz, 1H), 4.18 (d, J = 8.3 Hz, 2H), 4.11 (d, J = 8.3 Hz, 2H), 3.99 (s, 2H), 3.94 (s, 2H), 3.23 (t, J = 7.2 Hz, 3H), 2.28 (t, J = 7.2 Hz, 2H), 2.09-2.03 (m, 1H), 1.24 (s, 2H), 0.51-0.45 (m, 2H), 0.44-0.39 (m, 2H). |
| 83 | | 6-(difluoromethyl)-8-(2- | 521.3 | ¹H NMR (400 MHz, |
| | | methyl-2,6- | | DMSO-d₆) δ (ppm) 9.07 |
| | | diazaspiro[3.4]octan-6-yl)- | | (s, 1H), 7.54 (s, 1H), 7.29 |
| | | N-(1-((1- | | (d, J = 1.9 Hz, 1H), 6.98 (t, |
| | | methylcyclopropyl) | | J = 56.2 Hz, 1H), 6.72 (s, |
| | | sulfonyl)piperidin-4- | | 1H), 4.01 (s, 3H), 3.75 (d, |
| | | yl)quinazolin-2-amine | | J = 12.6 Hz, 2H), 3.52 (s, |
| | | | | 2H), 3.20-3.03 (m, 6H), |
| | | | | 2.24 (s, 3H), 2.13-2.00 (m, |
| | | | | 4H), 1.61-1.47 (m, 2H), |
| | | | | 1.43 (s, 3H), 1.21-1.13 (m, |
| | | | | 2H), 0.88-0.79 (m, 2H). |
| 84 | | N-(1-((cyclobutylmethyl) | 521.3 | ¹H-NMR (400 MHz, |
| | | sulfonyl)piperidin-4-yl)-6- | | DMSO-d₆) δ (ppm) 9.08 |
| | | (difluoromethyl)-8-(2,6- | | (s, 1H), 7.54 (s, 1H), 7.31 |
| | | diazaspiro[3.4]octan-6- | | (s, 1H), 6.99 (t, J = 56.1 |
| | | yl)quinazolin-2-amine | | Hz, 1H), 6.74 (s, 1H), |
| | | | | 4.10-3.50 (m, 12H), 3.16 |
| | | | | (d, J = 7.4 Hz, 2H), 2.96 (t, |
| | | | | J = 12.0 Hz, 2H), |
| | | | | 2.79-2.63 (m, 1H), |
| | | | | 2.24-2.16 (m, 2H), |
| | | | | 2.14-1.98 (m, 4H), |
| | | | | 1.94-1.76 (m, 4H), |
| | | | | 1.68-1.51 (m, 2H). |
| 85 | | N-(1-((cyclopropylmethyl) | 507.3 | ¹H NMR (400 MHz, |
| | | sulfonyl)piperidin-4-yl)-6- | | DMSO-d6) δ (ppm) 9.07 |
| | | (difluoromethyl)-8-(2,6- | | (s, 1H), 7.55 (s, 1H), 7.29 |
| | | diazaspiro[3.4]octan-2- | | (s, 1H), 7.12-6.85 (m, 1H), |
| | | yl)quinazolin-2-amine | | 6.55 (d, J = 6.9 Hz, 1H), |
| | | | | 4.12 (d, J = 9.9 Hz, 4H), |
| | | | | 3.87 (s, 1H), 3.65 (d, |
| | | | | J = 12.4 Hz, 2H), 3.47 (s, |
| | | | | 1H), 3.08-2.90 (m, 6H), |
| | | | | 2.87-2.79 (m, 1H), |
| | | | | 2.17-1.91 (m, 4H), |
| | | | | 1.63-1.49 (m, 2H), 1.23 (s, |
| | | | | 1H), 1.06-0.96 (m, 1H), |
| | | | | 0.63-0.58 (m, 2H), |
| | | | | 0.39-0.33 (m, 2H). |
| 86 | | N-(1-((cyclobutylmethyl) | 521.3 | ¹H-NMR (400 MHz, |
| | | sulfonyl)piperidin-4-yl)-6- | | Chloroform-d) δ (ppm) |
| | | (difluoromethyl)-8-(2,6- | | 8.99-8.88 (m, 1H), |
| | | diazaspiro[3.4]octan-2- | | 7.23-7.14 (m, 1H), |
| | | yl)quinazolin-2-amine | | 6.84-6.48 (m, 2H), |
| | | | | 5.38-5.20 (m, 1H), |
| | | | | 4.28-4.14 (m, 3H), 4.05 (s, |
| | | | | 1H), 3.80-3.71 (m, 2H), |
| | | | | 3.26-3.15 (m, 2H), |
| | | | | 3.11-2.96 (m, 6H), |
| | | | | 2.92-2.80 (m, 1H), |
| | | | | 2.29-2.18 (m, 4H), |
| | | | | 2.15-2.09 (m, 2H), |
| | | | | 2.05-1.82 (m, 5H), |
| | | | | 1.77-1.63 (m, 2H). |
| 87 | 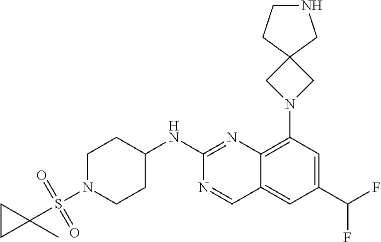 [SMILES: CC1(S(=O)(=O)N2CCC(Nc3ncc4cc(C(F)F)cc(N5CC6(CCNC6)C5)c4n3)CC2)CC1] | 6-(difluoromethyl)-N-(1- ((1-methylcyclopropyl) sulfonyl)piperidin-4-yl)-8- (2,6-diazaspiro[3.4]octan- 2-yl)quinazolin-2-amine | 507.3 | ¹H NMR (400 MHz, DMSO-d₆) δ (ppm) 9.08 (s, 1H), 7.58 (s, 1H), 7.33-7.27 (m, 1H), 6.98 (t, J = 56.1 Hz, 1H), 6.54 (d, J = 1.7 Hz, 1H), 4.12 (s, 4H), 3.91 (s, 1H), 3.75-3.68 (m, 2H), 3.48 (s, 1H), 3.15-3.05 (m, 2H), 3.01 (s, 2H), 2.85 (t, J = 6.9 Hz, 2H), 2.05-1.93 (m, 4H), 1.61-1.49 (m, 2H), 1.43 (s, 3H), 1.20-1.14 (m, 2H), 0.87-0.81 (m, 2H). |
| 88 | | 6-(difluoromethyl)-N-(1- | 543.3 | ¹H-NMR (400 MHz, |
| | | ((1-(difluoromethyl) | | DMSO-d₆) δ (ppm) 9.07 |
| | | cyclopropyl)sulfonyl) | | (s, 1H), 7.61-7.47 (m, 1H), |
| | | piperidin-4-yl)-8-(2,6- | | 7.38-7.23 (m, 1H), |
| | | diazaspiro[3.4]octan-6- | | 7.13-6.80 (m, 1H), 6.73 (s, |
| | | yl)quinazolin-2-amine | | 1H), 6.59-6.16 (m, 1H), |
| | | | | 4.01-3.89 (m, 3H), |
| | | | | 3.85-3.71 (m, 4H), |
| | | | | 3.64-3.50 (m, 4H), |
| | | | | 3.10-3.01 (m, 3H), |
| | | | | 2.17-2.09 (m, 2H), |
| | | | | 2.08-2.00 (m, 2H), |
| | | | | 1.62-1.51 (m, 2H), |
| | | | | 1.42-1.37 (m, 2H), |
| | | | | 1.32-1.26 (m, 2H). |
| 89 | | 6-(difluoromethyl)-8-(2- | 510.3 | ¹H NMR (400 MHz, |
| | | (2-methoxyethyl)-2- | | Chloroform-d) δ 8.98 (s, |
| | | azaspiro[3.3]heptan-6-yl)- | | 1H), 7.66 (d, J = 2.0 Hz, |
| | | N-(1- | | 1H), 7.58 (s, 1H), 6.72 (t, |
| | | (methylsulfonyl)piperidin- | | J = 56.4 Hz, 1H), 5.39 (d, |
| | | 4-yl)quinazolin-2-amine | | J = 7.3 Hz, 1H), 4.16-3.94 |
| | | | | (m, 2H), 3.87-3.76 (m, |
| | | | | 2H), 3.66-3.60 (m, 2H), |
| | | | | 3.46 (t, J = 5.4 Hz, 2H), |
| | | | | 3.36 (s, 5H), 3.13-3.01 (m, |
| | | | | 2H), 2.89 (s, 3H), |
| | | | | 2.79-2.64 (m, 4H), |
| | | | | 2.42-2.24 (m, 4H), |
| | | | | 1.83-1.67 (m, 2H). |
| 90 | | N-(1- | 542.3 | ¹H NMR (400 MHz, |
| | | (cyclopropylsulfonyl) | | Chloroform-d) δ (ppm) |
| | | piperidin-4-yl)-8-(2-(2,2- | | 8.90 (s, 1H), 7.84-7.48 (m, |
| | | difluoroethyl)-2- | | 2H), 6.92-6.58 (m, 1H), |
| | | azaspiro[3.3]heptan-6-yl)- | | 6.03-5.58 (m, 1H), 5.31 (s, |
| | | 6-(difluoromethyl) | | 1H), 4.18-3.95 (m, 2H), |
| | | quinazolin-2-amine | | 3.93-3.73 (m, 2H), |
| | | | | 3.66-3.51 (m, 1H), |
| | | | | 3.41-3.31 (m, 1H), |
| | | | | 3.22-3.00 (m, 2H), |
| | | | | 2.96-2.66 (m, 3H), |
| | | | | 2.45-2.10 (m, 3H), |
| | | | | 1.84-1.50 (m, 5H), |
| | | | | 1.35-1.17 (m, 3H), |
| | | | | 1.10-0.86 (m, 3H). |
| 91 | | 6-(difluoromethyl)-N-(1- | 542.3 | ¹H NMR (DMSO-d₆, 499 |
| | | ((1-(difluoromethyl) | | MHz) δ 9.19 (br s, 1H), |
| | | cyclopropyl)sulfonyl) | | 7.89 (s, 1H), 7.8-7.8 (m, |
| | | piperidin-4-yl)-8-(6- | | 1H), 7.64 (br d, 1H, |
| | | azaspiro[3.4]octan-2- | | J = 16.7 Hz), 7.0-7.2 (m, |
| | | yl)quinazolin-2-amine | | 1H), 6.2-6.5 (m, 1H), 5.76 |
| | | | | (s, 1H), 4.9-5.0 (m, 1H), |
| | | | | 3.9-4.1 (m, 2H), 3.7-3.8 |
| | | | | (m, 2H), 3.2-3.3 (m, 1H), |
| | | | | 3.1-3.2 (m, 1H), 3.0-3.1 |
| | | | | (m, 2H), 2.6-2.7 (m, 1H), |
| | | | | 2.5-2.6 (m, 1H), 2.44 (dt, |
| | | | | 1H, J = 2.6, 8.8 Hz), |
| | | | | 2.3-2.4 (m, 1H), 2.27 (br d, |
| | | | | 1H, J = 6.3 Hz), 2.14 (br t, |
| | | | | 1H, J = 7.0 Hz), 2.0-2.1 |
| | | | | (m, 1H), 2.0-2.0 (m, 1H), |
| | | | | 1.9-1.9 (m, 2H), 1.5-1.7 |
| | | | | (m, 2H), 1.41 (br s, 3H). |
| 92 | 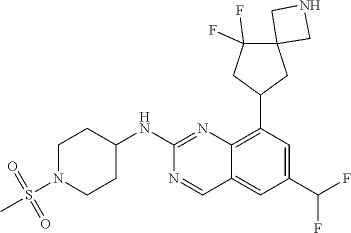 [SMILES: CS(=O)(=O)N1CCC(Nc2ncc3cc(C(F)F)cc(C4CC(F)(F)C5(CNC5)C4)c3n2)CC1] | 8-(8,8-difluoro-2,6- diazaspiro[3.4]octan-6-yl)- 6-(difluoromethyl)-N-(1- (methylsulfonyl)piperidin- 4-yl)quinazolin-2-amine | 503.2 | |
| 93 | | 6-(difluoromethyl)-N-(1- | 543.2 | ¹H-NMR (400 MHz, |
| | | ((1-(difluoromethyl) | | DMSO-d₆) δ (ppm) 9.08 |
| | | cyclopropyl)sulfonyl) | | (s, 1H), 7.58 (s, 1H), |
| | | piperidin-4-yl)-8-(2,6- | | 7.36-7.24 (m, 1H), |
| | | diazaspiro[3.4]octan-2- | | 7.20-6.79 (m, 1H), |
| | | yl)quinazolin-2-amine | | 6.64-6.17 (m, 2H), |
| | | | | 4.20-4.05 (m, 4H), 3.89 (s, |
| | | | | 1H), 3.78-3.65 (m, 2H), |
| | | | | 3.47 (s, 1H), 3.11-3.02 (m, |
| | | | | 2H), 2.97 (s, 1H), |
| | | | | 2.89-2.78 (m, 1H), |
| | | | | 2.17-2.09 (m, 1H), |
| | | | | 2.07-1.91 (m, 4H), |
| | | | | 1.63-1.50 (m, 2H), |
| | | | | 1.47-1.20 (m, 5H). |
| 94 | | 8-(5,5-difluoro-2,7- | 517.2 | |
| | | diazaspiro[3.5]nonan-2- | | |
| | | yl)-6-(difluoromethyl)-N- | | |
| | | (1-(methylsulfonyl) | | |
| | | piperidin-4-yl)quinazolin- | | |
| | | 2-amine | | |
| 95 | 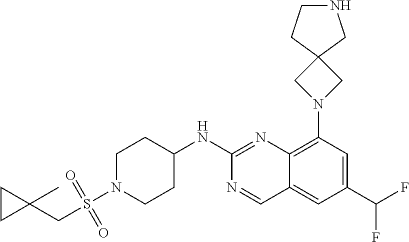 [SMILES: CC1(CS(=O)(=O)N2CCC(Nc3ncc4cc(C(F)F)cc(N5CC6(CCNC6)C5)c4n3)CC2)CC1] | 6-(difluoromethyl)-N-(1- (((1-methylcyclopropyl) methyl)sulfonyl)piperidin- 4-yl)-8-(2,6- diazaspiro[3.4]octan-2- yl)quinazolin-2-amine | 521.2 | ¹H-NMR (400 MHz, Chloroform-d) δ (ppm) 8.93 (s, 1H), 7.20 (s, 1H), 6.89-6.43 (m, 2H), 5.44-5.16 (m, 1H), 4.29-4.14 (m, 4H), 4.09-3.94 (m, 1H), 3.87-3.68 (m, 2H), 3.24 (s, 2H), 3.17-2.99 (m, 4H), 2.88 (s, 2H), 2.77-2.64 (m, 1H), 2.26-2.03 (m, 4H), 1.80-1.65 (m, 2H), 1.32 (s, 3H), 0.67-0.61 (m, 2H), 0.58-0.50 (m, 2H). |
| 96 | | 6-(difluoromethyl)-N-(1- | 535.2 | ¹H-NMR (400 MHz, |
| | | ((2-(1- | | Chloroform-d) δ (ppm) |
| | | methylcyclopropyl)ethyl) | | 8.93 (s, 1H), 7.20 (s, 1H), |
| | | sulfonyl)piperidin-4-yl)-8- | | 6.84-6.48 (m, 2H), |
| | | (2,6-diazaspiro[3.4]octan- | | 5.31-5.17 (m, 1H), |
| | | 2-yl)quinazolin-2-amine | | 4.30-4.14 (m, 4H), 4.05 (s, |
| | | | | 1H), 3.77 (d, J = 12.2 Hz, |
| | | | | 2H), 3.24 (s, 2H), |
| | | | | 3.16-3.00 (m, 6H), |
| | | | | 2.29-2.18 (m, 3H), |
| | | | | 2.16-2.11 (m, 3H), |
| | | | | 1.79-1.72 (m, 3H), 1.07 (s, |
| | | | | 3H), 0.41-4.30 (m, 4H). |
| 97 | | 6-(difluoromethyl)-8-(2,6- | 598.2 | ¹H NMR (400 MHz, |
| | | diazaspiro[3.4]octan-2-yl)- | | DMSO-d₆) δ (ppm) 9.15 |
| | | N-(1-((6- | | (d, J = 2.2 Hz, 1H), 9.05 (s, |
| | | (trifluoromethyl)pyridin- | | 1H), 8.52-8.45 (m, 1H), |
| | | 3-yl)sulfonyl)piperidin-4- | | 8.21 (d, J = 8.3 Hz, 1H), |
| | | yl)quinazolin-2-amine | | 7.53 (s, 1H), 7.28 (s, 1H), |
| | | | | 7.14-6.79 (m, 1H), |
| | | | | 6.55-6.50 (m, 1H), |
| | | | | 4.10-4.00 (m, 4H), 3.79 (s, |
| | | | | 1H), 3.69-3.58 (m, 2H), |
| | | | | 3.44-3.40 (m, 1H), 2.94 (s, |
| | | | | 1H), 2.84-2.64 (m, 3H), |
| | | | | 2.54 (s, 1H), 2.11-1.87 (m, |
| | | | | 5H), 1.66-1.56 (m, 2H). |
| 98 | | N-(1- | 509.3 | ¹H NMR (400 MHz, |
| | | (cyclopropylsulfonyl) | | DMSO-d₆) δ (ppm) 9.09 |
| | | piperidin-4-yl)-6- | | (s, 1H), 7.59 (s, 1H), 7.31 |
| | | (difluoromethyl)-8-(5-oxa- | | (d, J = 1.8 Hz, 1H), |
| | | 2,8-diazaspiro[3.5]nonan- | | 7.14-6.83 (m, 1H), 6.59 (d, |
| | | 2-yl)quinazolin-2-amine | | J = 1.7 Hz, 1H), 4.21 (d, |
| | | | | J = 8.8 Hz, 2H), 4.02-3.83 |
| | | | | (m, 3H), 3.69-3.58 (m, |
| | | | | 4H), 3.07-2.95 (m, 4H), |
| | | | | 2.73 (s, 2H), 2.65-.54 (m, |
| | | | | 1H), 2.10-1.99 (m, 2H), |
| | | | | 1.67-1.53 (m, 2H), 1.24 (s, |
| | | | | 1H), 1.05-0.90 (m, 4H). |
| 99 | | N-(1- | 416.3 | ¹H NMR (DMSO-d₆, 499 |
| | | (methylsulfonyl)piperidin- | | MHz) δ 9.08 (s, 1H), 7.63 |
| | | 4-yl)-8-(6- | | (br d, 1H, J = 8.2 Hz), 7.57 |
| | | azaspiro[3.4]octan-2- | | (br s, 1H), 7.4-7.5 (m, 1H), |
| | | yl)quinazolin-2-amine | | 7.20 (t, 1H, J = 7.5 Hz), |
| | | | | 3.9-4.1 (m, 3H), 3.6-3.7 |
| | | | | (m, 2H), 3.06 (br s, 1H), |
| | | | | 2.8-3.0 (m, 6H), 2.6-2.7 |
| | | | | (m, 2H), 2.3-2.5 (m, 2H), |
| | | | | 2.0-2.2 (m, 4H), 1.9-1.9 |
| | | | | (m, 1H), 1.73 (br t, 1H, |
| | | | | J = 7.1 Hz), 1.23 (s, 1H), |
| | | | | 1.2-1.3 (m, 1H). |
| 100 | 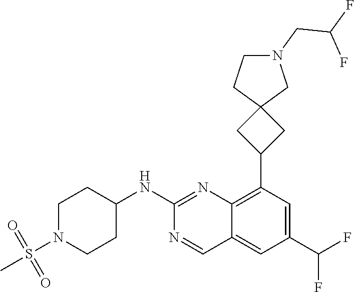 [SMILES: CS(=O)(=O)N1CCC(Nc2ncc3cc(C(F)F)cc(C4CC5(CCN(CC(F)F)C5)C4)c3n2)CC1] | 8-(6-(2,2-difluoroethyl)-6- azaspiro[3.4]octan-2-yl)- 6-(difluoromethyl)-N-(1- (methylsulfonyl)piperidin- 4-yl)quinazolin-2-amine | 530.4 | ¹H-NMR (400 MHz, Chloroform-d) δ (ppm) 9.12-8.92 (m, 1H), 7.78-7.58 (m, 2H), 6.96-6.52 (m, 1H), 6.12-5.70 (m, 1H), 5.53-5.25 (m, 1H), 4.29-4.05 (m, 2H), 3.97-3.70 (m, 2H), 3.15-2.96 (m, 3H), 2.95-2.79 (m, 7H), 2.78-2.69 (m, 2H), 2.62-2.48 (m, 2H), 2.28-2.14 (m, 3H), 1.97-1.55 (m, 4H). |
| 101 | | 6-(difluoromethyl)-N-(1- | 562.3 | ¹H NMR (400 MHz, |
| | | (methylsulfonyl)piperidin- | | Chloroform-d) δ 8.98 (s, |
| | | 4-yl)-8-(6-(3,3,3- | | 1H), 7.72-7.53 (m, 2H), |
| | | trifluoropropyl)-6- | | 6.72 (t, J = 56.4 Hz, 1H), |
| | | azaspiro[3.4]octan-2- | | 5.30 (d, J = 7.1 Hz, 1H), |
| | | yl)quinazolin-2-amine | | 4.2-4.02 (m, 2H), 3.78 (dd, |
| | | | | J = 10.9, 6.0 Hz, 2H), |
| | | | | 3.09-2.94 (m, 2H), 2.85 (s, |
| | | | | 3H), 2.76-2.65 (m, 4H), |
| | | | | 2.62 (s, 2H), 2.57-2.47 (m, |
| | | | | 2H), 2.40-2.23 (m, 6H), |
| | | | | 2.22-2.15 (m, 2H), |
| | | | | 1.81-1.69 (m, 2H) |
| 102 | | 6-(difluoromethyl)-N-(1- | 522.4 | ¹H NMR (400 MHz, |
| | | (methylsulfonyl)piperidin- | | Chloroform-d) δ 9.04-8.91 |
| | | 4-yl)-8-(6-(oxetan-3-yl)-6- | | (m, 1H), 7.71-7.50 (m, |
| | | azaspiro[3.4]octan-2- | | 2H), 6.71 (t, J = 59.2 Hz, |
| | | yl)quinazolin-2-amine | | 1H), 5.35 (s, 1H), |
| | | | | 4.75-4.57 (m, 3H), |
| | | | | 4.21-3.93 (m, 2H), |
| | | | | 3.85-3.62 (m, 2H), |
| | | | | 3.08-2.76 (m, 4H), |
| | | | | 2.74-2.44 (m, 5H), |
| | | | | 2.36-1.95 (m, 6H), |
| | | | | 1.84-1.54 (m, 2H), 1.26 (s, |
| | | | | 3H), 0.88 (s, 1H) |
| 103 | 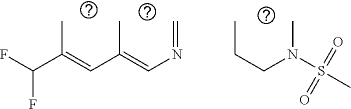 | 6-(difluoromethyl)-8-(6- (2-fluoro-2- methylpropyl)-6- azaspiro[3.4]octan-2-yl)- N-(1- (methylsulfonyl)piperidin- 4-yl)quinazolin-2-amine | 540.4 | ¹H-NMR (400 MHz, Chloroform-d) δ (ppm) 9.08-8.78 (m, 1H), 7.74-7.46 (m, 2H), 6.97-6.47 (m, 1H), 5.46-5.11 (m, 1H), 4.22-3.94 (m, 2H), 3.90-3.64 (m, 2H), 3.05-2.92 (m, 2H), 2.91-2.66 (m, 7H), 2.65-2.55 (m, 2H), 2.54-2.44 (m, 2H), 2.35-2.03 (m, 6H), 1.81-1.74 (m, 1H), 1.52-1.02 (m, 7H). |
| 104 | | N-(1- | 511.3 | ¹H NMR (400 MHz, |
| | | (cyclopropylsulfonyl) | | DMSO-d₆) δ (ppm) 9.10 |
| | | piperidin-4-yl)-6- | | (s, 1H), 7.60 (s, 1H), 7.33 |
| | | (difluoromethyl)-8-(8- | | (d, J = 1.8 Hz, 1H), 6.99 (t, |
| | | fluoro-2,6- | | J = 56.1 Hz, 1H), 6.59 (s, |
| | | diazaspiro[3.4]octan-2- | | 1H), 5.39-5.14 (m, 1H), |
| | | yl)quinazolin-2-amine | | 4.41 (d, J = 8.5 Hz, 1H), |
| | | | | 4.12-4.01 (m, 2H), |
| | | | | 3.99-3.77 (m, 2H), 3.64 (d, |
| | | | | J = 12.3 Hz, 2H), 3.48 (s, |
| | | | | 1H), 3.22-2.91 (m, 6H), |
| | | | | 2.63-2.54 (m, 1H), 2.04 (d, |
| | | | | J = 12.5 Hz, 2H), |
| | | | | 1.65-1.52 (m, 2H), |
| | | | | 1.04-0.89 (m, 4H). |
| 105 | 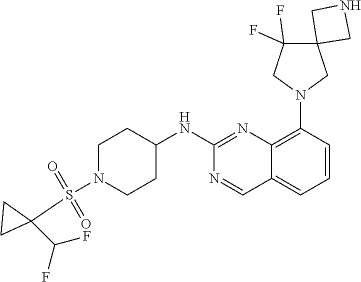 [SMILES: O=S(=O)(N1CCC(Nc2ncc3cccc(N4CC(F)(F)C5(CNC5)C4)c3n2)CC1)C1(C(F)F)CC1] | 8-(9,9-difluoro-2,6- diazaspiro[3.5]nonan-2- yl)-N-(1-((1- (difluoromethyl) cyclopropyl)sulfonyl) piperidin-4-yl)quinazolin- 2-amine | 543.2 | ¹H NMR (DMSO-d₆, 499 MHz) δ 9.00 (s, 1H), 7.3-7.4 (m, 1H), 7.0-7.2 (m, 2H), 6.54 (dd, 1H, J = 1.4, 7.4 Hz), 6.35 (t, 1H, J = 55.2 Hz), 4.15 (br d, 2H, J = 8.5 Hz), 3.97 (br d, 2H, J = 8.2 Hz), 3.8-3.9 (m, 1H), 3.72 (br d, 2H, J = 12.6 Hz), 3.0-3.1 (m, 4H), 2.80 (br s, 2H), 2.0-2.1 (m, 1H), 1.8-1.9 (m, 2H), 1.5-1.6 (m, 3H), 1.40 (br d, 3H, J = 1.6 Hz), 1.28 (br d, 2H, J = 2.7 Hz) |
| 106 | | 3-(2-(6-(difluoromethyl)- | 560.4 | ¹H NMR (400 MHz, |
| | | 2-((1- | | Methanol-d₄) δ 9.05 (d, |
| | | (methylsulfonyl)piperidin- | | J = 2.1 Hz, 1H), 7.78 (s, |
| | | 4-yl)amino)quinazolin-8- | | 1H), 7.66 (s, 1H), 6.85 (t, |
| | | yl)-6-azaspiro[3.4]octan- | | J = 56.3 Hz, 1H), 5.36 (t, |
| | | 6-yl)-2,2-difluoropropan- | | J = 4.8 Hz, 1H), 4.18-4.04 |
| | | 1-ol | | (m, 2H), 3.83-3.70 (m, |
| | | | | 4H), 3.08-2.97 (m, 4H), |
| | | | | 2.91 (s, 3H), 2.85 (t, |
| | | | | J = 6.8 Hz, 1H), 2.75 (d, |
| | | | | J = 3.6 Hz, 2H), 2.64-2.50 |
| | | | | (m, 2H), 2.28-2.18 (m, |
| | | | | 5H), 2.04 (d, J = 6.1 Hz, |
| | | | | 1H), 1.90 (d, J = 2.6 Hz, |
| | | | | 1H), 1.80-1.72 (m, 2H), |
| | | | | 1.62 (s, 1H). |
| 107 | | 8-(6- | 431.2 | |
| | | methoxyspiro[3.3]heptan- | | |
| | | 2-yl)-N-(1- | | |
| | | (methylsulfonyl)piperidin- | | |
| | | 4-yl)quinazolin-2-amine | | |
| 108 | | 6-(difluoromethyl)-8-(6- | 481.2 | |
| | | methoxyspiro[3.3]heptan- | | |
| | | 2-yl)-N-(1- | | |
| | | (methylsulfonyl)piperidin- | | |
| | | 4-yl)quinazolin-2-amine | | |
| 109 | | 8-(8,8-difluoro-2,6- | 453.3 | ¹H NMR (400 MHz, |
| | | diazaspiro[3.4]octan-6-yl)- | | DMSO-d₆) δ (ppm) 9.05 |
| | | N-(1- | | (s, 1H), 7.50-7.41 (m, 1H), |
| | | (methylsulfonyl)piperidin- | | 7.26-7.24 (m, 1H), |
| | | 4-yl)quinazolin-2-amine | | 7.12-7.08 (m, 1H), |
| | | | | 6.86-6.84 (m, 1H), |
| | | | | 4.10-4.03 (m, 4H), |
| | | | | 3.90-3.88 (m, 3H), |
| | | | | 3.61-3.52 (m, 4H), |
| | | | | 2.93-2.88 (m, 5H), |
| | | | | 2.07-2.04 (m, 2H), |
| | | | | 1.62-1.60 (m, 2H), |
| | | | | 1.22-1.20 (m, 1H). |
| 110 | | N-(1- | 432.2 | ¹H NMR (400 MHz, |
| | | (methylsulfonyl)piperidin- | | Chloroform-d) δ (ppm) |
| | | 4-yl)-8-(5-oxa-8- | | 9.17 (s, 1H), 7.62-7.48 (m, |
| | | azaspiro[3.5]nonan-2- | | 2H), 7.30-7.14 (m, 2H), |
| | | yl)quinazolin-2-amine | | 5.43-5.26 (m, 1H), 4.09 (s, |
| | | | | 1H), 3.91-3.70 (m, 5H), |
| | | | | 3.17 (s, 2H), 3.09-2.93 (m, |
| | | | | 4H), 2.90-2.81 (m, 3H), |
| | | | | 2.80-2.70 (m, 2H), |
| | | | | 2.38-2.20 (m, 4H), |
| | | | | 1.83-1.69 (m, 2H). |
| 111 | 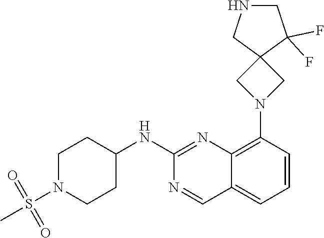 [SMILES: CS(=O)(=O)N1CCC(Nc2ncc3cccc(N4CC5(CNCC5(F)F)C4)c3n2)CC1] | 8-(8,8-difluoro-2,6- diazaspiro[3.4]octan-2-yl)- N-(1- (methylsulfonyl)piperidin- 4-yl)quinazolin-2-amine | 453.2 | ¹H-NMR (400 MHz, DMSO-d₆) δ (ppm) 9.01 (s, 1H), 7.31 (s, 1H), 7.14 (dd, J = 8.0, 1.3 Hz, 1H), 7.07 (t, J = 7.7 Hz, 1H), 6.60 (d, J = 7.4 Hz, 1H), 4.25 (d, J = 8.4 Hz, 2H), 4.02 (d, J = 8.5 Hz, 2H), 3.94-3.78 (m, 1H), 3.69-3.56 (m, 3H), 3.26 (s, 2H), 3.16 (t, J = 14.2 Hz, 2H), 2.97-2.80 (m, 5H), 2.11-1.97 (m, 2H), 1.68-1.53 (m, 2H). |
| 112 | | 3-(6-(6-(difluoromethyl)- | 546.4 | ¹H NMR (400 MHz, |
| | | 2-((1- | | Methanol-d₄) δ (ppm) 9.06 |
| | | (methylsulfonyl)piperidin- | | (s, 1H), 7.79 (s, 1H), 7.65 |
| | | 4-yl)amino)quinazolin-8- | | (s, 1H), 7.25 (d, J = 8.0 Hz, |
| | | yl)-2-azaspiro[3.3]heptan- | | 1H), 6.85 (t, J = 56.3 Hz, |
| | | 2-yl)-2,2-difluoropropan- | | 1H), 4.24 (s, 1H), 3.86 (s, |
| | | 1-ol | | 2H), 3.18-2.93 (m, 9H), |
| | | | | 2.92 (s, 3H), 2.72 (d, |
| | | | | J = 7.2 Hz, 3H), 2.22-2.16 |
| | | | | (m, 8H). |
| 113 | | 1-(difluoromethyl)-3-(6- | 477.1 | ¹H NMR (400 MHz, |
| | | (difluoromethyl)-2-((1- | | DMSO-d₆) δ (ppm) 9.20 |
| | | (methylsulfonyl)piperidin- | | (s, 1H), 7.91 (s, 1H), |
| | | 4-yl)amino)quinazolin-8- | | 7.82-7.64 (m, 2H), |
| | | yl)cyclobutan-1-ol | | 7.28-6.90 (m, 1H), |
| | | | | 6.26-5.80 (m, 2H), |
| | | | | 4.01-3.89 (m, 1H), |
| | | | | 3.83-3.70 (m, 1H), 3.60 (d, |
| | | | | J = 11.5 Hz, 2H), |
| | | | | 2.80-2.74 (m, 6H), 2.40 (s, |
| | | | | 1H), 2.32-2.20 (m, 2H), |
| | | | | 2.13-2.04 (m, 2H), |
| | | | | 1.68-1.56 (m, 2H). |
| 114 | | 1-cyclopropyl-3-(2-((1- | 485.2 | ¹H NMR (400 MHz, |
| | | (methylsulfonyl)piperidin- | | Chloroform-d) δ 9.02 (s, |
| | | 4-yl)amino)-6- | | 1H), 7.83 (s, 1H), 7.71 (s, |
| | | (trifluoromethyl) | | 1H), 5.44 (s, 1H), 4.13 (s, |
| | | quinazolin-8- | | 1H), 3.83 (d, J = 11.9 Hz, |
| | | yl)cyclobutan-1-ol | | 2H), 3.64-3.60 (m, 1H), |
| | | | | 3.02 (t, J = 11.8 Hz, 1H), |
| | | | | 2.86 (d, J = 3.2 Hz, 3H), |
| | | | | 2.61-2.48 (m, 2H), |
| | | | | 2.44-2.22 (m, 3H), |
| | | | | 1.86-1.71 (m, 2H), 1.59 (s, |
| | | | | 2H), 1.39-1.19 (m, 2H), |
| | | | | 0.67-0.60 (m, 2H), |
| | | | | 0.53-0.50 (m, 2H). |
| 115 | | 8-(8,8-difluoro-2,6- | 503.2 | ¹H-NMR (400 MHz, |
| | | diazaspiro[3.4]octan-2-yl)- | | DMSO-d₆) δ (ppm) 9.11 |
| | | 6-(difluoromethyl)-N-(1- | | (s, 1H), 7.62 (s, 1H), 7.36 |
| | | (methylsulfonyl)piperidin- | | (d, J = 1.9 Hz, 1H), 7.00 (t, |
| | | 4-yl)quinazolin-2-amine | | J = 56.1 Hz, 1H), 6.63 (s, |
| | | | | 1H), 4.30 (d, J = 8.6 Hz, |
| | | | | 2H), 4.08 (d, J = 8.6 Hz, |
| | | | | 2H), 3.86 (s, 1H), |
| | | | | 3.67-3.52 (m, 2H), |
| | | | | 3.34-3.28 (m, 3H), 3.17 (t, |
| | | | | J = 14.2 Hz, 2H), |
| | | | | 2.97-2.81 (m, 5H), |
| | | | | 2.14-1.93 (m, 2H), |
| | | | | 1.71-1.53 (m, 2H). |
| 116 | | 8-(8,8-difluoro-2,6- | 521.2 | ¹H NMR (400 MHz, |
| | | diazaspiro[3.4]octan-2-yl)- | | DMSO-d₆) δ 9.15 (s, 1H), |
| | | N-(1- | | 7.77 (s, 1H), 7.54 (d, |
| | | (methylsulfonyl)piperidin- | | J = 1.9 Hz, 1H), 6.63 (s, |
| | | 4-yl)-6-(trifluoromethyl) | | 1H), 4.34 (d, J = 8.7 Hz, |
| | | quinazolin-2-amine | | 2H), 4.11 (d, J = 8.7 Hz, |
| | | | | 2H), 3.86 (s, 1H), |
| | | | | 3.68-3.52 (m, 3H), 3.26 (s, |
| | | | | 2H), 3.17 (t, J = 14.2 Hz, |
| | | | | 2H), 2.97-2.81 (m, 5H), |
| | | | | 2.12-1.95 (m, 2H), |
| | | | | 1.70-1.53 (m, 2H). |
| 117 | | (3S,4R)-4-((8-(8,8- | 442.1 | ¹H NMR (400 MHz, |
| | | difluoro-2,6- | | DMSO-d₆) δ 8.97 (s, 1H), |
| | | diazaspiro[3.4]octan-6-yl)- | | 7.34-7.27 (m, 1H), |
| | | 6-(difluoromethyl) | | 6.94-6.88 (m, 1H), |
| | | quinazolin-2- | | 6.82-6.53 (m, 1H), 5.58 (s, |
| | | yl)amino)tetrahydro-2H- | | 1H), 4.56-4.42 (m, 1H), |
| | | pyran-3-ol | | 4.35-4.21 (m, 3H), |
| | | | | 4.17-4.11 (m, 3H), |
| | | | | 4.06-4.03 (m, 1H), |
| | | | | 3.94-3.92 (m, 2H), |
| | | | | 3.67-3.46 (m, 5H), |
| | | | | 2.42-2.25 (m, 1H), |
| | | | | 1.78-1.60 (m, 1H), |
| | | | | 1.35-1.22 (m, 1H). |
| 118 | | (3R,4R)-4-((8-(8,8- | 519.1 | ¹H NMR (400 MHz, |
| | | difluoro-2,6- | | DMSO-d₆) δ 9.14 (s, 1H), |
| | | diazaspiro[3.4]octan-6-yl)- | | 7.59 (s, 1H), 7.48 (s, 1H), |
| | | 6-(difluoromethyl) | | 7.20-6.83 (m, 2H), |
| | | quinazolin-2-yl) amino)-1- | | 4.37-3.91 (m, 7H), |
| | | (methylsulfonyl)piperidin- | | 3.90-3.48 (m, 7H), |
| | | 3-ol | | 3.00-2.86 (m, 4H), |
| | | | | 2.80-2.68 (m, 1H), |
| | | | | 2.29-2.13 (m, 1H), |
| | | | | 1.65-1.44 (m, 1H). |
| 119 | | 3-(difluoromethyl)-1-(2- | 494.1 | ¹H NMR (400 MHz, |
| | | ((1-((1-methyl-1H- | | DMSO-d₆) δ (ppm) 8.99 |
| | | pyrazol-4- | | (s, 1H), 8.32 (s, 1H), 7.77 |
| | | yl)sulfonyl)piperidin-4- | | (s, 1H), 7.32 (s, 1H), |
| | | yl)amino)quinazolin-8- | | 7.16-7.01 (m, 2H), 6.58 (d, |
| | | yl)azetidin-3-ol | | J = 7.4 Hz, 1H), 6.45 (s, |
| | | | | 1H), 6.34-5.97 (m, 1H), |
| | | | | 4.32 (d, J = 9.1 Hz, 2H), |
| | | | | 3.94 (s, 5H), 3.70 (s, 1H), |
| | | | | 3.49 (d, J = 11.4 Hz, 2H), |
| | | | | 2.48-2.43 (m, 1H), |
| | | | | 2.10-1.96 (m, 2H), |
| | | | | 1.71-1.56 (m, 2H), 1.24 (s, |
| | | | | 1H). |
| 120 | | 1-(difluoromethyl)-3-(2- | 427.1 | ¹H NMR (400 MHz, |
| | | ((1- | | DMSO-d₆) δ 9.09 (s, 1H), |
| | | (methylsulfonyl)piperidin- | | 7.67-7.59 (m, 2H), 7.44 (s, |
| | | 4-yl)amino)quinazolin-8- | | 1H), 7.26-7.17 (m, 1H), |
| | | yl)cyclobutan-1-ol | | 6.27-5.81 (m, 2H), |
| | | | | 4.03-3.88 (m, 1H), |
| | | | | 3.82-3.71 (m, 1H), |
| | | | | 3.67-3.54 (m, 2H), |
| | | | | 2.97-2.86 (m, 5H), |
| | | | | 2.83-2.74 (m, 2H), |
| | | | | 2.40-2.33 (m, 1H), |
| | | | | 2.29-2.19 (m, 1H), |
| | | | | 2.12-2.00 (m, 2H), |
| | | | | 1.67-1.55 (m, 2H). |
| 121 | | (1-(difluoromethyl)-3-(2- | 441.2 | ¹H NMR (400 MHz, |
| | | ((1- | | Chloroform-d) δ 8.99 (s, |
| | | methanesulfonylpiperidin- | | 1H), 7.65 (s, 1H), 7.60 (s, |
| | | 4-yl)amino)quinazolin-8- | | 2H), 5.43-5.15 (m, 2H), |
| | | yl)cyclobutyl]methanol | | 4.35 (s, 1H), 4.19-3.94 (m, |
| | | | | 2H), 3.82 (s, 2H), |
| | | | | 3.61-3.45 (s, 1H), 3.00 (t, |
| | | | | J = 11.1 Hz, 1H), 2.85 (s, |
| | | | | 3H), 2.68-2.44 (m, 1H), |
| | | | | 2.27 (d, J = 11.0 Hz, 2H), |
| | | | | 2.06-1.90 (m, 1H), 1.75 (s, |
| | | | | 2H), 1.56 (s, 2H), |
| | | | | 1.39-1.23 (m, 2H) |
| 122 | 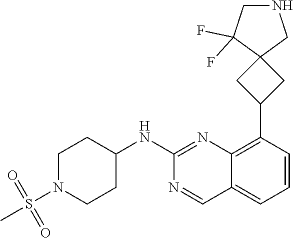 [SMILES: CS(=O)(=O)N1CCC(Nc2ncc3cccc(C4CC5(CNCC5(F)F)C4)c3n2)CC1] | 8-(8,8-difluoro-6- azaspiro[3.4]octan-2-yl)- N-(1- (methylsulfonyl)piperidin- 4-yl)quinazolin-2-amine | 452.2 | ¹H NMR (400 MHz, DMSO-d₆) δ 9.10 (s, 1H), 7.68-7.55 (m, 2H), 7.47 (s, 1H), 7.25-7.18 (m, 1H), 4.16-3.85 (m, 2H), 3.65-3.55 (m, 2H), 3.25-3.04 (m, 3H), 2.96-2.84 (m, 6H), 2.80-2.71 (m, 2H), 2.39-2.25 (m, 2H), 2.19-2.05 (m, 3H), 1.70-1.56 (m, 2H). |

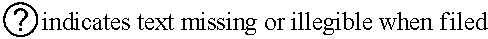

In some embodiments, compounds of the disclosure are below in Table 2.

TABLE 2

| Cmpd | | |
|---|---|---|
| No. | Structure | Name |
| 123 | | N-(5-(6-ethyl-2,6- |
| | | diazaspiro[3.3]heptan-2- |
| | | yl)pyridin-2-yl)-8-(6- |
| | | methoxy-2- |
| | | azaspiro[3.3]heptan-2- |
| | | yl)quinazolin-2-amine |
| 124 | | 2-(2-((5-(6-ethyl-2,6- |
| | | diazaspiro[3.3]heptan-2- |
| | | yl)pyridin-2- |
| | | yl)amino)quinazolin-8-yl)- |
| | | 2-azaspiro[3.3]heptane-6- |
| | | carbonitrile |
| 125 | | 8-(6-methyl-2,6- |
| | | diazaspiro[3.3]heptan-2- |
| | | yl)-2-((2- |
| | | (methylsulfonyl)isoindolin- |
| | | 5-yl)amino)quinazoline- |
| | | 6-carbonitrile |
| 126 | | 8-(1,1-difluoro-5- |
| | | azaspiro[2.4]heptan-5-yl)- |
| | | 2-((1-(2-methyl-2- |
| | | azaspiro[3.3]heptan-6-yl)- |
| | | 1H-pyrazol-4- |
| | | yl)amino)quinazoline-6- |
| | | carbonitrile |
| 127 | | 8-(1,1-difluoro-5- |
| | | azaspiro[2.4]heptan-5-yl)- |
| | | 2-((4-(6-methyl-2,6- |
| | | diazaspiro[3.3]heptan-2- |
| | | yl)phenyl)amino)quinazoli |
| | | ne-6-carbonitrile |
| 128 | | 8-(2,2-dioxido-2-thia-6- |
| | | azaspiro[3.3]heptan-6-yl)- |
| | | 2-((4-(6-methyl-2,6- |
| | | diazaspiro[3.3]heptan-2- |
| | | yl)phenyl)amino)quinazoli |
| | | ne-6-carbonitrile |
| 129 | | 7-methyl-2-(2-((1- |
| | | (methylsulfonyl)piperidin- |
| | | 4-yl)amino)quinazolin-8- |
| | | yl)-2,7- |
| | | diazaspiro[4.5]decan-8- |
| | | one |
| 130 | | 1-(7-(2-((4-(4- |
| | | methylpiperazin-1- |
| | | yl)phenyl)amino)quinazoli |
| | | n-8-yl)-2,7- |
| | | diazaspiro[4.4]nonan-2- |
| | | yl)ethan-1-one |
| 131 | | 1-(7-(6-(difluoromethyl)- |
| | | 2-((1- |
| | | (methylsulfonyl)piperidin- |
| | | 4-yl)amino)quinazolin-8- |
| | | yl)-2,7- |
| | | diazaspiro[4.4]nonan-2- |
| | | yl)ethan-1-one |
| 132 | | 1-(7-(6-(difluoromethyl)- |
| | | 2-((4-(4-methylpiperazin-1- |
| | | yl)phenyl)amino)quinazoli |
| | | n-8-yl)-2,7- |
| | | diazaspiro[4.4]nonan-2- |
| | | yl)ethan-1-one |
| 133 | | 1-(7-(6-(difluoromethyl)- |
| | | 2-((5-(6-ethyl-2,6- |
| | | diazaspiro[3.3]heptan-2- |
| | | yl)pyridin-2- |
| | | yl)amino)quinazolin-8-yl)- |
| | | 2,7-diazaspiro[4.4]nonan- |
| | | 2-yl)ethan-1-one |
| 134 | | 1-(7-(6-(difluoromethyl)- |
| | | 2-((1-isopropyl-1H- |
| | | pyrazol-4- |
| | | yl)amino)quinazolin-8-yl)- |
| | | 2,7-diazaspiro [4.4]nonan- |
| | | 2-yl)ethan-1-one |
| 135 | | 2-(2-((5-(6-ethyl-2,6- |
| | | diazaspiro[3.3]heptan-2- |
| | | yl)pyridin-2- |
| | | yl)amino)quinazolin-8-yl)- |
| | | 2-azaspiro[3.3]heptan-6-ol |
| 136 | | 8-(6,6-difluoro-2- |
| | | azaspiro[3.3]heptan-2-yl)- |
| | | N-(5-(6-ethyl-2,6- |
| | | diazaspiro[3.3]heptan-2- |
| | | yl)pyridin-2-yl)quinazolin- |
| | | 2-amine |
| 137 | | 6-(difluoromethyl)-N- |
| | | ((1R,5S,6s)-3-methyl-3- |
| | | azabicyclo[3.1.0]hexan-6- |
| | | yl)-8-(7-(methylsulfonyl)- |
| | | 2,7-diazaspiro[4.4]nonan- |
| | | 2-yl)quinazolin-2-amine |
| 138 | | 8-(1,1-difluoro-5- |
| | | azaspiro[2.4]heptan-5-yl)- |
| | | 6-(difluoromethyl)-N- |
| | | ((1R,5S,6s)-3-methyl-3- |
| | | azabicyclo[3.1.0]hexan-6- |
| | | yl)quinazolin-2-amine |
| 139 | | 6-(6-(difluoromethyl)-2- |
| | | (((1R,5S,6s)-3-methyl-3- |
| | | azabicyclo[3.1.0]hexan-6- |
| | | yl)amino)quinazolin-8-yl)- |
| | | 2-thia-6- |
| | | azaspiro[3.4]octane 2,2- |
| | | dioxide |
| 140 | | 6-(difluoromethyl)-8-(2- |
| | | methyl-2- |
| | | azaspiro[3.3]heptan-6-yl)- |
| | | N-(1- |
| | | (methylsulfonyl)piperidin- |
| | | 4-yl)quinazolin-2-amine |
| 141 | | 6-(difluoromethyl)-N-(1- |
| | | (methylsulfonyl)piperidin- |
| | | 4-yl)-8-(6- |
| | | azaspiro[3.4]octan-2- |
| | | yl)quinazolin-2-amine |
| 142 | | 6-(difluoromethyl)-8-(2- |
| | | (2-methoxyethyl)-2- |
| | | azaspiro[3.3]heptan-6-yl)- |
| | | N-(1- |
| | | (methylsulfonyl)piperidin- |
| | | 4-yl)quinazolin-2-amine |
| 143 | | 8-(2-cyclopropyl-2- |
| | | azaspiro[3.3]heptan-6-yl)- |
| | | 6-(difluoromethyl)-N-(1- |
| | | (methylsulfonyl)piperidin- |
| | | 4-yl)quinazolin-2-amine |
| 144 | | N-(1- |
| | | (cyclopropylsulfonyl)piper |
| | | idin-4-yl)-8-(2-(2,2- |
| | | difluoroethyl)-2- |
| | | azaspiro[3.3]heptan-6-yl)- |
| | | 6-(difluoromethyl)quinazolin- |
| | | 2-amine |
| 145 | | N-(1- |
| | | (cyclopropylsulfonyl)piperidin- |
| | | 4-yl)-6- |
| | | (difluoromethyl)-8-(6- |
| | | azaspiro[3.4]octan-2- |
| | | yl)quinazolin-2-amine |
| 146 | | 6-(difluoromethyl)-8-(6- |
| | | methyl-6- |
| | | azaspiro[3.4]octan-2-yl)- |
| | | N-(1-((1- |
| | | methylcyclopropyl)sulfonyl)piperidin- |
| | | 4-yl)quinazolin-2-amine |
| 147 | | 6-(difluoromethyl)-N-(1- |
| | | ((1- |
| | | methylcyclopropyl)sulfonyl)piperidin- |
| | | 4-yl)-8-(6- |
| | | azaspiro[3.4]octan-2- |
| | | yl)quinazolin-2-amine |
| 148 | | 6-(difluoromethyl)-N-(1- |
| | | ((1- |
| | | methylcyclopropyl)sulfonyl)piperidin- |
| | | 4-yl)-8-(7- |
| | | azaspiro[3.5]nonan-2- |
| | | yl)quinazolin-2-amine |
| 149 | | 6-(difluoromethyl)-N-(1- |
| | | ((1- |
| | | methylcyclopropyl)sulfonyl)piperidin- |
| | | 4-yl)-8-(2- |
| | | azaspiro[3.4]octan-6- |
| | | yl)quinazolin-2-amine |
| 150 | | 6-(difluoromethyl)-8-(2- |
| | | methyl-2- |
| | | azaspiro[3.4]octan-6-yl)- |
| | | N-(1-((1- |
| | | methylcyclopropyl)sulfonyl)piperidin- |
| | | 4-yl)quinazolin-2-amine |

### Biochemical Assays

### CDK1/Cyclin B1 ADP-Glo Kinase Assay

The purpose of CDK1/Cyclin B1 assay is to evaluate the inhibition (% inhibition and IC₅₀ values) of small molecule inhibitors by using a Luminescent based ADP-Glo assay. CDK1/Cyclin B1 catalyzes the production of ADP from ATP. ADP-Glo assay monitors ADP producing biochemical reactions. ADP-Glo is performed in 2 steps upon completion of kinase reaction: a combined termination of kinase reaction and depletion of remaining ATP in the first step, and conversion of generated ADP to ATP and the newly produced ATP to light output using luciferase/luciferin reaction in the second step. The luminescent signal generated is proportional to the ADP concentration produced and is correlated with the kinase activity. CDK1/Cyclin B1 was purchased from Carna (Cat 04-102). Typical reaction solutions (10 uL final reaction volume) contained 2% DMSO (f inhibitor), 10 mM MgCl2, 1 mM EGTA, 0.05% BSA, 2 mM DTT, 80 uM ATP (ATP Km=78.6 uM), 0.01% Brig-35, 0.75 uM substrate, and 4.917 nM CDK1/Cyclin Bl enzyme complex in 50 mM HEPES buffer at pH 7.5. The assay was initiated with the addition of ATP-containing substrate solution, following a 30-minute pre-incubation of enzyme and inhibitor at room temperature in the reaction mixture. The reaction was stopped after 90 minutes at room temperature by the addition of 10 uL of ADP-GLO Reagent. After a 90 minute incubation, 20 uL of Kinase Detection Reagent was added. Samples were incubated for 40 minutes, after which plate well luminescence was measured on a Envision microplate reader. The IC₅₀ determinations were made from a plot of the fractional velocity as a function of inhibitor concentration fit to the 4 parameters IC₅₀ equation.

### CDK2/Cyclin E1 Full Length ADP-Glo Kinase Assay

The purpose of CDK2/Cyclin E1 assay is to evaluate the inhibition (% inhibition and IC₅₀ values) of small molecule inhibitors by using a Luminescent based ADP-Glo assay. CDK2/Cyclin E1 full length catalyzes the production of ADP from ATP. ADP-Glo assay monitors ADP producing biochemical reactions. ADP-Glo is performed in 2 steps upon completion of kinase reaction: a combined termination of kinase reaction and depletion of remaining ATP in the first step, and conversion of generated ADP to ATP and the newly produced ATP to light output using luciferase/luciferin reaction in the second step. The luminescent signal generated is proportional to the ADP concentration produced and is correlated with the kinase activity. CDK2/Cyclin E1 was purchased from Eurofins (Cat 14-475M). Typical reaction solutions (10 uL final reaction volume) contained 2% DMSO (f inhibitor), 10 mM MgCI2, 1 mM EGTA, 0.05% BSA, 2 mM DTT, 20 uM ATP (ATP Km=64.78 uM), 0.01% Brig-35, 0.75 uM substrate, and 0.328 nM wild-type full length CDK2/Cyclin E1 enzyme complex in 50 mM HEPES buffer at pH 7.5. The assay was initiated with the addition of ATP-containing substrate solution, following a 30-minute pre-incubation of enzyme and inhibitor at room temperature in the reaction mixture. The reaction was stopped after 90 minutes at room temperature by the addition of 10 uL of ADP-GLO Reagent. After a 90 minute incubation, 20 uL of Kinase Detection Reagent was added. Samples were incubated for 40 minutes, after which plate well luminescence was measured on a Envision microplate reader. The IC₅₀ determinations were made from a plot of the fractional velocity as a function of inhibitor concentration fit to the 4 parameters IC₅₀ equation.

### CDK4/Cyclin D1 CHEF Assay

The purpose of CDK4/Cyclin Dl assay is to evaluate the inhibition (% inhibition and IC₅₀ values) of small molecule inhibitors by using a Chelation-Enhance Fluorescence (CHEF) assay. In a CHEF assay, phosphorylation of a peptide substrate results in proportional increase in fluorescence. CHEF kinase assay use peptide substrates containing a synthetic alpha-amino acid with a side chain bearing an 8-hydroxyquinoline derivative (sulfonamido-oxide, Sox). Upon phosphorylation of a nearby serine, threonine or tyrosine and in the presence of Mg(II), the spectral properties of the Sox residue are altered, emitting 485 nm wavelength light when excited with a 360 nm wavelength light source. CDK4/Cyclin D1 catalyzes the phosphoryl transfer to the SOX-labeled substrate peptide AQT0258 from Assayquant Technologies. Typical reaction solutions contained 2% DMSO (+/−inhibitor), 10 mM MgCl2, 1 mM DTT, 200 uM ATP (ATP Km=195.2 uM), 0.012% Brig-35, 10 uM AQT0258 peptide, 0.02% BSA, 1% Glycerol, 0.55 mM EGTA, 2.5 nM CDK4/Cyclin D1 in 54 mM HEPES buffer at pH 7.5. The reaction was initiated with the addition of substrate solution, following a 30-minute pre-incubation of enzyme and inhibitor at 22° C. in the reaction mix. Reactions were allowed to proceed for 3 hrs at 22° C., followed by fluorescence read of the reaction. The IC₅₀ determinations were made from a plot of the fractional velocity as a function of inhibitor concentration fit to the 4 parameters IC₅₀ equation.

### CDK4/Cyclin D1 Mobility Shift Assay (MSA)

The purpose CDK4/Cyclin D1 assay is to evaluate the inhibition (% inhibition and IC₅₀ values) in the presence of small molecule inhibitors by using a fluorescence based microfluidic mobility shift assay. CDK4/Cyclin D1 catalyzes the production of ADP from ATP that accompanies the phosphoryl transfer to the substrate peptide 5-FAM-Dyrktide (5-FAM-RRRFRPASPLRGPPK) (Perkin Elmer Peptide 34). The mobility shift assay (MSA) electrophoretically separates the fluorescently labelled peptides (substrate and phosphorylated product) following the kinase reaction. Both substrate and product are measured, and the ratio of these values is used to generate % conversion of substrate to product by the LabChip EZ Reader. Typical reaction solutions contained 2% DMSO (+/−inhibitor), 10 mM MgCl2, 1 mM EGTA, 0.05% BSA, 2 mM DTT, 0.2 mM ATP, 0.01% Brig-35, 1.5 uM 5-FAM-Dyrktide, 2.5 nM CDK4/Cyclin D1 in 50 mM HEPES buffer at pH 7.5. The reaction was initiated with the addition of substrate solution, following a 30-minute pre-incubation of enzyme and inhibitor at 22° C. in the reaction mix. The reaction was stopped after 180 minutes by the addition of 75 uL of 500 mM EDTA and measured on a Perkin Elmer EZ reader instrument. IC₅₀ determinations were made from a plot of the fractional velocity as a function of inhibitor concentration fit to the 4 parameters IC₅₀ equation.

### CDK6/Cyclin D3 ADP-Glo Kinase Assay

The purpose of the CDK6/Cyclin D3 assay is to evaluate the inhibition (% inhibition and IC₅₀ values) in the presence of small molecule inhibitors by using a Luminescent based ADP-Glo assay. CDK6/Cyclin D3 catalyzes the production of ADP from ATP. ADP-Glo assay monitors ADP producing biochemical reactions. ADP-Glo is performed in 2 steps upon completion of kinase reaction: a combined termination of kinase reaction and depletion of remaining ATP in the first step, and conversion of generated ADP to ATP and the newly produced ATP to light output using luciferase/luciferin reaction in the second step. The luminescent signal generated is proportional to the ADP concentration produced and is correlated with the kinase activity. CDK6/Cyclin D3 was purchased from Carna. Typical reaction solutions (10 uL final reaction volume) contained 2% DMSO (f inhibitor), 10 mM MgCI2, 1 mM EGTA, 0.05% BSA, 2 mM DTT, 100 uM ATP (ATP Km=291.7 uM), 0.01% Brig-35, 0.75 uM substrate, and 5 nM wild-type CDK6/Cyclin D3 enzyme complex in 50 mM HEPES buffer at pH 7.5. The assay was initiated with the addition of ATP-containing substrate solution, following a 30-minute pre-incubation of enzyme and inhibitor at room temperature in the reaction mixture. The reaction was stopped after 90 minutes at room temperature by the addition of 10 uL of ADP-GLO Reagent. After a 90-minute incubation, 20 uL of Kinase Detection Reagent was added. Samples were incubated for 40 minutes, after which plate well luminescence was measured on a Envision microplate reader. The IC₅₀ determinations were made from a plot of the fractional velocity as a function of inhibitor concentration fit to the 4 parameters IC₅₀ equation.

### Cell Growth Inhibition

MCF-7 and OVCAR-3 cells were used to evaluate the anti-proliferation activity of the CDK inhibitors. MCF-7 (ATCC, HTB-22) cells are epithelial cells from a female patient with ER+ metastatic adenocarcinoma. OVCAR-3 (ATCC, HTB-161) cells were derived from malignant ascites of a patient with ovarian cancer and are known to have CCNE1 amplification. Both cell lines were maintained in RPMI media supplemented with 10% fetal bovine serum. For cell growth inhibition assay, CDK inhibitors in DMSO solution were dispensed with either Echo 655 (Beckman Coulter) or Tecan D300e (HP) into 384-well plates (Corning #3765) and the 384-well plates were UV-sterilized prior to the assay. The inhibitors were typically tested in the 10-10,000 nM concentration range with half-log serial dilutions. MCF-7 or OVCAR-3 (500 cells/30 μL/well) were added to each well using Multidrop Combi (ThermoFisher) using standard cassettes. The assay plates with cells were cultured at 37° C., 5% CO₂ for 6 days. At the end of the 6-day treatment, 30 μL of CellTiterGlo 2.0 (Promega) was added to each well and the luminescent signal was read using CLARIOstar plus (BMG). The percentage of cell growth inhibition (% CGI) was calculated using the following formula % CGI=100−100×luminescenceₛₐₘₚₗₑ/luminescence_{control}. The half maximal inhibitory concentration (IC50) was determined by nonlinear curve fitting (four parameters, variable slope).

Certain compounds of the disclosure have IC₅₀ values as in Table 3.

TABLE 3
All IC₅₀ values in Table 3 are reported as the following:

| | | | CDK4/ | CDK4/ | | | |
|---|---|---|---|---|---|---|---|
| | CDK1 | CDK2/ | CyclinD1 | CyclinD1 | CDK6/ | MCF-7 | OVCAR-3 |
| Cmpd | Cyclin B1 | CyclinE1 | MSA | AQT | CyclinD3 | Cell | Cell |
| # | IC₅₀ nM | IC₅₀ nM | IC₅₀ nM | IC₅₀ nM | IC₅₀ nM | IC₅₀ nM | IC₅₀ nM |
| 1 | ++++ | ++++ | ++++ | | ++++ | +++ | ++ |
| 2 | ++ | ++++ | ++++ | | ++++ | | |
| 3 | ++ | ++++ | ++++ | | ++++ | + | + |
| 4 | ++ | ++++ | ++++ | | ++++ | ++ | + |
| 5 | + | +++ | ++ | | ++ | | |
| 6 | + | ++++ | ++++ | | ++++ | + | + |
| 7 | + | ++++ | +++ | | ++ | | |
| 8 | + | +++ | ++ | | ++ | | |
| 9 | + | +++ | ++ | | +++ | | |
| 10 | +++ | ++++ | ++++ | | ++++ | + | + |
| 11 | + | ++ | ++ | | + | | |
| 12 | + | +++ | +++ | | ++ | | |
| 13 | + | ++++ | +++ | | ++ | | |
| 14 | +++ | ++++ | ++++ | | ++++ | + | + |
| 15 | +++ | ++++ | ++++ | | +++ | +++ | ++ |
| 16 | + | ++++ | ++ | | + | | |
| 17 | + | ++++ | +++ | | ++ | | |
| 18 | ++++ | ++++ | ++++ | | ++++ | ++ | ++ |
| 19 | +++ | ++++ | ++++ | | ++++ | ++ | ++ |
| 20 | + | ++++ | +++ | | ++ | | |
| 21 | + | +++ | +++ | | + | | |
| 22 | ++ | ++++ | ++++ | | ++++ | | |
| 23 | + | ++++ | ++++ | | ++++ | | |
| 24 | +++ | ++++ | ++++ | | ++++ | ++ | ++ |
| 25 | + | ++++ | ++++ | | +++ | | |
| 26 | ++ | ++++ | ++++ | | ++++ | + | + |
| 27 | ++ | ++++ | ++++ | | ++++ | + | + |
| 28 | + | ++++ | ++++ | | ++++ | | |
| 29 | + | +++ | ++ | | + | | |
| 30 | ++ | ++++ | ++++ | | +++ | | |
| 31 | +++ | ++++ | ++++ | | +++ | ++ | ++ |
| 32 | + | ++++ | +++ | | ++ | | |
| 33 | + | 5174.3 | ++++ | | ++++ | + | + |
| 34 | ++ | ++++ | ++++ | | +++ | | |
| 35 | +++ | ++++ | ++++ | | ++++ | ++ | + |
| 36 | ++ | ++++ | ++++ | | ++ | + | + |
| 37 | ++ | ++++ | ++++ | | ++++ | + | + |
| 38 | + | +++ | ++++ | | +++ | ++ | ++ |
| 39 | ++ | ++++ | ++++ | | ++ | + | + |
| 40 | ++ | +++ | +++ | | +++ | + | + |
| 41 | + | + | +++ | | ++ | + | + |
| 42 | + | + | ++++ | | +++ | +++ | + |
| 43 | + | + | +++ | | ++ | ++ | + |
| 44 | + | + | ++++ | | +++ | | + |
| 45 | ++ | ++++ | ++++ | | ++++ | + | + |
| 46 | + | + | ++++ | | +++ | ++ | + |
| 47 | + | + | +++ | | +++ | + | + |
| 48 | + | + | ++++ | | ++++ | ++ | + |
| 49 | + | + | ++++ | | +++ | | |
| 50 | +++ | ++++ | ++++ | | ++++ | ++ | + |
| 51 | + | ++ | ++++ | | ++++ | ++ | ++ |
| 52 | ++ | ++ | ++++ | | ++ | +++ | ++ |
| 53 | + | + | ++++ | | ++++ | ++ | + |
| 54 | + | + | ++++ | | +++ | + | + |
| 55 | + | ++ | +++ | | ++ | + | + |
| 56 | + | + | | +++ | + | + | + |
| 57 | + | ++++ | | ++++ | ++++ | ++ | ++ |
| 58 | ++ | ++++ | | ++++ | ++++ | ++ | ++ |
| 59 | + | ++ | | +++ | ++ | + | + |
| 60 | ++ | +++ | | ++++ | ++++ | ++ | ++ |
| 61 | ++ | ++++ | | +++ | ++ | + | + |
| 62 | +++ | ++++ | | ++++ | ++++ | ++++ | ++ |
| 63 | ++++ | ++++ | | ++++ | ++++ | ++ | ++ |
| 64 | + | ++ | | ++++ | + | ++ | + |
| 65 | + | ++++ | | ++++ | ++++ | ++ | ++ |
| 66 | ++ | ++++ | | ++++ | ++++ | ++ | ++ |
| 67 | ++++ | ++++ | | ++++ | ++++ | ++++ | ++++ |
| 68 | ++++ | ++++ | | ++++ | +++ | ++ | ++ |
| 69 | +++ | ++++ | | ++++ | ++++ | +++ | ++ |
| 70 | ++++ | ++++ | | ++++ | ++++ | +++ | +++ |
| 71 | + | +++ | | ++++ | ++ | ++ | ++ |
| 72 | ++++ | ++++ | | ++++ | ++++ | ++ | + |
| 73 | ++++ | ++++ | | ++++ | ++++ | +++ | ++ |
| 74 | ++++ | ++++ | | ++++ | ++++ | +++ | +++ |
| 75 | ++++ | ++++ | | ++++ | ++++ | ++ | ++ |
| 76 | ++ | ++++ | | ++++ | ++++ | ++ | ++ |
| 77 | ++++ | ++++ | | ++++ | ++++ | +++ | ++ |
| 78 | +++ | ++++ | | ++++ | ++++ | ++ | + |
| 79 | + | +++ | | ++++ | ++++ | ++ | ++ |
| 80 | ++++ | ++++ | | ++++ | ++++ | +++ | +++ |
| 81 | +++ | ++++ | | ++++ | ++++ | ++ | ++ |
| 82 | ++ | ++++ | | ++++ | ++++ | ++ | + |
| 83 | +++ | ++++ | | ++++ | ++++ | ++ | ++ |
| 84 | +++ | ++++ | | ++++ | ++++ | ++ | ++ |
| 85 | ++++ | ++++ | | ++++ | ++++ | +++ | +++ |
| 86 | ++++ | ++++ | | ++++ | ++++ | ++ | ++ |
| 87 | ++++ | ++++ | | ++++ | ++++ | ++ | +++ |
| 88 | ++++ | ++++ | | ++++ | ++++ | +++ | +++ |
| 89 | ++++ | ++++ | | ++++ | ++++ | +++ | ++++ |
| 90 | ++++ | ++++ | | ++++ | ++++ | ++ | +++ |
| 91 | ++++ | ++++ | | ++++ | ++++ | +++ | ++++ |
| 92 | ++++ | ++++ | | ++++ | ++++ | ++ | ++ |
| 93 | ++++ | ++++ | | ++++ | ++++ | +++ | ++++ |
| 94 | ++++ | ++++ | | ++++ | ++++ | ++ | ++ |
| 95 | ++++ | ++++ | | ++++ | ++++ | ++ | ++ |
| 96 | ++++ | ++++ | | ++++ | ++++ | ++ | ++ |
| 97 | ++++ | ++++ | | ++++ | ++++ | +++ | ++++ |
| 98 | ++++ | ++++ | | ++++ | ++++ | +++ | ++ |
| 99 | ++++ | ++++ | | ++++ | ++++ | ++ | +++ |
| 100 | ++++ | ++++ | | ++++ | ++++ | +++ | ++ |
| 101 | ++++ | ++++ | | ++++ | ++++ | +++ | ++ |
| 102 | ++++ | ++++ | | ++++ | ++++ | +++ | +++ |
| 103 | ++++ | ++++ | | ++++ | ++++ | +++ | ++ |
| 104 | ++++ | ++++ | | ++++ | ++++ | +++ | +++ |
| 105 | +++ | ++++ | | ++++ | +++ | + | + |
| 106 | ++++ | ++++ | | ++++ | ++++ | ++ | ++ |
| 107 | ++ | ++++ | | ++++ | ++++ | | |
| 108 | ++++ | ++++ | | ++++ | ++++ | | |
| 109 | ++++ | ++++ | | ++++ | ++++ | ++ | ++ |
| 110 | ++++ | ++++ | | ++++ | ++++ | ++ | ++ |
| 111 | ++ | ++++ | | ++++ | ++++ | ++ | ++ |
| 112 | ++++ | ++++ | | ++++ | ++++ | +++ | ++++ |
| 113 | ++++ | ++++ | | ++++ | ++++ | +++ | +++ |
| 114 | ++++ | ++++ | | ++++ | ++++ | + | + |
| 115 | ++++ | ++++ | | ++++ | ++++ | ++ | ++ |
| 116 | ++ | ++++ | | ++++ | ++++ | ++ | + |
| 117 | + | ++++ | | ++++ | ++++ | ++ | + |
| 118 | ++ | ++++ | | ++++ | ++++ | | + |
| 119 | ++++ | ++++ | | ++++ | ++++ | | ++ |
| 120 | ++++ | ++++ | | ++++ | ++++ | ++ | +++ |
| 121 | +++ | ++++ | | ++++ | ++++ | + | ++ |
| 122 | ++++ | ++++ | | ++++ | ++++ | | |

++++ = IC₅₀ < 200 nM;
+++ = 200 nM < IC₅₀ < 500 nM;
++ = 500 nM < IC₅₀ < 2000 nM ;
+ = IC₅₀ > 2000 nM

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

We claim:

1. A compound, or a pharmaceutically acceptable salt or solvate thereof, having the structure of Formula (I): 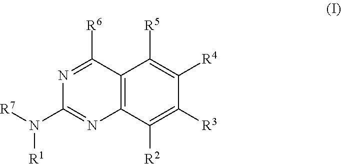
wherein,
R¹ is selected from optionally substituted pyrazole, optionally substituted indazole, optionally substituted tetrahydroisoquinoline, optionally substituted pyrrolpyrimidine, optionally substituted 2-pyridine, optionally substituted azabicyclo[3.1.0]hexane, optionally substituted indole, optionally substituted isoindole, and optionally substituted azetidine;
R² is selected from optionally substituted cycloalkyl and optionally substituted heterocycle;
each of R³, R⁴, R⁵, R⁶, is independently selected from hydrogen, halogen, —CN, optionally substituted C₁₋₄ alkyl, optionally substituted C₃₋₄ carbocycle, and optionally substituted 3- to 4-membered heterocycloalkyl;
R⁷ is selected from hydrogen and optionally substituted C₁₋₄ alkyl;
wherein if R¹ is an optionally substituted pyrazole, R² is not piperidine.

2. The compound, or a pharmaceutically acceptable salt or solvate thereof, of claim 1, wherein R¹ is selected from optionally substituted piperidine, optionally substituted indazole, optionally substituted tetrahydroisoquinoline, optionally substituted of pyrrolpyrimidine, optionally substituted tetrahydroisoquinoline, optionally substituted 2-pyridine, optionally substituted azabicyclo[3.1.0]hexane, optionally substituted indole, optionally substituted isoindole, and optionally substituted azetidine.

3. The compound, or a pharmaceutically acceptable salt or solvate thereof, of claim 1, wherein R¹ is selected from optionally substituted piperidine, optionally substituted 2-pyridine, optionally substituted azabicyclo[3.1.0]hexane, optionally substituted indole, optionally substituted isoindole, and optionally substituted azetidine.

4. The compound, or pharmaceutically acceptable salt thereof, of claim 1, wherein R² is 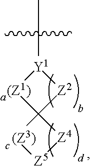
Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle; and
each R¹¹ is independently selected from hydrogen and optionally substituted C₁₋₄ alkyl.

5. The compound, or a pharmaceutically acceptable salt or solvate thereof, of claim 1 having the structure of one or more of the following Formula: 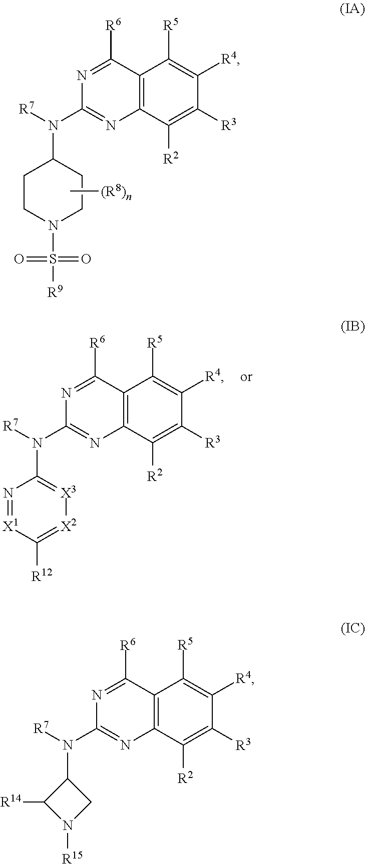
wherein,
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl;
n is selected from 0 to 9;
each of X¹, X², and X³ is independently selected from N and CR³;
R¹² is selected from hydrogen, halogen, —CN, —NO₂, optionally substituted alkyl, optionally substituted alkenyl, optionally substituted alkynyl, optionally substituted carbocycle, and optionally substituted heterocycle, or R² comes together with R¹³ to form an optionally substituted ring; and
each R¹³ is independently selected from hydrogen, halogen, —CN, and optionally substituted C₁₋₄ alkyl.
R¹⁴ is selected from hydrogen, halogen, —CN, optionally substituted C₁₋₄ alkyl, optionally substituted C₃₋₆ carbocycle, and optionally substituted 3- to 6-membered heterocycloalkyl, or R¹⁴ and R⁵ come together to form an optionally substituted heterocycle; and
R⁵ is selected from —S(O)₂R¹⁶—, optionally substituted C₁₋₄ alkyl, optionally substituted C₃₋₆ carbocycle, and optionally substituted 3- to 6-membered heterocycloalkyl.

6. The compound, or a pharmaceutically acceptable salt or solvate thereof, of claim 5, wherein R² is optionally substituted heterocycle.

7. The compound, or pharmaceutically acceptable salt thereof, of claim 1, wherein R² is selected from optionally substituted C₃₋₆ cycloalkyl, optionally substituted pyrrolidine, optionally substituted piperidine, optionally substituted pyrazole, optionally substituted azetidine, optionally substituted oxetane, and optionally substituted morpholine.

8. The compound, or pharmaceutically acceptable salt thereof, of claim 1, wherein R² is substituted with —CN, —SO₂R², —NR^{2a}, oxo, C₁₋₃ alkyl, C₁₋₃ hydroxyalkyl, C₃₋₆ cycloalkyl, C₁₋₃ alkylene-C₃₋₆ cycloalkyl, oxetane, or azetidine, wherein R^{2a} is selected from C₁₋₆ alkyl.

9. The compound, or a pharmaceutically acceptable salt or solvate thereof, of claim 1 having the structure of one or more of the following Formula: 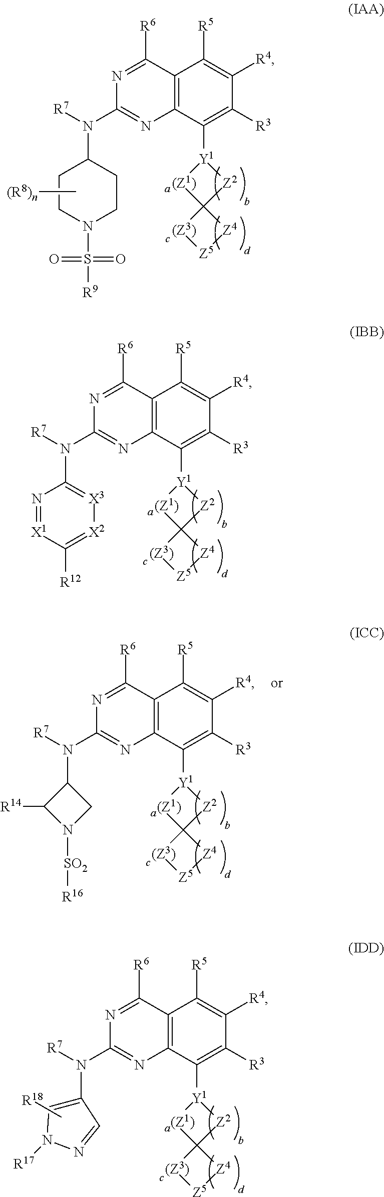
wherein,
Y¹ is selected from —N— and —CR¹⁰—;
each of Z¹, Z², Z³, Z⁴ and Z⁵ are independently selected from —C(R¹⁰)₂—, —C(O)—, —NR¹¹—, —N(C(O)R¹⁰)—, —NS(O₂)R¹¹, —O—, —S—, —S(O)—, and —S(O)₂—, wherein Z⁵ is additionally selected from a bond;
each of a, b, c, and d are independently selected from 1, 2, 3, and 4;
R⁸ is selected from halogen, —CN, and optionally substituted C₁₋₄ alkyl;
R⁹ is selected from optionally substituted C₁₋₄ alkyl, optionally substituted C₃₋₆ carbocycle, and 3- to 6-membered heterocycloalkyl;
n is selected from 0 to 9;
X¹, X², and X³ are each CH;
each R¹⁰ is independently selected from hydrogen, halogen, —CN, —OH, —O—C₁₋₄ alkyl, optionally substituted alkyl, optionally substituted cycloalkyl, and optionally substituted heterocycloalkyl, or two R¹⁰ substituents come together to form an optionally substituted heterocycle or an optionally substituted carbocycle, or R¹⁰ and R¹¹ substituents come together to form an optionally substituted heterocycle;
each R¹¹ is independently selected from hydrogen and optionally substituted C₁₋₄ alkyl;
R¹⁶ is selected from optionally substituted C₁₋₄ alkyl, optionally substituted C₃₋₆ carbocycle, and optionally substituted 3- to 6-membered heterocycloalkyl;
R¹⁷ is selected from optionally substituted C₁₋₄ alkyl, optionally substituted C₃₋₆ carbocycle, and optionally substituted 3- to 6-membered heterocycloalkyl, or R³ and R¹⁴ come together to form an optionally substituted heterocycle; and
R¹⁸ is selected from halogen, —CN, optionally substituted C₁₋₄ alkyl, optionally substituted C₃₋₆ carbocycle, and optionally substituted 3- to 6-membered heterocycloalkyl.

10. The compound, or pharmaceutically acceptable salt thereof, of claim 9, wherein Y¹ is —N—.

11. The compound, or pharmaceutically acceptable salt thereof, of claim 9, wherein Y¹ is —CR¹⁰—.

12. The compound, or pharmaceutically acceptable salt thereof, of claim 9, wherein each of Z¹, Z², Z³, Z⁴ and Z⁵ are independently selected from —C(R¹⁰)₂—, —NR¹¹—, —N(C(O)R¹⁰)—, —NS(O₂)R¹¹, —O—, and —S(O)₂—, wherein Z⁵ is additionally selected from a bond.

13. The compound, or pharmaceutically acceptable salt thereof, of claim 9, wherein each of a, b, c, and d are independently selected from 1, 2, and 3.

14. The compound, or pharmaceutically acceptable salt thereof, of claim 9, wherein each R¹⁰ is independently selected from hydrogen, halogen, —CN, —OH, —O—C₁₋₄ alkyl, optionally substituted C₁₋₃ alkyl, and optionally substituted C₃₋₆ cycloalkyl.

15. The compound, or pharmaceutically acceptable salt thereof, of claim 9, wherein each R¹¹ is independently selected from hydrogen and optionally substituted C₁₋₂ alkyl.

16. The compound, or pharmaceutically acceptable salt thereof, of claim 1, wherein R¹ is selected from optionally substituted azabicyclo[3.1.0]hexane, optionally substituted isoindole, and optionally substituted indole.

17. The compound, or pharmaceutically acceptable salt thereof, of claim 1, wherein R¹ is substituted with —SO₂R^{1a} or C₁₋₃ alkyl, wherein R^{1a} is selected from C₁₋₆ alkyl.

18. The compound, or pharmaceutically acceptable salt thereof, of claim 1, wherein R² is selected from optionally substituted heterocycle and optionally substituted cycloalkyl.

19. The compound, or pharmaceutically acceptable salt thereof, of claim 18, wherein R² is selected from optionally substituted azetidine, optionally substituted pyrrolidine, optionally substituted piperidine, optionally substituted piperazine, and optionally substituted morpholine.

20. The compound, or pharmaceutically acceptable salt thereof, of claim 1, wherein R² is substituted with halogen, —SO₂R^{2a}, —NR^{2a}, —C(O)CH₃, —CN, optionally substituted 3- to 6-membered hterocycloalkyl, optionally substituted C₃₋₅ carbocycle, oxo, and optionally substituted C₁₋₃ alkyl, wherein R² is selected from C₁₋₆ alkyl.

21. The compound or pharmaceutically acceptable salt according to claim 1, wherein R² is selected from

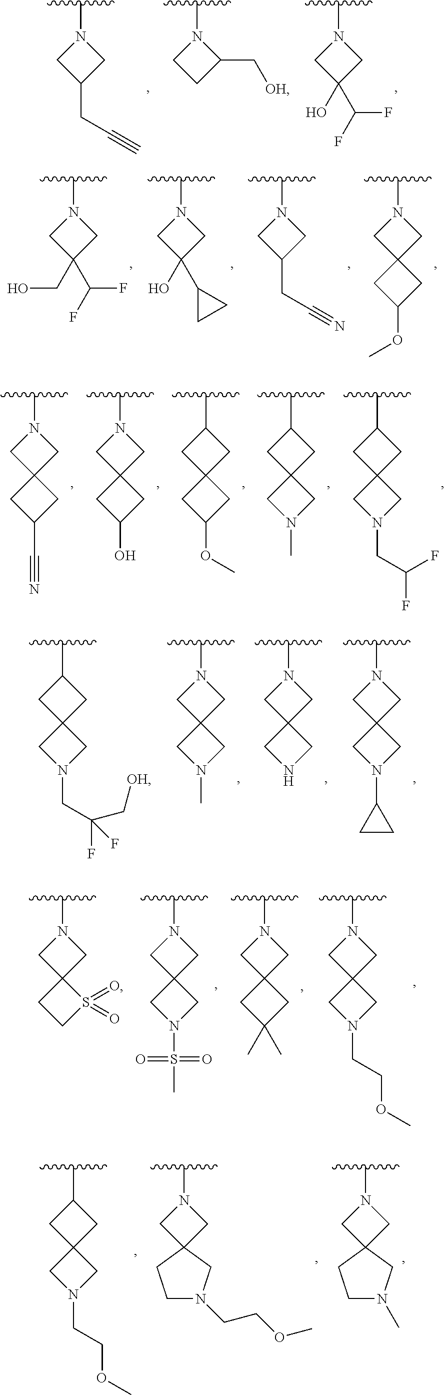

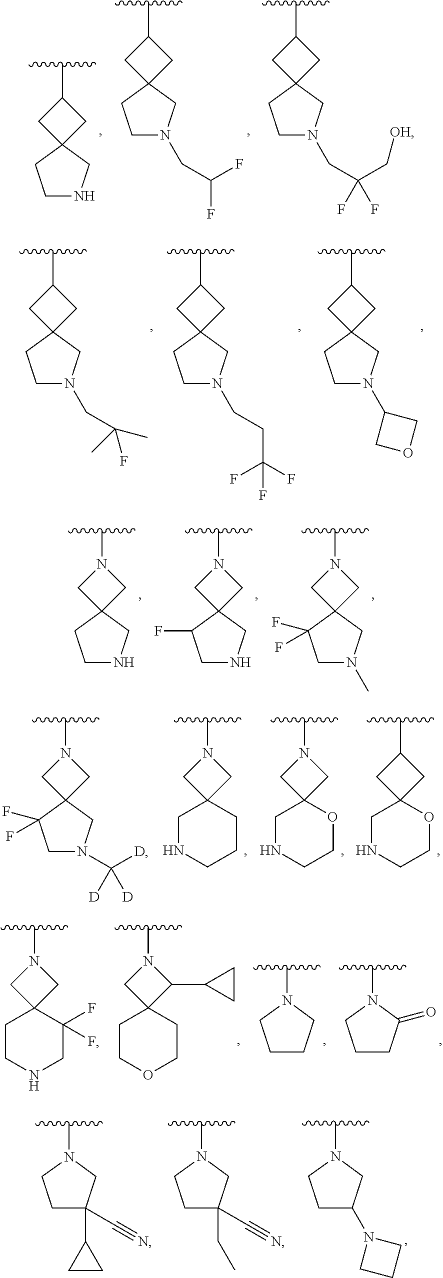

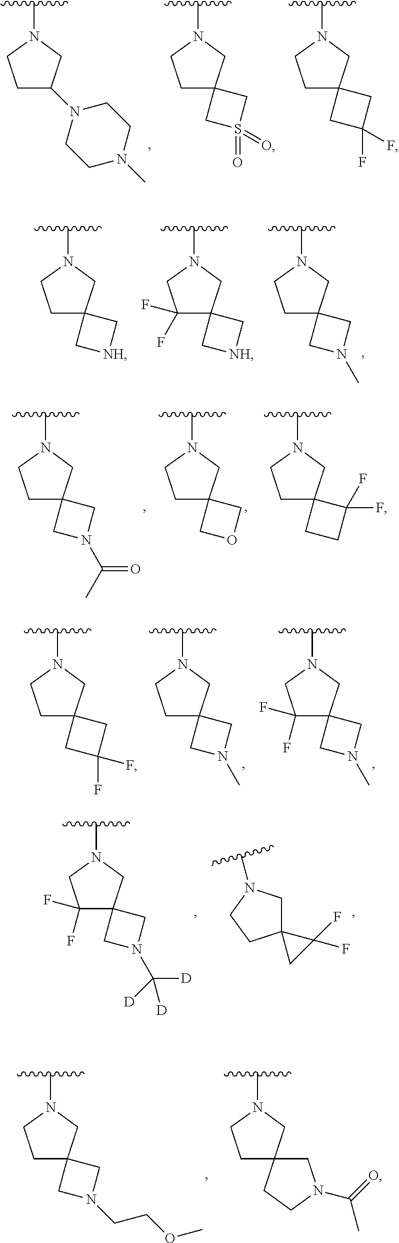

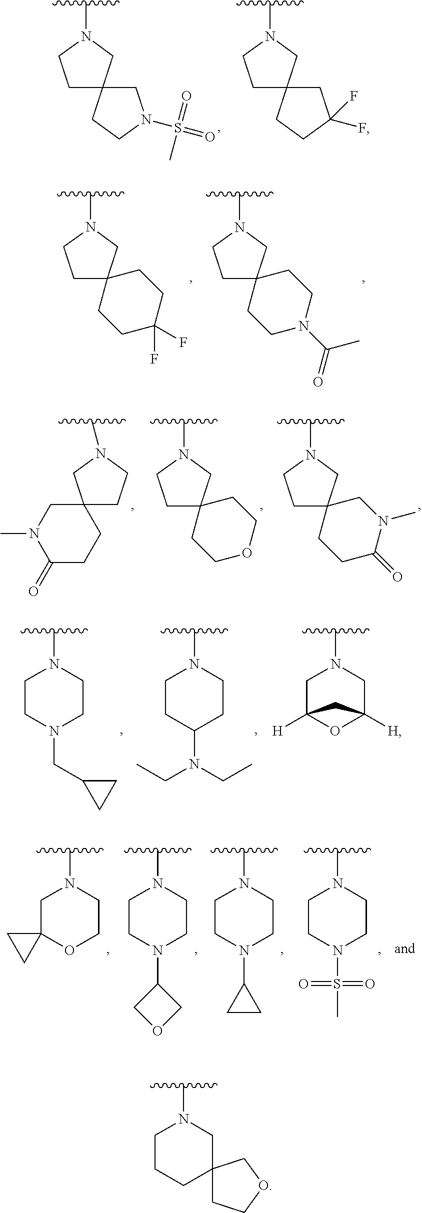

22. The compound, or pharmaceutically acceptable salt thereof, of claim 21, wherein R² is selected from

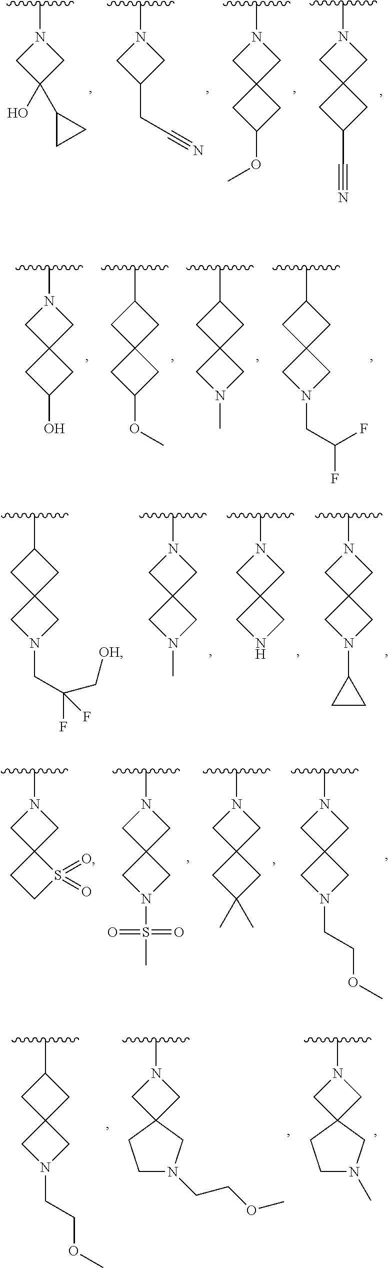

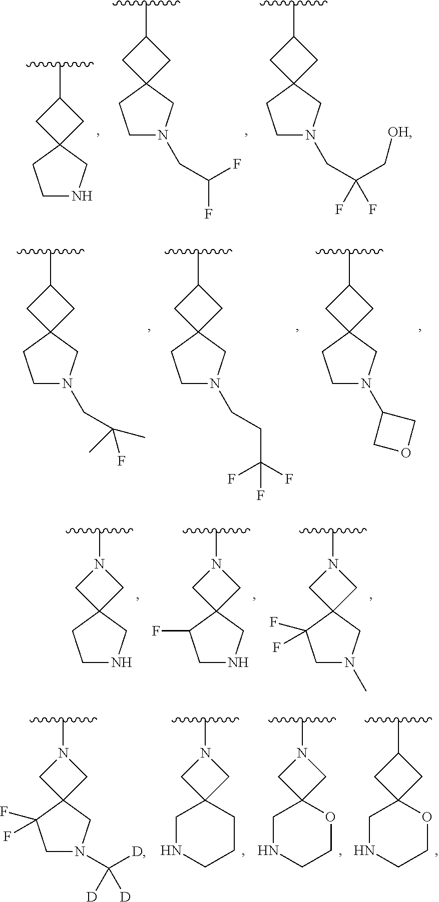

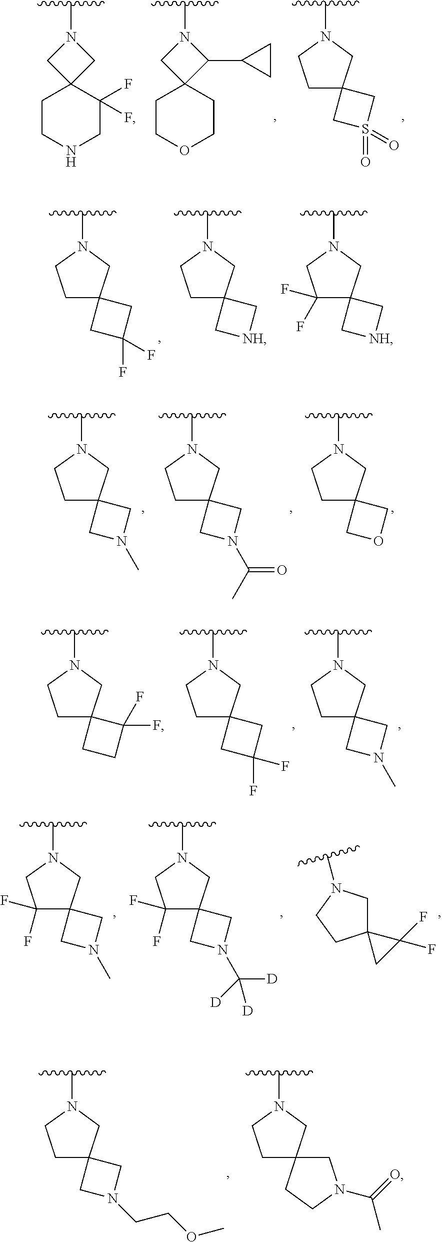

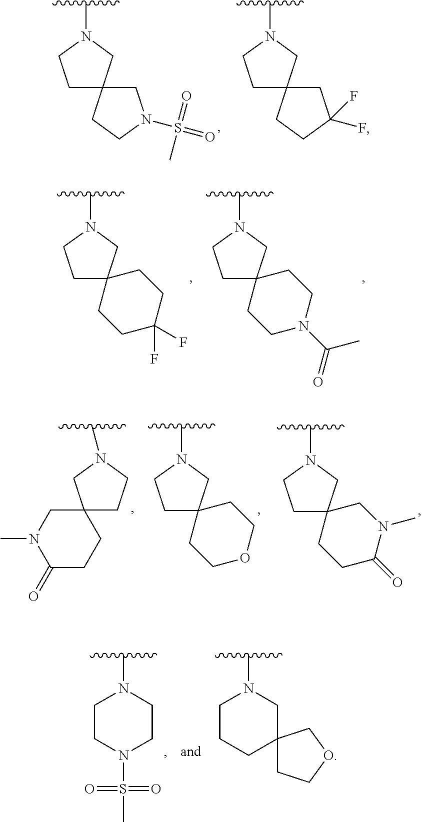

23. The compound or pharmaceutically acceptable salt of claim 1, wherein R³ is selected from hydrogen, halogen, —CN, optionally substituted C₃₋₄ carbocycle, and optionally substituted 3- to 4 membered heterocycloalkyl.

24. The compound, or pharmaceutically acceptable salt thereof, of claim 23, wherein R³ is selected from hydrogen, fluoro, —CN, cyclopropyl, cyclobutyl, optionally substituted oxetane, and optionally substituted azetidine.

25. The compound, or pharmaceutically acceptable salt thereof, of claim 1, wherein R⁴ is selected from hydrogen, halogen, —CN, optionally substituted C₁ alkyl, optionally substituted C₃₋₄ carbocycle, and optionally substituted 3- to 4 membered heterocycloalkyl.

26. The compound, or pharmaceutically acceptable salt thereof, of claim 25, wherein R⁴ is selected from hydrogen, —CN, —CHF₂, —CF₃, cyclopropyl, cyclobutyl, optionally substituted oxetane, and optionally substituted azetidine.

27. The compound or pharmaceutically acceptable salt of claim 1, wherein R⁵ is selected from hydrogen, halogen, —CN, optionally substituted C₃₋₄ carbocycle, and optionally substituted 3- to 4 membered heterocycloalkyl.

28. The compound, or pharmaceutically acceptable salt thereof, of claim 27, wherein R⁵ is selected from hydrogen, fluoro, —CN, cyclopropyl, cyclobutyl, optionally substituted oxetane, and optionally substituted azetidine.

29. The compound, or pharmaceutically acceptable salt thereof, of claim 1 wherein R⁶ is selected from hydrogen, halogen, —CN, optionally substituted C₃₋₄ carbocycle, and optionally substituted 3- to 4 membered heterocycloalkyl.

30. The compound, or pharmaceutically acceptable salt thereof, of claim 29, wherein R⁶ is selected from hydrogen, fluoro, —CN, cyclopropyl, cyclobutyl, optionally substituted oxetane, and optionally substituted azetidine.

31. The compound or pharmaceutically acceptable salt of claim 1, wherein R⁷ is hydrogen.

32. The compound, or pharmaceutically acceptable salt thereof, of claim 1 selected from the compounds in Table I.

33. A pharmaceutical composition comprising a compound or pharmaceutically acceptable salt of claim 1 and a pharmaceutically acceptable excipient.

34. A method of treating cancer, comprising administering to a subject in need thereof a compound or pharmaceutically acceptable salt of claim 1.

35. The method of claim 34 wherein the cancer is a solid tumor.

36. The method of claim 34, wherein the cancer is selected from ovarian cancer, breast cancer, colon cancer, and brain cancer.

37. A method of inhibiting a cyclin dependent kinase (CDK) in a cell with a compound or pharmaceutically acceptable salt claim 1.

38. The method of claim 37, wherein the CDK is selected from CDK 2, CDK 4, CD6, or any combination thereof.

39. The method of claim 37, wherein the CDK is selected from CDK 2/4, CDK 2/6, CDK 4/6, and CDK 2/4/6.

40. The method of claim 37, wherein the CDK is CDK 2/4/6.
